(19) United States
(12) Patent Application Publication
(10) Pub. No.: US 2020/0369626 A1
(43) Pub. Date: Nov. 26, 2020

(54) COMPOSITIONS OF SPHINGOSINE-1-PHOSPHATE RECEPTOR 2 (S1PR2) BINDING AGENTS AND USES THEREOF

(71) Applicants: Zhude Tu, St. Louis, MO (US); Zonghua Luo, St. Louis, MO (US); Hui Liu, St. Louis, MO (US); Robyn Klein, St. Louis, MO (US)

(72) Inventors: Zhude Tu, St. Louis, MO (US); Zonghua Luo, St. Louis, MO (US); Hui Liu, St. Louis, MO (US); Robyn Klein, St. Louis, MO (US)

(73) Assignee: Washington University, St. Louis, MO (US)

(21) Appl. No.: 16/863,869

(22) Filed: Apr. 30, 2020

Related U.S. Application Data
(60) Provisional application No. 62/848,810, filed on May 16, 2019.
(60) Provisional application No. 62/840,740, filed on Apr. 30, 2019.

(51) Int. Cl. C07D 239/96; G01N 33/60; C07D 401/12; C07D 211/48; C07D 205/04; C07D 207/12; A61K 51/04; C07B 59/00; C07D 405/12; C07D 295/195; C07D 211/46; C07D 491/113; C07D 413/04; C07D 217/06; C07C 313/04

(52) U.S. Cl. CPC C07D 239/96; C07C 2601/14; C07D 401/12; C07D 211/48; C07D 205/04; C07D 207/12; A61K 51/0459; C07B 59/002; A61K 51/0455; A61K 51/0446; A61K 51/0444; C07D 405/12; C07D 295/195; C07D 211/46; C07D 491/113; C07D 413/04; C07D 217/06; C07C 313/04; C07B 59/004; A61K 51/0402; C07B 2200/05; G01N 33/60

(57) ABSTRACT

Described are compounds for the binding of S1PR2, and imaging agents and pharmaceutical compositions including said compounds. Also described are methods employing the compounds, imaging agents, and pharmaceutical compositions. These methods include imaging and/or treatment of diseases associated with S1PR2.

### CROSS-REFERENCE TO RELATED APPLICATIONS

This application claims benefit of U.S. Provisional Application Ser. No. 62/840,740, filed Apr. 30, 2019 and 62/848,810, filed May 16, 2019, the entire disclosures of which are incorporated herein by reference.

### STATEMENT REGARDING FEDERALLY SPONSORED RESEARCH OR DEVELOPMENT

This invention was made with government support under DESC0008432 awarded by Department of Energy. The government has certain rights in the invention.

### FIELD OF THE INVENTION

The present disclosure generally relates to compounds for binding S1PR2, and composition and methods for using said compounds.

### BACKGROUND OF THE INVENTION

Sphingosine-1-phosphate receptor 2 (S1PR2) belongs to a class of G protein-coupled S1PRs (S1PR1-5) that regulate fundamental biological processes by binding with S1P. It plays an important role in demyelinating diseases of the central nervous system, anaphylaxis, inflammation, and cancer. An increased expression of S1PR2 has been observed in disease-susceptible regions of both female SJL mice with experimental autoimmune encephalomyelitis (EAE) and patients with multiple sclerosis (MS) compared to S1PR2 expression in male counterparts. Also, activation of sphingosine-kinase-1/S1P/S1PR2 signaling pathway contributes to the development of diabetic nephropathy, which is characterized by the progressive damage and death of glomerular podocytes resulting in exudative lesions in glomeruli, renal sclerosis, and renal fibrosis. S1PR2 expression in the kidney was increased in the experimental models of diabetes. Despite this importance of S1PR2 in biological processes and its association with multiple diseases, only a few lead compounds for S1PR2 interaction have been reported. Identifying potent and highly selective S1PR2 radiotracers is imperative for better understanding the biological role of S1PR2 and evaluating therapeutic approaches for treating MS using S1P2 inhibition strategy.

### SUMMARY OF THE INVENTION

Various aspects of the present invention relate to compounds of Formula (I), or salts thereof:

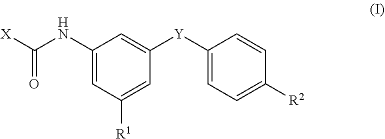

wherein

R¹ is hydrogen, substituted or unsubstituted alkyl, halo, substituted or unsubstituted alkoxy, cyano, substituted or unsubstituted nitrogen containing aryl, substituted or unsubstituted nitrogen containing cycloalkyl, or Y—R³;

R² is —CON(R⁴)₂, halo, —SO₂R⁴, substituted or unsubstituted alkoxy, or —COOH;

R³ is substituted or unsubstituted aryl, substituted or unsubstituted nitrogen containing aryl, or substituted or unsubstituted nitrogen containing cycloalkyl;

Y is —O—, —S—, —N(R⁴)—, —C(O)—, —CH(OH)—;

R⁴ is hydrogen, tert-butyloxycarbonyl (BOC), or a C1 to C6 alkyl;

X is:

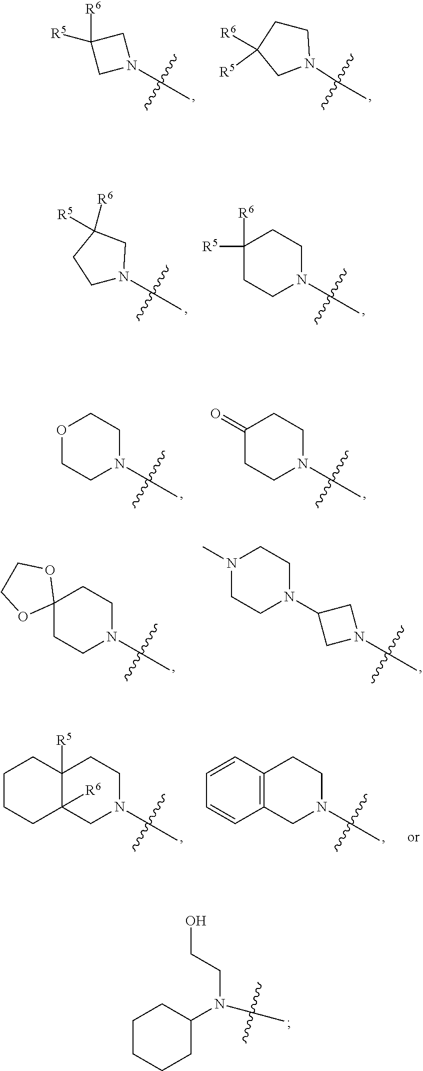

and

R⁵ and R⁶ are each independently hydrogen, hydroxyl, substituted or unsubstituted alkyl, substituted or unsubstituted alkoxyl, or substituted or unsubstituted nitrogen-containing heterocycloalkyl.

Other aspects of the present invention relate to compounds of Formula (III), or salts thereof:

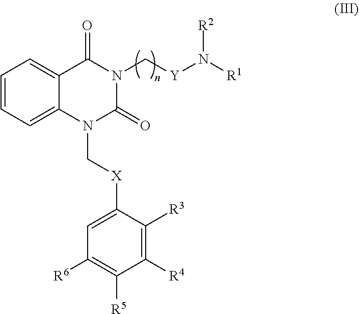

wherein

Y is —CH₂—, or —C(O)—;

R¹ and R² are each independently hydrogen, substituted or unsubstituted alkyl, substituted or unsubstituted alkoxy, substituted or unsubstituted cycloalkyl, substituted or unsubstituted heterocycloalkyl, substituted or unsubstituted aryl, substituted or unsubstituted heteroaryl, or are each alkyl and together form a 4 to 6 membered ring,

n is an integer between 1 and 10;

X is —C(O)— or —C(O)N(H)—; and

R³, R⁴, R⁵, and R⁶ are each independently hydrogen, halo, substituted or unsubstituted alkyl, or substituted or unsubstituted alkoxy.

Other aspects of the present invention relate to imaging agents including said compounds and a reporting group.

Other aspects of the present invention relate to pharmaceutical compositions including said compounds or imaging agents and at least one pharmaceutically acceptable excipient.

Further aspects of the present invention relate to methods of treating anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and/or lung fibrosis in a subject in need thereof, the methods comprising administering to the subject a therapeutically effective amount of any of said compounds.

Further aspects of the present invention relate to methods of imaging a target in a subject, the methods comprising administering said imaging agents or a composition comprising said imaging agent and detecting the imaging agent in the subject.

Further aspects of the present invention relate to methods for determining an efficacy of a therapeutic agent in a subject comprising administering any of said compound or said imaging agents to the subject, evaluating S1PR2 expression in the subject, treating the subject for a disease associated with increased S1PR2 expression, and reevaluating the S1PR2 expression in the subject.

Further aspects of the present invention relate to methods for evaluating a potential therapeutic, the methods comprising determining an S1PR2 binding potency of said compounds.

Further aspects of the present invention relate to methods of diagnosing or monitoring an S1PR2 associated disease, disorder or condition in a mammal comprising administering said imagining agent to the mammal and detecting the imaging agent, wherein the S1PR2 associated disease disorder or condition is selected from the group consisting of anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and lung fibrosis.

Further aspects of the present invention relate to methods of quantifying S1PR2 expression in a mammalian brain or central nervous system comprising administering said imaging agent to the mammal and detecting the imaging agent in the mammal.

### BRIEF DESCRIPTION OF THE DRAWINGS

FIG. 1 shows representative competitive binding curves of JTE-013 and 2c toward S1PR2.

FIG. 2 shows representative competitive binding curves of compounds JTE-013, 28b, 28c, 28d, 28e, and 28g toward S1PR2.

FIG. 3 shows representative saturation binding curves for [¹²⁵I]34 (0.2-20 nM). Total, non-specific, and specific bindings to the human recombinant S1PR2 membrane were plotted in the corresponding Scatchard plot.

FIG. 4 shows representative curves for compounds binding S1PR2 in a radioligand competitive binding assay using [¹²⁵I]34 as the radioligand.

FIG. 5 shows in vitro autoradiography of [¹²⁵I]34 on kidney sections from control and chronic diabetic rats (STZ). A: Blood glucose (mg/dL) from control and chronic diabetic rats; B: Representative autoradiography images of control and diabetic kidney sections with and without blocking agent JTE-013 (10 μM). C: Quantification of average autoradiography signal (PSL/mm²) from each group of kidney sections incubated with [¹²⁵I]34 (STZ n=5, control n=4, p=0.009).

FIG. 6 shows immunostaining of S1PR2 in the kidney sections of control and STZ treated rats (200×).

FIG. 7 shows ELISA analysis of sphingosine-1-phosphate receptor 2 in the kidney tissue homogenates of control and chronic diabetic (STZ) rats.

FIG. 8 shows a biodistribution study of [¹¹C]2c (TZ59-104).

FIG. 9 shows clinical score over time in multiple sclerosis model mice post immunization with 2c (TZ59-104).

### DETAILED DESCRIPTION OF THE INVENTION

Various aspects of the present invention are directed to compounds having potency and selectivity for S1PR2. Particular aspects of the invention relate to compositions and methods employing said compounds, such as for imaging and treatment of diseases associated with S1PR2. Unlike certain existing compounds that must be phosphorylated in vivo to bind to S1PR and are quickly metabolized, various compounds described herein can bind S1PR2 receptors directly, with high affinity and specificity. For example, these compounds can be incorporated into imaging agents, or into compositions used to diagnose, treat, or assess therapeutic efficacy of drugs for diseases, disorders, or conditions associated with increased S1PR2 expression. Such diseases, disorders, or conditions associated with increased S1PR2 expression can include anaphylaxis, cancer, demyelination in CNS disease, diabetes, diabetic pulmonary fibrosis, diseases of the central nervous system (CNS), inflammation, inflammation disease, inflammatory response in MS, multiple sclerosis (MS), liver fibrosis, or lung fibrosis.

In some aspects, the compounds described herein can be labeled with one or more positron emission tomography (PET) isotopes to serve as a PET radiotracers for measuring S1PR2 expression levels to assess inflammatory response in multiple sclerosis (MS) and other inflammation diseases.

### Compounds

In various embodiments, compounds of the present invention include those of Formula (I), or a salt thereof:

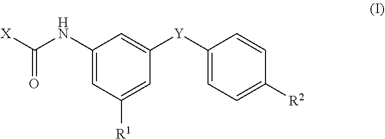

wherein

R¹ is hydrogen, substituted or unsubstituted alkyl, halo, substituted or unsubstituted alkoxy, cyano, substituted or unsubstituted nitrogen containing aryl, substituted or unsubstituted nitrogen containing cycloalkyl, or Y—R³;

R² is —CON(R⁴)₂, halo, —SO₂R⁴, substituted or unsubstituted alkoxy, or —COOH;

R³ is substituted or unsubstituted aryl, substituted or unsubstituted nitrogen containing aryl, or substituted or unsubstituted nitrogen containing cycloalkyl;

Y is —O—, —S—, —N(R⁴)—, —C(O)—, —CH(OH)—;

R⁴ is hydrogen, tert-butyloxycarbonyl (BOC), or a C1 to C6 alkyl;

X is:

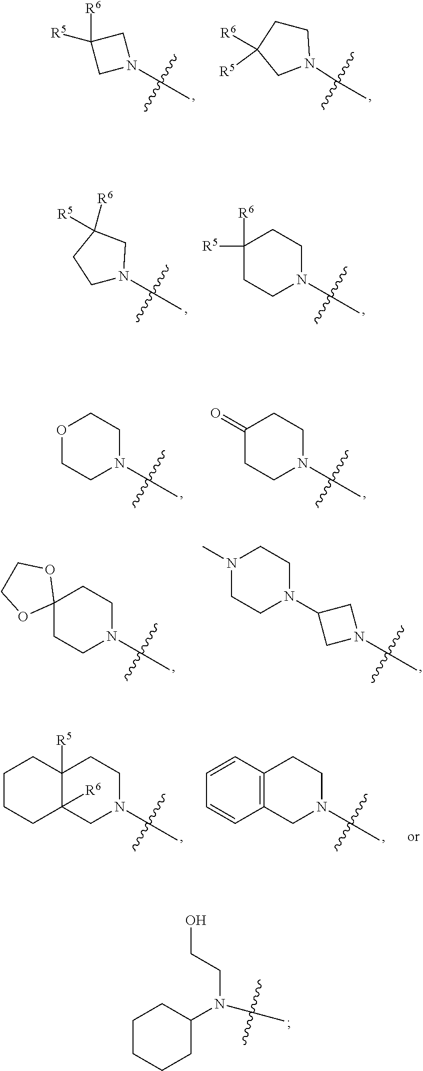

and

R⁵ and R⁶ are each independently hydrogen, hydroxyl, substituted or unsubstituted alkyl, substituted or unsubstituted alkoxyl, or substituted or unsubstituted nitrogen-containing heterocycloalkyl.

In various embodiments, R¹ of Formula (I) is Y—R³, halo, C1 to C3 alkyl, haloalkyl, haloalkoxy, cyano,

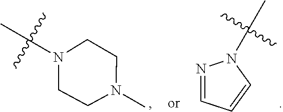

In some embodiments, R¹ is, —CF₃, F, methyl, —OCH2CH2CH2F, —OCH2CH2F, —CN,

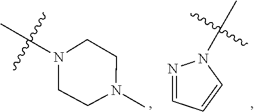

### or Y—R³.

In various embodiments R³ of Formula (I) is selected from the group consisting of:

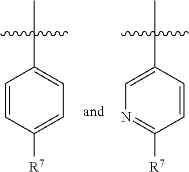

wherein R⁷ is halo, —SO₂R⁴,

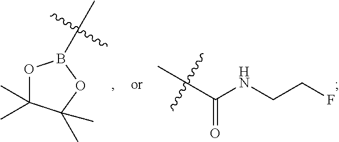

and R⁴ is hydrogen or a C1-C6 alkyl. In some embodiments, R⁷ is selected from the group consisting of: F, SO₂CH₃,

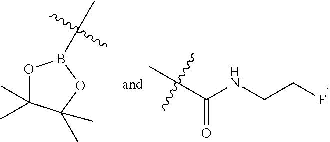

In various embodiments, R³ of Formula (I) is selected from the group consisting of

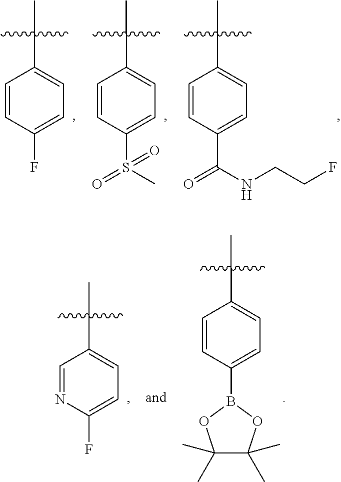

In various embodiments, Y of Formula (I) is —O—, —S—, —C(O)—, —C(OH)—, —N(H)— or —N(BOC)—. In some embodiments, Y is —O— or —S—.

In various embodiments, R² of Formula (I) is selected from the group consisting of —CON(R⁴)₂, F, —SO₂CH₃, alkoxy, haloalkoxy, or —COOH. In some embodiments, R² is selected from the group consisting of —C(O)NH₂, —C(O)NHCH₃, —C(O)N(CH₃)₂, F, —SO₂CH₃, —OCH₃, and —OCH₂CH₂F.

In various embodiments, R⁵ and R⁶ of Formula (I) are each independently hydrogen, substituted or unsubstituted C1 to C6 alkyl, hydroxyl, or alkoxy. In some embodiments, R⁵ and R⁶ are each independently hydrogen, —OH, 2-(ethyl)butyl, isobutyl, isopropyl, methyl, ethyl, propyl, butyl, 4-methylpentan-2-onyl, or C1 to C6 hydroxyalkyl.

In various embodiments, X of Formula (I) is selected from the group consisting of:

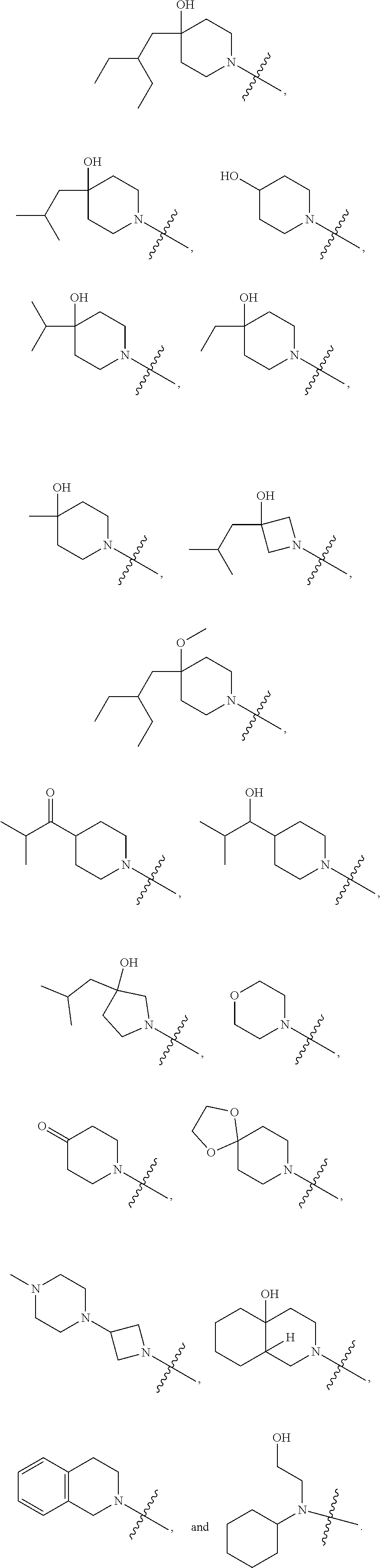

In various embodiments, the invention is directed to a compound of Formula (II) or a salt thereof:

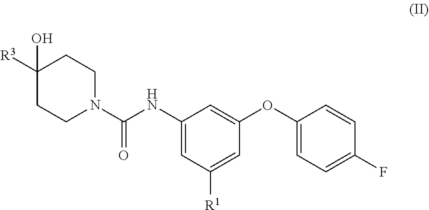

wherein

R¹ is —CF₃,

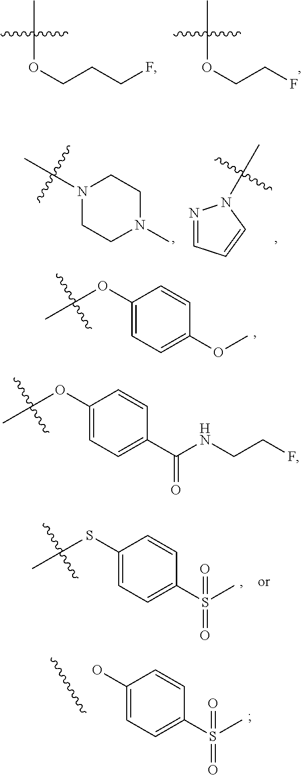

and

R³ is 2-(ethyl)butyl, isobutyl, isopropyl, methyl, or ethyl.

In various embodiments, the compound of Formula (I) is selected from the group consisting of:

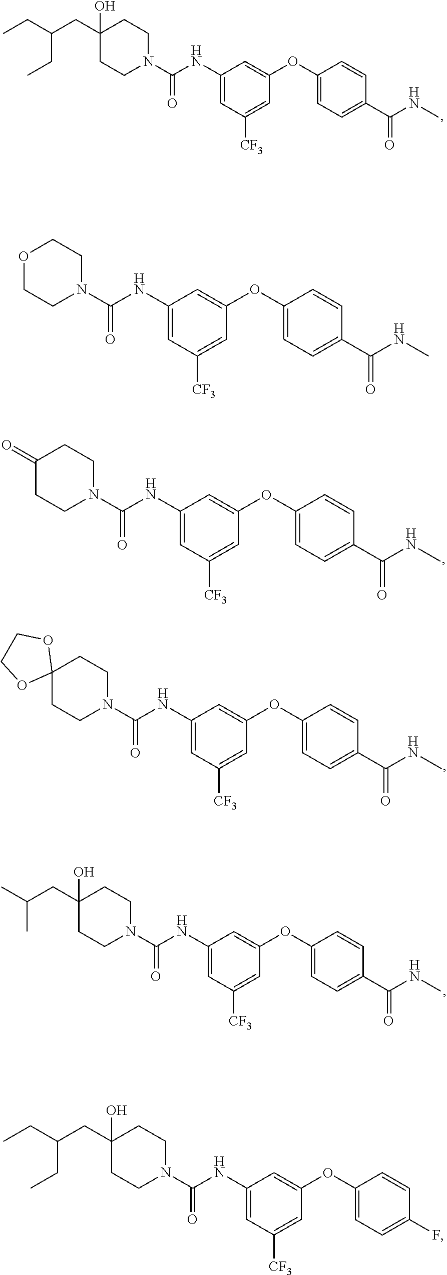

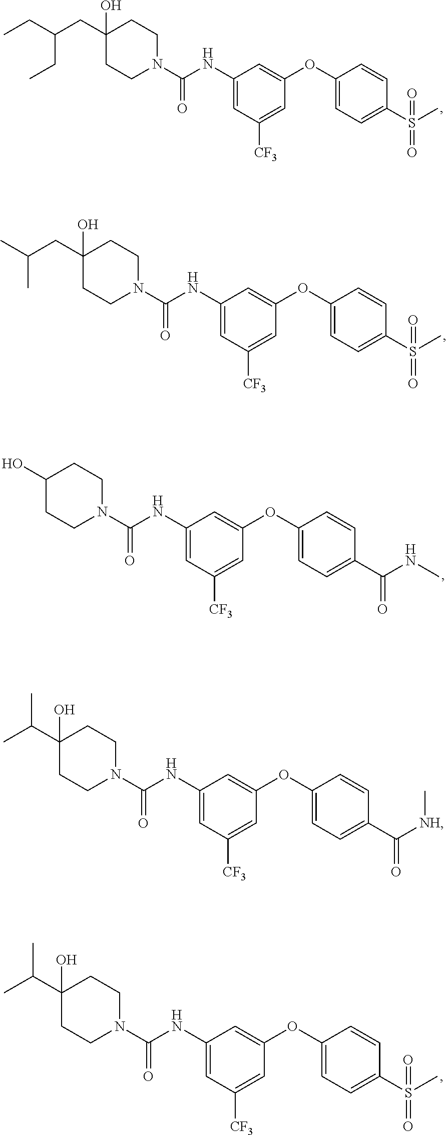

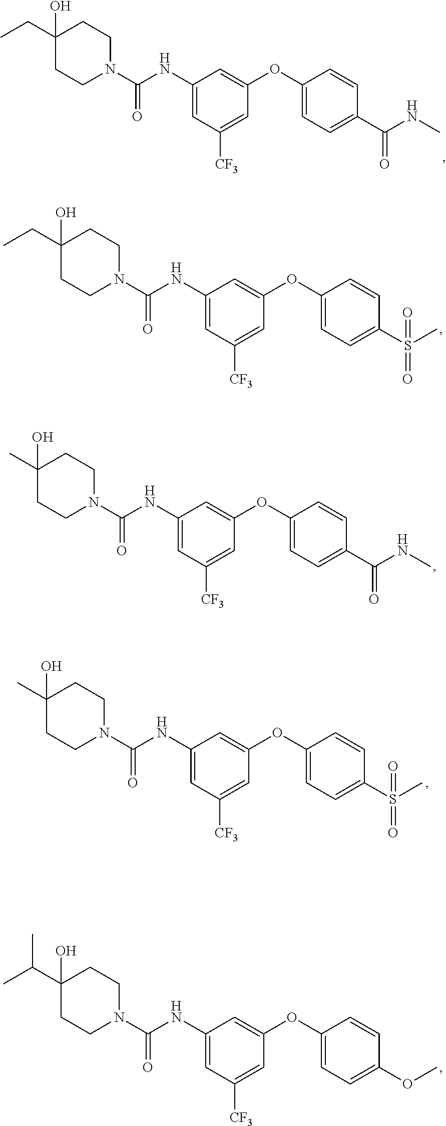

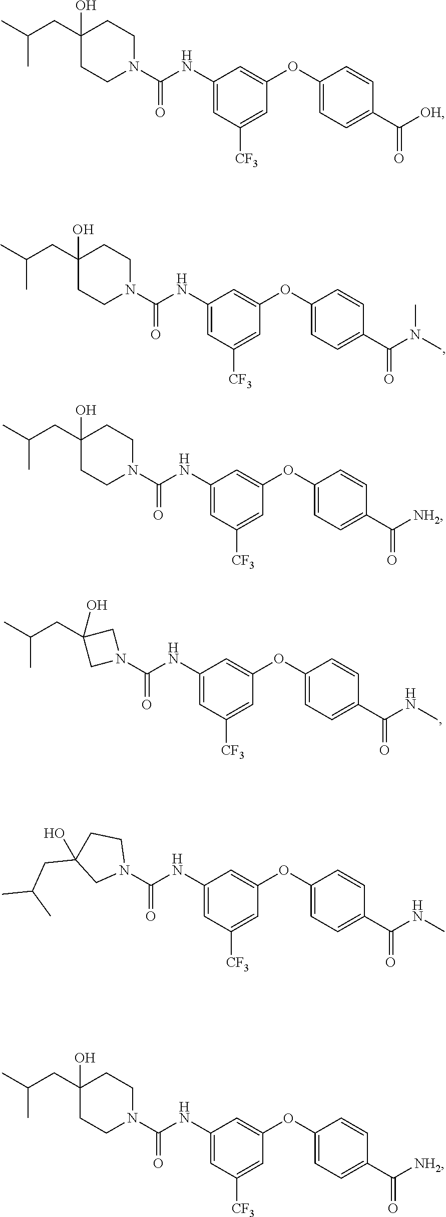

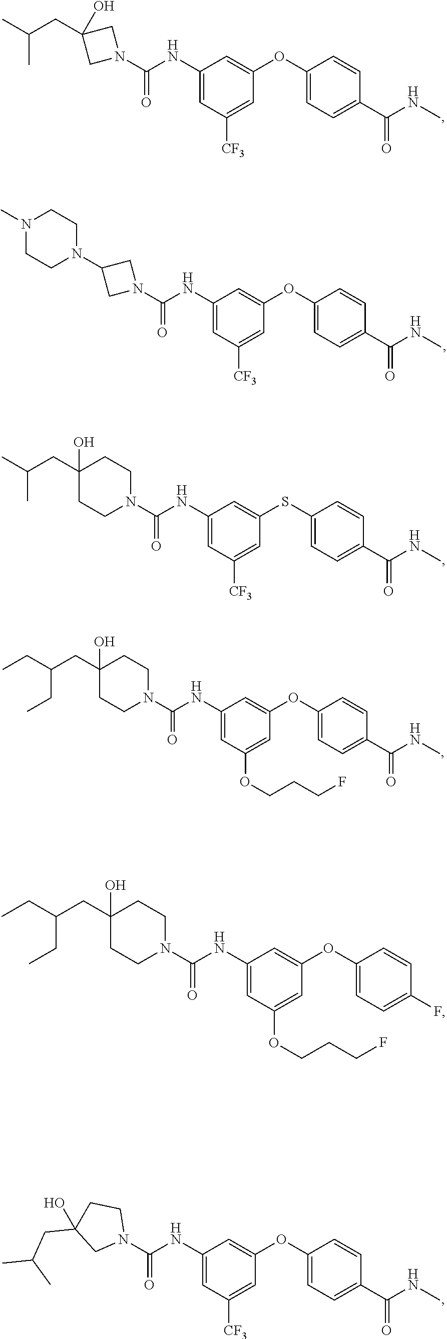

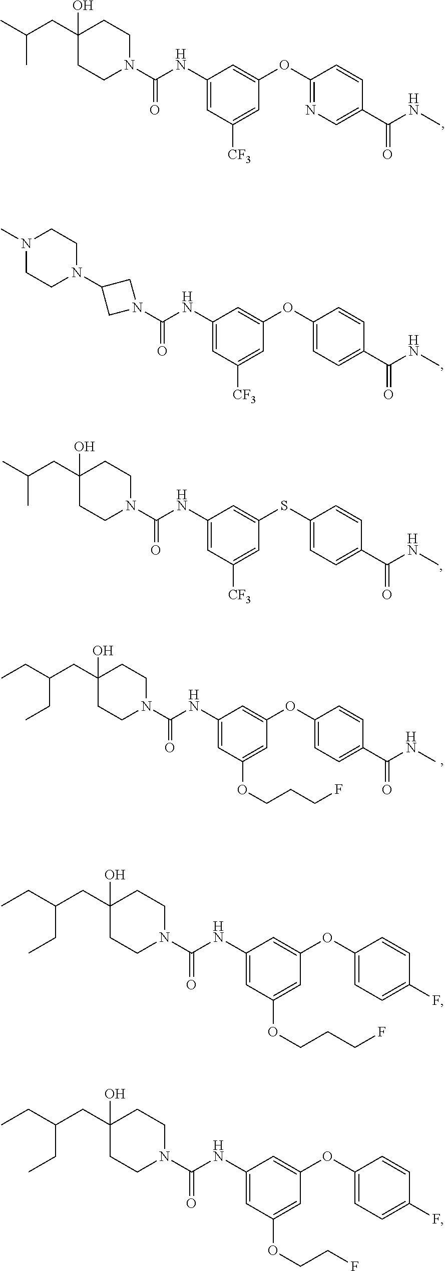

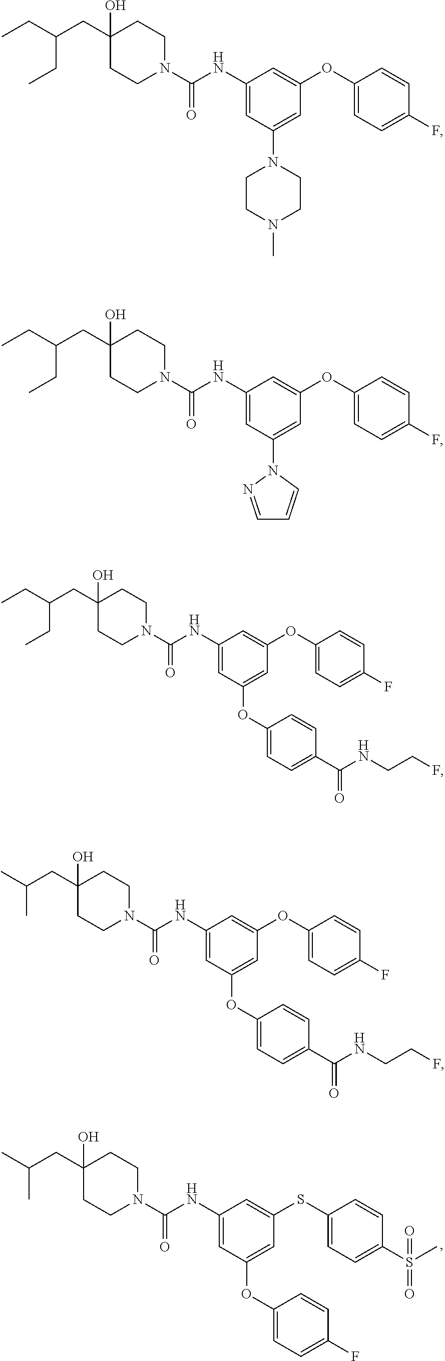 [SMILES: CCC(CC)CC1(O)CCN(C(=O)Nc2cc(Oc3ccc(F)cc3)cc(N3CCN(C)CC3)c2)CC1 ; CCC(CC)CC1(O)CCN(C(=O)Nc2cc(Oc3ccc(F)cc3)cc(-n3cccn3)c2)CC1 ; CCC(CC)CC1(O)CCN(C(=O)Nc2cc(Oc3ccc(F)cc3)cc(Oc3ccc(C(=O)NCCF)cc3)c2)CC1 ; CC(C)CC1(O)CCN(C(=O)Nc2cc(Oc3ccc(F)cc3)cc(Oc3ccc(C(=O)NCCF)cc3)c2)CC1 ; CC(C)CC1(O)CCN(C(=O)Nc2cc(Oc3ccc(F)cc3)cc(Sc3ccc(S(C)(=O)=O)cc3)c2)CC1]

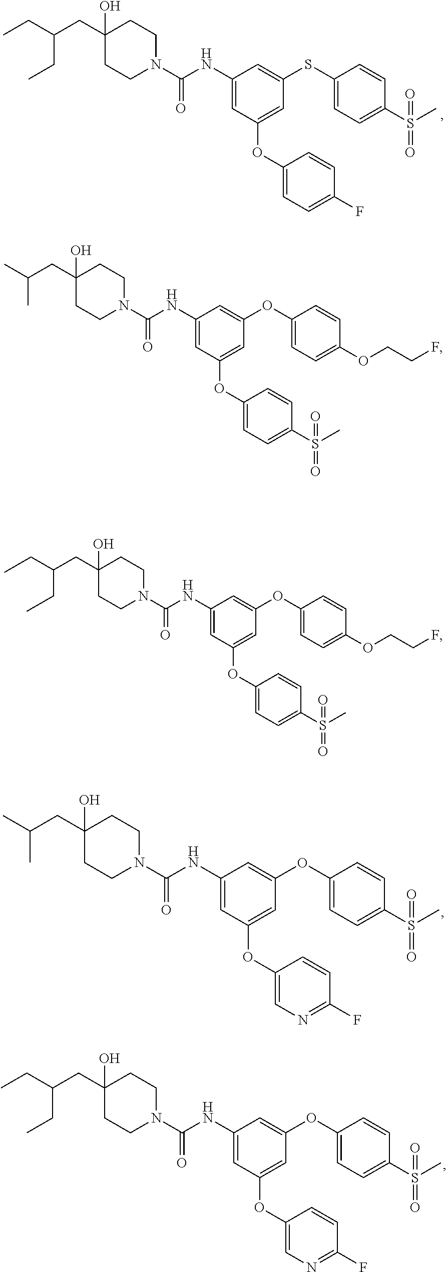

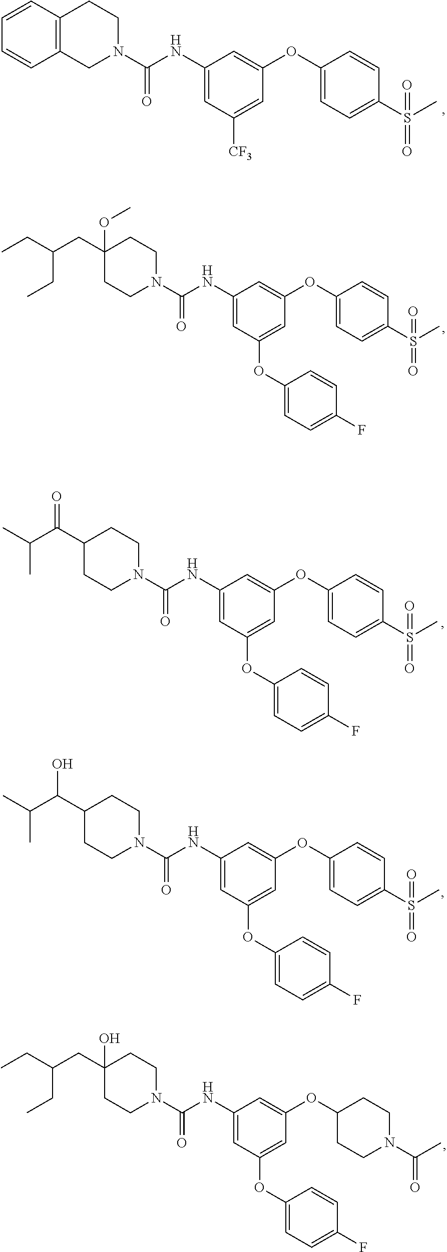

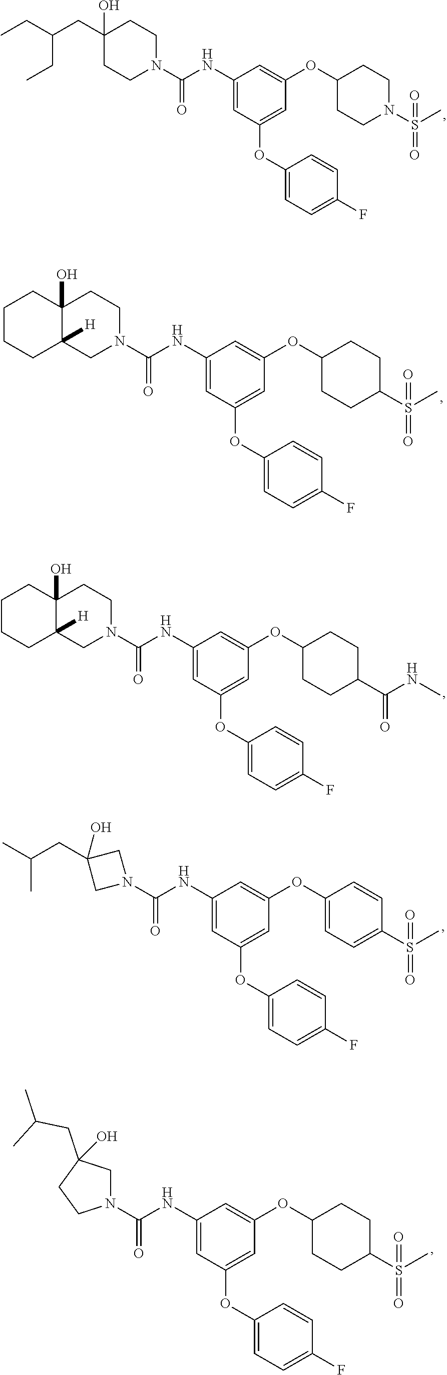

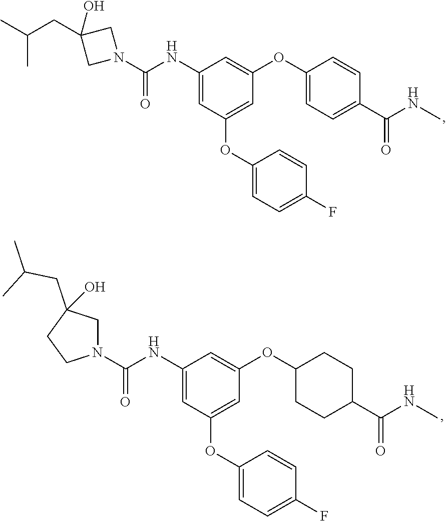

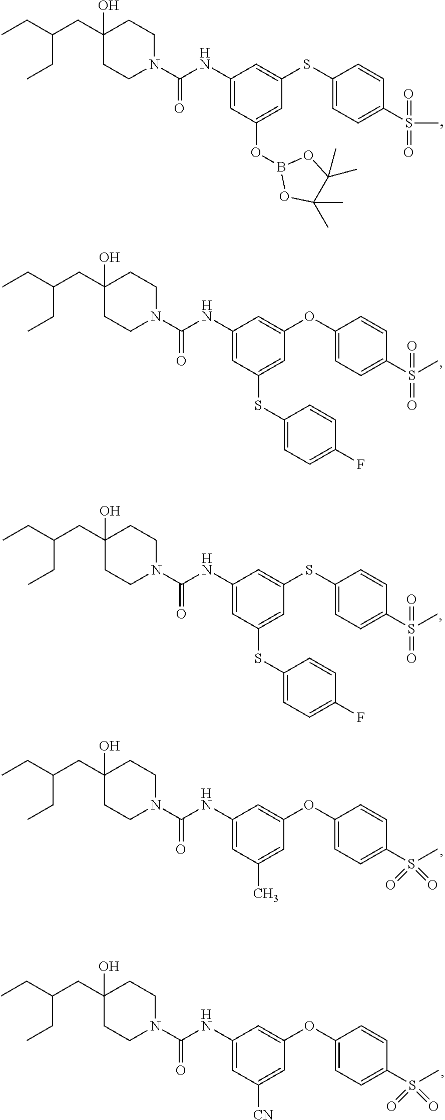

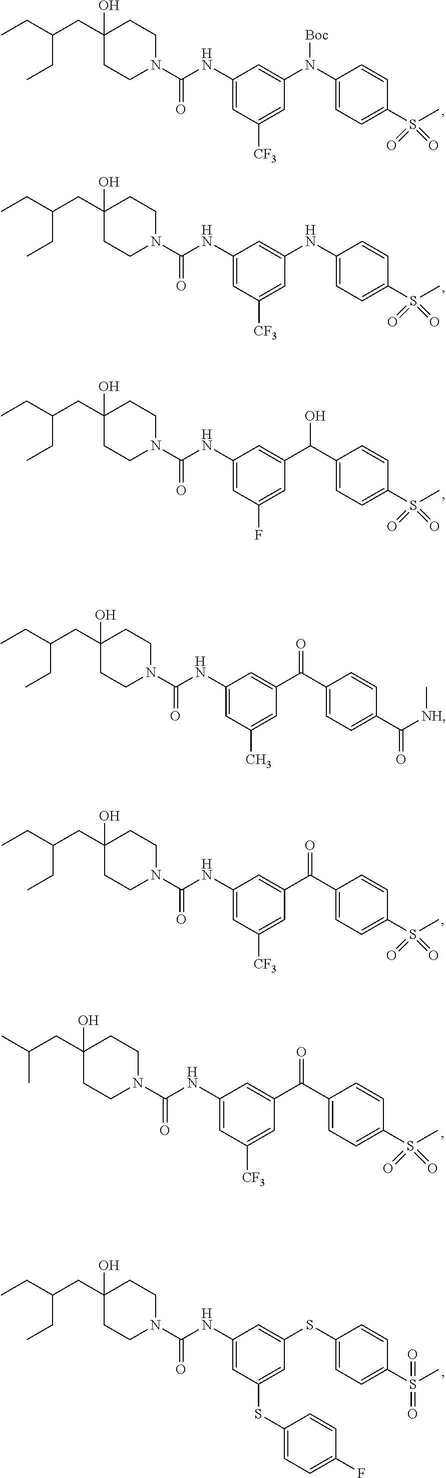

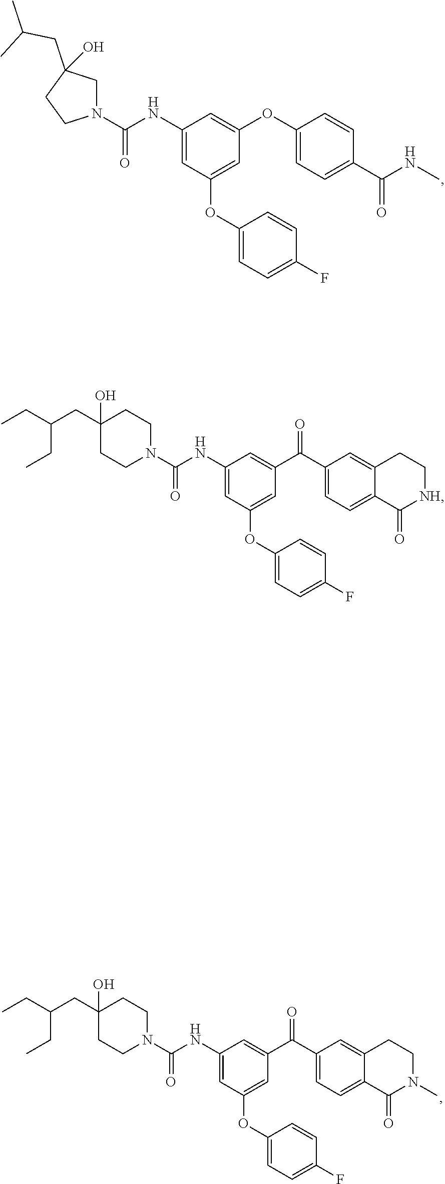

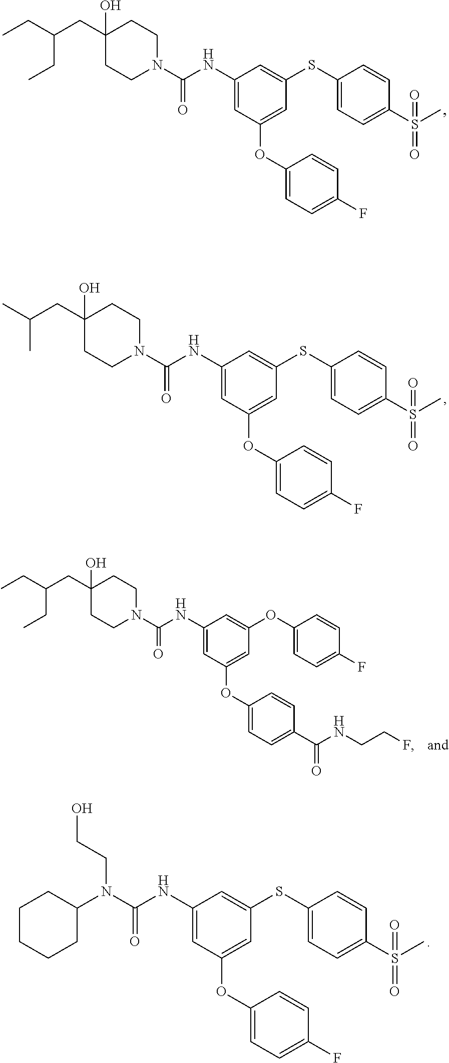

In various embodiments, the compound of Formula (I) is selected from the group consisting of:

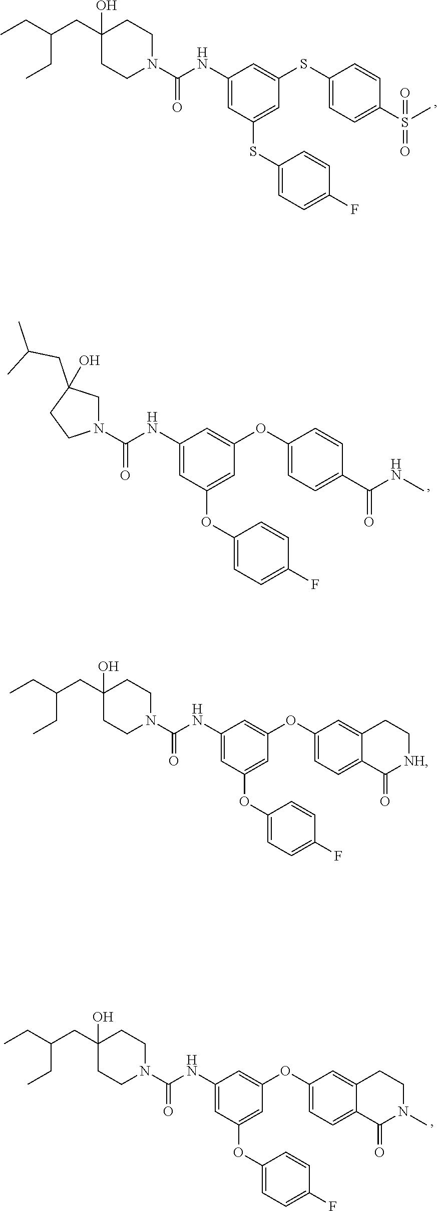

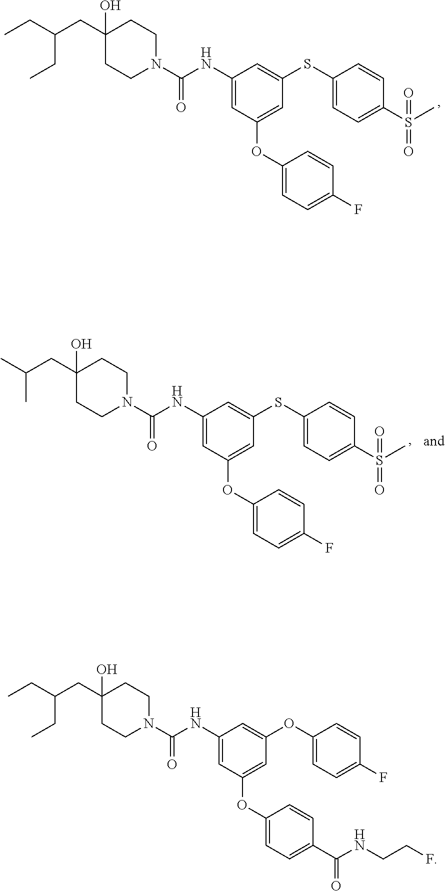

In various embodiments, the invention is directed to a compound of Formula (III), or a salt thereof:

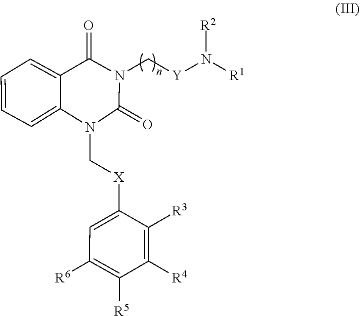

wherein

Y is —CH₂—, or —C(O)—;

R¹ and R² are each independently hydrogen, substituted or unsubstituted alkyl, substituted or unsubstituted alkoxy, substituted or unsubstituted cycloalkyl, substituted or unsubstituted heterocycloalkyl, substituted or unsubstituted aryl, substituted or unsubstituted heteroaryl, or are each alkyl and together form a 4 to 6 membered ring;

n is an integer between 1 and 10;

X is —C(O)— or —C(O)N(H)—; and

R³, R⁴, R⁵, and R⁶ are each independently hydrogen, halo, substituted or unsubstituted alkyl, or substituted or unsubstituted alkoxy.

In various embodiments, Y of Formula (III) is —C(O)—.

In various embodiments, n of Formula (III) is an integer between 1 and 5 or an integer between 1 and 3.

In various embodiments, X of Formula (III) is —C(O)N(H)—.

In various embodiments, at least one of R¹ and R² of Formula (III) is hydrogen or methyl.

In various embodiments, (a) at least one of R¹ and R² of Formula (III) is selected from the group consisting of unsubstituted C1 to C6 alkyl, hydroxyalkyl,

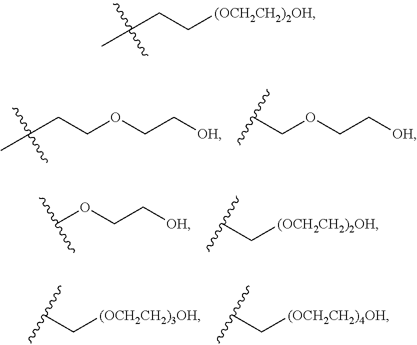

alkoxy, cyclopentyl, substituted or unsubstituted phenyl, or substituted or unsubstituted nitrogen containing ring or (b) R¹ and R² together form a 5-membered ring. In some embodiments, wherein at least one of R¹ and R² is methyl, cyclopentyl,

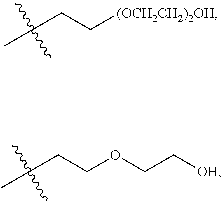

hydroxypropyl, monosubstituted phenyl, or disubstituted nitrogen containing ring. In some embodiments, at least one of R¹ and R² is methyl, cyclopentyl,

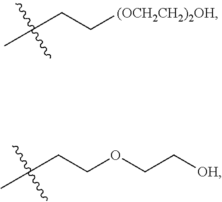

hydroxypropyl, methoxyphenyl, ethoxyphenyl, hydroxyphenyl, iodophenyl, 3-fluoropropoxyphenyl, or 2,6-dichloro-4-pyridinyl.

In various embodiments, at least one of R³, R⁴, R⁵, and R⁶ of Formula (III) is halo or alkoxy. In some embodiments for Formula (III), at least two of R³, R⁴, R⁵, and R⁶ are halo or alkoxy.

In various embodiments, R³, R⁴, R⁵ and R⁶ of Formula (III) are each independently methoxy, ethoxy, hydrogen or halo.

In various embodiments, the compound of Formula (III) is selected from the group consisting of:

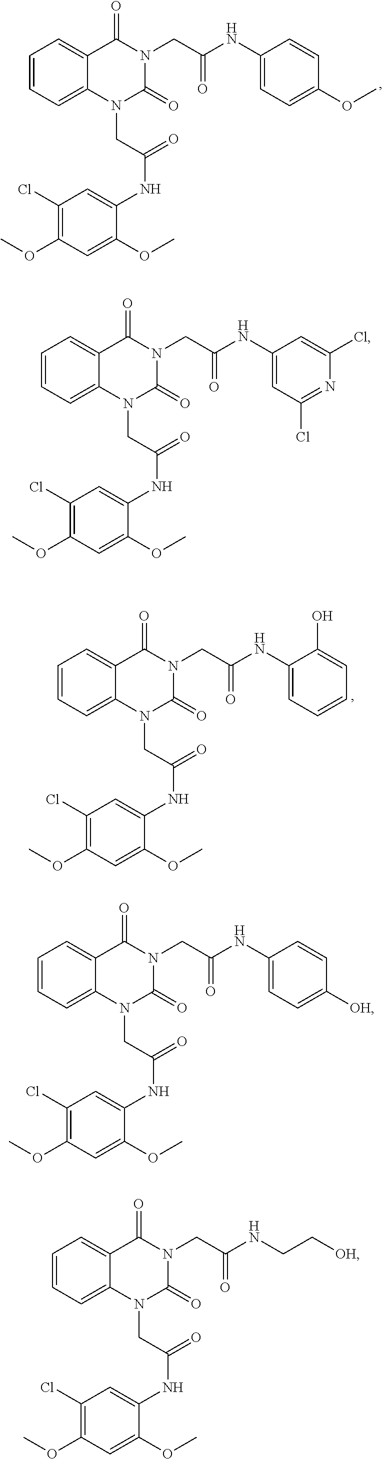

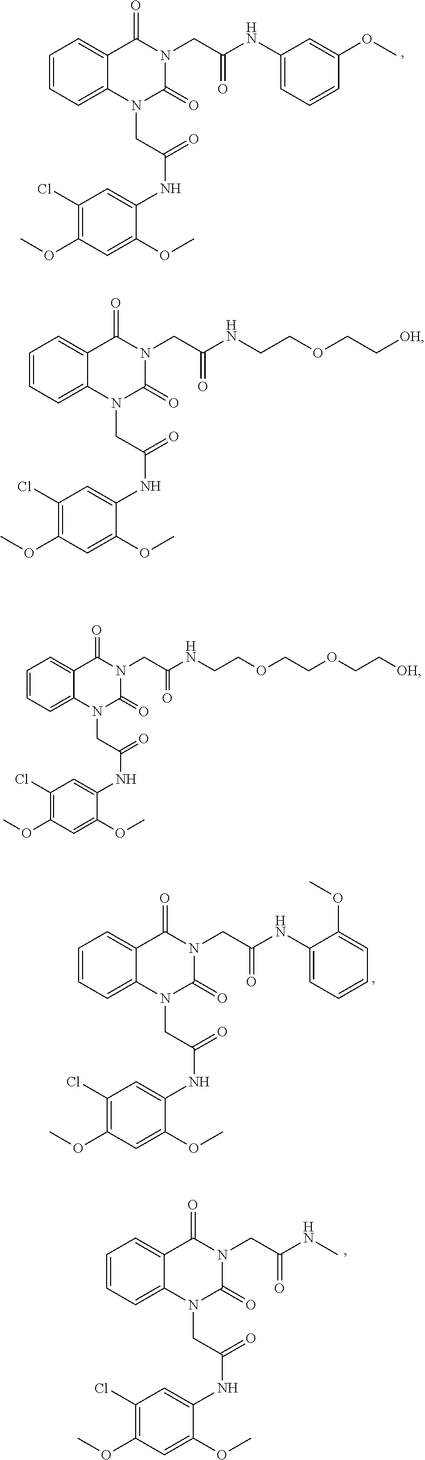

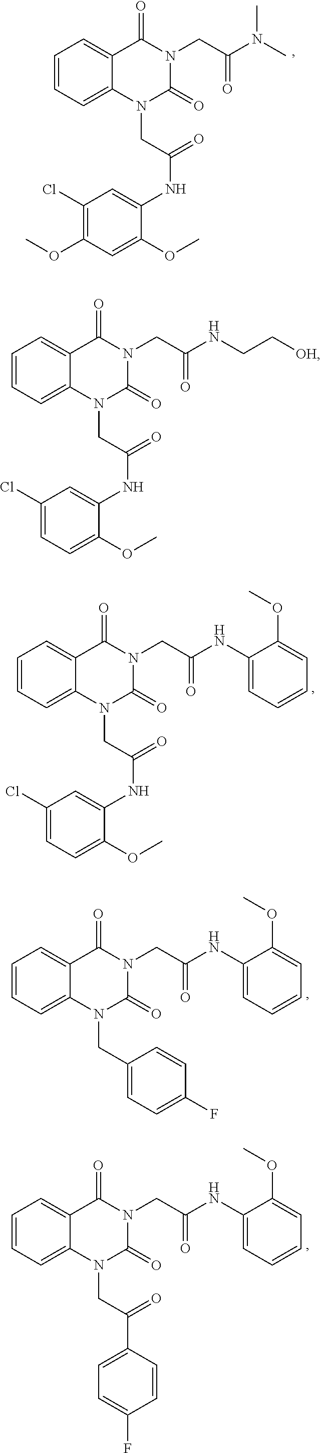

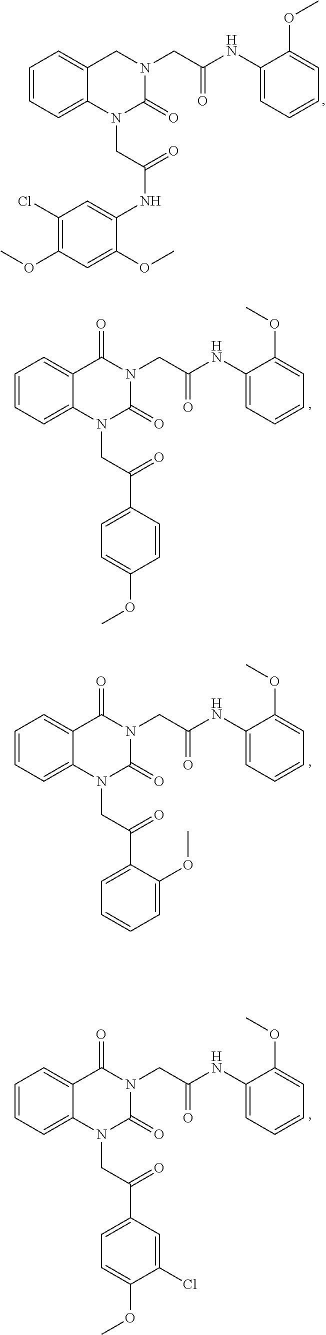

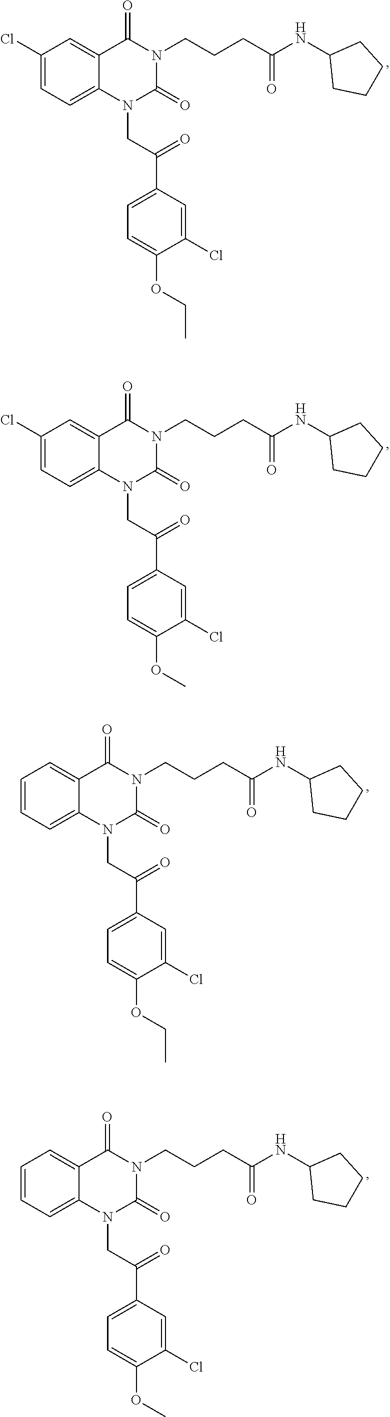 [SMILES: CCOc1ccc(C(=O)Cn2c(=O)n(CCCC(=O)NC3CCCC3)c(=O)c3cc(Cl)ccc32)cc1Cl ; COc1ccc(C(=O)Cn2c(=O)n(CCCC(=O)NC3CCCC3)c(=O)c3cc(Cl)ccc32)cc1Cl ; CCOc1ccc(C(=O)Cn2c(=O)n(CCCC(=O)NC3CCCC3)c(=O)c3ccccc32)cc1Cl ; COc1ccc(C(=O)Cn2c(=O)n(CCCC(=O)NC3CCCC3)c(=O)c3ccccc32)cc1Cl]

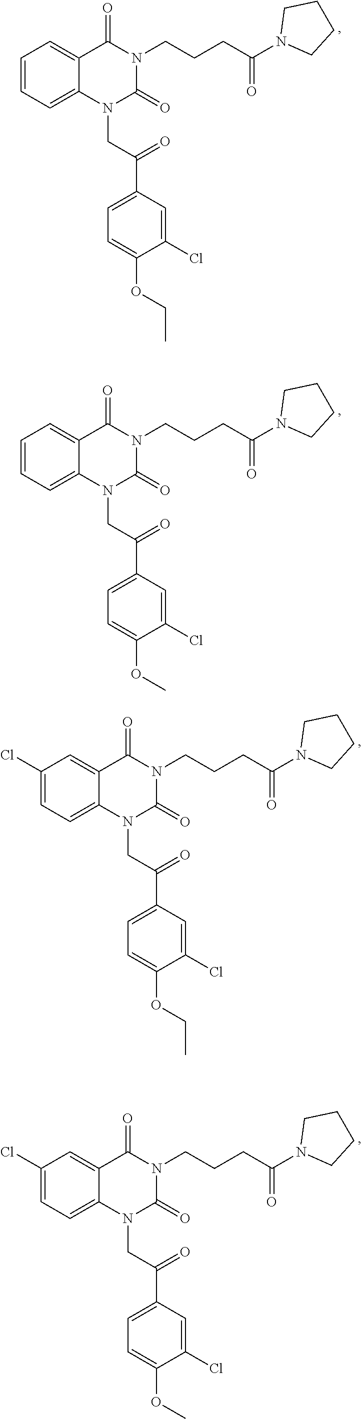 [SMILES: CCOc1ccc(C(=O)Cn2c(=O)n(CCCC(=O)N3CCCC3)c(=O)c3ccccc32)cc1Cl ; COc1ccc(C(=O)Cn2c(=O)n(CCCC(=O)N3CCCC3)c(=O)c3ccccc32)cc1Cl ; CCOc1ccc(C(=O)Cn2c(=O)n(CCCC(=O)N3CCCC3)c(=O)c3cc(Cl)ccc32)cc1Cl ; COc1ccc(C(=O)Cn2c(=O)n(CCCC(=O)N3CCCC3)c(=O)c3cc(Cl)ccc32)cc1Cl]

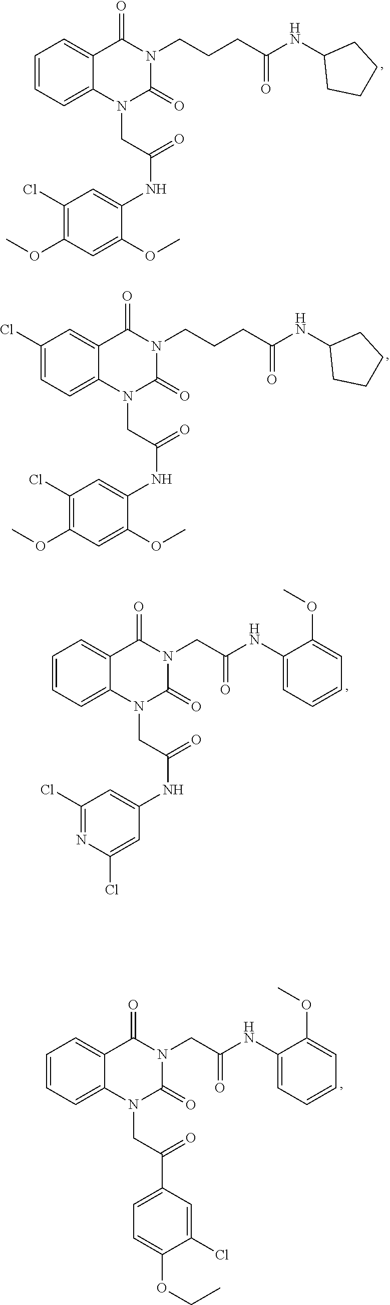

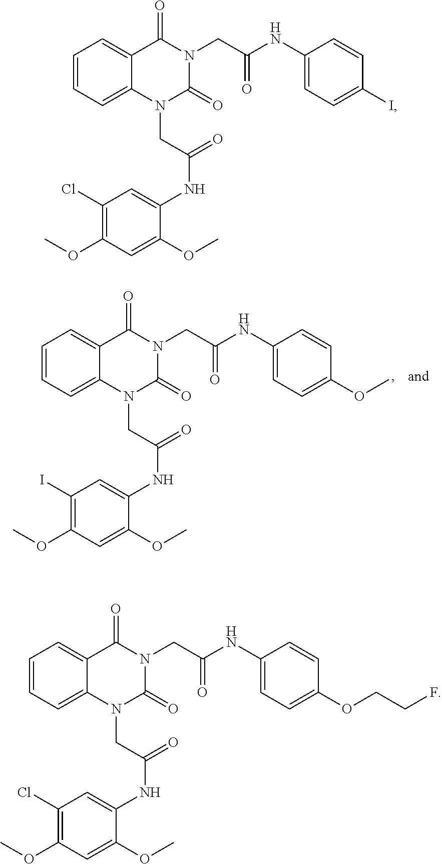

In various embodiments, the compound of Formula (III) is selected from the group consisting of:

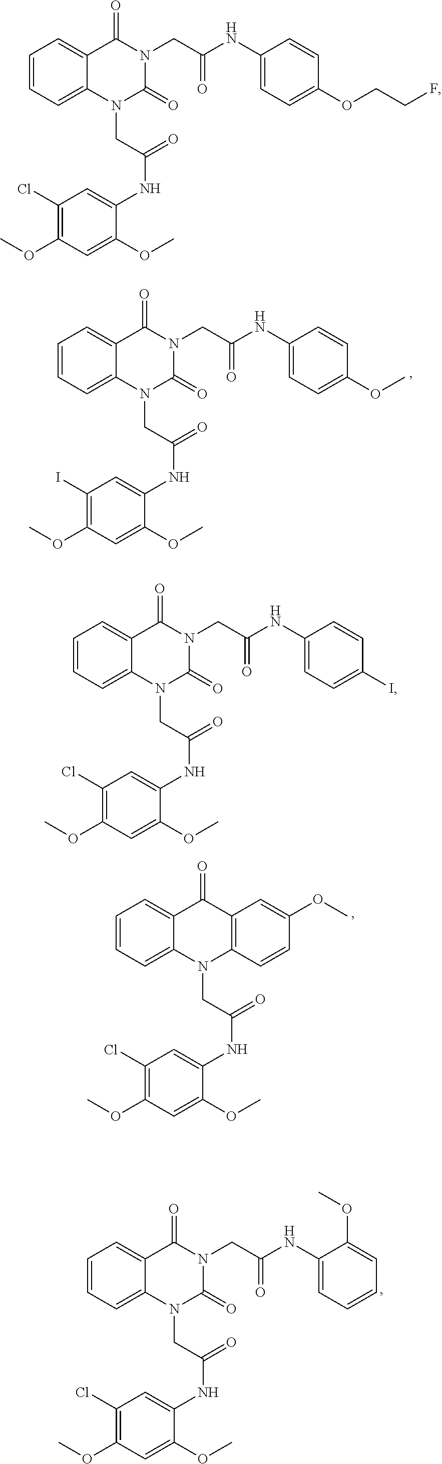

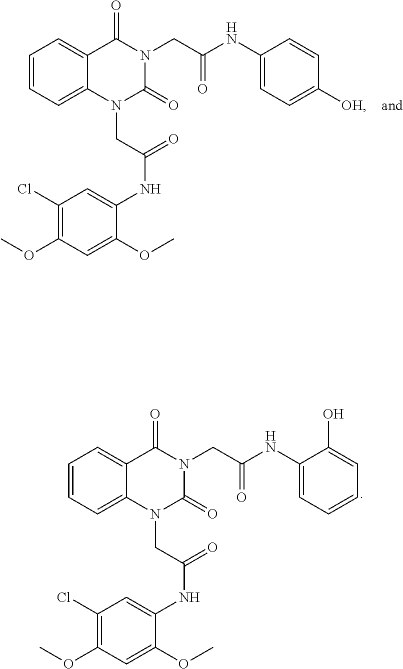 [SMILES: COc1cc(OC)c(NC(=O)Cn2c(=O)n(CC(=O)Nc3ccc(O)cc3)c(=O)c3ccccc32)cc1Cl ; "and" ; COc1cc(OC)c(NC(=O)Cn2c(=O)n(CC(=O)Nc3ccccc3O)c(=O)c3ccccc32)cc1Cl]

In various embodiments, the invention is directed to a compound selected from the group consisting of:

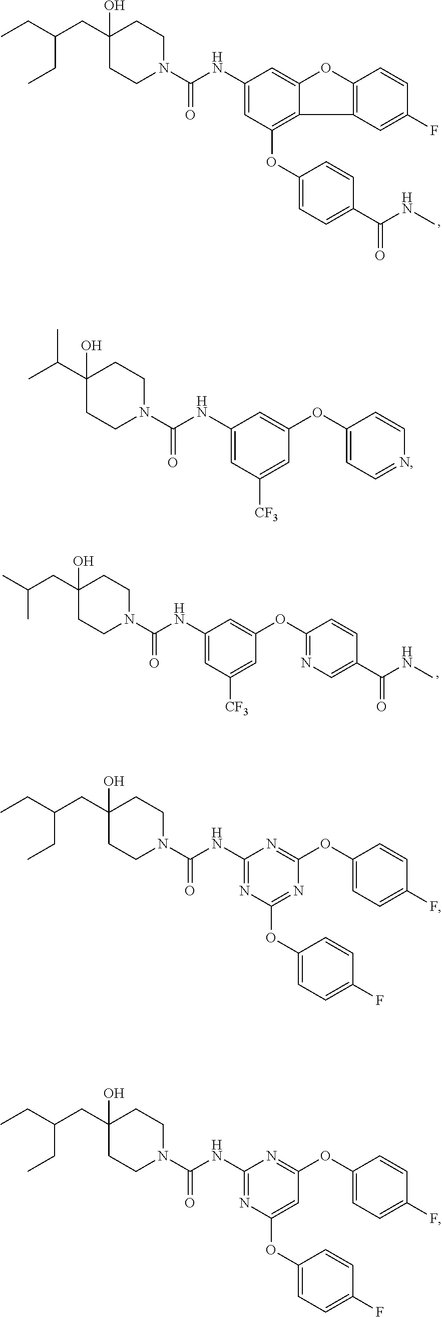

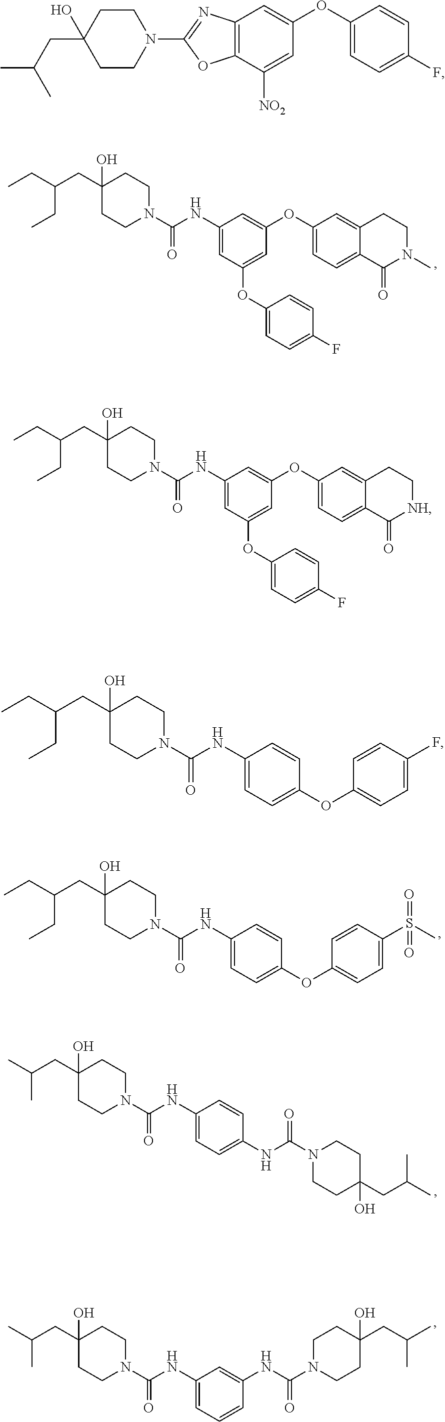

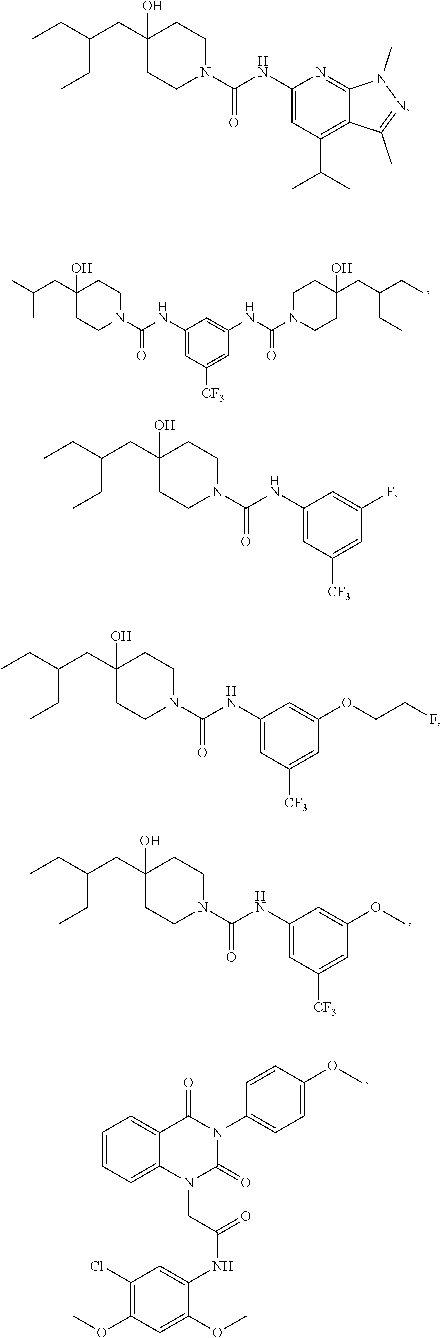

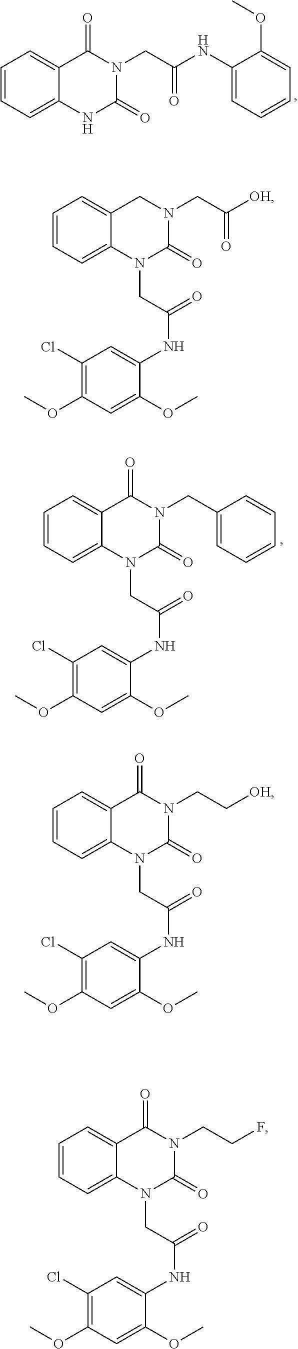 [SMILES: COc1ccccc1NC(=O)Cn1c(=O)[nH]c2ccccc2c1=O ; COc1cc(OC)c(NC(=O)CN2C(=O)N(CC(=O)O)Cc3ccccc32)cc1Cl ; COc1cc(OC)c(NC(=O)Cn2c(=O)n(Cc3ccccc3)c(=O)c3ccccc32)cc1Cl ; COc1cc(OC)c(NC(=O)Cn2c(=O)n(CCO)c(=O)c3ccccc32)cc1Cl ; COc1cc(OC)c(NC(=O)Cn2c(=O)n(CCF)c(=O)c3ccccc32)cc1Cl]

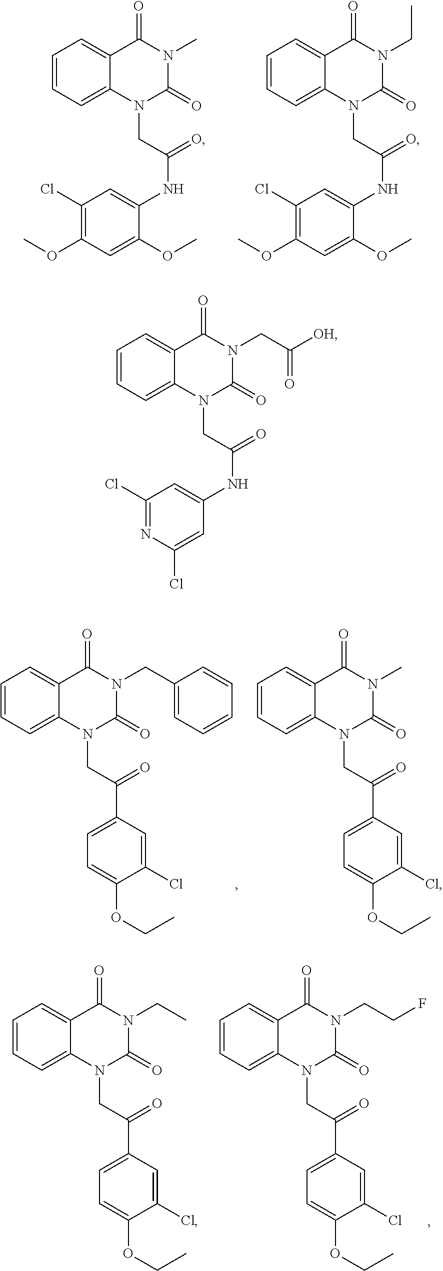

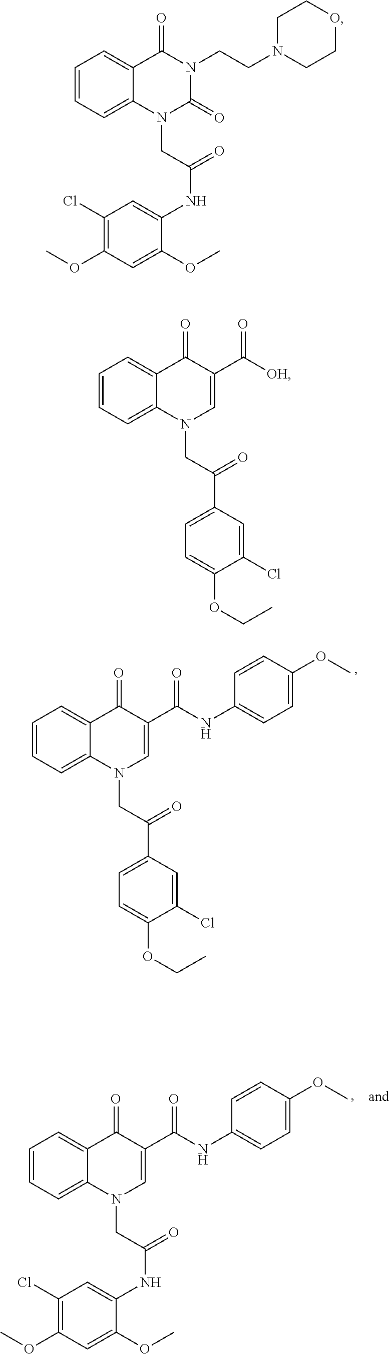

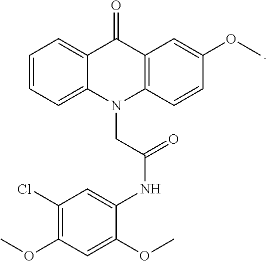

In various embodiments, the compound binds, modulates, or inhibits an S1PR2 receptor.

In various embodiments, the invention is directed towards an S1PR2 binding agent of formula

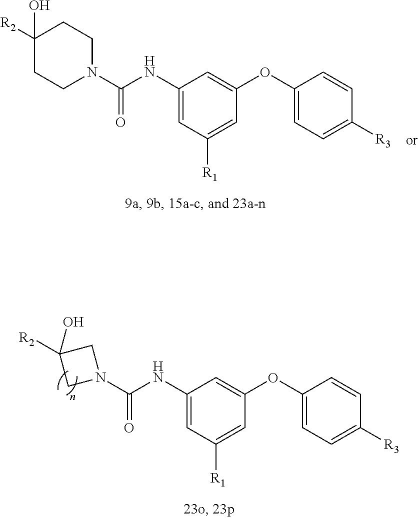

wherein

R₁ is selected from: OCH₂CH₂F, O(CH₂)₃F,

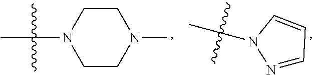

### or CF₃.

R₂ is selected from 2-(ethyl)butyl, methyl, ethyl, isopropyl, or isobutyl; or

R₃ is selected from F, OCH₃, CONH₂, CONHCH₃, CON(CH₃)₂, COOCH₃, SO₂CH₃, or CONHCH₃.

In various embodiments, the invention is directed towards an S1PR2 binding agent of formula

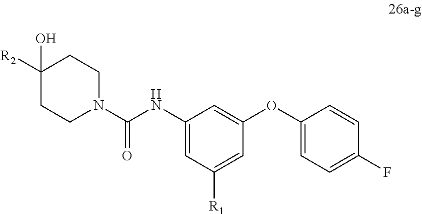

wherein

R₁ is selected from

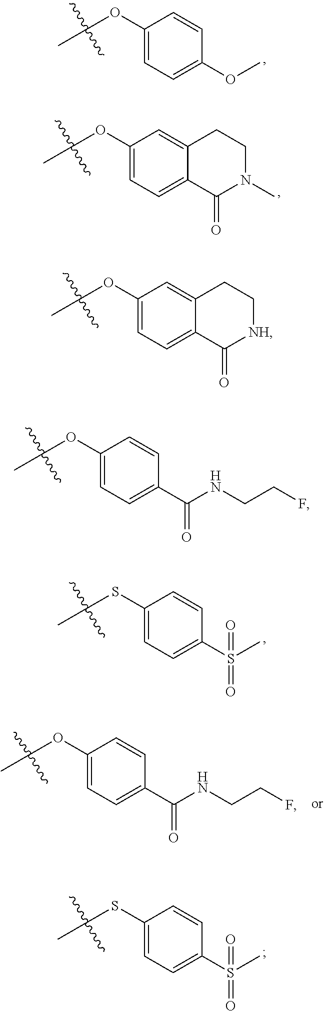

or

R₂ is selected from 2-(ethyl)butyl or isobutyl.

In various embodiments, the invention is directed towards an S1PR2 binding agent of formula

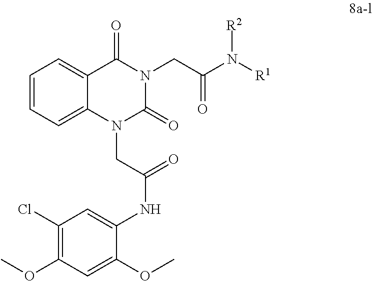

wherein

R¹ is selected from H,

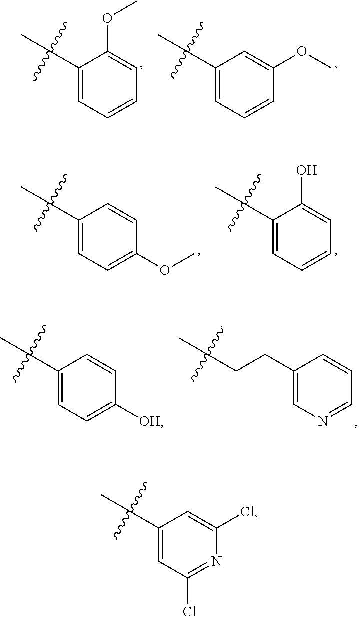

CH₃, CH₂CH₂OH, (CH₂CH₂O)₂H, or CH₃, or

R² is selected from H or CH₃.

In various embodiments, the invention is directed towards an S1PR2 binding agent of formula

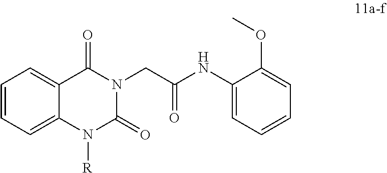

wherein

R is selected from

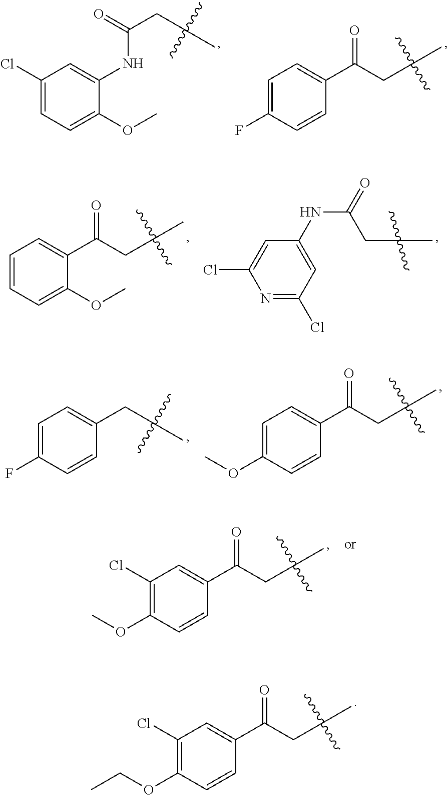

In various embodiments, the invention is directed towards an S1PR2 binding agent selected from one of the group consisting of:

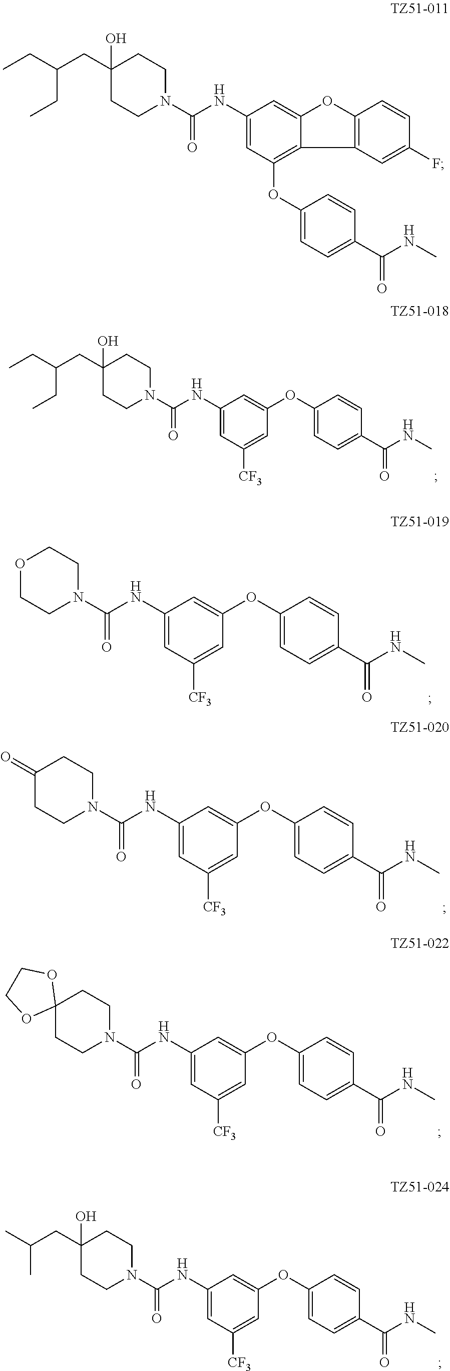

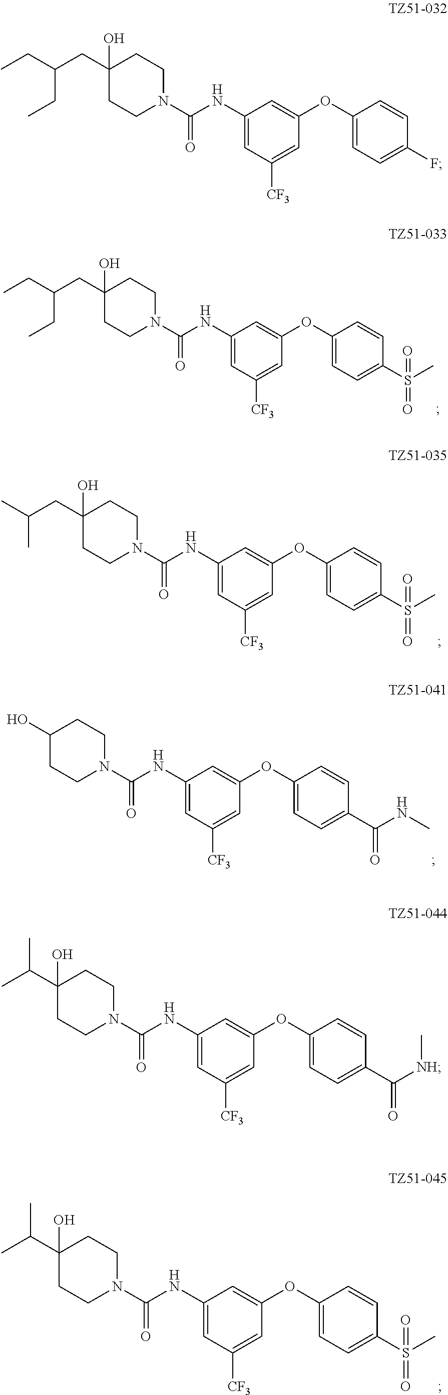

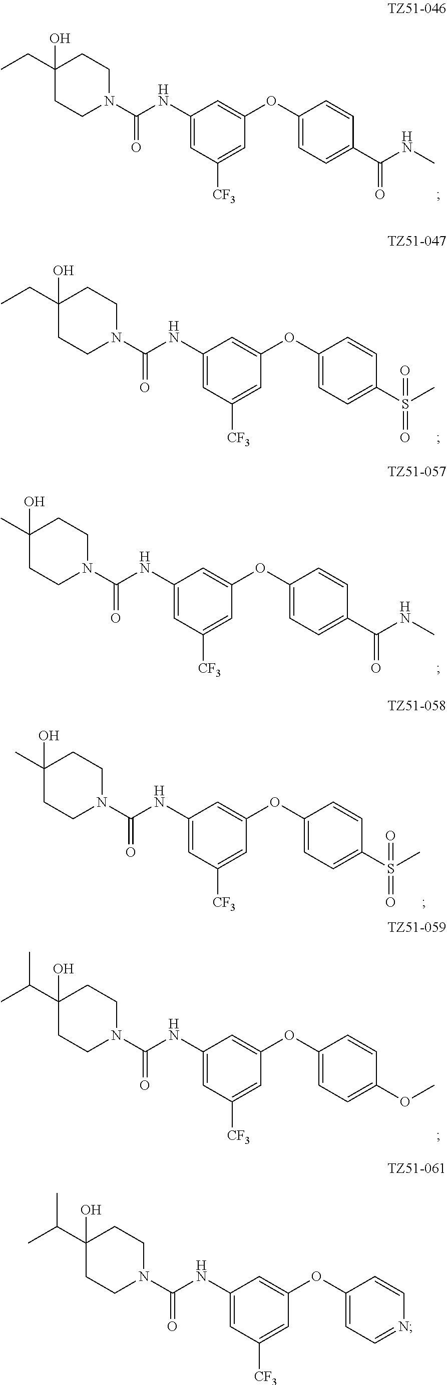

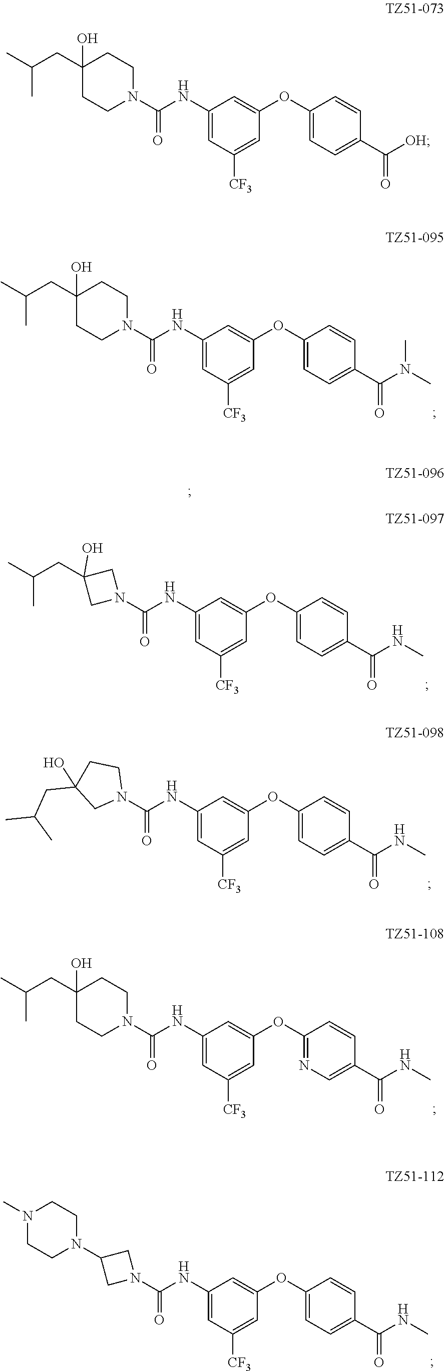

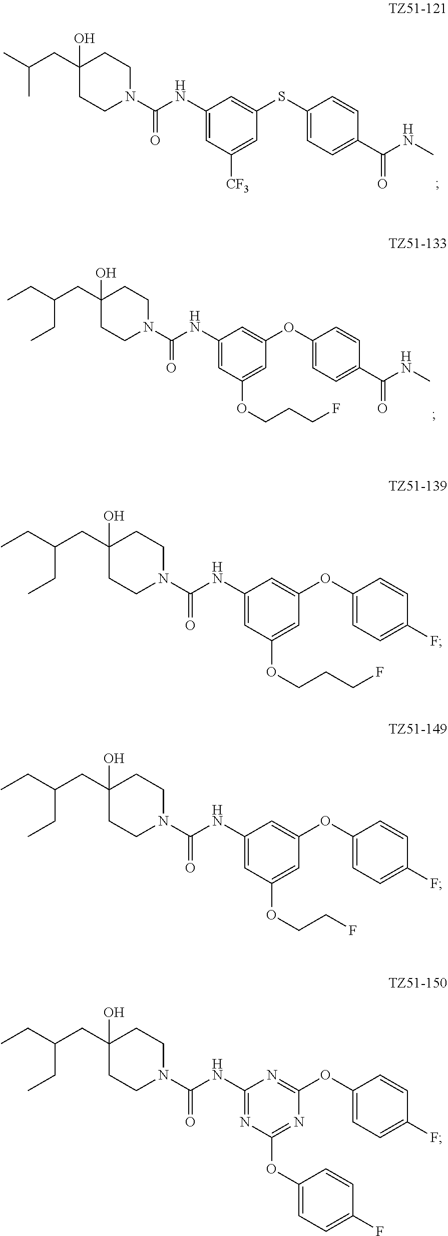

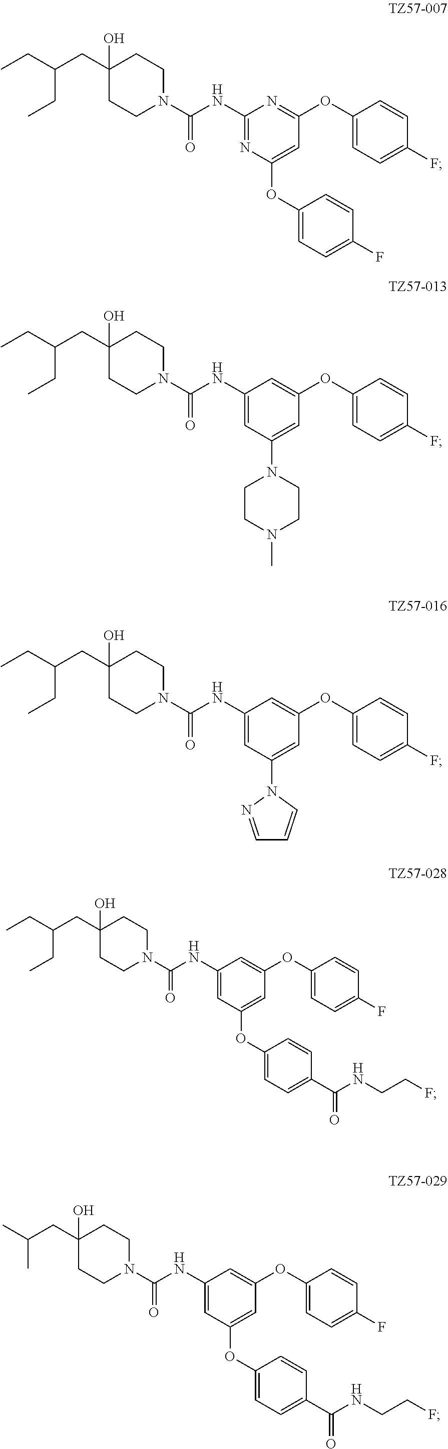 [SMILES: CCC(CC)CC1(O)CCN(C(=O)Nc2nc(Oc3ccc(F)cc3)cc(Oc3ccc(F)cc3)n2)CC1 ; CCC(CC)CC1(O)CCN(C(=O)Nc2cc(Oc3ccc(F)cc3)cc(N3CCN(C)CC3)c2)CC1 ; CCC(CC)CC1(O)CCN(C(=O)Nc2cc(Oc3ccc(F)cc3)cc(-n3cccn3)c2)CC1 ; CCC(CC)CC1(O)CCN(C(=O)Nc2cc(Oc3ccc(F)cc3)cc(Oc3ccc(C(=O)NCCF)cc3)c2)CC1 ; CC(C)CC1(O)CCN(C(=O)Nc2cc(Oc3ccc(F)cc3)cc(Oc3ccc(C(=O)NCCF)cc3)c2)CC1]

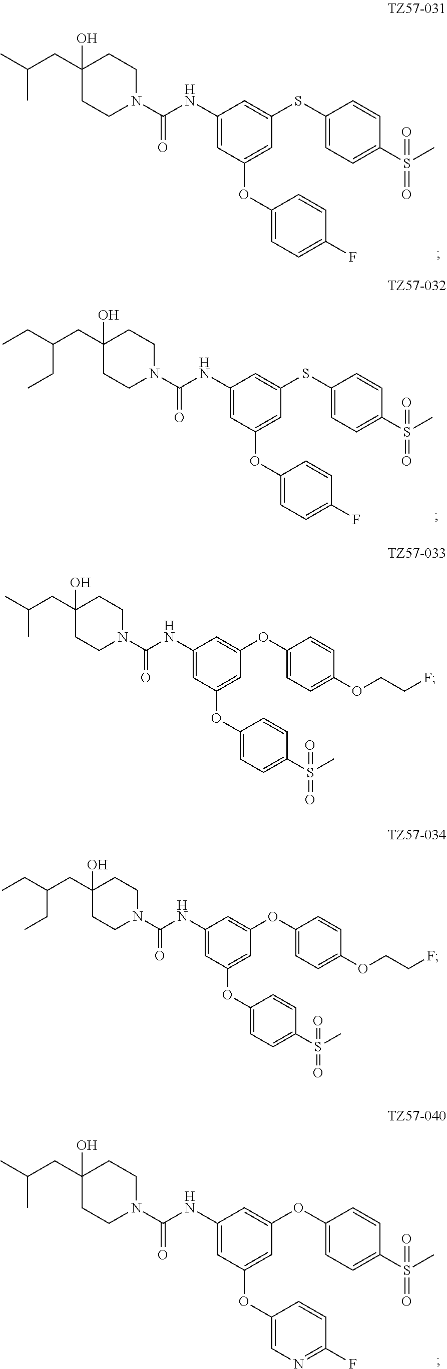

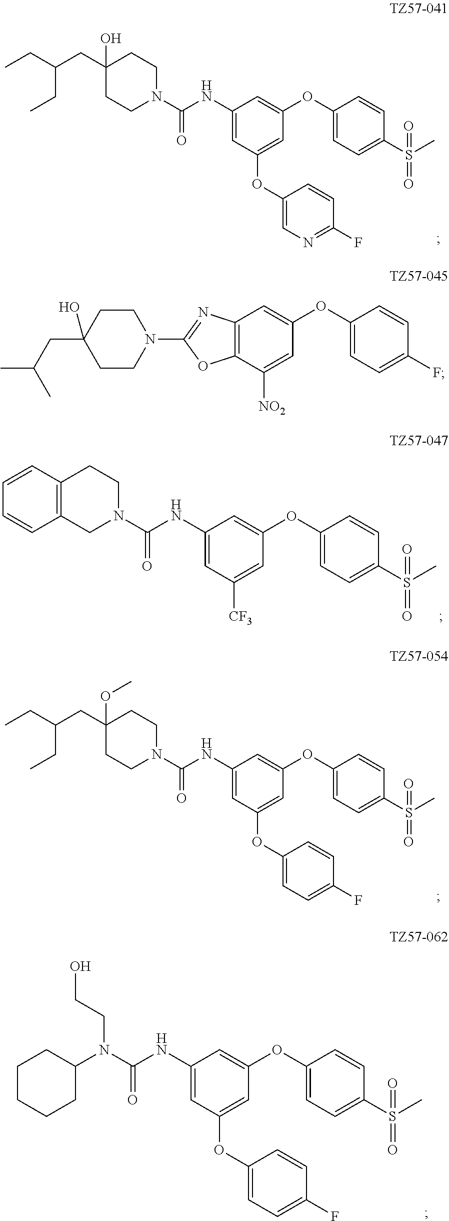

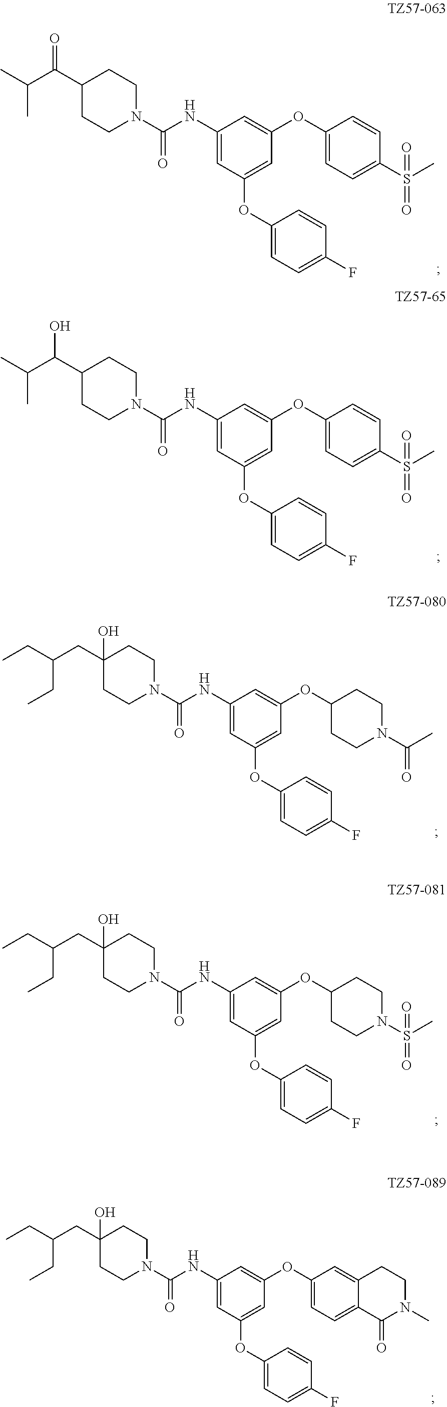

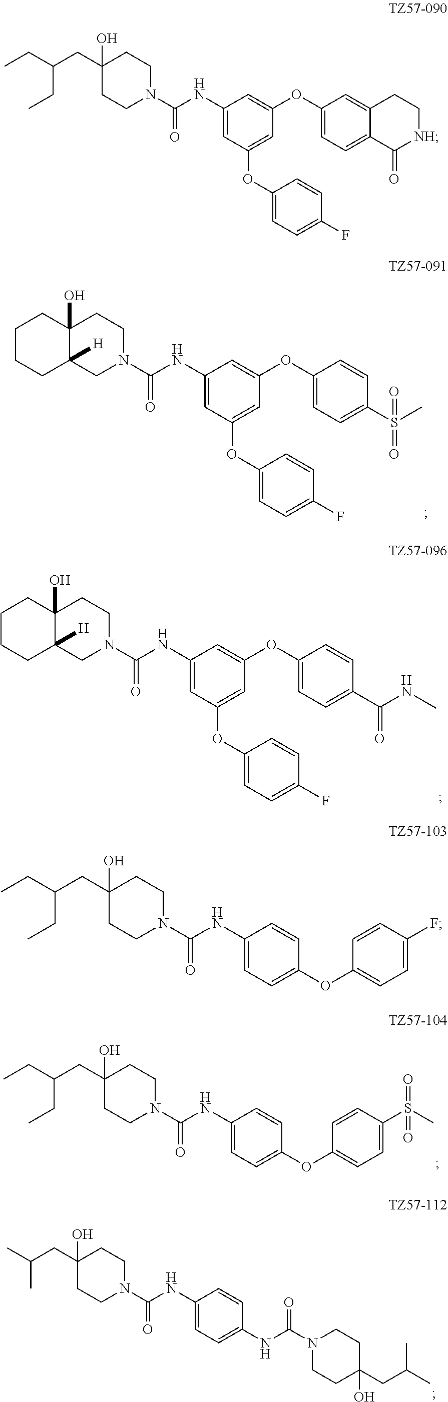

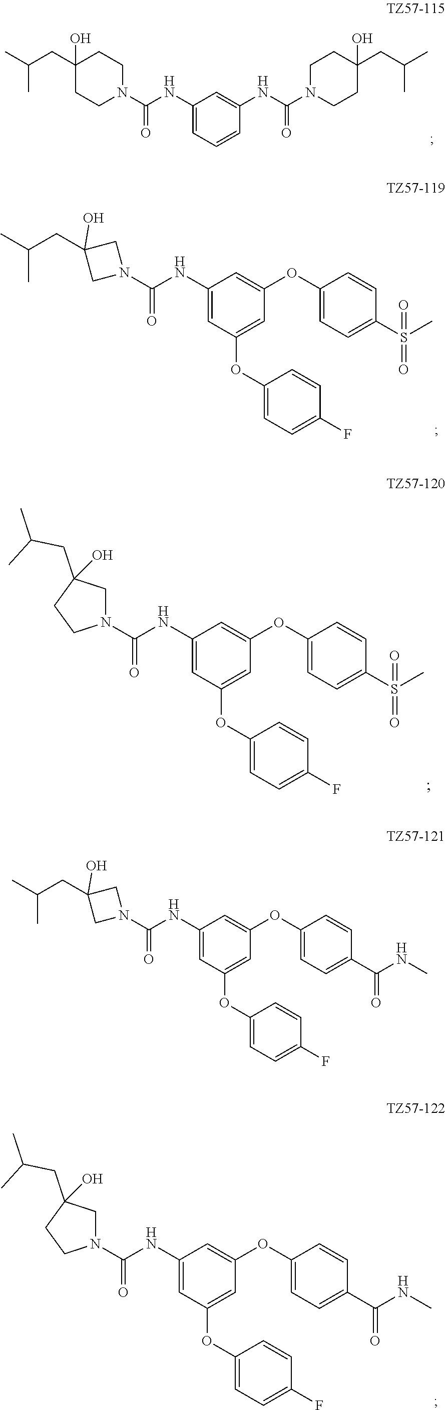

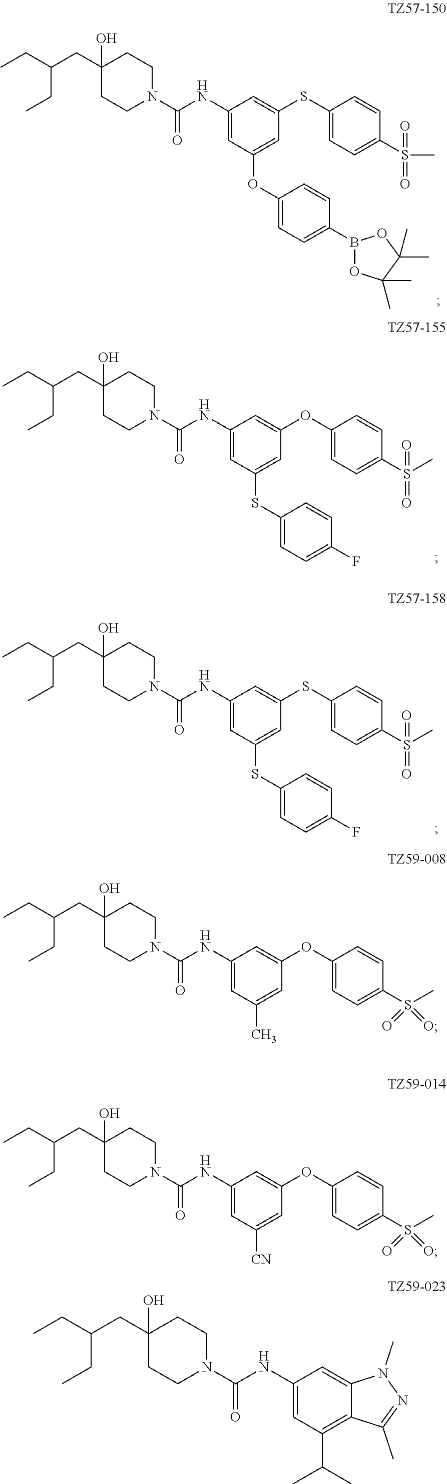

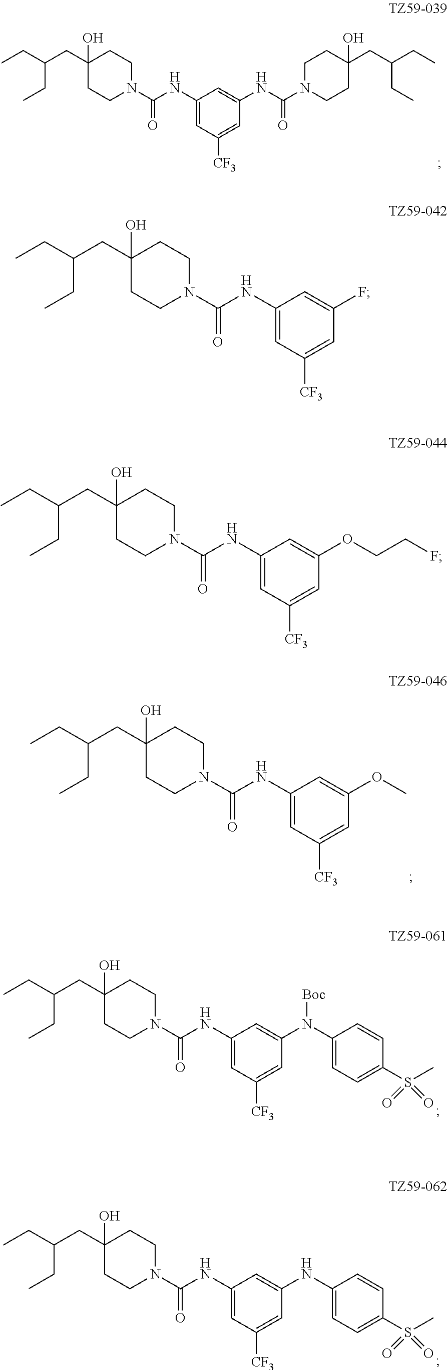

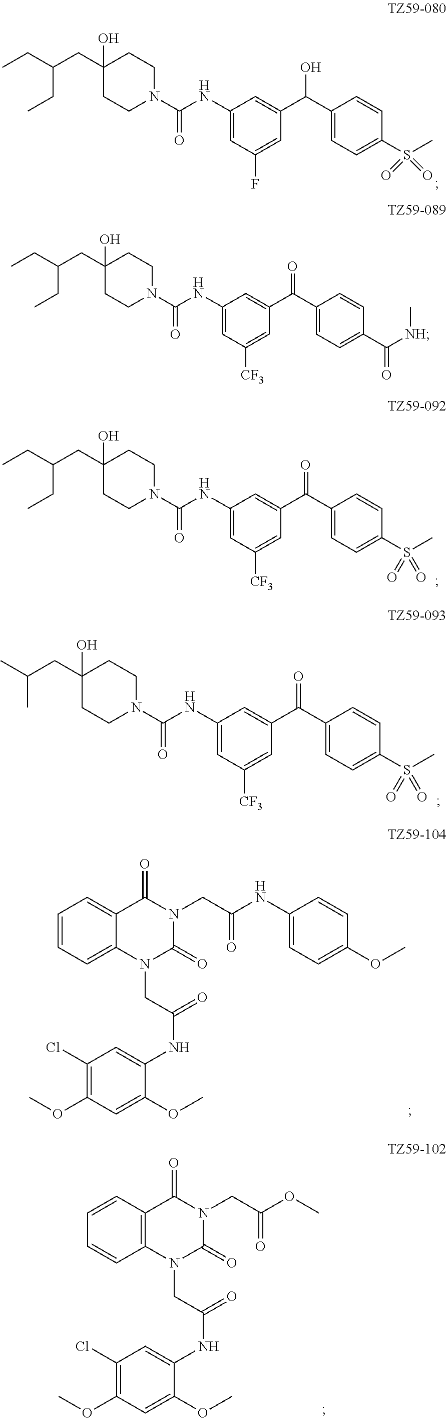

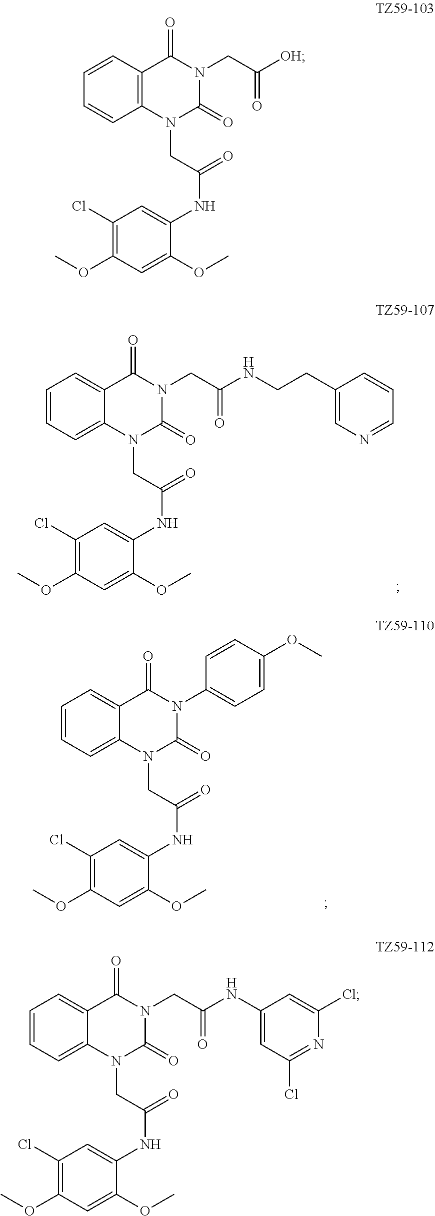

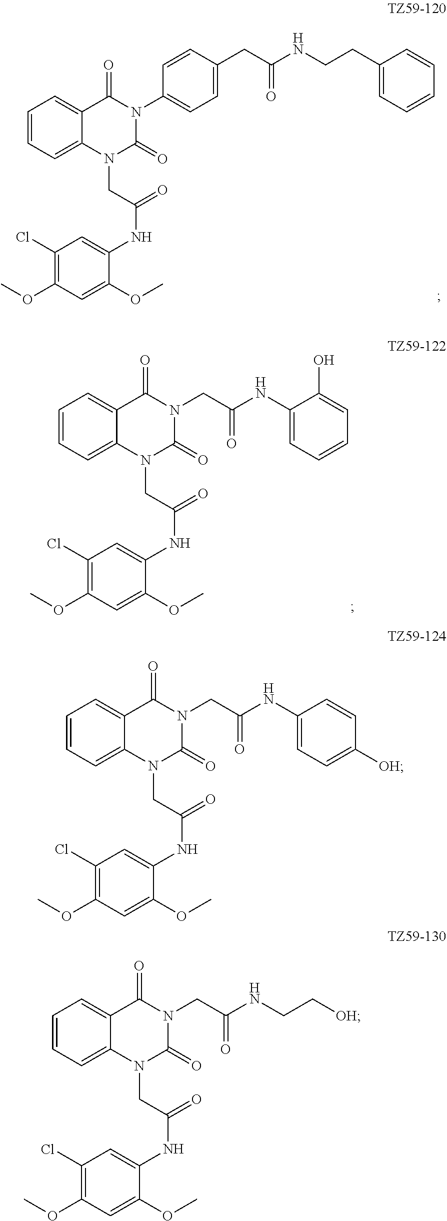 [SMILES: COc1cc(OC)c(NC(=O)Cn2c(=O)n(-c3ccc(CC(=O)NCCc4ccccc4)cc3)c(=O)c3ccccc32)cc1Cl ; COc1cc(OC)c(NC(=O)Cn2c(=O)n(CC(=O)Nc3ccccc3O)c(=O)c3ccccc32)cc1Cl ; COc1cc(OC)c(NC(=O)Cn2c(=O)n(CC(=O)Nc3ccc(O)cc3)c(=O)c3ccccc32)cc1Cl ; COc1cc(OC)c(NC(=O)Cn2c(=O)n(CC(=O)NCCO)c(=O)c3ccccc32)cc1Cl]

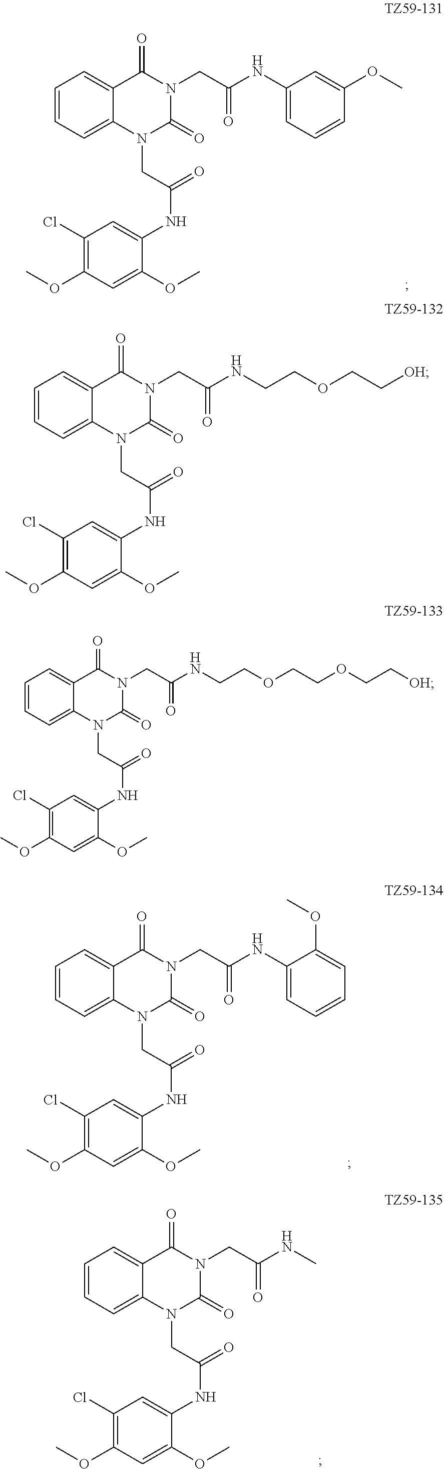

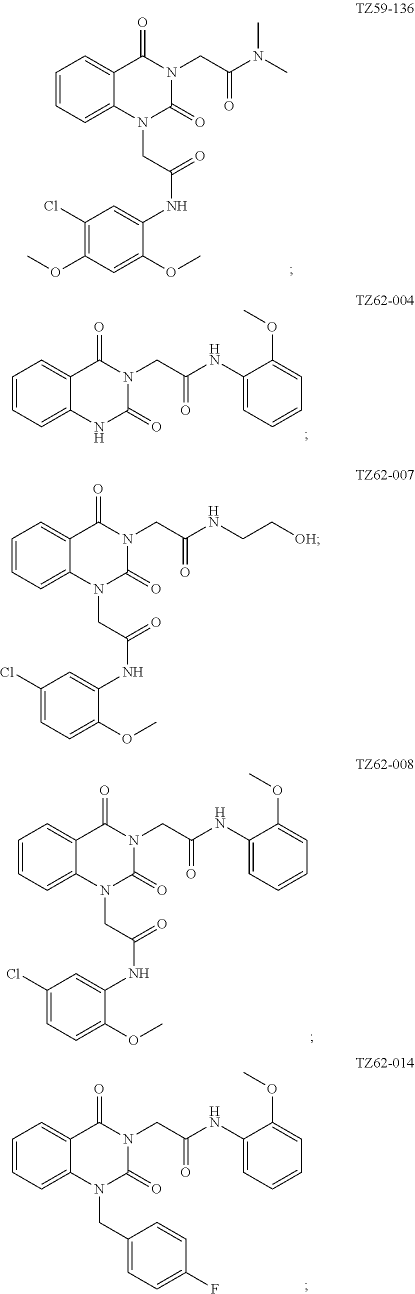

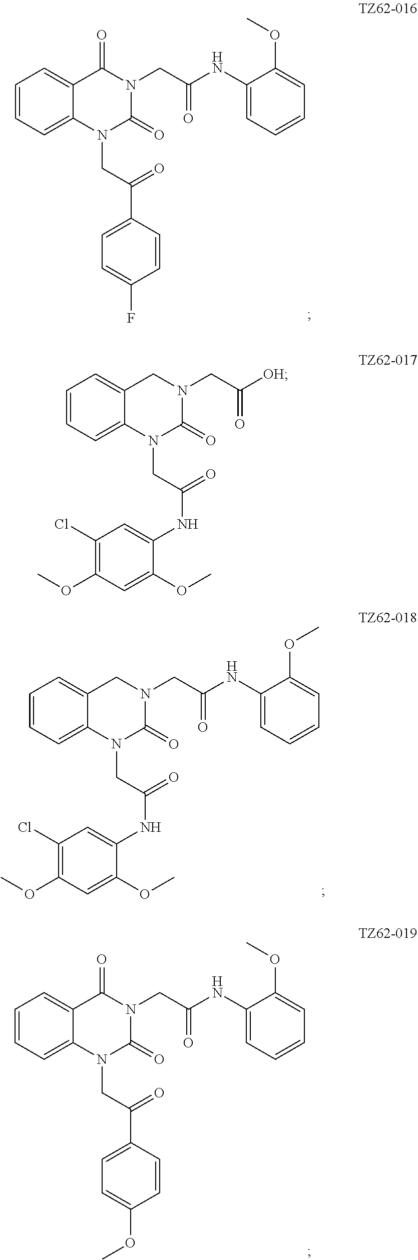 [SMILES: COc1ccccc1NC(=O)Cn1c(=O)c2ccccc2n(CC(=O)c2ccc(F)cc2)c1=O ; COc1cc(OC)c(NC(=O)CN2C(=O)N(CC(=O)O)Cc3ccccc32)cc1Cl ; COc1cc(OC)c(NC(=O)CN2C(=O)N(CC(=O)Nc3ccccc3OC)Cc3ccccc32)cc1Cl ; COc1ccc(C(=O)Cn2c(=O)n(CC(=O)Nc3ccccc3OC)c(=O)c3ccccc32)cc1]

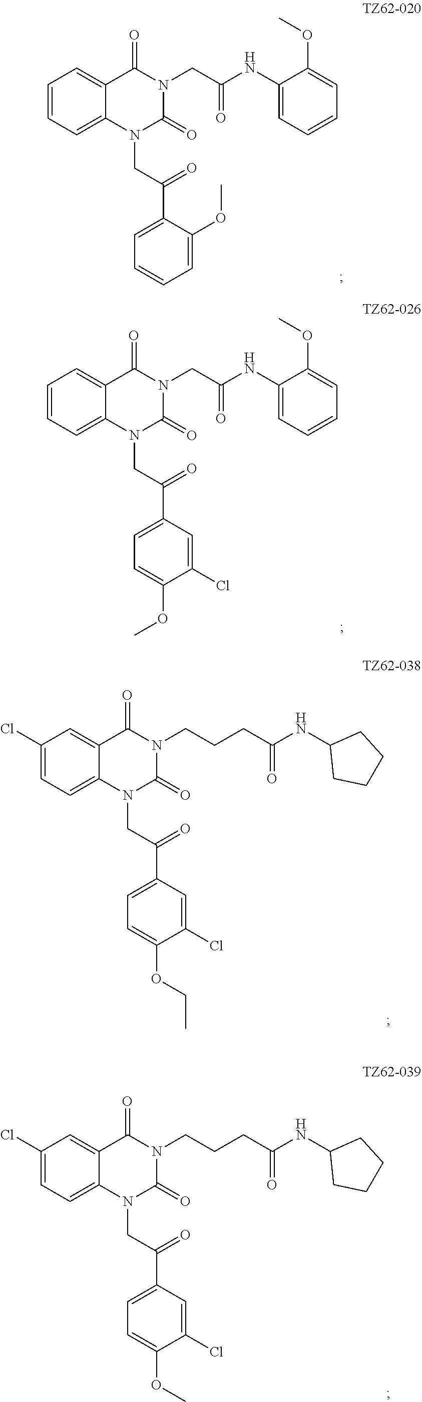 [SMILES: COc1ccccc1NC(=O)Cn1c(=O)c2ccccc2n(CC(=O)c2ccccc2OC)c1=O ; COc1ccc(C(=O)Cn2c(=O)n(CC(=O)Nc3ccccc3OC)c(=O)c3ccccc32)cc1Cl ; CCOc1ccc(C(=O)Cn2c(=O)n(CCCC(=O)NC3CCCC3)c(=O)c3cc(Cl)ccc32)cc1Cl ; COc1ccc(C(=O)Cn2c(=O)n(CCCC(=O)NC3CCCC3)c(=O)c3cc(Cl)ccc32)cc1Cl]

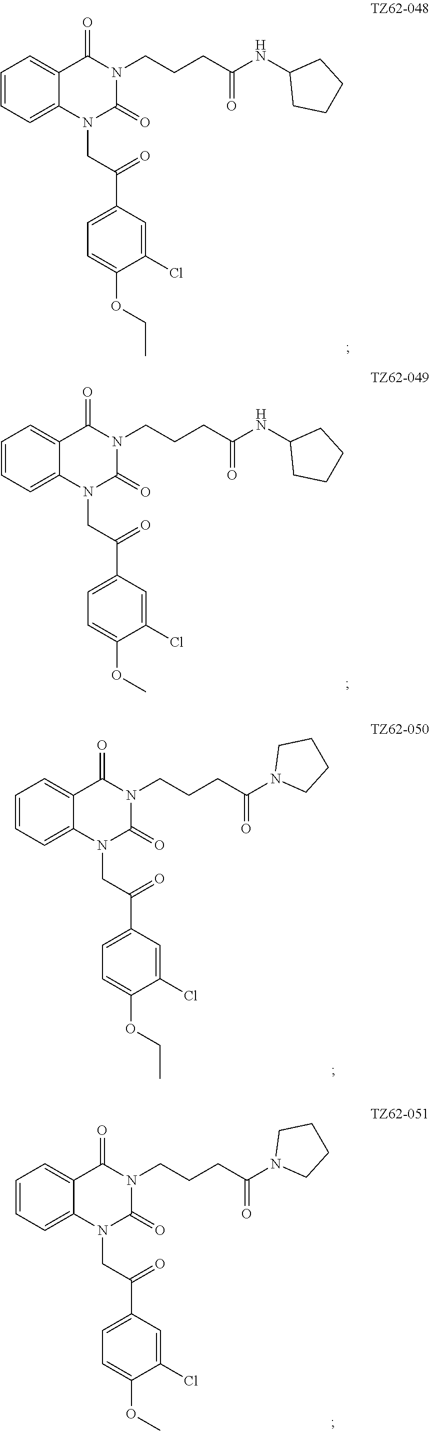 [SMILES: CCOc1ccc(C(=O)Cn2c(=O)n(CCCC(=O)NC3CCCC3)c(=O)c3ccccc32)cc1Cl ; COc1ccc(C(=O)Cn2c(=O)n(CCCC(=O)NC3CCCC3)c(=O)c3ccccc32)cc1Cl ; CCOc1ccc(C(=O)Cn2c(=O)n(CCCC(=O)N3CCCC3)c(=O)c3ccccc32)cc1Cl ; COc1ccc(C(=O)Cn2c(=O)n(CCCC(=O)N3CCCC3)c(=O)c3ccccc32)cc1Cl]

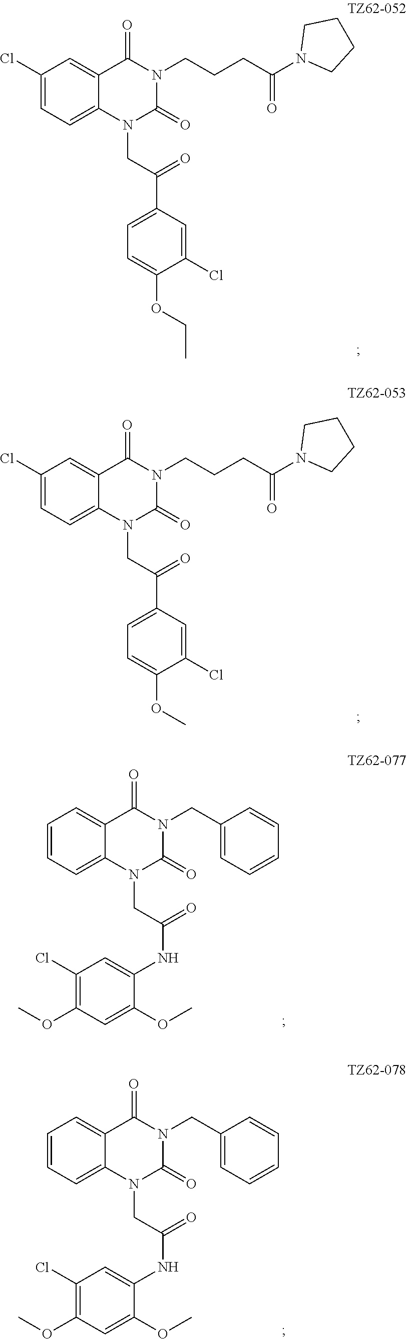 [SMILES: CCOc1ccc(C(=O)Cn2c(=O)n(CCCC(=O)N3CCCC3)c(=O)c3cc(Cl)ccc32)cc1Cl ; COc1ccc(C(=O)Cn2c(=O)n(CCCC(=O)N3CCCC3)c(=O)c3cc(Cl)ccc32)cc1Cl ; COc1cc(OC)c(NC(=O)Cn2c(=O)n(Cc3ccccc3)c(=O)c3ccccc32)cc1Cl ; COc1cc(OC)c(NC(=O)Cn2c(=O)n(Cc3ccccc3)c(=O)c3ccccc32)cc1Cl]

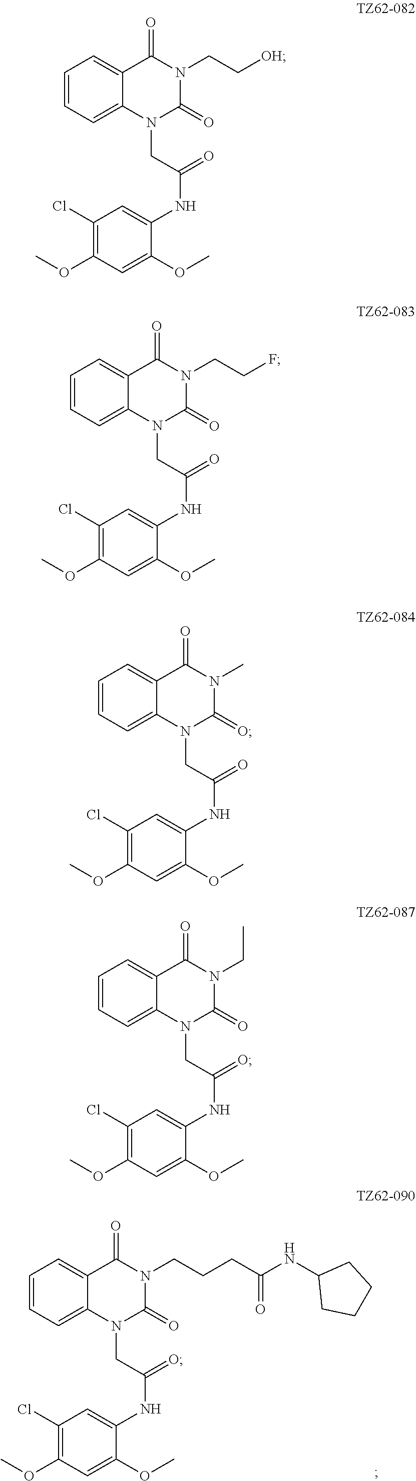

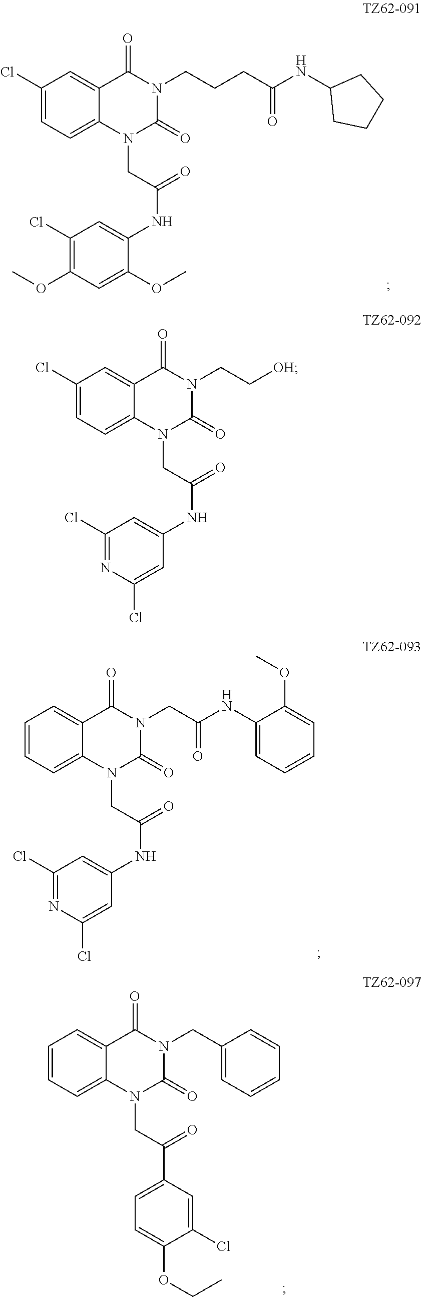

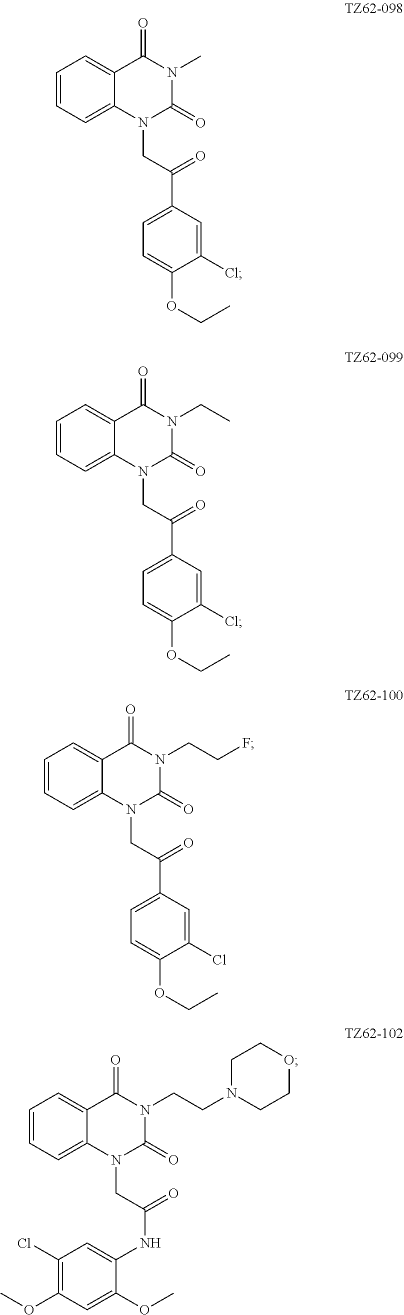 [SMILES: CCOc1ccc(C(=O)Cn2c(=O)n(C)c(=O)c3ccccc32)cc1Cl ; CCOc1ccc(C(=O)Cn2c(=O)n(CC)c(=O)c3ccccc32)cc1Cl ; CCOc1ccc(C(=O)Cn2c(=O)n(CCF)c(=O)c3ccccc32)cc1Cl ; COc1cc(OC)c(NC(=O)Cn2c(=O)n(CCN3CCOCC3)c(=O)c3ccccc32)cc1Cl]

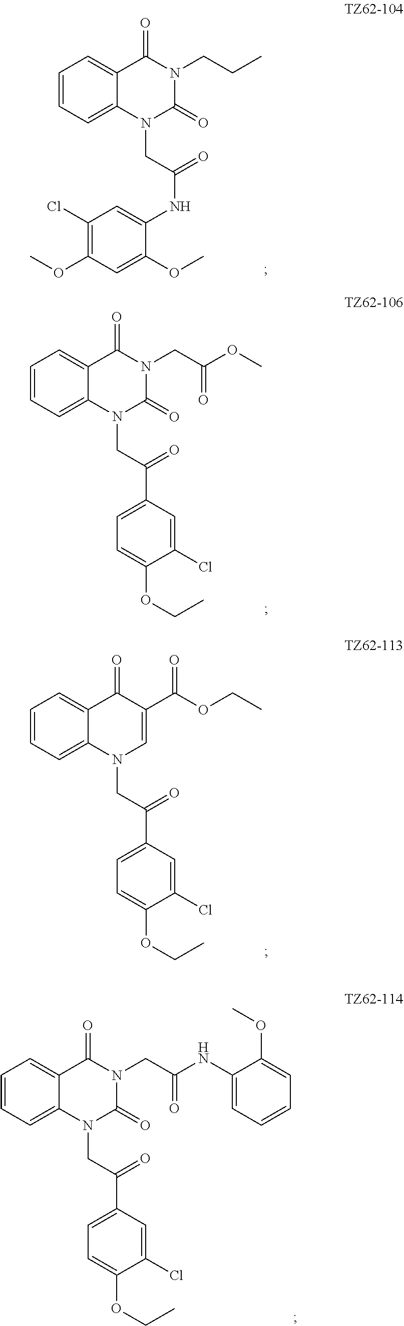

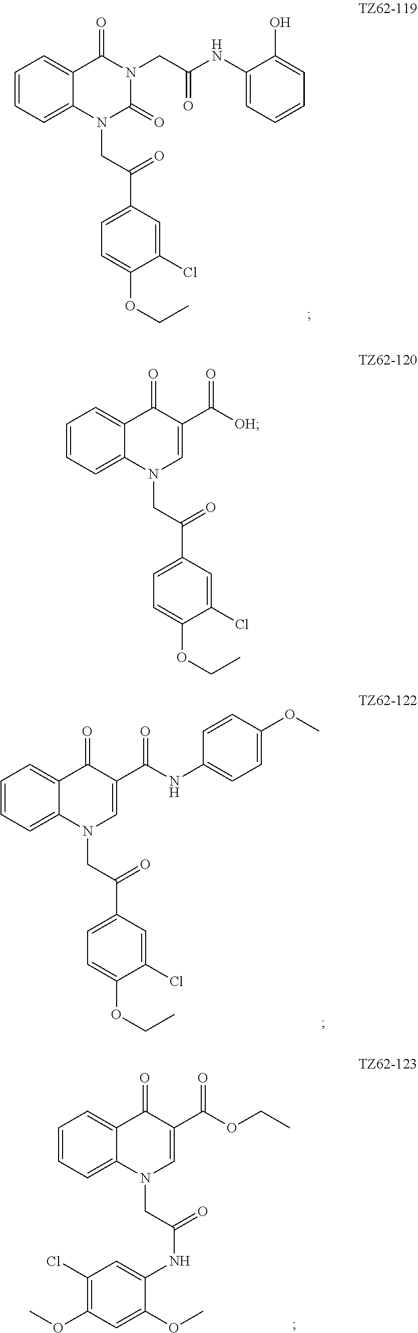

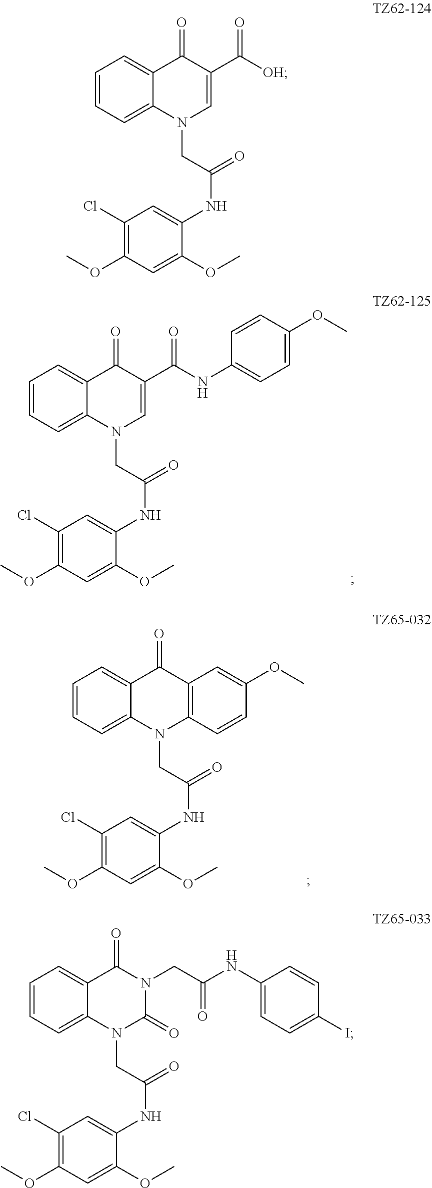

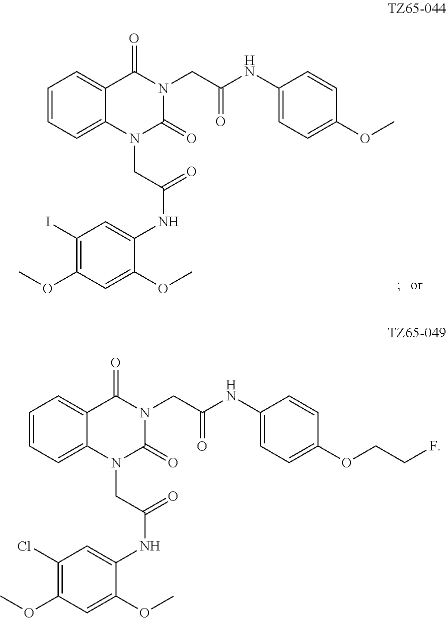

In various embodiments, the S1PR2 binding agent is not

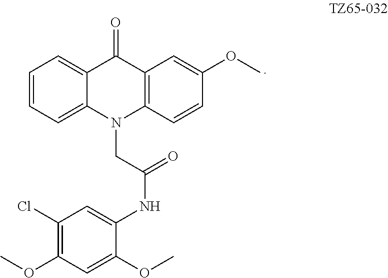

In various embodiments, the invention is directed towards an imaging agent comprising an S1PR2 binding agent as described herein.

In various embodiments, the invention is directed towards an imaging agent comprising an S1PR2 binding agent, wherein the S1PR2 binding agent is an analog of bis(aryloxy)benzene and quinazolinone. In some embodiments, the imaging agent is a PET tracer. In some embodiments, the imaging agent is a PET radiotracer labeled with an ¹¹O or an ¹⁸F.

### Imaging Agents

In various embodiments, the invention is directed to an imaging agent comprising a compound as described herein and a reporting group.

In various embodiments, the reporting group comprises a radioisotope, chromophore, photoluminescent moiety, a bioluminescent moiety, and/or a chemiluminescent moiety. In some embodiments, the reporting group comprises the radioisotope and the radioisotope is a synthetic radioisotope. In some embodiments, the radioisotope is ¹¹C, ¹⁸F, ¹²³I, ¹²⁵I, ¹³¹I, ⁷⁶Br or ³H. In some embodiments, the radioisotope is ¹¹C or ¹⁸F. In some embodiments, the reporting group is compatible with CT, PET, SPECT, MRI, MRS, ultrasound, and/or photoacoustics.

A compound or imaging agent including a radioisotope may be referred to as “radiolabeled”. “Radiolabeled” includes compounds where one or more atoms are replaced or substituted by an atom having an atomic mass or mass number different from the atomic mass or mass number typically found in nature (i.e., naturally occurring). One non-limiting example is ¹⁹F, which allows detection of a molecule which contains this element without enrichment to a higher degree than what is naturally occurring. Compounds carrying the substituent ¹⁹F may thus also be referred to as “labelled” or the like. The term radiolabeled may be interchangeably used with “isotopically-labelled”, “labelled”, “isotopic tracer group”, “isotopic marker”, “isotopic label”, “detectable isotope”, or “radioligand”.

Examples of suitable, non-limiting radiolabel groups can include: ²H (D or deuterium), ³H (T or tritium), ¹¹C, ¹³C, ¹⁴C ⁶⁴Cu, ⁶⁷Cu, ¹⁷⁷Lu, ¹³N, ¹⁵N, ¹⁵O, ¹⁷O, ¹⁸O, ¹⁸F, ⁸⁹Sr, ³⁵S, ¹⁵³Sm, ³⁶Cl, ⁸²Br, ⁷⁵Br, ⁷⁶Br, ⁷⁷Br, ¹²³I, ¹²⁴I, ¹²⁵I, ¹³¹I, ¹¹¹In, ⁶⁷Ga, ⁶⁸Ga, ¹⁷⁷Lu, ¹⁸⁶Re, ¹⁸⁸Re, ²⁰¹Tl, ^{199m}Tc, ⁹⁰Y, or ⁸⁹Zr. It is to be understood that an isotopically labeled compound needs only to be enriched with a detectable isotope to, or above, the degree which allows detection with a technique suitable for the particular application, e.g., in a detectable compound labeled with ¹¹C, the carbon-atom of the labeled group of the labeled compound may be constituted by ¹²C or other carbon-isotopes in a fraction of the molecules. The radionuclide that is incorporated in the radiolabeled compounds will depend on the specific application of that radiolabeled compound. For example, “heavy” isotope-labeled compounds (e.g., compounds containing deuterons/heavy hydrogen, heavy nitrogen, heavy oxygen, heavy carbon) can be useful for mass spectrometric and NMR based studies. As another example, for in vitro labelling or in competition assays, compounds that incorporate ³H, ¹⁴C, or ¹²⁵I can be useful. For in vivo imaging applications ¹¹C, ¹³C, ¹⁸F, ¹⁹F, ¹²⁰I, ¹²³I, ¹³¹I, ⁷⁵Br, or ⁷⁶Br can generally be useful. In one embodiment, the radiolabel is ¹¹C. In an alternative embodiment, the radiolabel is ¹⁴C. In a yet further alternative embodiment, the radiolabel is ¹³C.

### Formulations

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

In various embodiments, the invention is directed towards a pharmaceutical composition comprising a compound as described herein or an imaging agent as described herein and at least one pharmaceutically acceptable excipient.

In various embodiments, the composition comprises from about 0.001 mg to about 10 g of the compound or imaging agent in the pharmaceutical composition.

### Administration

Agents and compositions described herein can be administered according to methods described herein in a variety of means known to the art. Administration can be parenteral, pulmonary, oral, topical, intradermal, intramuscular, intraperitoneal, intravenous, subcutaneous, intranasal, epidural, ophthalmic, buccal, or rectal administration.

Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

In various embodiments, the invention is directed towards a method of treating anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and/or lung fibrosis in a subject in need thereof, the method comprising administering to the subject a therapeutically effective amount of a compound as described herein.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

In various embodiments, the method of treating comprises treating multiple sclerosis. In some embodiments, the method comprises treating an inflammatory response in multiple sclerosis.

In various embodiments, the effective amount of the compound is sufficient to upregulate, downregulate, or alter S1PR2 activity. In some embodiments, the method further comprises modifying or regulating intracellular calcium mobilization. In some embodiments, the method further comprises modifying or regulating glucose uptake.

In various embodiments, the invention is directed towards a method of imaging a target in a subject, the method comprising administering an imaging agent as described herein or a composition comprising the imaging agent and detecting the imaging agent in the subject.

In various embodiments, detecting the imaging agent comprises imaging with positron emission tomography (PET) imaging, and single photon emission computed tomography (SPECT) imaging, mass spectrometry, gamma imaging, magnetic resonance imaging (MRI), magnetic resonance spectroscopy, fluorescence spectroscopy, CT, ultrasound, or X-ray. In some embodiments, the imaging comprises using positron emission tomography.

In various embodiments, the method of imaging further comprises monitoring S1PR2 expression in the subject.

In various embodiments, the method of imaging further comprises assessing an inflammatory response in a disease.

In various embodiments, the target comprises a kidney or a kidney cell.

### Screening

The subject compounds also find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.)

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

In various embodiments, the invention is directed towards a method of determining an efficacy of a therapeutic agent in a subject comprising: administering a compound as described herein or the imaging agent as described herein to the subject; evaluating S1PR2 expression in the subject; treating a subject for a disease associated with increased S1PR2 expression; and reevaluating the S1PR2 expression in the subject.

In various embodiments, the method of evaluating the potential therapeutic includes determining an S1PR2 binding potency of the compound as described herein.

### Diagnosis

The compounds and imaging agents described herein may be used as tracers to assess the expression level and distribution of S1PR2 in patients with MS and other S1PR2 related diseases such as lung fibrosis, liver fibrosis, diabetes, and cancer, etc. Determining the expression level and distribution of S1PR2 can be useful for early diagnosis and for monitoring response to therapy in these diseases.

In various embodiments, the invention is directed towards a method of diagnosing or monitoring an S1PR2 associated disease, disorder or condition in a mammal comprising administering an imagining agent as described herein to the mammal and detecting the imaging agent, wherein the S1PR2 associated disease disorder or condition is selected from the group consisting of anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and lung fibrosis. In some embodiments, the S1PR2 associated disease, disorder or condition is multiple sclerosis.

In various embodiments, the invention is directed towards a method of quantifying S1PR2 expression in a mammalian brain or central nervous system comprising administering an imaging agent as described herein to the mammal and detecting the imaging agent in the mammal.

### Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Sphingosine-1-phosphate (S1P) is a metabolite and signaling molecule responsible for modulating vascular integrity and cellular processes. S1P binds to Sphingosine-1-phosphate receptor 2 (S1PR2), a G-protein coupled receptor. S1PR2 plays an important role in regulating vascular permeability and the blood brain barrier (BBB) during demyelinating diseases of the CNS. S1PR2 is widely expressed in a number of tissues with evidence of greater expression in female vs male mice and human patients with MS.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, includes —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, includes saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, includes a functional group consisting of a carbon atom double bonded to an oxygen atom and single bonded to a hydroxyl group (—COOH). The “carboxyl” can be optionally substituted.

The term “alkenyl”, as used herein, unless otherwise indicated, includes alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, includes alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, includes a functional group derived from an aliphatic carboxylic acid, by removal of the hydroxyl (—OH) group. The “acyl” can be optionally substituted.

The term “alkoxyl” or “alkoxy”, as used herein, unless otherwise indicated, includes O-alkyl groups wherein alkyl is as defined above and O represents oxygen. Representative alkoxyl groups include, but are not limited to, —O— methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O— cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, includes a non-aromatic, saturated, partially saturated, or unsaturated, monocyclic or fused, spiro or unfused bicyclic or tricyclic hydrocarbon referred to herein containing a total of from 3 to 10 carbon atoms, preferably 3 to 8 ring carbon atoms. Examples of cycloalkyls include, but are not limited to, 03-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also includes -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted.

The term “heterocyclic” or “heterocycloalkyl”, or “heteroaryl”, as used herein, unless otherwise indicated, includes an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocycle” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, includes a —CN group. The “cyano” can be optionally substituted.

The term “alcohol”, as used herein, unless otherwise indicated, includes a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Examples 1-93: Synthesis

### Example 1: General Procedures

Commercially available starting materials, reagents, and solvents were used as received. Unless otherwise indicated, all reactions were conducted in oven-dried glassware. In general, anhydrous reactions were performed under nitrogen. Reactions were monitored by thin-layer chromatography (TLC) carried out on pre-coated glass plates of silica gel (0.25 mm) 60 F₂₅₄ from EMD Chemicals Inc. Visualization was accomplished with ultraviolet light (UV 254 nm), or by shaking the TLC plate in a sealed jar containing silica gel and iodine. Flash column chromatography was performed using 230-400 mesh silica gel purchased from Silicycle. All work-up and purification procedures were carried out with reagent grade solvents in the air. Yields refer to isolate yield unless otherwise stated. Melting points were determined on a MEL-TEMP 3.0 apparatus. ¹H NMR and ¹³C NMR spectra were recorded on Varian 400 MHz instrument. Chemical shifts are reported in parts per million (ppm) and are calibrated using residual undeuterated solvent as an internal reference (CDCl₃: δ 7.26 ppm; CD₃OD: δ 3.31 ppm; DMSO: δ 2.50 ppm). Data are reported as follows: chemical shift, multiplicity, coupling constants (Hz), and integration. High resolution positive ion mass (HRMS) analyses were conducted on a Bruker MaXis 4G Q-TOF mass spectrometer with electrospray ionization source.

### Example 2: Synthetic Scheme of TZ 59 Compounds

The synthesis of TZ59-102, TZ59-103, TZ59-112, TZ59-107, TZ59-122, TZ59-124, TZ59-130, TZ59-131, TZ59-132, TZ59-134, TZ59-135, and TZ59-136 was accomplished as shown in Scheme 2. Briefly, N-protected of anthranilic acid, TZ59-098 was performed by reacting of anthranilic acid with CbzCl in the presence of NaHCO₃ in THF. Subsequently, the condensation reaction of TZ59-098 with methyl glycinate was proceeded with TBTU, HOBt, and DIPEA in DCM gave TZ59-099, which was cyclized in 1 N NaOH/MeOH (V/V, 3/20) to give TZ59-100. The nucleophilic substitution of TZ59-100 and TZ59-101, which was prepared by treating 5-chloro-2,4-dimethoxyaniline with bromoacetyl bromide gave TZ59-102. The hydrolysis of TZ59-102 gave acid TZ59-103, which could be coupled with different amines to give corresponding target compounds as shown in scheme 2.

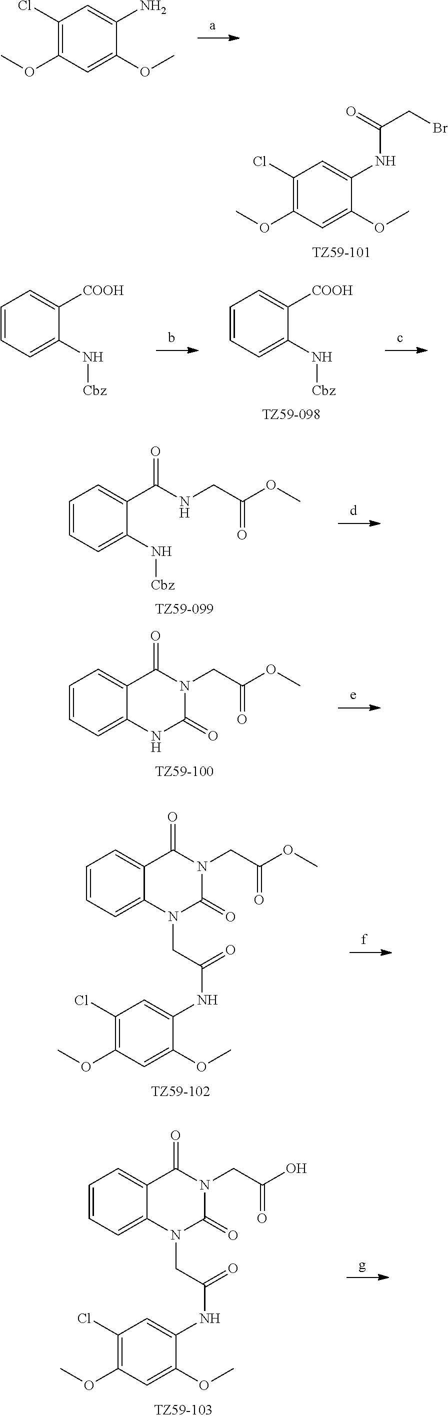

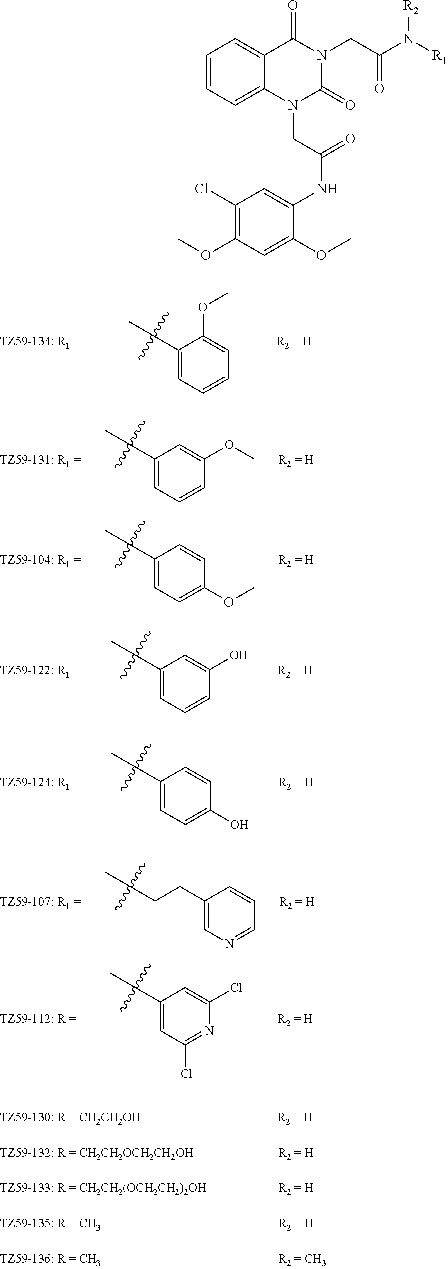

### Scheme 1.

Syntheses of 2a-l. Reagents and conditions: (a) Bromoacetyl bromide, Et3N, DCM, 0° C.-RT, (b) CbzCl, NaHCO3, THF; 0° C., (c) methyl glycinate, TBTU, HOBt, DIPEA, DCM, RT; (d) 1N NaOH/MeOH, RT; (e) TZ59-101, K2CO3, DMF, RT; (f) 5 M NaOH, MeOH, 50° C., HCl; (g) amines, HATU, DIPEA, DMSO, RT.dichloromethane, 0° C.-RT, (c) 2-bromo-N-(5-chloro-2,4-dimethoxyphenyl)acetamide, K₂CO₃, DMF, RT; (e) amines, HATU, DIPEA, DMF, RT.

### N-(5-Chloro-2,4-dimethoxyphenyl)-2-(3-(2-((2-methoxyphenyl)amino)-2-oxoethyl)-2,4-dioxo-3,4-dihydroquinazolin-1(2H)-yl)acetamide

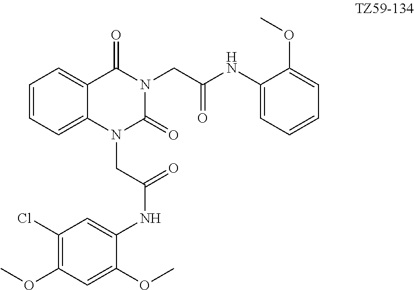 [SMILES: COc1cc(OC)c(NC(=O)Cn2c(=O)n(CC(=O)Nc3ccccc3OC)c(=O)c3ccccc32)cc1Cl]

To a round-bottomed flask equipped with a magnetic stir bar were added TZ59-103 (89 mg, 0.20 mmol), 2-methoxyaniline (27 mg, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-134.

### N-(5-Chloro-2,4-dimethoxyphenyl)-2-(3-(2-((3-methoxyphenyl)amino)-2-oxoethyl)-2,4-dioxo-3,4-dihydroquinazolin-1(2H)-yl)acetamide

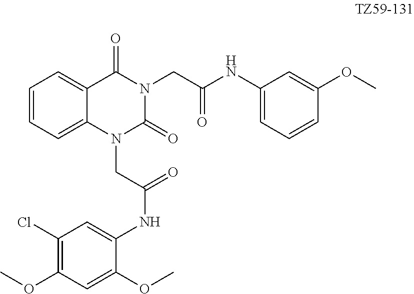

To a round-bottomed flask equipped with a magnetic stir bar were added TZ59-103 (89 mg, 0.20 mmol), 3-methoxyaniline (27 mg, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-131.

### N-(5-Chloro-2,4-dimethoxyphenyl)-2-(3-(2-((4-methoxyphenyl)amino)-2-oxoethyl)-2,4-dioxo-3,4-dihydroquinazolin-1(2H)-yl)acetamide

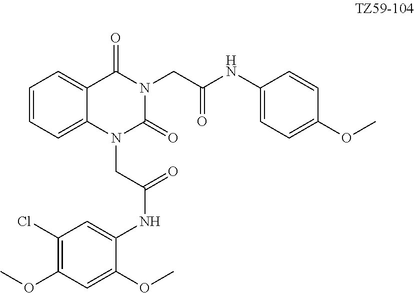

To a round-bottomed flask equipped with a magnetic stir bar were added TZ59-103 (100 mg, 0.22 mmol), 4-methoxyaniline (33 mg, 0.27 mmol), HATU (125 mg, 0.30 mmol), DIPEA (71 mg, 0.55 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-104. ¹H NMR (400 MHz, DMSO) δ 10.11 (s, 1H), 9.69 (s, 1H), 8.05 (d, J=7.8 Hz, 1H), 7.88 (s, 1H), 7.76 (t, J=7.8 Hz, 1H), 7.43 (d, J=8.7 Hz, 2H), 7.38-7.28 (m, 2H), 6.88-6.81 (m, 3H), 5.04 (s, 2H), 4.71 (s, 2H), 3.88 (s, 3H), 3.84 (s, 3H), 3.68 (s, 3H).

### N-(5-Chloro-2,4-dimethoxyphenyl)-2-(3-(2-((3-hydroxyphenyl)amino)-2-oxoethyl)-2,4-dioxo-3,4-dihydroquinazolin-1(2H)-yl)acetamide

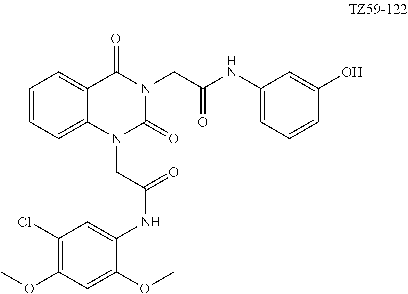 [SMILES: COc1cc(OC)c(NC(=O)Cn2c(=O)n(CC(=O)Nc3cccc(O)c3)c(=O)c3ccccc32)cc1Cl]

To a round-bottomed flask equipped with a magnetic stir bar were added TZ59-103 (70 mg, 0.15 mmol), 3-aminophenol (17 mg, 0.16 mmol), HATU (86 mg, 0.23 mmol), DIPEA (48 mg, 0.38 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-122. (50 mg, 62%) ¹H NMR (400 MHz, DMSO) δ 10.13 (s, 1H), 9.70 (s, 1H), 9.35 (s, 1H), 8.06 (d, J=7.9 Hz, 1H), 7.89 (s, 1H), 7.76 (t, J=7.9 Hz, 1H), 7.41-7.28 (m, 2H), 7.10-7.01 (m, 2H), 6.91 (d, J=7.9 Hz, 1H), 6.84 (s, 1H), 6.42 (d, 1H), 5.05 (s, 2H), 4.72 (s, 2H), 3.89 (s, 3H), 3.84 (s, 3H).

### N-(5-Chloro-2,4-dimethoxyphenyl)-2-(3-(2-((4-hydroxyphenyl)amino)-2-oxoethyl)-2,4-dioxo-3,4-dihydroquinazolin-1(2H)-yl)acetamide

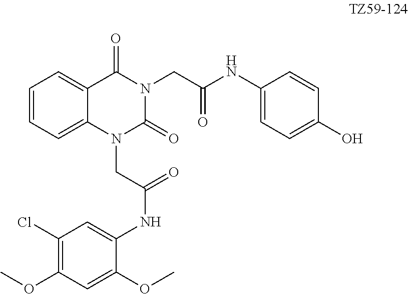 [SMILES: COc1cc(OC)c(NC(=O)Cn2c(=O)n(CC(=O)Nc3ccc(O)cc3)c(=O)c3ccccc32)cc1Cl]

To a round-bottomed flask equipped with a magnetic stir bar were added TZ59-103 (70 mg, 0.15 mmol), 4-aminophenol (17 mg, 0.16 mmol), HATU (86 mg, 0.23 mmol), DIPEA (48 mg, 0.38 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-124. ¹H NMR (400 MHz, DMSO) δ 9.98 (s, 1H), 9.70 (s, 1H), 9.16 (s, 1H), 8.06 (d, J=7.3 Hz, 1H), 7.88 (s, 1H), 7.76 (t, J=7.4 Hz, 1H), 7.39-7.32 (m, 1H), 7.30 (d, J=8.5 Hz, 2H), 6.84 (s, 1H), 6.65 (d, J=8.8 Hz, 2H), 5.04 (s, 2H), 4.70 (s, 2H), 3.89 (s, 3H), 3.84 (s, 3H).

### N-(5-Chloro-2,4-dimethoxyphenyl)-2-(2,4-dioxo-3-(2-oxo-2-((2-(pyridin-3-yl)ethyl)amino)ethyl)-3,4-dihydroquinazolin-1(2H)-yl)acetamide

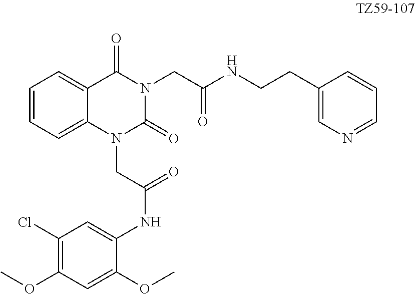 [SMILES: COc1cc(OC)c(NC(=O)Cn2c(=O)n(CC(=O)NCCc3cccnc3)c(=O)c3ccccc32)cc1Cl]

To a round-bottomed flask equipped with a magnetic stir bar were added TZ59-103 (100 mg, 0.22 mmol), 2-(pyridin-3-yl)ethan-1-amine (33 mg, 0.27 mmol), HATU (125 mg, 0.30 mmol), DIPEA (71 mg, 0.55 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-107. (67 mg. 55%)

### N-(5-Chloro-2,4-dimethoxyphenyl)-2-(3-(2-((2,6-dichloropyridin-4-yl)amino)-2-oxoethyl)-2,4-dioxo-3,4-dihydroquinazolin-1(2H)-yl)acetamide

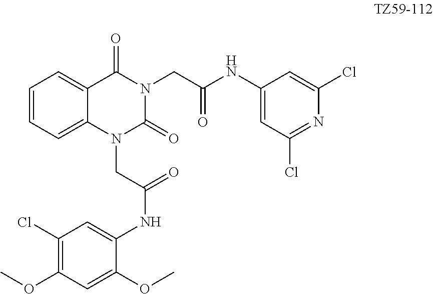 [SMILES: COc1cc(OC)c(NC(=O)Cn2c(=O)n(CC(=O)Nc3cc(Cl)nc(Cl)c3)c(=O)c3ccccc32)cc1Cl]

To a round-bottomed flask equipped with a magnetic stir bar were added TZ59-103 (100 mg, 0.22 mmol), 2,6-dichloropyridin-4-amine (40 mg, 0.25 mmol), HATU (125 mg, 0.33 mmol), DIPEA (71 mg, 0.55 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-112.

### N-(5-Chloro-2,4-dimethoxyphenyl)-2-(3-(2-((2-hydroxyethyl)amino)-2-oxoethyl)-2,4-dioxo-3,4-dihydroquinazolin-1(2H)-yl)acetamide

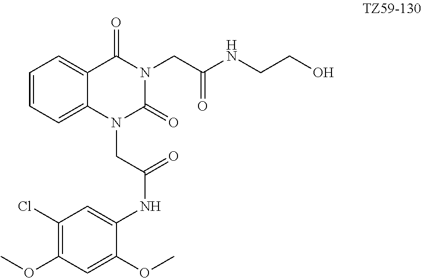 [SMILES: COc1cc(OC)c(NC(=O)Cn2c(=O)n(CC(=O)NCCO)c(=O)c3ccccc32)cc1Cl]

To a round-bottomed flask equipped with a magnetic stir bar were added TZ59-103 (89 mg, 0.20 mmol), 2-aminoethan-1-ol (13 mg, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-130. (76 mg, 77%)

### N-(5-Chloro-2,4-dimethoxyphenyl)-2-(3-(2-((2-(2-hydroxyethoxy)ethyl)amino)-2-oxoethyl)-2,4-dioxo-3,4-dihydroquinazolin-1(2H)-yl)acetamide

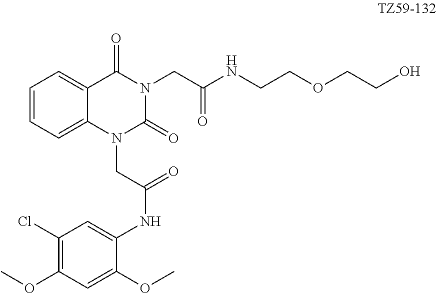 [SMILES: COc1cc(OC)c(NC(=O)Cn2c(=O)n(CC(=O)NCCOCCO)c(=O)c3ccccc32)cc1Cl]

To a round-bottomed flask equipped with a magnetic stir bar were added TZ59-103 (89 mg, 0.20 mmol), 2-(2-aminoethoxy)ethan-1-ol (23 mg, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-132. (59 mg, 55%) ¹H NMR (400 MHz, DMSO) δ 9.68 (s, 1H), 8.18 (t, J=5.4 Hz, 1H), 8.04 (d, J=6.7 Hz, 1H), 7.87 (s, 1H), 7.75 (t, J=7.9 Hz, 1H), 7.37-7.27 (m, 2H), 6.84 (s, 1H), 5.02 (s, 2H), 4.56-4.51 (m, 3H), 3.89 (s, 3H), 3.84 (s, 3H), 3.51-3.44 (m, 2H), 3.43-3.36 (m, 3H), 3.25-3.16 (m, 2H).

### N-(5-Chloro-2,4-dimethoxyphenyl)-2-(3-(2-((2-(2-(2-hydroxyethoxy)ethoxy)ethyl)amino)-2-oxoethyl)-2,4-dioxo-3,4-dihydroquinazolin-1(2H)-yl)acetamide

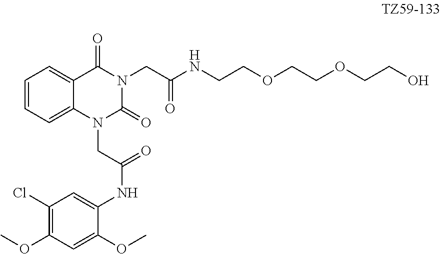 [SMILES: COc1cc(OC)c(NC(=O)Cn2c(=O)n(CC(=O)NCCOCCOCCO)c(=O)c3ccccc32)cc1Cl]

To a round-bottomed flask equipped with a magnetic stir bar were added TZ59-103 (89 mg, 0.20 mmol), 2-(2-(2-aminoethoxy)ethoxy)ethan-1-ol (33 mg, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-133. (59 mg, 55%) ¹H NMR (400 MHz, DMSO) δ 9.67 (s, 1H), 8.19 (t, J=5.5 Hz, 1H), 8.04 (d, J=7.8 Hz, 1H), 7.87 (s, 1H), 7.75 (t, J=7.2 Hz, 1H), 7.39-7.26 (m, 2H), 6.84 (s, 1H), 5.02 (s, 2H), 4.58-4.48 (m, 3H), 3.89 (s, 3H), 3.84 (s, 3H), 3.52-3.49 (m, 3H), 3.48-3.43 (m, 2H), 3.42-3.35 (m, 4H), 3.24-3.16 (m, 2H).

### N-(5-Chloro-2,4-dimethoxyphenyl)-2-(3-(2-(methylamino)-2-oxoethyl)-2,4-dioxo-3,4-dihydroquinazolin-1(2H)-yl)acetamide

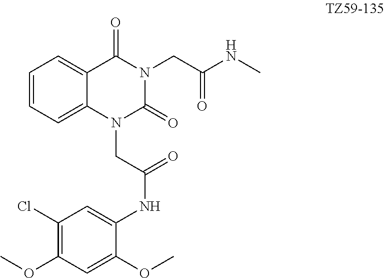

To a round-bottomed flask equipped with a magnetic stir bar were added TZ59-103 (89 mg, 0.20 mmol), methylamine in THF (2 M) (110 μL, 0.22 mmol), HATU (114 mg, 0.30 mmol), DIPEA (65 mg, 0.50 mmol) and DMSO (3.0 mL). The reaction was stirred at room temperature for overnight until the reaction was completed as determined by TLC. Then, the reaction mixture was diluted with water and filtered, the precipitate was collected and washed with ethanol as product TZ59-135. ¹H NMR (400 MHz, DMSO) δ 9.67 (s, 1H), 8.08-7.97 (m, 2H), 7.87 (s, 1H), 7.75 (t, J=7.9 Hz, 1H), 7.39-7.25 (m, 2H), 6.84 (s, 1H), 5.02 (s, 2H), 4.50 (s, 2H), 3.89 (s, 3H), 3.84 (s, 3H), 2.56 (d, J=4.3 Hz, 3H).

### 2-(1-(2-((5-Chloro-2,4-dimethoxyphenyl)amino)-2-oxoethyl)-2,4-dioxo-1,4-dihydroquinazolin-3(2H)-yl)-N,N-dimethylacetamide

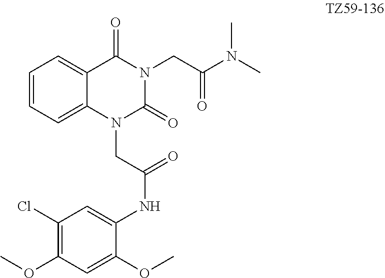

To a round-bottomed flask equipped with a magnetic stir bar were added TZ59-103 (89 mg, 0.20 mmol), ethylamine in THF (2 M) (110 μL, 0.22 mmol), HATU (114 mg, 0.30 mmol), DIPEA (65 mg, 0.50 mmol) and DMSO (3.0 mL). The reaction was stirred at room temperature overnight until the reaction was completed as determined by TLC. Then, the reaction mixture was diluted with water and filtered, the precipitate was collected and washed with ethanol as product TZ59-136.

### Example 3: Synthetic Scheme of 4a-f

Compounds 4a-f and precursors were synthesized by Scheme 2.

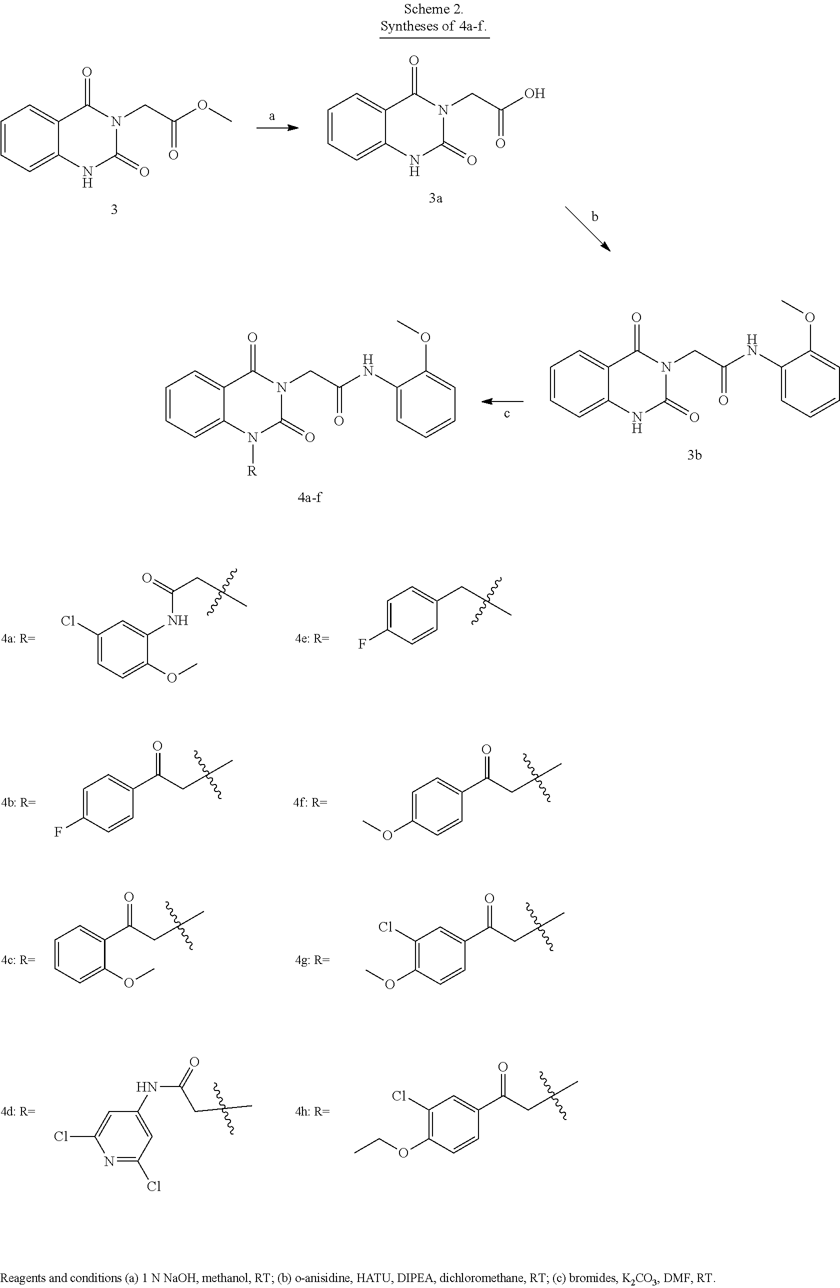

### Example 4: Synthetic Scheme of 11a-b and Precursors

Compounds 11a-b and precursors were synthesized by Scheme 3.

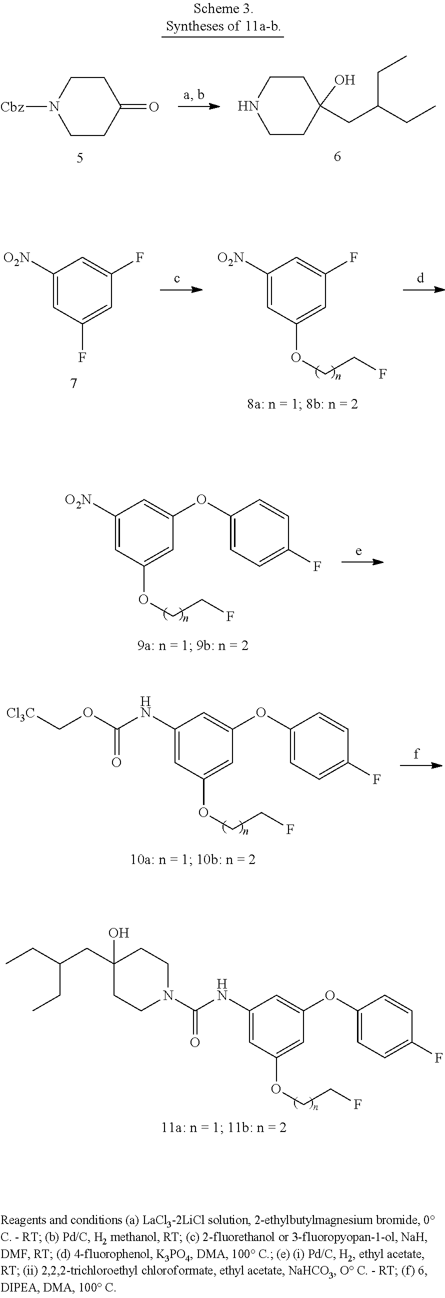

### Example 5: Synthesis of 4-(2-ethylbutyl)piperidin-4-ol (6)

To a dried round two neck bottomed flask equipped with a magnetic stir bar were added 0.6 M LaCl₃-2LiCl in THF (12.5 mL, 7.5 mmol) under nitrogen, 0.25 M 2-ethylbutylmagnesium bromide in THF (30 mL, 7.5 mmol) was added slowly through syringe at 0° C. After stirring at room temperature for 3 h, a solution of benzyl 4-oxopiperidine-1-carboxylate (5) (1.2 g, 5.0 mmol) in THF (5.0 mL) was added into the mixture. The reaction was stirred for another 18 h until the reaction was completed as determined by TLC and then quenched with 25% acetic acid. The mixture was extracted with ethyl acetate, the ethyl acetate layer was washed with saturated brine and dried over anhydrous MgSO4. After filtration and concentration, the crude product was used directly for the next step without purification.

To a round-bottomed flask equipped with a magnetic stir bar were added above crude product, 10% Pd/C (0.2 g) and methanol (10.0 mL). The reaction was bubbled with hydrogen gas for 6 h at room temperature until the reaction was completed as determined by TLC and then filtered through celite. The filtrate was concentrated under reduced pressure to afford yellow oil product 6. Yield: 81%. ¹H NMR (400 MHz, CDCl₃) δ 3.18 (s, 1H), 2.98 (d, J=33.4 Hz, 4H), 1.96 (s, 1H), 1.63 (d, J=24.9 Hz, 4H), 1.46-1.30 (m, 7H), 0.86 (t, J=7.1 Hz, 6H).

### Example 6: General Procedure to Synthesize 8a-b

A solution of 2-fluoroethanol or 3-fluoropyopan-1-ol (1.0 eq) in DMF (0.25 M) was stirred and cooled to 0° C., then NaH (2.0 eq) was added. After stirring for 15 min, a solution of 1,3-difluoro-5-nitrobenzene (7) (1.0 eq) in DMF (0.5 M) was added to the mixture and stirred at room temperature for 12 h until the reaction was completed as determined by TLC. The reaction then was diluted with water and extracted with ethyl acetate. The organic layer was washed with saturated brine and dried over anhydrous MgSO₄. After filtration and concentration, the crude product was purified on a silica gel column, eluted with hexane/ethyl acetate to afford 8a-b.

### Example 7: Synthesis of 1-Fluoro-3-(2-fluoroethoxy)-5-nitrobenzene (8a)

Compound 8a was eluted with hexane/ethyl acetate (10/1, V/V) as yellow oil. Yield: 33%. ¹H NMR (400 MHz, CDCl₃) δ 7.60-7.55 (m, 2H), 6.99 (dt, J=9.6, 2.3 Hz, 1H), 4.87-4.83 (m, 1H), 4.75-4.71 (m, 1H), 4.35-4.31 (m, 1H), 4.29-4.25 (m, 1H).

### Example 8: Synthesis of 1-Fluoro-3-(3-fluoropropoxy)-5-nitrobenzene (8b)

Compound 8b was eluted with hexane/ethyl acetate (10/1, V/V) as yellow oil. Yield: 35%. ¹H NMR (400 MHz, CDCl₃) δ 7.58-7.56 (m, 1H), 7.55-7.50 (m, 1H), 6.98-6.92 (m, 1H), 4.71 (t, J=5.7 Hz, 1H), 4.59 (t, J=5.7 Hz, 1H), 4.17 (t, J=6.1 Hz, 2H), 2.29-2.14 (m, 2H).

### Example 9: General Procedure to Synthesize 9a-b

To a round-bottomed flask equipped with a magnetic stir bar was added 8a-b (1.0 eq), 4-fluorophenol (1.2 eq), potassium phosphate (2.0 eq), and DMA (1.0 M). The reaction vessel was immersed in a 100° C. preheated oil bath for 12 h until the reaction was completed as determined by TLC. After cooling, the reaction was diluted with water and extracted with ethyl acetate. The organic layer was washed with saturated brine and dried over anhydrous MgSO4. After filtration and concentration, the crude product was pure enough for the next step.

### Example 10: Synthesis of 1-(2-Fluoroethoxy)-3-(4-fluorophenoxy)-5-nitrobenzene (9a)

Yellow oil, yield: 99%. ¹H NMR (400 MHz, CDCl₃) δ 7.45 (t, J=2.1 Hz, 1H), 7.37 (t, J=2.1 Hz, 1H), 7.14-6.99 (m, 4H), 6.83 (t, J=2.3 Hz, 1H), 4.83-4.79 (m, 1H), 4.71-4.68 (m, 1H), 4.31-4.26 (m, 1H), 4.24-4.20 (m, 1H).

### Example 11: Synthesis of 1-(4-Fluorophenoxy)-3-(3-fluoropropoxy)-5-nitrobenzene (9b)

Yellow oil, yield: 91%. ¹H NMR (400 MHz, CDCl₃) δ 7.46 (s, 1H), 7.34 (s, 1H), 7.14-6.98 (m, 4H), 6.80 (s, 1H), 4.70 (t, J=5.9 Hz, 1H), 4.58 (t, J=5.9 Hz, 1H), 4.19-4.11 (m, 2H), 2.25-2.14 (m, 2H).

### Example 12: General Procedure to Synthesize 10a-b

To a round-bottomed flask equipped with a magnetic stir bar were added 9a-b (1.0 eq), 10% Pd/C, and ethyl acetate (0.05 M). The reaction was bubbled with hydrogen gas for 12 h at room temperature until the reaction was completed as determined by TLC. The mixture then was filtered through celite. To the filtrate was added NaHCO₃ (2.0 eq) followed by adding 2,2,2-trichloroethyl chloroformate (1.0 eq) slowly through syringe under nitrogen at 0° C. The reaction was warmed to room temperature and stirred for 3 h until the reaction was completed as determined by TLC. The mixture then was washed with water, saturated brine and dried over anhydrous MgSO₄. After filtration and concentration, the crude product was used directly for the next step without further purification.

### Example 13: General Procedure to Synthesize 11a-b

To a round-bottomed flask equipped with a magnetic stir bar were added 10a-b (1.0 eq), 6 (1.2 eq), DIPEA (2.0 eq), and DMA. The reaction vessel was immersed in a 100° C. preheated oil bath for 12 h until the reaction was completed as determined by TLC. After cooling, the reaction mixture was diluted with water and extracted with ethyl acetate. The ethyl acetate layer was washed with 1 N HCl, saturated brine and dried over anhydrous MgSO₄. After filtration and concentration, the crude product was purified on a silica gel column, eluted with hexane/ethyl acetate to afford 11 a-b.

### Example 14: Synthesis of 4-(2-Ethylbutyl)-N-(3-(2-fluoroethoxy)-5-(4-fluorophenoxy)phenyl)-4-hydroxypiperidine-1-carboxamide (11a)

Compound 11a was eluted with hexane/ethyl acetate (2/3, V/V) as yellow oil. Yield: 97%. ¹H NMR (400 MHz, CDCl₃) δ 7.08-6.74 (m, 5H), 6.57 (s, 1H), 6.51 (s, 1H), 6.21 (s, 1H), 4.78-4.72 (m, 1H), 4.67-4.60 (m, 1H), 4.22-4.16 (m, 1H), 4.15-4.09 (m, 1H), 3.83-3.72 (m, 2H), 3.35-3.22 (m, 2H), 1.64-1.57 (m, 4H), 1.42-1.31 (m, 8H), 0.85 (t, J=6.7 Hz, 6H). ¹³C NMR (101 MHz, CDCl₃) δ 158.93 (d, J=243.4 Hz), 159.90, 159.04, 154.32, 152.31 (d, J=2.0 Hz), 141.31, 120.89 (d, J=8.1 Hz), 116.25 (d, J=24.2 Hz), 102.18, 100.41, 99.87, 81.74 (d, J=171.7 Hz), 70.17, 67.23 (d, J=21.2 Hz), 46.74, 40.59, 37.00, 35.37, 27.30, 10.81. HRMS (ESI) m/z [M+H]⁺ calcd. for C₂₆H₃₅F₂N₂O₄ 477.2559, found 477.2551.

### Example 15: Synthesis of 4-(2-Ethylbutyl)-N-(3-(4-fluorophenoxy)-5-(3-fluoropropoxy)phenyl)-4-hydroxypiperidine-1-carboxamide (11b)

Compound 11b was eluted with hexane/ethyl acetate (1/1, V/V) as yellow oil. Yield: 98%, yellow oil. ¹H NMR (400 MHz, CDCl₃) δ 7.10-6.95 (m, 4H), 6.92-6.89 (m, 1H), 6.47 (s, 1H), 6.36 (s, 1H), 6.19 (s, 1H), 4.66 (t, J=5.8 Hz, 1H), 4.54 (t, J=5.8 Hz, 1H), 4.04 (t, J=6.1 Hz, 2H), 3.78 (d, J=12.8 Hz, 2H), 3.33-3.24 (m, 2H), 2.18-2.06 (m, 2H), 1.63-1.58 (m, 4H), 1.41-1.34 (m, 7H), 1.08 (s, 1H), 0.85 (t, J=6.8 Hz, 6H). ¹³C NMR (101 MHz, CDCl₃) δ 160.28, 158.93, 158.85 (d, J=242.4 Hz), 154.47, 152.45 (d, J=2.0 Hz), 141.37, 120.77 (d, J=8.1 Hz), 116.21 (d, J=23.2 Hz), 101.95, 100.60, 99.58, 80.65 (d, J=164.6 Hz), 70.20, 63.63 (d, J=6.1 Hz), 46.75, 40.44, 37.05, 35.35, 30.26 (d, J=20.2 Hz), 27.29, 10.81. HRMS (ESI) m/z [M+H]⁺ calcd. for C₂₇H₃₇F₂N₂O₄ 491.2716, found 491.2709.

### Example 16: Synthetic Scheme of 17a-c

Compounds 17a-c and precursors were synthesized by Scheme 4.

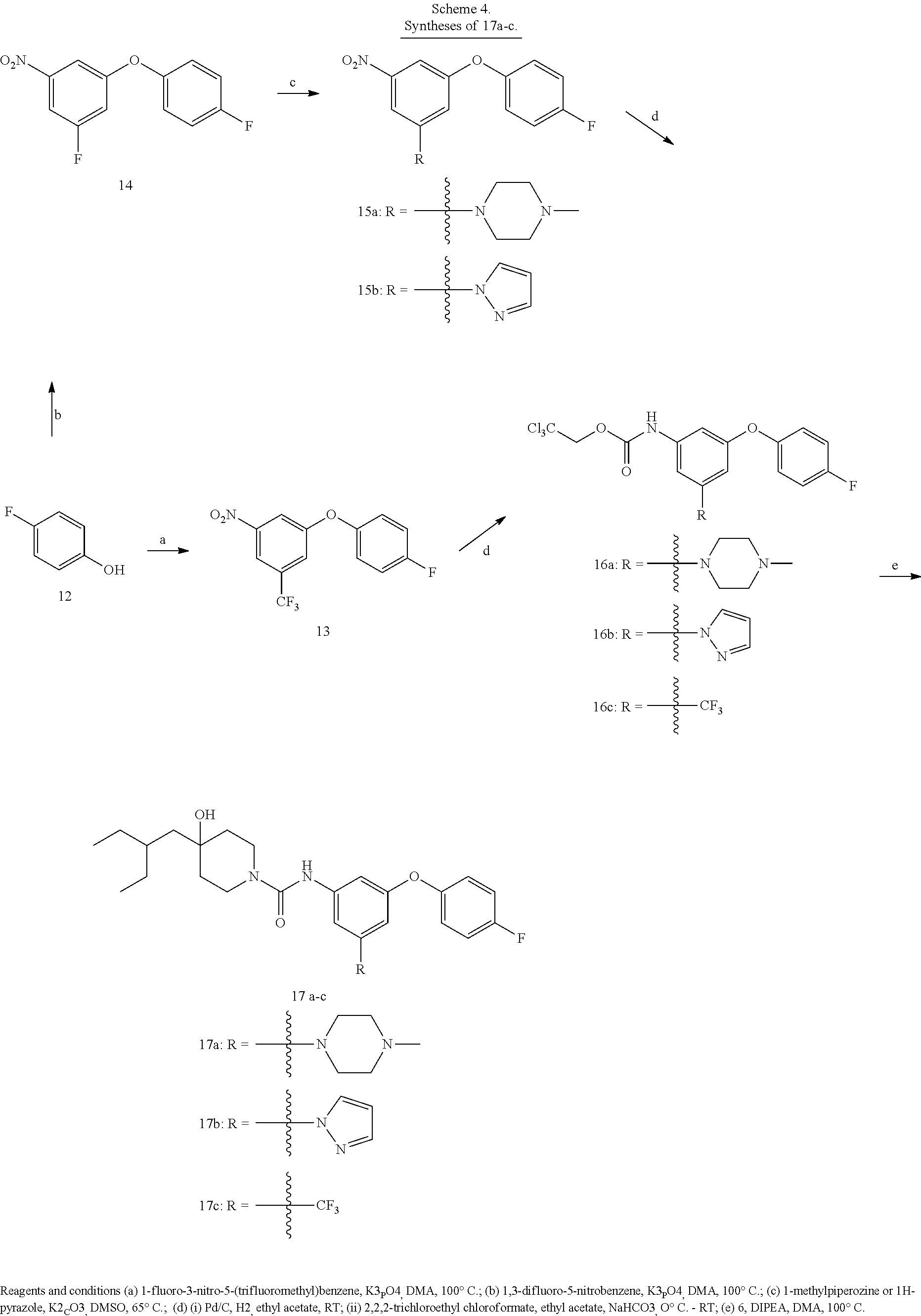

The syntheses of 17a-c were achieved by following Scheme 4. Commercially available 4-fluorophenol (12) reacted with 1-fluoro-3-nitro-5-(trifluoromethyl)benzene or 1,3-difluoro-5-nitrobenzene afforded 13 or 14, respectively. Intermediate 14 was coupled with 1-methylpiperozine or 1H-pyrazole using K₂CO₃ as a base in dimethyl sulfoxide (DMSO) to afford 15a or 15b. After palladium-catalyzed reduction of 13 and 15a-b, the resulting anilines were reacted with 2,2,2-trichloroethyl chloroformate to afford 16a-c. The target compounds 17a-c was prepared by reacting the carbamates 16a-c with 4-(2-ethylbutyl)piperidin-4-ol (6) as described for 11a-b.

### Example 17: Synthesis of 1-(4-Fluorophenoxy)-3-nitro-5-(trifluoromethyl)benzene (13)

To a round-bottomed flask equipped with a magnetic stir bar was added 1-fluoro-3-nitro-5-(trifluoromethyl)benzene (2.5 g, 12 mmol), 4-fluorophenol (12) (1.1 g, 10 mmol), potassium phosphate (4.2 g, 20 mmol), and DMA (15 mL). The reaction vessel was immersed in a 100° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was purified on a silica gel column, eluted with hexane/ethyl acetate (50/1, V/V) to afford 13. Yield: 93%. ¹H NMR (400 MHz, CDCl₃) δ 8.17 (s, 1H), 7.89 (t, J=2.1 Hz, 1H), 7.52 (s, 1H), 7.21-7.12 (m, 2H), 7.12-7.05 (m, 2H).

### Example 18: Synthesis of 1-fluoro-3-(4-fluorophenoxy)-5-nitrobenzene (14)

To a round-bottomed flask equipped with a magnetic stir bar were added 1,3-difluoro-5-nitrobenzene (7) (8.0 g, 50 mmol), 4-fluorophenol (12) (6.2 g, 55 mmol), Cs₂CO₃ (17.9 g, 55 mmol), and DMA (80 mL). The reaction vessel was immersed in a 65° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was pure enough for the next step. Yield: 95%. ¹H NMR (400 MHz, CDCl₃) δ 7.61 (d, J=8.1 Hz, 1H), 7.54 (s, 1H), 7.13-7.03 (m, 4H), 6.96 (d, J=9.2 Hz, 1H).

### Example 19: General Procedure to Synthesize 15a-b

To a round-bottomed flask equipped with a magnetic stir bar was added 14 (1.0 eq), 1-methylpiperazine or 1H-pyrazole (1.0 eq), K₂CO₃ (1.0 eq), and DMSO (2.0 M). The reaction vessel was immersed in a 65° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO4. After filtration and concentration, the crude product was purified on a silica gel column, eluted with hexane/ethyl acetate to afford 15a-b.

### Example 20: Synthesis of 1-(3-(4-Fluorophenoxy)-5-nitrophenyl)-4-methylpiperazine (15a)

Compound 15a was eluted with hexane/ethyl acetate (1/5, V/V) as yellow oil. Yield: 42%. ¹H NMR (400 MHz, CDCl₃) δ 7.46 (s, 1H), 7.14-6.97 (m, 5H), 6.79 (s, 1H), 3.34-3.22 (m, 4H), 2.61-2.51 (m, 4H), 2.36 (s, 3H).

### Example 21: Synthesis of 1-(3-(4-Fluorophenoxy)-5-nitrophenyl)-1H-pyrazole (15b)

Compound 15b was eluted with hexane/ethyl acetate (5/1, V/V) as yellow semi-solid. Yield: 53%. ¹H NMR (400 MHz, CDCl₃) δ 8.22 (s, 1H), 7.98 (d, J=2.4 Hz, 1H), 7.73 (d, J=12.4 Hz, 2H), 7.62 (s, 1H), 7.18-7.05 (m, 4H), 6.53 (s, 1H).

### Example 22: General Procedure to Synthesize 16a-c

To a round-bottomed flask equipped with a magnetic stir bar were added 13 or 15a-b (1.0 eq), 10% Pd/C, and ethyl acetate (0.2 M). The reaction was bubbled with hydrogen gas for 12 h at room temperature until the reaction was completed as determined by TLC. The mixture then was filtered through celite. To the filtrate was added NaHCO₃ (2.0 eq) followed by adding 2,2,2-trichloroethyl chloroformate (1.0 eq) slowly through syringe under nitrogen at 0° C. The reaction was warmed to room temperature and stirred for 3 h until the reaction was completed as determined by TLC. Then, the mixture was washed with water, saturated brine and dried over anhydrous MgSO₄. After filtration and concentration, the crude product was used directly for the next step without further purification.

### Example 23: General Procedure to Synthesize 17a-c

To a round-bottomed flask equipped with a magnetic stir bar were added 16a-c (1.0 eq), 6 (1.2 eq), DIPEA (2.0 eq) and DMA (0.5 M). The reaction vessel was immersed in a 100° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was purified on a silica gel column to afford 17a-c.

### Example 24: Synthesis of 4-(2-Ethylbutyl)-N-(3-(4-fluorophenoxy)-5-(4-methylpiperazin-1-yl)phenyl)-4-hydroxypiperidine-1-carboxamide (17a)

Compound 17a was eluted with ethyl acetate/methanol (5/1, V/V) as yellow oil. Yield: 20%, ¹H NMR (400 MHz, CDCl₃) δ 7.00-6.91 (m, 5H), 6.45 (s, 1H), 6.32 (s, 1H), 6.21 (s, 1H), 3.80-3.71 (m, 2H), 3.31-3.20 (m, 2H), 3.16 (s, 4H), 2.50 (s, 4H), 2.31 (s, 3H), 1.65-1.56 (m, 4H), 1.40-1.25 (m, 8H), 0.83 (t, J=6.8 Hz, 6H). ¹³C NMR (101 MHz, CDCl₃) δ 158.69, 158.68 (d, J=242.4), 154.78, 153.02 (d, J=2.02), 152.93, 141.33, 120.40 (d, J=8.08), 116.19 (d, J=23.2), 102.20, 100.93, 100.78, 70.22, 54.99, 48.58, 46.86, 46.11, 40.54, 37.14, 35.42, 27.37, 10.92. HRMS (ESI) m/z [M+H]⁺ calcd. for C₂₉H₄₂FN₄O₃ 513.3235, found 513.3231.

### Example 25: Synthesis of 4-(2-Ethylbutyl)-N-(3-(4-fluorophenoxy)-5-(1H-pyrazol-1-yl)phenyl)-4-hydroxypiperidine-1-carboxamide (17b)

Compound 17b was eluted with hexane/ethyl acetate (2/1, V/V) as yellow semi-solid. Yield: 30%, ¹H NMR (400 MHz, CDCl₃) δ 7.86 (s, 1H), 7.65 (s, 1H), 7.50 (s, 1H), 7.08-6.90 (m, 6H), 6.80 (s, 1H), 6.41 (s, 1H), 3.85-3.70 (m, 2H), 3.32-3.21 (m, 2H), 1.57 (s, 4H), 1.42-1.30 (m, 7H), 1.26 (s, 1H), 0.84 (t, J=6.8 Hz, 6H). ¹³C NMR (101 MHz, CDCl₃) δ 159.17 (d, J=243.4 Hz), 159.12, 154.56, 152.32 (d, J=3.0 Hz), 141.87, 141.52, 141.21, 127.21, 121.03 (d, J=8.8 Hz), 116.54 (d, J=23.2 Hz), 107.82, 107.19, 105.13, 103.44, 70.31, 46.89, 40.58, 37.18, 35.47, 27.43, 10.96. HRMS (ESI) m/z [M+H]⁺ calcd. for C₂₇H₃₄FN₄O₃ 481.2609, found 481.2603.

### Example 26: Synthesis of 4-(2-Ethylbutyl)-N-(3-(4-fluorophenoxy)-5-(trifluoromethyl)phenyl)-4-hydroxypiperidine-1-carboxamide (17c)

Compound 17c was eluted with hexane/ethyl acetate (2/1, V/V) as yellow solid. Yield: 44%, M.P. 108-111° C. ¹H NMR (400 MHz, CDCl₃) δ 7.23 (d, J=21.6 Hz, 2H), 7.04-6.84 (m, 5H), 6.75 (s, 1H), 3.84-3.67 (m, 2H), 3.32-3.12 (m, 2H), 1.81 (s, 1H), 1.62-1.44 (m, 4H), 1.37-1.21 (m, 7H), 0.90-0.72 (m, 6H). ¹³C NMR (101 MHz, CDCl₃) δ 159.18 (d, J=244.4 Hz), 158.54, 154.26, 151.73, 141.51, 132.12 (d, J=32.3 Hz), 123.55 (d, J=273.7 Hz), 121.04 (d, J=8.1 Hz), 116.53 (d, J=23.2 Hz), 112.04, 110.77, 108.63, 70.14, 46.73, 40.43, 37.01, 35.32, 27.27, 10.78. HRMS (ESI) m/z [M+Na]⁺ calcd. for C₂₅H₃₀F₄N₂NaO₃ 505.2090, found 505.2078.

### Example 27: Synthetic Scheme of 21a-f and Precursors

Compounds 21a-f and precursors were synthesized by Scheme 5.

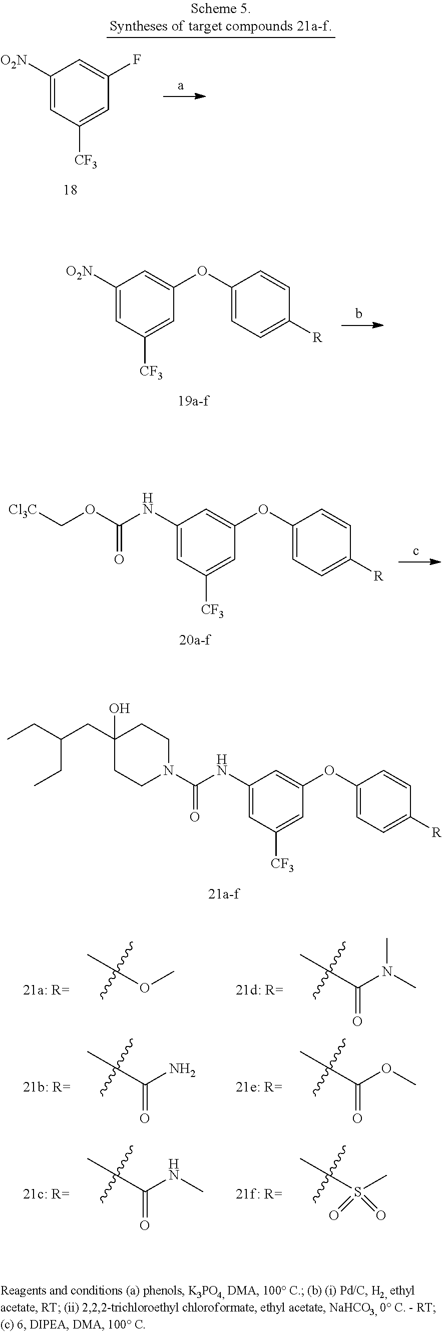

Compounds 21a-f were synthesized with different substitution groups at 4′-position as shown in Scheme 5. Briefly, commercially available 1-fluoro-3-nitro-5-(trifluoromethyl)benzene (18) reacted with differently substituted phenols in the presence of K₃PO₄ in DMA afforded 19a-f. The palladium-catalyzed hydrogenation of 19a-f gave anilines, followed by condensation with 2,2,2-trichloroethyl chloroformate to afford key intermediates 20a-f. Coupling of intermediates 20a-f with 4-(2-ethylbutyl)piperidin-4-ol (6) yielded the target compounds 21a-f.

### Example 28: General Procedure to Synthesize 19a-f

To a round-bottomed flask equipped with a magnetic stir bar was added 1-fluoro-3-nitro-5-(trifluoromethyl)benzene (18) (1.0 eq), 4-substituted phenol (1.0 eq), potassium phosphate (2 eq), and DMA (2.0 M). The reaction vessel was immersed in a 100° C. preheated oil bath for 12 h until the reaction was completed as determined by TLC. After cooling, the reaction was diluted with water and extracted with ethyl acetate. The organic layer was washed with saturated brine and dried over anhydrous MgSO₄. After filtration and concentration, the crude product was purified on a silica gel column, eluted with hexane/ethyl acetate to afford 19a-f.

### Example 29: Synthesis of 1-(4-Methoxyphenoxy)-3-nitro-5-(trifluoromethyl)benzene (19a)

Coupling of 1-fluoro-3-nitro-5-(trifluoromethyl)benzene (18) (1.0 g, 4.8 mmol) with 4-methoxyphenol (0.5 g, 4.0 mmol) yielded 19a (1.4 g, 99%), eluted with hexane/ethyl acetate (20/1, V/V). ¹H NMR (400 MHz, CDCl₃) δ 8.13 (s, 1H), 7.86 (s, 1H), 7.50 (s, 1H), 7.10-6.92 (m, 5H), 3.85 (s, 3H).

### Example 30: Synthesis of 4-(3-Nitro-5-(trifluoromethyl)phenoxy)benzamide (19b)

Coupling of 1-fluoro-3-nitro-5-(trifluoromethyl)benzene (18) (1.1 g, 5.0 mmol) with 4-hydroxybenzamide (0.7 g, 5.0 mmol) yielded 19b (1.6 g, 99%), eluted with hexane/ethyl acetate (1/2, V/V). ¹H NMR (400 MHz, CDCl₃) δ 8.18 (s, 1H), 7.93 (s, 1H), 7.85 (d, J=8.9 Hz, 2H), 7.53 (s, 1H), 7.08 (d, J=8.8 Hz, 2H).

### Example 31: Synthesis of N-methyl-4-(3-nitro-5-(trifluoromethyl)phenoxy)benzamide (19c)

Coupling of 1-fluoro-3-nitro-5-(trifluoromethyl)benzene (18) (1.8 g, 8.7 mmol) with 4-hydroxy-N-methylbenzamide (1.1 g, 7.3 mmol) yielded 19c (2.5 g, 99%), eluted with hexane/ethyl acetate (1/2, V/V). ¹H NMR (400 MHz, CDCl₃) δ 8.23 (s, 1H), 7.97 (s, 1H), 7.86 (d, J=8.8 Hz, 2H), 7.58 (s, 1H), 7.13 (d, J=8.9 Hz, 2H), 6.13 (s, 1H), 3.04 (d, J=4.9 Hz, 3H).

### Example 32: Synthesis of N, N-dimethyl-4-(3-nitro-5-(trifluoromethyl)phenoxy)benzamide (19d)

Coupling of 1-fluoro-3-nitro-5-(trifluoromethyl)benzene (18) (1.1 g, 5.0 mmol) with 4-hydroxy-N,N-dimethylbenzamide (0.8 g, 5.0 mmol) yielded 19d (1.8 g, 99%), eluted with hexane/ethyl acetate (1/1, V/V). ¹H NMR (400 MHz, CDCl₃) δ 8.22 (s, 1H), 7.98 (s, 1H), 7.60-7.51 (m, 3H), 7.15-7.08 (m, 2H), 3.09 (s, 6H).

### Example 33: Synthesis of Methyl 4-(3-nitro-5-(trifluoromethyl)phenoxy)benzoate (19e)

Coupling of 1-fluoro-3-nitro-5-(trifluoromethyl)benzene (18) (1.1 g, 5.0 mmol) with methyl-4-hydroxybenzoate (0.8 g, 5.0 mmol) yielded 19e (1.0 g, 60%), eluted with hexane/ethyl acetate (10/1, V/V). ¹H NMR (400 MHz, CDCl₃) δ 8.25 (s, 1H), 8.13 (d, J=8.2 Hz, 2H), 8.00 (s, 1H), 7.60 (s, 1H), 7.12 (d, J=8.9 Hz, 2H), 3.94 (s, 3H).

### Example 34: Synthesis of 1-(4-(Methylsulfonyl)phenoxy)-3-nitro-5-(trifluoromethyl)benzene (19f)

Coupling of 1-fluoro-3-nitro-5-(trifluoromethyl)benzene (18) (2.5 g, 12.0 mmol) with 4-(methylsulfonyl)phenol (1.7 g, 10.0 mmol) yielded 19f (2.8 g, 78%), eluted with hexane/ethyl acetate (3/2, V/V). ¹H NMR (400 MHz, CDCl₃) δ 8.33-8.30 (m, 1H), 8.07-8.01 (m, 3H), 7.67-7.63 (m, 1H), 7.26-7.20 (m, 2H), 3.11 (s, 3H).

### Example 35: General Procedure to Synthesize 20a-f

To a round-bottomed flask equipped with a magnetic stir bar were added 19a-f (1.0 eq), 10% Pd/C and ethyl acetate (0.2 M). The reaction was bubbled with hydrogen gas for 12 h at room temperature until the reaction was completed as determined by TLC. The mixture then was filtered through celite. To the filtrate was added NaHCO₃ (2.0 eq) followed by adding 2,2,2-trichloroethyl chloroformate (1.0 eq) slowly through syringe under nitrogen at 0° C. The reaction was warmed to room temperature and stirred for 3 h until the reaction was completed as determined by TLC. Then, the mixture was washed with water, saturated brine and dried over anhydrous MgSO₄. After filtration and concentration, the crude product was used directly without further purification.

### Example 36: General Procedure to Synthesize 21a-f

To a round-bottomed flask equipped with a magnetic stir bar were added 20a-f (1.0 eq), 6 (1.2 eq), DIPEA (2.0 eq), and DMA (1.0 M). The reaction vessel was immersed in a 100° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was purified on a silica gel column to afford 21a-f.

### Example 37: Synthesis of 4-(2-Ethylbutyl)-4-hydroxy-N-(3-(4-methoxyphenoxy)-5-(trifluoromethyl)phenyl)piperidine-1-carboxamide (21a)

Coupling of 20a (229 mg, 0.5 mmol) with 6 (86 mg, 0.6 mmol) in the presence of DIPEA afforded 21a (200 mg, 81%), eluted with hexane/ethyl acetate (1/1, V/V). White solid, M.P. 79-81° C. ¹H NMR (400 MHz, CDCl₃) δ 7.36 (s, 1H), 7.15 (s, 1H), 6.99 (d, J=9.0 Hz, 2H), 6.90 (d, J=9.0 Hz, 2H), 6.82 (s, 1H), 6.46 (s, 1H), 3.90-3.83 (m, 2H), 3.81 (s, 3H), 3.32-3.22 (m, 2H), 1.65-1.58 (m, 5H), 1.35-1.18 (m, 6H), 1.04 (s, 1H), 0.93 (d, J=6.9 Hz, 6H). ¹³C NMR (101 MHz, CDCl₃) 159.35, 156.33, 154.18, 149.02, 141.31, 132.07 (d, J=33.3 Hz), 123.60 (d, J=266.6 Hz), 121.13, 115.01, 111.32, 110.19, 108.23, 71.48, 55.57, 45.60, 40.34, 38.02, 33.75, 28.06, 11.34. HRMS (ESI) m/z [M+Na]⁺ calcd. for C₂₆H₃₃F₃N₂NaO₄ 517.2290, found 517.2299.

### Example 38: Synthesis of N-(3-(4-Carbamoylphenoxy)-5-(trifluoromethyl)phenyl)-4-(2-ethylbutyl)-4-hydroxypiperidine-1-carboxamide (21b)

Coupling of 20b (236 mg, 0.5 mmol) with 6 (95 mg, 0.6 mmol) in the presence of DIPEA afforded 21b (100 mg, 39%), eluted with ethyl acetate. White solid, M.P. 98-101° C. ¹H NMR (400 MHz, DMSO) δ 8.84 (s, 1H), 7.94 (d, J=8.3 Hz, 3H), 7.76 (s, 1H), 7.49 (s, 1H), 7.34 (s, 1H), 7.11 (d, J=8.2 Hz, 2H), 6.92 (s, 1H), 3.85-3.70 (m, 2H), 3.14 (t, J=11.6 Hz, 2H), 1.55-1.40 (m, 5H), 1.36-1.22 (m, 7H), 0.90 (d, J=6.3 Hz, 6H). ¹³C NMR (101 MHz, DMSO) δ 167.05, 158.32, 156.82, 154.08, 143.62, 130.52 (d, J=32.3 Hz), 129.92, 129.83, 123.81 (d, J=273.7 Hz), 118.38, 111.99, 110.55, 107.92, 68.38, 45.25, 42.06, 38.87, 34.95, 28.75, 11.30. HRMS (ESI) m/z [M+Na]⁺ calcd. for C₂₆H₃₂F₃N₃NaO₄ 530.2243, found 530.2251.

### Example 39: Synthesis of 4-(2-Ethylbutyl)-4-hydroxy-N-(3-(4-(methylcarbamoyl)-phenoxy)-5 (trifluoromethyl)phenyl)piperidine-1-carboxamide (21c)

Coupling of 20c (243 mg, 0.5 mmol) with 6 (112 mg, 0.6 mmol) in the presence of DIPEA afforded 21c (117 mg, 45%), eluted with hexane/ethyl acetate (1/7, V/V). White solid, M.P. 98-100° C. ¹H NMR (400 MHz, DMSO) δ 8.84 (s, 1H), 8.41 (d, J=4.4 Hz, 1H), 7.89 (d, J=8.6 Hz, 2H), 7.76 (s, 1H), 7.50 (s, 1H), 7.12 (d, J=8.6 Hz, 2H), 6.91 (s, 1H), 3.79 (d, J=13.1 Hz, 2H), 3.13 (t, J=11.4 Hz, 2H), 2.78 (d, J=4.4 Hz, 3H), 1.52-1.33 (m, 5H), 1.32-1.21 (m, 7H), 0.80 (t, J=7.3 Hz, 6H). ¹³C NMR (101 MHz, DMSO) δ 166.22, 158.58, 157.24, 154.48, 144.05, 130.94 (d, J=31.3 Hz), 130.58, 129.77, 124.24 (d, J=270.7 Hz), 118.85, 112.44, 110.97, 108.35, 68.87, 46.53, 37.13, 35.06, 31.09, 27.18, 26.67, 11.15. HRMS (ESI) m/z [M+Na]⁺ calcd. for C₂₇H₃₄F₃N₃NaO₄ 544.2399, found 544.2391.

### Example 40: Synthesis of N-(3-(4-(Dimethylcarbamoyl)phenoxy)-5-(trifluoromethyl)phenyl)-4-(2-ethylbutyl)-4-hydroxypiperidine-1-carboxamide (21d)

Coupling of 20d (250 mg, 0.5 mmol) with 6 (95 mg, 0.6 mmol) in the presence of DIPEA afforded 21d (180 mg, 67%), eluted with hexane/ethyl acetate (1/10, V/V). White solid, M.P. 82-84° C. ¹H NMR (400 MHz, CDCl₃) δ 7.82 (s, 1H), 7.56 (s, 1H), 7.33 (d, J=6.0 Hz, 2H), 7.13 (s, 1H), 6.97 (d, J=6.1 Hz, 2H), 6.89 (s, 1H), 3.90-3.65 (m, 2H), 3.30-2.86 (m, 8H), 1.56-1.44 (m, 6H), 1.40-1.30 (m, 5H), 1.25 (s, 1H), 0.93 (d, J=5.6 Hz, 6H). ¹³C NMR (101 MHz, CDCl₃) δ 171.43, 157.69, 157.31, 154.67, 142.48, 132.35 (d, J=33.3 Hz), 131.41, 129.12, 123.80 (d, J=273.7 Hz), 118.95, 112.28, 111.52, 109.57, 70.14, 45.07, 42.54, 37.27, 35.01, 34.62, 28.38, 11.62. HRMS (ESI) m/z [M+Na]⁺ calcd. for C₂₈H₃₆F₃N₃NaO₄ 558.2556, found 558.2661.

### Example 41: Synthesis of Methyl 4-(3-(4-(2-ethylbutyl)-4-hydroxypiperidine-1-carboxamido)-5-(trifluoromethyl)phenoxy)benzoate (21e)

Coupling of 20e (487 mg, 1.0 mmol) with 6 (189 mg, 1.2 mmol) in the presence of DIPEA afforded 21e (280 mg, 54%), eluted with hexane/ethyl acetate (1/1, V/V). White solid, M.P. 80-82° C. ¹H NMR (400 MHz, CDCl₃) δ 7.68 (d, J=8.3 Hz, 2H), 7.42 (s, 1H), 7.00 (d, J=8.3 Hz, 2H), 6.93 (s, 2H), 6.31 (s, 1H), 3.93-3.83 (m, 2H), 3.30-3.18 (m, 2H), 2.98 (s, 3H), 1.60-1.54 (m, 5H), 1.30-1.24 (m, 6H), 1.10 (s, 1H), 0.92 (d, J=6.8 Hz, 6H). ¹³C NMR (101 MHz, CDCl₃) δ 166.51, 160.64, 156.63, 154.08, 141.72, 132.49 (d, J=33.3 Hz), 131.79, 125.27, 123.44 (d, J=272.7 Hz), 117.92, 113.65, 111.88, 110.46, 70.13, 52.07, 45.97, 42.47, 37.10, 34.83, 27.28, 11.02. HRMS (ESI) m/z [M+Na]⁺ calcd. for C₂₇H₃₃F₃N₂NaO₅ 545.2239, found 545.2230.

### Example 42: Synthesis of 4-(2-Ethylbutyl)-4-hydroxy-N-(3-(4-(methylsulfonyl)phenoxy)-5-(trifluoromethyl)phenyl)piperidine-1-carboxamide (21f)

Coupling of 20f (253 mg, 0.5 mmol) with 6e (112 mg, 0.6 mmol) in the presence of DIPEA afforded 21f (160 mg, 59%), eluted with hexane/ethyl acetate (1/2, V/V). White solid, M.P. 86-88° C. ¹H NMR (400 MHz, CDCl₃) δ 7.79 (d, J=7.9 Hz, 2H), 7.47-7.36 (m, 2H), 7.22-7.16 (m, 1H), 7.01 (d, J=7.9 Hz, 2H), 6.85 (s, 1H), 3.83-3.65 (m, 2H), 3.25-3.10 (m, 2H), 2.98 (s, 3H), 1.56-1.37 (m, 5H), 1.36-1.19 (m, 7H), 0.76 (t, J=6.9 Hz, 6H). ¹³C NMR (101 MHz, CDCl₃) δ 161.52, 155.62, 154.22, 142.24, 134.54, 132.48 (d, J=32.3 Hz), 129.73, 123.39 (d, J=273.7 Hz), 118.23, 114.22, 112.59, 110.56, 70.07, 46.71, 44.71, 40.42, 37.00, 35.29, 27.25, 10.79. HRMS (ESI) m/z [M+Na]⁺ calcd. for C₂₆H₃₃F₃N₂NaO₅S 565.1960, found 565.1944.

### Example 43: Synthetic Scheme of 22a-d, 24a-b, 25a-j, and Precursors

Compounds 22a-d, 24a-b, 25a-j, and precursors were synthesized by Scheme 6.

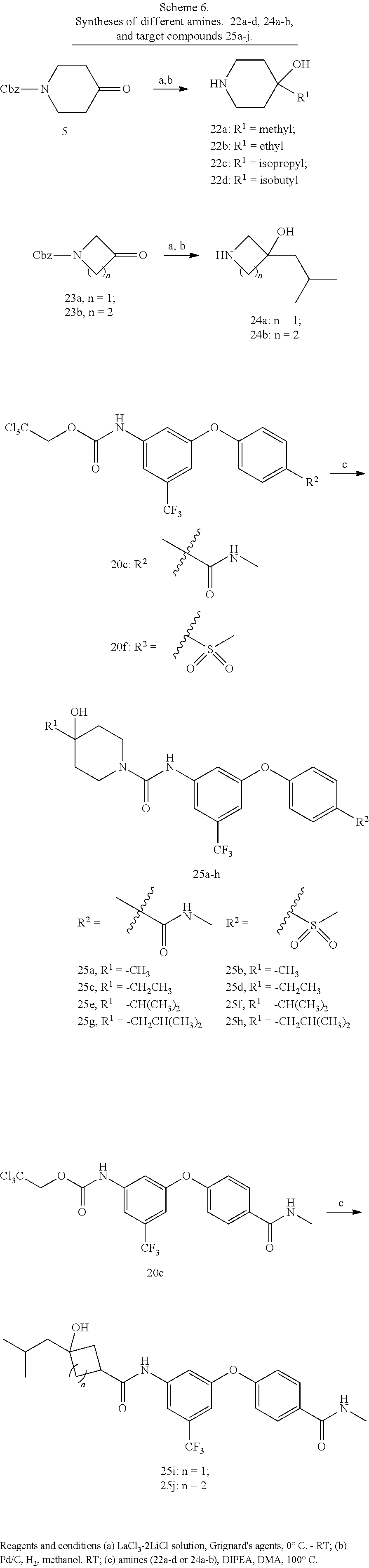

Compounds 25a-j were synthesized by following Scheme 6. The amines (22a-d and 24a-b) were prepared ahead using the same procedure as described for compound 6 with corresponding Grignard's agents and ketones. The subsequent coupling two carbamates 20c and 20f with corresponding amines (22a-d and 24a-b) afforded compounds 25a-j.

### Example 44: General Procedure to Synthesize 22a-d and 24a-b

To a dried round two neck bottomed flask equipped with a magnetic stir bar were added 0.6 M LaCl₃-2LiCl in THF (1.5 eq) under nitrogen, Grignard reagents in THF (1.5 eq) was added slowly through syringe at 0° C. After stirring at room temperature for 3 h, a solution of ketones (5 or 23a-b) (1.0 eq) in THF (1.0 M) was added into the mixture. The reaction was stirred for another 18 h until the reaction was completed as determined by TLC and then quenched with 25% acetic acid. The mixture was extracted with ethyl acetate, the ethyl acetate layer was washed with saturated brine and dried over anhydrous MgSO₄. After filtration and concentration, the crude product was used directly for the next step without purification.

To a round-bottomed flask equipped with a magnetic stir bar were added above crude product, 10% Pd/C and methanol (0.5 M). The reaction was bubbled with hydrogen gas for 6 h at room temperature until the reaction was completed as determined by TLC and then filtered through celite. The filtrate was concentrated under reduced pressure to afford yellow oil product 22a-d and 24a-b.

### Example 45: Synthesis of 4-Methylpiperidin-4-ol (22a)

Yield: 93%. ¹H NMR (400 MHz, CDCl₃) δ 5.86 (s, 1H), 3.03-2.87 (m, 2H), 2.48-2.34 (m, 2H), 1.68-1.55 (m, 4H), 1.26 (s, 3H).

### Example 46: Synthesis of 4-Ethylpiperidin-4-ol (22b)

Yield: 98%. ¹H NMR (400 MHz, CDCl₃) δ 5.82 (s, 1H), 3.00-2.86 (m, 2H), 2.44-2.23 (m, 2H), 1.72-1.39 (m, 7H), 0.95-0.80 (m, 3H).

### Example 47: Synthesis of 4-Isopropylpiperidin-4-ol (22c)

Yield: 97%. ¹H NMR (400 MHz, CDCl₃) δ 3.06-2.84 (m, 4H), 1.67-1.51 (m, 5H), 0.90 (d, J=6.9 Hz, 6H).

### Example 48: Synthesis of 4-Isobutylpiperidin-4-ol (22d)

Yield: 95%. ¹H NMR (400 MHz, CDCl₃) δ 3.44 (s, 1H), 3.38-3.17 (m, 4H), 1.88-1.76 (m, 4H), 1.48-1.40 (m, 2H), 1.24-1.17 (m, 2H), 0.95 (d, J=6.1 Hz, 6H).

### Example 49: Synthesis of 3-Isobutylazetidin-3-ol (24a)

Yield: 93%. ¹H NMR (400 MHz, CDCl₃) δ 3.95-3.87 (m, 2H), 3.75-3.65 (m, 2H), 1.96-1.82 (m, 1H), 1.75-1.58 (m, 2H), 0.91 (d, J=5.8 Hz, 6H).

### Example 50: Synthesis of 3-Isobutylpyrrolidin-3-ol (24b)

Yield: 92%. ¹H NMR (400 MHz, CDCl₃) δ 3.75-3.41 (m, 4H), 2.12-1.78 (m, 4H), 1.71-1.49 (m, 3H), 1.02-0.91 (m, 6H).

### Example 51: General Procedure to Synthesize 25a-j

To a round-bottomed flask equipped with a magnetic stir bar were added 20c or 20f (1.0 eq), 22a-d or 24a-b (1.2 eq), DIPEA (2.0 eq), and DMA (1.0 M). The reaction vessel was immersed in a 100° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was purified on a silica gel column to afford 25a-j.

### Example 52: Synthesis of 4-Hydroxy-4-methyl-N-(3-(4-(methylcarbamoyl)phenoxy)-5-(trifluoromethyl)phenyl)piperidine-1-carboxamide (25a)

Coupling of 20c (243 mg, 0.5 mmol) with 22a (69 mg, 0.6 mmol) in the presence of DIPEA afforded 25a (160 mg, 62%), eluted with hexane/ethyl acetate (1/5, V/V). White solid, M.P. 103-106° C. ¹H NMR (400 MHz, CDCl₃) δ 7.91 (d, J=8.8 Hz, 2H), 7.50 (s, 1H), 7.40 (s, 1H), 7.27 (s, 1H), 7.12 (d, J=8.7 Hz, 2H), 6.98 (s, 1H), 6.61 (s, 1H), 3.79-3.72 (m, 2H), 3.42-3.32 (m, 2H), 3.06 (s, 3H), 1.67-1.61 (m, 4H), 1.31 (s, 3H). ¹³C NMR (101 MHz, DMSO) δ 165.80, 158.15, 156.81, 154.07, 143.60, 130.51 (d, J=32.3 Hz), 130.16, 129.33, 123.79 (d, J=273.7 Hz), 118.42, 112.05, 110.59, 107.92, 66.02, 40.34, 38.21, 29.70, 26.23. HRMS (ESI) m/z [M+Na]⁺ calcd. for C₂₂H₂₅F₃N₃O₄ 452.1792, found 452.1787.

### Example 53: Synthesis of 4-Hydroxy-4-methyl-N-(3-(4-(methylsulfonyl)phenoxy)-5-(trifluoromethyl)phenyl)piperidine-1-carboxamide (25b)

Coupling of 20f (253 mg, 0.5 mmol) with 22a (69 mg, 0.6 mmol) in the presence of DIPEA afforded 25b (160 mg, 68%), eluted with hexane/ethyl acetate (1/2, V/V). White solid, M.P. 94-96° C. ¹H NMR (400 MHz, CDCl₃) δ 7.68 (d, J=7.4 Hz, 2H), 7.42 (s, 1H), 7.03-6.91 (m, 4H), 6.32 (s, 1H), 3.84-3.69 (m, 2H), 3.41-3.25 (m, 2H), 2.98 (s, 3H), 1.63-1.56 (m, 5H), 1.28 (s, 3H). ¹³C NMR (101 MHz, CDCl₃) δ 161.50, 155.71, 154.15, 142.11, 134.66, 132.59 (d, J=32.3 Hz), 129.74, 123.37 (d, J=273.7 Hz), 118.26, 114.14, 112.54, 110.71, 67.66, 44.67, 40.71, 38.25, 30.18. HRMS (ESI) m/z [M+H]⁺ calcd. for C₂₁H₂₄F₃N₂O₅S 473.1353, found 473.1345.

### Example 54: Synthesis of 4-Ethyl-4-hydroxy-N-(3-(4-(methylcarbamoyl)phenoxy)-5-(trifluoromethyl)phenyl)piperidine-1-carboxamide (25c)

Coupling of 20c (243 mg, 0.5 mmol) with 22b (78 mg, 0.6 mmol) in the presence of DIPEA afforded 25c (160 mg, 69%), eluted with ethyl acetate. White solid, M.P. 104-107° C. ¹H NMR (400 MHz, CDCl₃) δ 7.70 (d, J=8.5 Hz, 2H), 7.42 (s, 1H), 7.28 (s, 1H), 7.01 (d, J=8.4 Hz, 2H), 6.94 (s, 1H), 6.84 (s, 1H), 6.24 (s, 1H), 3.86-3.76 (m, 2H), 3.35-3.24 (m, 2H), 2.99 (d, J=4.8 Hz, 3H), 1.61-1.53 (m, 6H), 1.12 (s, 1H), 0.93 (t, J=7.5 Hz, 3H). ¹³C NMR (101 MHz, DMSO) δ 165.79, 158.14, 156.81, 154.06, 143.61, 130.50 (d, J=32.3 Hz), 130.16, 129.33, 123.79 (d, J=273.7 Hz), 118.42, 112.04, 110.61, 107.89, 67.83, 40.05, 35.85, 34.78, 26.22, 7.14. HRMS (ESI) m/z [M+H]⁺ calcd. for C₂₃H₂₇F₃N₃O₄ 466.1948, found 466.1939.

### Example 55: Synthesis of 4-Ethyl-4-hydroxy-N-(3-(4-(methylsulfonyl)phenoxy)-5-(trifluoromethyl)phenyl)piperidine-1-carboxamide (25d)

Coupling of 20f (253 mg, 0.5 mmol) with 22b (78 mg, 0.6 mmol) in the presence of DIPEA afforded 25d (240 mg, 99%), eluted with hexane/ethyl acetate (1/2, V/V). White solid, M.P. 89-91° C. ¹H NMR (400 MHz, CDCl₃) δ 7.91 (d, J=8.7 Hz, 2H), 7.50 (s, 1H), 7.41 (s, 1H), 7.12 (d, J=8.7 Hz, 2H), 6.97 (s, 1H), 6.66 (s, 1H), 3.86-3.75 (m, 2H), 3.38-3.27 (m, 2H), 3.06 (s, 3H), 1.65-1.54 (m, 6H), 1.11 (s, 1H), 0.94 (t, J=7.5 Hz, 3H). ¹³C NMR (101 MHz, CDCl₃) δ 161.51, 155.68, 154.16, 142.17, 134.64, 132.56 (d, J=33.3 Hz), 129.73, 123.38 (d, J=273.7 Hz), 118.23, 114.15, 112.56, 110.68, 110.67, 44.67, 40.45, 36.04, 35.52, 6.96. HRMS (ESI) m/z [M+H]⁺ calcd. for C₂₂H₂₆F₃N₂O₅S 487.1509, found 487.1502.

### Example 56: Synthesis of 4-Hydroxy-4-isopropyl-N-(3-(4-(methylcarbamoyl)phenoxy)-5-(trifluoromethyl)phenyl)piperidine-1-carboxamide (25e)

Coupling of 20c (243 mg, 0.5 mmol) with 22c (86 mg, 0.6 mmol) in the presence of DIPEA afforded 25e (108 mg, 44%), eluted with ethyl acetate. White solid, M.P. 100-102° C. ¹H NMR (400 MHz, CDCl₃) δ 8.03 (d, J=8.5 Hz, 2H), 7.40 (d, J=11.8 Hz, 2H), 7.26 (s, 1H), 7.02 (d, J=8.5 Hz, 2H), 6.95 (s, 1H), 6.52 (s, 1H), 3.91 (s, 3H), 3.83-3.76 (m, 2H), 3.38-3.28 (m, 2H), 1.90-1.80 (m, 1H), 1.65-1.62 (m, 2H), 1.45-1.41 (m, 2H), 1.06 (s, 1H), 0.98 (d, J=6.6 Hz, 6H). ¹³C NMR (101 MHz, CDCl₃) δ 168.00, 158.89, 157.12, 154.51, 141.94, 132.28 (d, J=33.3 Hz), 130.07, 128.89, 123.53 (d, J=374.7 Hz), 118.55, 112.60, 111.58, 109.88, 71.42, 40.25, 38.00, 33.77, 26.75, 16.33. HRMS (ESI) m/z [M+H]⁺ calcd. for C₂₄H₂₉F₃N₃O₄ 480.2105, found 480.2097.

### Example 57: Synthesis of 4-Hydroxy-4-isopropyl-N-(3-(4-(methylsulfonyl)phenoxy)-5-(trifluoromethyl)phenyl)piperidine-1-carboxamide (25f)

Coupling of 20f (253 mg, 0.5 mmol) with 22c (86 mg, 0.6 mmol) in the presence of DIPEA afforded 25f (200 mg, 80%), eluted with hexane/ethyl acetate (1/2, V/V). White solid, M.P. 92-104° C. ¹H NMR (400 MHz, CDCl₃) δ 7.91 (d, J=8.8 Hz, 2H), 7.50 (s, 1H), 7.41 (s, 1H), 7.12 (d, J=8.8 Hz, 2H), 6.97 (s, 1H), 6.63 (s, 1H), 3.91-3.84 (m, 2H), 3.32-3.23 (m, 2H), 3.06 (s, 3H), 1.66-1.58 (m, 5H), 1.06 (s, 1H), 0.93 (d, J=6.9 Hz, 6H). ¹³C NMR (101 MHz, CDCl₃) δ 161.50, 155.71, 154.08, 142.14, 134.68, 132.59 (d, J=33.3 Hz), 129.74, 123.38 (d, J=271.7 Hz), 118.23, 114.12, 112.48, 110.69, 71.43, 44.67, 40.34, 38.02, 33.75, 16.36. HRMS (ESI) m/z [M+H]⁺ calcd. for C₂₃H₂₈F₃N₂O₅S 501.1659, found 501.1666.

### Example 58: Synthesis of 4-Hydroxy-4-isobutyl-N-(3-(4-(methylcarbamoyl)phenoxy)-5-(trifluoromethyl)phenyl)piperidine-1-carboxamide (25g)

Coupling of 20c (243 mg, 0.5 mmol) with 22d (94 mg, 0.6 mmol) in the presence of DIPEA afforded 25g (159 mg, 64%), eluted with hexane/ethyl acetate (1/7, V/V). White solid, M.P. 190-192° C. ¹H NMR (400 MHz, DMSO) 8.80 (s, 1H), 8.38 (s, 1H), 7.86 (d, J=7.5 Hz, 2H), 7.72 (s, 1H), 7.47 (s, 1H), 7.09 (d, J=7.4 Hz, 2H), 6.88 (s, 1H), 3.85-3.65 (m, 2H), 3.20-3.00 (m, 2H), 2.75 (s, 3H), 2.47 (s, 1H), 1.85-1.70 (m, 1H), 1.52-1.21 (m, 6H), 0.87 (d, J=5.5 Hz, 6H). ¹³C NMR (101 MHz, DMSO) δ 166.21, 158.57, 157.24, 154.50, 147.82, 144.04, 140.77, 131.09, 130.58, 129.77, 118.86, 112.45, 109.69 (d, J=267.7 Hz), 68.79, 51.67, 40.48, 37.29, 26.67, 25.37, 23.17. HRMS (ESI) m/z [M+Na]⁺ calcd. for C₂₅H₃₀F₃N₃NaO₄ 516.2086, found 516.2069.

### Example 59: Synthesis of 4-Hydroxy-4-isobutyl-N-(3-(4-(methylsulfonyl)phenoxy)-5-(trifluoromethyl)phenyl)piperidine-1-carboxamide (25h)

Coupling of 20f (253 mg, 0.5 mmol) with 22d (94 mg, 0.6 mmol) in the presence of DIPEA afforded 25h (210 mg, 82%), eluted with hexane/ethyl acetate (1/2, V/V). White solid, M.P. 87-90° C. ¹H NMR (400 MHz, CDCl₃) δ 7.90 (s, 2H), 7.50 (s, 1H), 7.42 (s, 1H), 7.12 (s, 2H), 6.97 (s, 1H), 6.72 (s, 1H), 3.87-3.72 (m, 2H), 3.41-3.24 (m, 2H), 3.06 (s, 3H), 1.94-1.70 (m, 1H), 1.71-1.40 (m, 7H), 1.07-0.90 (m, 6H). ¹³C NMR (101 MHz, CDCl₃) δ 161.52, 155.64, 154.20, 142.21, 134.58, 132.51 (d, J=32.3 Hz), 129.73, 123.38 (d, J=275.7 Hz), 118.22, 114.17, 112.55, 110.60, 70.03, 51.89, 44.67, 40.42, 37.05, 24.82, 23.24. HRMS (ESI) m/z [M+Na]⁺ calcd. for C₂₄H₂₉F₃N₂NaO₅S 537.1647, found 537.1641.

### Example 60: Synthesis of 3-Hydroxy-3-isobutyl-N-(3-(4-(methylcarbamoyl)phenoxy)-5-(trifluoromethyl)phenyl)azetidine-1-carboxamide (25i)

Coupling of 20c (243 mg, 0.5 mmol) with 24a (78 mg, 0.6 mmol) in the presence of DIPEA afforded 25i (110 mg, 47%), eluted with hexane/ethyl acetate (1/5, V/V). White solid, M.P. 107-109° C. ¹H NMR (400 MHz, DMSO) δ 8.80 (s, 1H), 8.42 (s, 1H), 7.90 (d, J=7.4 Hz, 2H), 7.77 (s, 1H), 7.52 (s, 1H), 7.12 (d, J=7.5 Hz, 2H), 6.92 (s, 1H), 3.95-3.65 (m, 4H), 2.78 (s, 3H), 1.91-1.77 (m, 1H), 1.65-1.45 (m, 2H), 1.22 (s, 1H), 0.88 (d, J=5.3 Hz, 6H). ¹³C NMR (101 MHz, DMSO) 166.22, 158.46, 157.45, 156.37, 143.47, 131.11 (d, J=32.3 Hz), 130.68, 129.79, 124.19 (d, J=273.7 Hz), 118.98, 111.63, 110.24, 108.36, 69.11, 63.86, 47.30, 26.67, 24.52, 23.50. HRMS (ESI) m/z [M+H]⁺ calcd. for C₂₃H₂₇F₃N₃O₄ 466.1948, found 466.1941.

### Example 61: Synthesis of 3-Hydroxy-3-isobutyl-N-(3-(4-(methylcarbamoyl)phenoxy)-5-(trifluoromethyl)phenyl)pyrrolidine-1-carboxamide (25j)

Coupling of 20c (243 mg, 0.5 mmol) with 24b (86 mg, 0.6 mmol) in the presence of DIPEA afforded 25j (100 mg, 42%), eluted with hexane/ethyl acetate (1/5, V/V). White solid, M.P. 113-116° C. ¹H NMR (400 MHz, DMSO) δ 8.48 (d, J=45.8 Hz, 2H), 8.11-7.43 (m, 4H), 7.13 (s, 2H), 6.93 (s, 1H), 3.55-3.40 (m, 2H), 2.90-2.67 (m, 2H), 2.10-1.68 (m, 3H), 1.60-1.37 (m, 2H), 1.24 (s, 1H), 0.93 (s, 6H). ¹³C NMR (101 MHz, DMSO) δ 165.81, 158.17, 156.81, 153.43, 143.45, 130.52 (d, J=32.3 Hz), 130.15, 129.35, 123.82 (d, J=271.7 Hz), 118.41, 111.94, 110.49, 107.89, 58.16, 47.16, 45.37, 44.48, 26.25, 24.37, 24.18, 24.13. HRMS (ESI) m/z [M+H]⁺ calcd. for C₂₄H₂₉F₃N₃O₄ 480.2105, found 480.2097.

### Example 62: Synthetic Scheme of 28a-g and Precursors

Compounds 28a-g and precursors were synthesized by scheme 7.

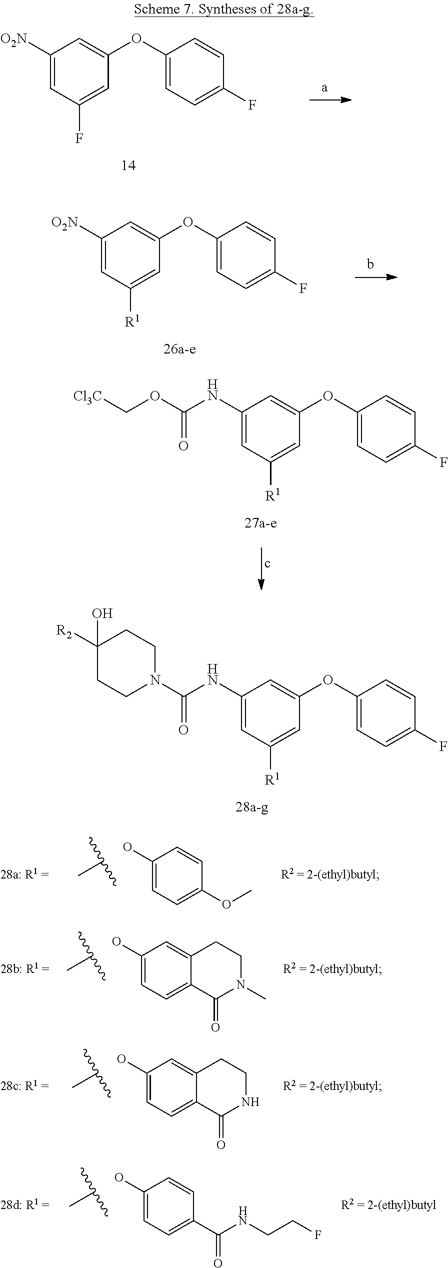

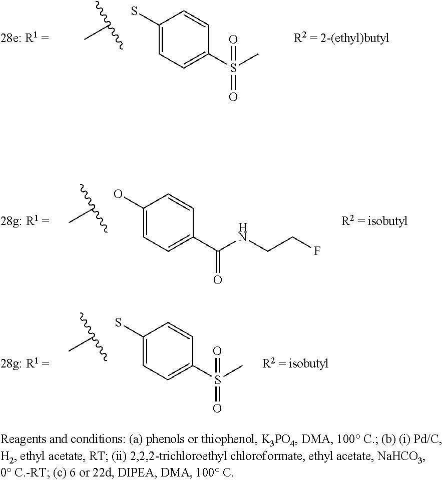

Analogues 28a-g were synthesized by following Scheme 7. Briefly, compound 14 reacted with 4-methoxylphenol, 6-hydroxy-2-methyl-3,4-dihydroisoquinolin-1(2H)-one, 6-hydroxy-3,4-dihydroisoquinolin-1(2H)-one, N-(2-fluoroethyl)-4-hydroxybenzamide, or 4-(methylsulfonyl)benzene-thiol in the presence of K₃PO₄ gave compounds 26a-e, followed by palladium-catalyzed hydrogenation afforded corresponding anilines. The resulting anilines were treated with 2,2,2-trichloroethyl chloroformate and NaHCO₃ to afford intermediates 27a-e. The subsequent condensation reaction of 27a-e with 6 or 22d in the presence of DIPEA at 100° C. in DMA yielded target compounds 28a-g.

### Example 63: General Procedure to Synthesize 26a-e

To a round-bottomed flask equipped with a magnetic stir bar was added 14 (1.0 eq), corresponding phenols (1.0 eq), K₃PO₄ (2.0 eq) and DMA (1.0 M). The reaction vessel was immersed in a 100° C. preheated oil bath for 12 h until the reaction was completed as determined by TLC. After cooling, the reaction was diluted with water and extracted with ethyl acetate. The organic layer was washed with saturated brine and dried over anhydrous MgSO₄. After filtration and concentration, the crude product was purified on a silica gel column, eluted with hexane/ethyl acetate to afford 26a-e.

### Example 64: Synthesis of 1-(4-Fluorophenoxy)-3-(4-methoxyphenoxy)-5-nitrobenzene (26a)

Compound 26a was eluted with hexane/ethyl acetate (10/1, V/V). Yield: 93%. ¹H NMR (400 MHz, CDCl₃) δ 7.34 (dd, J=4.8, 2.1 Hz, 2H), 7.12-6.96 (m, 6H), 6.96-6.89 (m, 2H), 6.88-6.84 (m, 1H), 3.80 (s, 3H).

### Example 65: Synthesis of 6-(3-(4-Fluorophenoxy)-5-nitrophenoxy)-2-methyl-3,4-dihydroisoquinolin-1(2H)-one (26b)

Compound 26b was pure enough. Yield: 99%. ¹H NMR (400 MHz, CDCl₃) δ 8.12 (d, J=8.6 Hz, 1H), 7.49 (d, J=12.3 Hz, 1H), 7.24 (s, 1H), 7.17-6.90 (m, 6H), 6.83 (s, 1H), 3.59 (t, J=6.6 Hz, 2H), 3.16 (s, 3H), 3.00 (t, J=6.6 Hz, 2H).

### Example 66: Synthesis of 6-(3-(4-Fluorophenoxy)-5-nitrophenoxy)-3,4-dihydroisoquinolin-1(2H)-one (26c)

Compound 26c was pure enough. Yield: 40%. ¹H NMR (400 MHz, CDCl₃) δ 8.11 (s, 1H), 7.74-7.35 (m, 2H), 7.19-6.78 (m, 6H), 6.65 (s, 1H), 3.74-3.47 (m, 2H), 3.11-2.84 (m, 2H).

### Example 67: Synthesis of N-(2-Fluoroethyl)-4-(3-(4-fluorophenoxy)-5-nitrophenoxy)benzamide (26d)

Compound 26d was eluted with hexane/ethyl acetate (3/2, V/V). Yield: 31%. ¹H NMR (400 MHz, CDCl₃) δ 7.85 (d, J=8.7 Hz, 2H), 7.51-7.45 (m, 2H), 7.14-7.10 (m, 4H), 7.08-7.04 (m, 2H), 6.95 (t, J=2.2 Hz, 1H), 6.48 (s, 1H), 4.68 (t, J=4.7 Hz, 1H), 4.56 (t, J=4.7 Hz, 1H), 3.83 (dd, J=10.2, 5.1 Hz, 1H), 3.76 (dd, J=10.2, 5.1 Hz, 1H).

### Example 68: Synthesis of (3-(4-Fluorophenoxy)-5-nitrophenyl)(4-(methylsulfonyl-)phenyl)sulfane (26e)

Compound 26e was eluted with hexane/ethyl acetate (2/1, V/V). Yield: 15%. ¹H NMR (400 MHz, CDCl₃) δ 7.92-7.88 (m, 3H), 7.65 (s, 1H), 7.47 (d, J=8.3 Hz, 2H), 7.28 (s, 1H), 7.16-7.02 (m, 5H), 3.08 (s, 3H).

### Example 69: General Procedure to Synthesize 27a-e

To a round-bottomed flask equipped with a magnetic stir bar were added 26a-e (1.0 eq), 10% Pd/C and ethyl acetate. The reaction was bubbled with hydrogen gas for 16 h at room temperature until the reaction was completed as determined by TLC. The mixture then was filtered through celite. To the filtrate was added NaHCO₃ (2.0 eq) followed by adding 2,2,2-trichloroethyl chloroformate (1.0 eq) slowly through syringe under nitrogen at 0° C. The reaction was warmed to room temperature and stirred for 3 h until the reaction was completed as determined by TLC. Then, the mixture was washed with water, saturated brine and dried over anhydrous MgSO₄. After filtration and concentration, the crude product was directly used without purification.

### Example 70: Synthesis of 2,2,2-Trichloroethyl (3-(4-fluorophenoxy)-5-(4-methoxyphenoxy)phenyl)carbamate (27a)

Yield: 96%. ¹H NMR (400 MHz, CDCl₃) δ 7.05-6.97 (m, 6H), 6.90-6.87 (m, 2H), 6.79 (s, 1H), 6.76 (s, 1H), 6.67 (s, 1H), 6.32 (t, J=2.2 Hz, 1H), 4.76 (s, 2H), 3.80 (s, 3H).

### Example 71: Synthesis of 2,2,2-Trichloroethyl (3-(4-fluorophenoxy)-5-((2-methyl-1-oxo-1,2,3,4-tetrahydroisoquinolin-6-yl)oxy)phenyl)carbamate (27b)

Yield: 96%, ¹H NMR (400 MHz, CDCl₃) δ 8.04 (d, J=8.5 Hz, 1H), 7.24 (s, 1H), 7.14-6.96 (m, 4H), 6.96-6.82 (m, 3H), 6.81-6.75 (m, 1H), 6.39 (s, 1H), 4.77 (s, 2H), 3.55 (t, J=6.6 Hz, 2H), 3.14 (s, 3H), 2.96 (t, J=6.5 Hz, 2H).

### Example 72: Synthesis of 2,2,2-Trichloroethyl (3-(4-fluorophenoxy)-5-((1-oxo-1,2,3,4-tetrahydroisoquinolin-6-yl)oxy)phenyl)carbamate (27c)

Yield: 95%, ¹H NMR (400 MHz, CDCl₃) δ 8.05-7.99 (m, 1H), 7.16 (s, 1H), 7.09-7.00 (m, 4H), 6.96-6.91 (m, 1H), 6.91-6.86 (m, 2H), 6.84-6.79 (m, 1H), 6.41 (s, 1H), 6.13 (s, 1H), 4.78 (s, 2H), 3.60-3.51 (m, 2H), 2.96 (t, J=6.1 Hz, 2H).

### Example 73: Synthesis of 2,2,2-Trichloroethyl (3-(4-((2-fluoroethyl)carbamoyl)phenoxy)-5-(4-fluorophenoxy)phenyl)carbamate (27d)

Yield: 90%. ¹H NMR (400 MHz, CDCl₃) δ 7.77 (d, J=8.7 Hz, 2H), 7.10-6.97 (m, 7H), 6.88 (s, 1H), 6.83 (s, 1H), 6.45 (s, 1H), 6.39 (t, J=2.1 Hz, 1H), 4.77 (s, 2H), 4.66 (t, J=4.7 Hz, 1H), 4.54 (t, J=4.7 Hz, 1H), 3.81 (dd, J=10.3, 5.0 Hz, 1H), 3.74 (dd, J=10.2, 5.1 Hz, 1H).

### Example 74: Synthesis of 2,2,2-Trichloroethyl (3-(4-fluorophenoxy)-5-((4-(methyl-sulfonyl)phenyl)thio)phenyl)carbamate (27e)

Yield: 99%. ¹H NMR (400 MHz, CDCl₃) δ 7.81 (d, J=8.5 Hz, 2H), 7.36 (d, J=8.6 Hz, 2H), 7.12 (s, 1H), 7.08-6.98 (m, 6H), 6.90 (s, 1H), 4.79 (s, 2H), 3.04 (s, 3H).

### Example 75: General Procedure to Synthesize 28a-g

To a round-bottomed flask equipped with a magnetic stir bar were added 27a-e (1.0 eq), 6 or 22d (1.2 eq), DIPEA (2.0 eq) and DMA. The reaction vessel was immersed in a 100° C. preheated oil bath for 12 h until the reaction was completed as determined by TLC. After cooling, the reaction was diluted with water and extracted with ethyl acetate. The ethyl acetate layer was washed with saturated brine and dried over anhydrous MgSO₄. After filtration and concentration, the crude product was purified on a silica gel column, eluted with hexane/ethyl acetate to afford 28a-g.

### Example 76: Synthesis of 4-(2-Ethylbutyl)-N-(3-(4-fluorophenoxy)-5-(4-methoxyphenoxy)phenyl)-4-hydroxypiperidine-1-carboxamide (28a)

Compound 28a was eluted with hexane/ethyl acetate (1/1, V/V) as white solid. Yield: 52%, M.P. 79-80° C. ¹H NMR (400 MHz, CDCl₃) δ 6.98 (tt, J=10.2, 3.0 Hz, 6H), 6.88-6.83 (m, 2H), 6.75 (t, J=2.0 Hz, 1H), 6.64 (t, J=2.0 Hz, 1H), 6.57 (s, 1H), 6.25 (t, J=2.2 Hz, 1H), 3.79 (s, 3H), 3.72 (d, J=12.9 Hz, 2H), 3.31-3.21 (m, 2H), 1.59 (dd, J=8.8, 3.8 Hz, 4H), 1.36 (dd, J=15.0, 8.8 Hz, 7H), 0.84 (t, J=7.2 Hz, 6H). ¹³C NMR (101 MHz, CDCl3) δ 160.07, 159.12, 158.91 (d, J=243.4), 156.09, 154.05, 152.28 (d, J=3.0), 149.38, 141.13, 121.07, 120.76 (d, J=8.1), 116.26 (d, J=23.2), 114.84, 103.44, 103.22, 102.33, 70.12, 55.60, 46.71, 40.74, 36.90, 35.36, 27.30, 10.82. HRMS (ESI) m/z [M+H]⁺ calcd. for C₃₁H₃₈FN₂O₅ 537.2795, found 537.2750.

### Example 77: Synthesis of 4-(2-Ethylbutyl)-N-(3-(4-fluorophenoxy)-5-((2-methyl-1-oxo-1,2,3,4-tetrahydroisoquinolin-6-yl)oxy)phenyl)-4-hydroxypiperidine-1-carboxamide (28b)

Compound 28b was eluted with hexane/ethyl acetate (1/3, V/V) as white solid. Yield: 24%, M.P. 80-82° C. ¹H NMR (400 MHz, CDCl₃) δ 8.00 (d, J=8.4 Hz, 1H), 7.07-6.95 (m, 3H), 6.94-6.84 (m, 2H), 6.81 (s, 1H), 6.75 (s, 1H), 6.57 (s, 1H), 6.32 (s, 1H), 3.85-3.71 (m, 2H), 3.53 (t, J=6.5 Hz, 2H), 3.34-3.20 (m, 2H), 3.12 (s, 3H), 2.94 (t, J=6.5 Hz, 2H), 1.62-1.52 (m, 4H), 1.42-1.30 (m, 7H), 1.16 (s, 1H), 0.84 (t, J=6.6 Hz, 6H). ¹³C NMR (101 MHz, CDCl₃) δ 164.49, 159.90, 159.14, 158.92 (d, J=243.4 Hz), 157.12, 154.47, 152.17 (d, J=2.0 Hz), 142.20, 140.19, 130.13, 124.12, 120.82 (d, J=8.1 Hz), 116.48, 116.28 (d, J=23.2 Hz), 116.00, 105.62, 105.11, 103.78, 70.06, 47.98, 46.73, 40.38, 37.05, 35.28, 35.04, 27.91, 27.26, 10.80. HRMS (ESI) m/z [M+Na]⁺ calcd. for C₃₄H₄₀FN₃NaO₅ 612.2850, found 612.2846.

### Example 78: Synthesis of 4-(2-Ethylbutyl)-N-(3-(4-fluorophenoxy)-5-((1-oxo-1,2,3,4-tetrahydroisoquinolin-6-yl)oxy)phenyl)-4-hydroxypiperidine-1-carboxamide (28c)

Compound 28c was eluted with ethyl acetate as white solid. Yield: 26%, M.P. 81-83° C. ¹H NMR (400 MHz, CDCl₃) δ 7.99 (d, J=8.5 Hz, 1H), 7.07-6.96 (m, 3H), 6.96-6.89 (m, 1H), 6.88-6.77 (m, 2H), 6.56 (s, 1H), 6.33 (s, 1H), 5.95 (s, 1H), 3.85-3.71 (m, 2H), 3.61-3.48 (m, 2H), 3.34-3.18 (m, 2H), 2.99-2.87 (m, 2H), 1.65-1.52 (m, 5H), 1.44-1.31 (m, 7H), 1.15 (s, 1H), 0.84 (s, 6H). ¹³C NMR (101 MHz, CDCl₃) δ 166.00, 160.41, 159.24, 158.97 (d, J=243.4 Hz), 157.03, 154.45, 152.13 (d, J=2.0 Hz), 142.15, 141.14, 130.07, 123.60, 120.88 (d, J=8.1 Hz), 116.52, 116.32 (d, J=23.2 Hz), 116.30, 105.61, 105.10, 103.90, 70.13, 46.72, 40.41, 40.09, 37.06, 35.32, 28.43, 27.27, 10.80. HRMS (ESI) m/z [M+Na]⁺ calcd. for C₃₃H₃₈FN₃NaO₅ 598.2693, found 598.2680.

### Example 79: Synthesis of 4-(2-Ethylbutyl)-N-(3-(4-((2-fluoroethyl)carbamoyl)phenoxy)-5-(4-fluorophenoxy)phenyl)-4-hydroxypiperidine-1-carboxamide (28d)

Compound 28d was eluted with hexane/ethyl acetate (7/3, V/V) as white solid. Yield: 88%, M.P. 79-82° C. ¹H NMR (400 MHz, CDCl₃) δ 7.67 (d, J=7.6 Hz, 2H), 7.14-6.93 (m, 6H), 6.90 (s, 1H), 6.86-6.76 (m, 3H), 6.31 (s, 1H), 4.68-4.46 (m, 2H), 3.84-3.62 (m, 4H), 3.26-3.12 (m, 2H), 1.58-1.44 (m, 4H), 1.38-1.22 (m, 8H), 0.88-0.78 (m, 6H). ¹³C NMR (101 MHz, DMSO) δ 166.16, 159.35, 158.84 (d, J=241.39 Hz), 158.94, 157.41, 154.60, 152.46, 144.02, 129.81, 129.59, 121.50 (d, J=8.1 Hz), 118.39, 117.00 (d, J=24.2 Hz), 104.51, 104.03, 102.45, 82.60 (d, J=166.7 Hz), 68.88, 46.52, 40.38, 37.18, 35.07, 27.18, 11.15. HRMS (ESI) m/z [M+Na]⁺ calcd. for C₃₃H₃₉F₂N₃NaO₅ 618.2755, found 618.2727.

### Example 80: Synthesis of 4-(2-Ethylbutyl)-N-(3-(4-fluorophenoxy)-5-((4-(methylsulfonyl)phenyl)thio)phenyl)-4-hydroxypiperidine-1-carboxamide (28e)

Compound 28e was eluted with hexane/ethyl acetate (2/3, V/V) as white solid. Yield: 27%, M.P. 78-80° C. ¹H NMR (400 MHz, CDCl₃) δ 7.76 (d, J=8.3 Hz, 2H), 7.31 (d, J=8.2 Hz, 2H), 7.21 (s, 1H), 7.14 (s, 1H), 7.05-6.93 (m, 4H), 6.72 (s, 1H), 6.59 (s, 1H), 3.85-3.70 (m, 2H), 3.24-3.15 (m, 2H), 3.02 (s, 3H), 1.65-1.53 (m, 4H), 1.44-1.28 (m, 7H), 1.14 (s, 1H), 0.84 (t, J=6.7 Hz, 6H). ¹³C NMR (101 MHz, CDCl₃) δ 159.06 (d, J=243.4 Hz), 159.00, 154.25, 151.94 (d, J=2.0 Hz), 145.85, 142.15, 137.33, 132.65, 127.90, 127.79, 120.92 (d, J=9.1 Hz), 119.08, 116.86, 116.45 (d, J=24.2 Hz), 110.06, 70.11, 46.72, 44.50, 40.39, 37.03, 35.31, 27.28, 10.82. HRMS (ESI) m/z [M+Na]⁺ calcd. for C₃₁H₃₇FN₂NaO₅S₂ 623.2026, found 623.2018.

### Example 81: Synthesis of N-(3-(4-((2-fluoroethyl)carbamoyl)phenoxy)-5-(4-fluorophenoxy)phenyl)-4-hydroxy-4-isobutylpiperidine-1-carboxamide (28f)

Compound 28f was eluted with hexane/ethyl acetate (1/2, V/V) as white solid. Yield: 70%, M.P. 81-83° C. ¹H NMR (400 MHz, CDCl₃) δ 7.67 (d, J=8.2 Hz, 2H), 7.06-6.90 (m, 7H), 6.86-6.75 (m, 3H), 6.31 (s, 1H), 4.68-4.46 (m, 1H), 4.55-4.46 (m, 1H), 3.82-3.60 (m, 4H), 3.19 (t, J=11.5 Hz, 2H), 1.87-1.74 (m, 1H), 1.60-1.46 (m, 4H), 1.39-1.28 (m, 3H), 0.93 (d, J=6.5 Hz, 6H). ¹³C NMR (101 MHz, CDCl₃) δ 167.44, 159.72, 159.28, 158.99 (d, J=243.4 Hz), 157.35, 154.55, 152.04 (d, J=2.0 Hz), 142.05, 128.97, 128.89, 120.96 (d, J=8.1 Hz), 118.10, 116.34 (d, J=23.2 Hz), 105.01, 104.83, 103.55, 82.54 (d, J=167.7 Hz), 70.03, 51.86, 40.54, 40.31, 37.06, 24.84, 23.22. HRMS (ESI) m/z [M+Na]⁺ calcd. for C₃₁H₃₅F₂N₃NaO₅ 590.2442, found 590.2430.

### Example 82: Synthesis of N-(3-(4-Fluorophenoxy)-5-((4-(methylsulfonyl)phenyl)-thio)phenyl)-4-hydroxy-4-isobutylpiperidine-1-carboxamide (28g)

Compound 28g was eluted with hexane/ethyl acetate (2/3, V/V) as white solid. Yield: 35%, M.P. 79-81° C. ¹H NMR (400 MHz, CDCl₃) δ 7.77 (d, J=5.6 Hz, 2H), 7.41-7.15 (m, 4H), 7.12-6.95 (m, 4H), 6.88 (s, 1H), 6.75 (s, 1H), 3.90-3.68 (m, 2H), 3.40-3.18 (m, 2H), 3.04 (s, 3H), 1.95-1.75 (m, 1H), 1.70-1.50 (m, 4H), 1.46-1.28 (m, 3H), 0.99 (s, 6H). ¹³C NMR (101 MHz, CDCl₃) δ 159.10 (d, J=244.4 Hz), 159.07, 154.15, 151.93 (d, J=3.0 Hz), 145.75, 141.99, 137.46, 132.85, 127.99, 127.83, 120.95 (d, J=8.1 Hz), 118.96, 116.95, 116.48 (d, J=23.2 Hz), 109.95, 70.13, 51.94, 44.53, 40.43, 37.10, 24.84, 23.27. HRMS (ESI) m/z [M+Na]⁺ calcd. for C₂₉H₃₃FN₂NaO₅S₂ 595.1713, found 595.1693.

### Example 83: Synthesis Scheme of 34 and 38

The syntheses of 34 and 38 and precursors are depicted in Scheme 8.

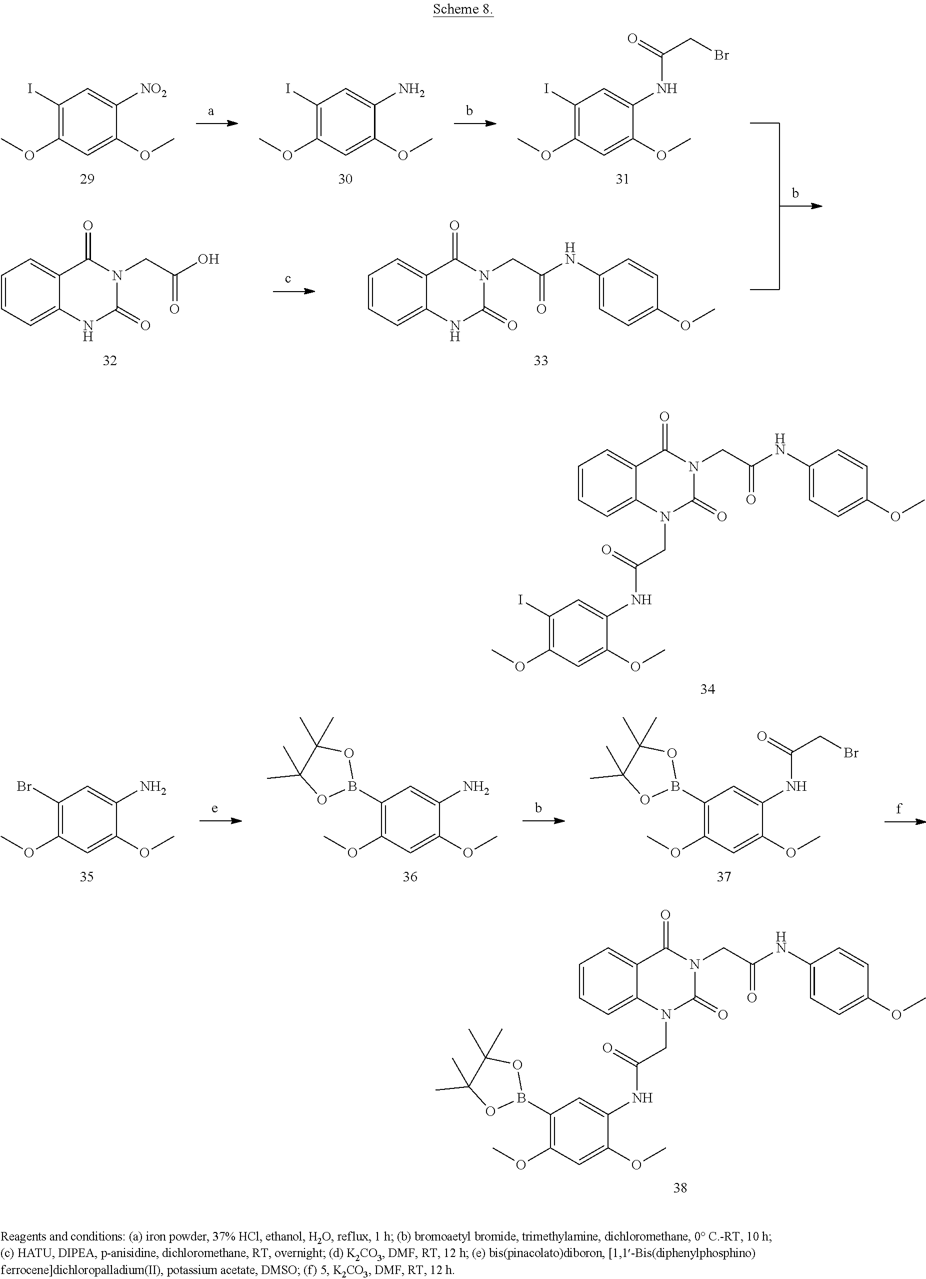

### Example 84: General Procedure to Synthesize 34 and 38

The commercially available 1-iodo-2,4-dimethoxy-5-nitrobenzene (29) was treated with iron powder and hydrochloride (HCl) to yield aniline 30, which reacted with bromoacetyl bromide gave bromide 31. Meanwhile, the 2-(2,4-dioxo-1,4-dihydroquinazolin-3(2H)-yl)acetic acid (32) was coupled with p-anisidine to afford intermediate 33 using 1-[bis(dimethylamino)methylene]-1H-1,2,3-triazolo[4,5-b]pyridinium 3-oxid hexafluorophosphate (HATU) as the condensation agent. Then, the nucleophilic substitution of bromide 31 and intermediate 33 gave the reference standard 34. The precursor 38 was prepared using a similar procedure described for 34. 5-Bromo-2,4-dimethoxyaniline (35) was converted to borate 36 by Pd-catalyzed Miyaura Borylation reaction with bis(pinacolato)diboron. After treating with bromoacetyl bromide and coupling with intermediate 33, the borate precursor 38 was obtained. The structures of all the compounds were confirmed by nuclear magnetic resonance (NMR) spectroscopy.

### Example 85: Synthesis of 5-iodo-2,4-dimethoxyaniline (30)

To a mixture of 1-iodo-2,4-dimethoxy-5-nitrobenzene (1.10 g, 3.6 mmol), iron powder (0.84 g, 15.0 mmol), ethanol (17.5 mL), and H₂O (7.5 mL) was added 37% HCl (0.21 mL) diluted in ethanol (3.0 mL) and H₂O (1.5 mL) dropwise. The reaction mixture was refluxed for 1 h and monitored by TLC. After cooling to the room temperature (RT), the mixture was filtered and the filtrate was concentrated. To the residue was added 5% Na₂CO₃ solution (20 mL), then extracted with ethyl acetate (15 mL×3). The combined ethyl acetate was washed with saturated brine (20 mL) and dried over anhydrous MgSO₄. After filtering and concentration, the crude product 30 was obtained and used directly. (0.8 g, 81%) ¹H NMR (400 MHz, CDCl₃) δ 7.10 (s, 1H), 6.44 (s, 1H), 3.85 (s, 3H), 3.82 (s, 3H), 3.54 (br, 2H).

### Example 86: Synthesis of 2-bromo-N-(5-iodo-2,4-dimethoxyphenyl)acetamide (31)

To a solution of 30 (0.80 g, 2.9 mmol) in dichloromethane (20 mL) was added bromoacetyl bromide (0.60 g, 3.0 mmol) dropwise at 0° C. After stirring for 5 min, triethylamine (0.60 g, 5.8 mmol) was added and the reaction mixture was warmed to RT and stirred overnight. The reaction was monitored by TLC. Then, the mixture was concentrated and purified by flash chromatography, eluted with hexane/ethyl acetate (3/2, V/V) to afford the product 31 (0.43 g, 37%) as gray solid. ¹H NMR (400 MHz, CDCl₃) δ 8.67 (s, 1H), 8.53 (s, 1H), 6.46 (s, 1H), 4.01 (s, 2H), 3.93 (s, 3H), 3.88 (s, 3H).

### Example 87: Synthesis of 2-(2,4-dioxo-1,4-dihydroquinazolin-3(2H)-yl)-N-(4-methoxyphenyl)acetamide (33)

A mixture of 2-(2,4-dioxo-1,4-dihydroquinazolin-3(2H)-yl)acetic acid (1.10 g, 5.0 mmol), p-anisidine (0.67 g, 5.5 mmol), 1-[bis(dimethylamino)methylene]-1H-1,2,3-triazolo[4,5-b]pyridinium 3-oxide hexafluorophosphate (HATU) (2.85 g, 7.5 mmol), N,N-diisopropylethylamine (DIPEA) (1.61 g, 12.5 mmol), and dichloromethane (20 mL) was stirred at RT overnight. Then, the mixture was concentrated and the residue was treated with 1 N HCl (50 mL), the undissolved solid was collected and dried to afford the product 5 (1.0 g, 61%) as white solid. ¹H NMR (400 MHz, DMSO-d6) δ 11.56 (s, 1H), 10.11 (s, 1H), 7.94 (d, J=7.9 Hz, 1H), 7.69 (t, J=7.7 Hz, 1H), 7.47 (d, J=8.5 Hz, 2H), 7.22 (d, J=8.2 Hz, 2H), 6.88 (d, J=8.6 Hz, 2H), 4.67 (s, 2H), 3.71 (s, 3H).

### Example 88: Synthesis of N-(5-iodo-2,4-dimethoxyphenyl)-2-(3-(2-((4-methoxyphenyl)amino)-2-oxoethyl)-2,4-dioxo-3,4-dihydroquinazolin-1(2H)-yl)acetamide (34)

A mixture of 33 (163 mg, 0.5 mmol), 31 (200 mg, 0.5 mmol), K₂CO₃ (138 mg, 1.0 mmol), and dimethylformamide (DMF) (3.0 mL) was stirred at RT overnight. Then, the mixture was diluted with H₂O (10 mL), the formed precipitate was collected and washed with acetone (10 mL). After air drying, the standard compound 34 was obtained (120 mg, 37%) as white solid. Mp 260-261° C. ¹H NMR (400 MHz, DMSO-d6) δ 10.16 (s, 1H), 9.69 (s, 1H), 8.21 (s, 1H), 8.10 (d, J=7.8 Hz, 1H), 7.80 (t, J=7.9 Hz, 1H), 7.47 (d, J=8.5 Hz, 2H), 7.43-7.32 (m, 2H), 6.88 (d, J=8.4 Hz, 2H), 6.77 (s, 1H), 5.07 (s, 2H), 4.75 (s, 2H), 3.91 (s, 3H), 3.85 (s, 3H), 3.72 (s, 3H). ¹³C NMR (101 MHz, DMSO-d6) δ 167.01, 166.11, 162.42, 156.72, 153.01, 151.97, 151.28, 141.70, 137.06, 133.35, 129.41, 124.62, 124.22, 122.02, 121.70, 116.24, 116.00, 115.38, 112.43, 99.45, 57.87, 57.83, 56.61, 47.74, 45.45.

### Example 89: Synthesis of 2,4-dimethoxy-5-(4,4,5,5-tetramethyl-1,3,2-dioxaborolan-2-yl)aniline (36)

A mixture of 5-bromo-2,4-dimethoxyaniline (0.46 g, 2.0 mmol), bis(pinacolato)diboron (1.0 g, 4.0 mmol), [1,1′-bis(diphenylphosphino)ferrocene]palladium(II) dichloride (Pd(dppf)Cl₂) (0.15 g, 0.2 mmol), potassium acetate (1.18 g, 12.0 mmol), and dimethyl sulfoxide (DMSO) (4.0 mL) was stirred at 90° C. overnight. The reaction was monitored by TLC. After cooling to RT, the mixture was diluted with ethyl acetate (10 mL) and filtered through Celite®. The filtrate was washed with H₂O (20 mL×2), saturated brine (20 mL), and dried over anhydrous MgSO₄. After filtering and concentrating, the crude product was purified by flash chromatography, eluted with hexane/ethyl acetate (2/1, V/V) to afford the product 7 (0.39 g, 70%). ¹H NMR (400 MHz, CDCl₃) δ 7.07 (s, 1H), 6.43 (s, 1H), 3.87 (s, 3H), 3.79 (s, 3H), 3.48 (s, 2H), 1.33 (s, 12H).

### Example 90: Synthesis of 2-bromo-N-(2,4-dimethoxy-5-(4,4,5,5-tetramethyl-1,3,2-dioxaborolan-2-yl)phenyl)acetamide (37)

To a solution of 36 (390 mg, 1.4 mmol) in dichloromethane (15 mL) was added bromoacetyl bromide (280 mg, 1.4 mmol) dropwise at 0° C. After stirring for 5 min, trimethylamine (280 mg, 2.8 mmol) was added and the reaction was warmed to RT and stirred overnight. The reaction was monitored by TLC. Then, the mixture was concentrated and purified by flash chromatography, eluted with hexane/ethyl acetate (1/1, V/V) to afford the intermediate 37 (160 mg, 29%). ¹H NMR (400 MHz, CDCl₃) δ 8.47-8.35 (m, 2H), 6.43 (s, 1H), 4.00 (s, 2H), 3.90 (s, 3H), 3.82 (s, 3H), 1.31 (s, 12H).

### Example 91: Synthesis of N-(2,4-dimethoxy-5-(4,4,5,5-tetramethyl-1,3,2-dioxaborolan-2-yl)phenyl)-2-(3-(2-((4-methoxyphenyl)amino)-2-oxoethyl)-2,4-dioxo-3,4-dihydroquinazolin-1(2H)-yl)acetamide (38)

A mixture of 37 (160 mg, 0.4 mmol), 33 (130 mg, 0.4 mmol), K₂CO₃ (110 mg, 0.8 mmol), and DMF (3.0 mL) was stirred at RT overnight. Then, the mixture was diluted with ethyl acetate (10 mL), washed with H₂O (20 mL×2), saturated brine (20 mL), and dried over anhydrous MgSO₄. After filtering and concentration, the crude product was purified by flash chromatography, eluted with hexane/ethyl acetate (1/3, V/V) to afford the product 38 (100 mg, 39%) as white solid. MP: 270-271° C. ¹H NMR (400 MHz, CDCl₃) δ 8.40 (s, 1H), 8.22 (d, J=7.7 Hz, 1H), 8.11 (s, 1H), 7.68 (t, J=7.3 Hz, 1H), 7.59 (s, 1H), 7.38 (t, J=9.4 Hz, 3H), 7.28 (t, J=7.6 Hz, 1H), 6.79 (d, J=9.1 Hz, 2H), 6.35 (s, 1H), 4.91 (s, 4H), 3.83-3.70 (m, 9H), 1.28 (s, 12H). ¹³C NMR (101 MHz, DMSO-d6) δ 165.95, 165.05, 161.36, 155.66, 151.96, 150.92, 150.22, 140.64, 136.01, 132.30, 128.35, 123.56, 123.17, 120.96, 120.65, 115.18, 114.94, 114.32, 111.37, 98.39, 56.82, 56.77, 55.55, 46.68, 44.39.

### Example 92: Radiosynthesis of [¹²⁵I]34

The radiosynthesis of [¹²⁵]34 was accomplished by following Scheme 9.

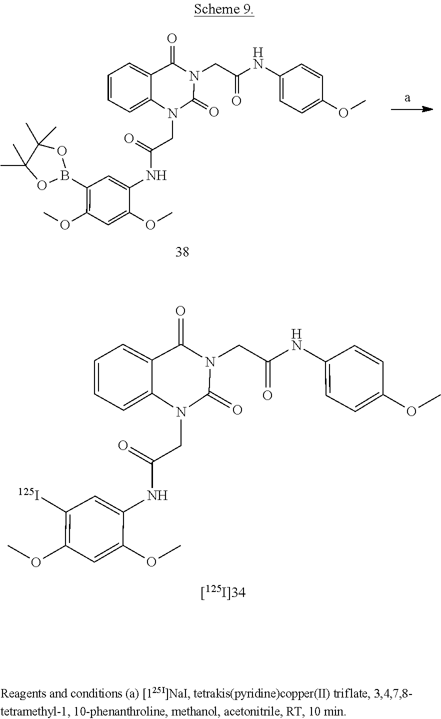

Sodium [¹²⁵I]iodide (specific activity ˜629GBq/mg) in NaOH (10⁻⁵ M) was purchased from Perkin Elmer Life and Analytical Sciences (Boston, Mass.). Preparative high-performance liquid chromatography (HPLC) was performed on a semi-preparative reverse-phase C18 analytical column (Agilent ZORBAX Eclipse XDB-C18, 250×9.4 mm, 5 μm) with UV wavelength at 254 nm and flow rate at 4 mL/min; analytical HPLC was performed on a reverse-phase C18 analytical column (Agilent ZORBAX Eclipse XDB-C18, 250×4.6 mm, 5 μm) with UV wavelength at 254 nm and flow rate at 1 mL/min; radioactive detection was carried out using a Bioscan Flowcount radioactive detector (Bioscan Inc, Washington D.C.); preparative and analytical HPLC were run in 0.1 M ammonium formate (pH 4.5) in acetonitrile.

To a 1.5 mL of microcentrifuge tube was added 40 μL of precursor (38) in acetonitrile (15 μmol/L) and 10 μL of tetrakis(pyridine)copper(II) triflate/3,4,7,8-tetramethyl-1,10-phenanthroline in methanol (1/1, 12 μmol/L), followed by adding ˜40 MBq of [¹²⁵I]NaI (pH 8-11). The reaction mixture was vortexed intermittently and incubated at room temperature for 10 min prior to loading onto a semi-preparative HPLC reverse phase column (Agilent ZORBAX Eclipse XDB-C18, 250×9.4 mm, 5 μm). The HPLC mobile phase was 55% 0.1 M ammonium formate (pH 4.5) in acetonitrile and the retention time for the product was ˜21 min. The collected HPLC fraction was diluted with 50 mL of sterile water and the activity was trapped on a Sep-Pak C18 Plus Short Cartridge (Waters, Milford, Mass.) followed by washing with 20 mL of sterile water. The final radioactive product [¹²⁵I]34 was eluted out from the C-18 cartridge using 0.6 mL of ethanol. The product activity was ˜18.5 MBq and the product was authenticated by co-injecting the cold standard compound. The averaged radiochemical yield was 47±8% (n=3). The radiochemical purity was determined by analytical HPLC on a reverse-phase analytical column (Agilent ZORBAX Eclipse XDB-C18, 250×4.6 mm, 5 μm). The mobile phase was 30% 0.1 M ammonium formate (pH 4.5) in acetonitrile and the retention time for the product was ˜6.5 min.

The borate precursor 38 was readily radiolabeled with 1-125 using the copper-catalyzed reaction at RT using [¹²⁵I]NaI as a radioactive source. [¹²⁵I]34 was obtained in >99% radiochemical purity and with a radiochemical yield of ˜47% (n=3). The identification of [¹²⁵I]34 was confirmed by the co-injection of an aliquot of the radioactive product with cold reference standard 34. The specific activity (SA) of the [¹²⁵I]34 was calculated using the theoretical maximum SA of 1-125 source ([¹²⁵I]NaI) as ˜93 GBq/μmol.

### Example 93: Radiosynthesis of [¹¹C]2c

The radiosynthesis of the [¹¹C]2c was accomplished by O-[¹¹C]methylation of phenol precursor with [¹¹C]CH₃I under basic condition (5 N NaOH) in DMSO, heated at 90° C. (Scheme 10), followed by purification using a semi-preparative reverse-phase HPLC combined with solid-phase extraction (SPE).

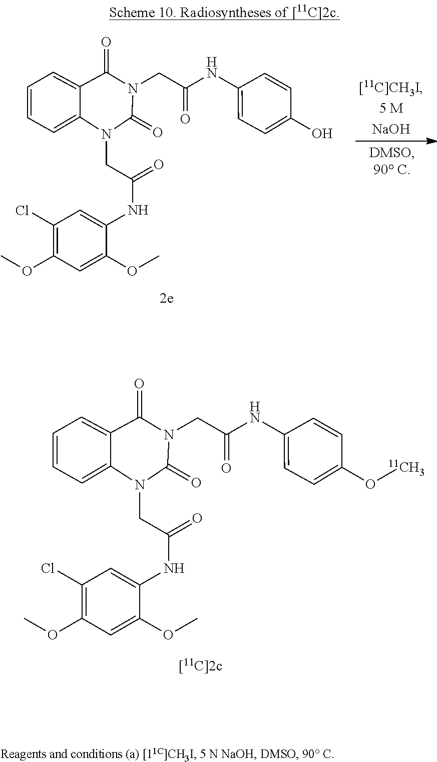

The radiosynthesis of [¹¹C]2c was accomplished with good radiochemical yield (30-40%), high chemical and radiochemical purity (>99%), and high specific activity (>1.0 Ci/μmol, decay corrected to end of synthesis, EOS).

### Example 94: General Procedure for In Vitro S1PR2 Binding Assay

The in vitro binding potency of newly synthesized analogues and reference compound JTE-013 were evaluated using a competitive [³²P]S1P binding assay based on cell membranes.

[³²P]S1P was freshly prepared and dissolved in DMSO, which was further diluted to 0.3-0.6 nM with assay buffer (50 mM HEPES-Na, pH 7.5, 5 mM MgCl₂, 1 mM CaCl₂, 0.5% fatty acid-free BSA). The test compounds were dissolved in DMSO and diluted into test samples with increasing concentrations with assay buffer. Commercial cell membranes expressing recombinant human S1PR were diluted with assay buffer to make a 20-40 μg/mL of solution. To a 96-well plate was added 50 μL of cell membranes, 50 μL of test compounds, and 50 μL of [³²P]S1P. Each well has a final volume of 150 μL containing 0.1-0.2 nM of [³²P]S1P, 1-2 μg of membrane protein (S1PRs), and different concentrations (0.01-1000 nM) of test compounds. The plate was incubated for 60 min at room temperature with shaking and terminated by collecting the membranes onto 96-well glass fiber (GF/B) filtration plates (Millipore, Billerica, Mass.). Each filter was washed with 200 μL of assay buffer for a total of five washes. The filter bound radionuclide was measured by a Beckman LS 3801 scintillation counter using Cherenkov counting. The IC₅₀ values were fitted from GraphPad Prism 6 using one site Nonlinear Regression. The results were determined in at least two independent experiments, each run was performed in duplicate; for compounds with IC₅₀<100 nM, at least three independent experiments were performed, each run was performed in duplicate.

### Example 95: In Vitro S1PR2 Binding Assay for 2a-l and 4a-h

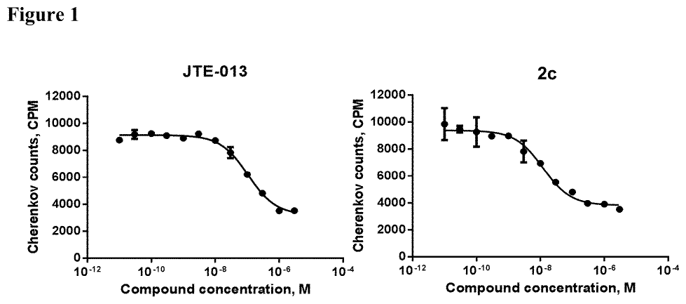

In vitro binding of newly synthesized compounds 2a-l and 4a-h toward S1PR2 was determined by the above general protocol. Results are shown in Table 1. Compounds 2a, 2c, 2d, and 2e showed best binding potency with IC₅₀ values of 6.3±0.9, 5.7±0.5, 4.8±0.5, and 2.6±0.3 nM, respectively. The representative competitive binding curves of JTE-013 and 2c were showed in FIG. 1.

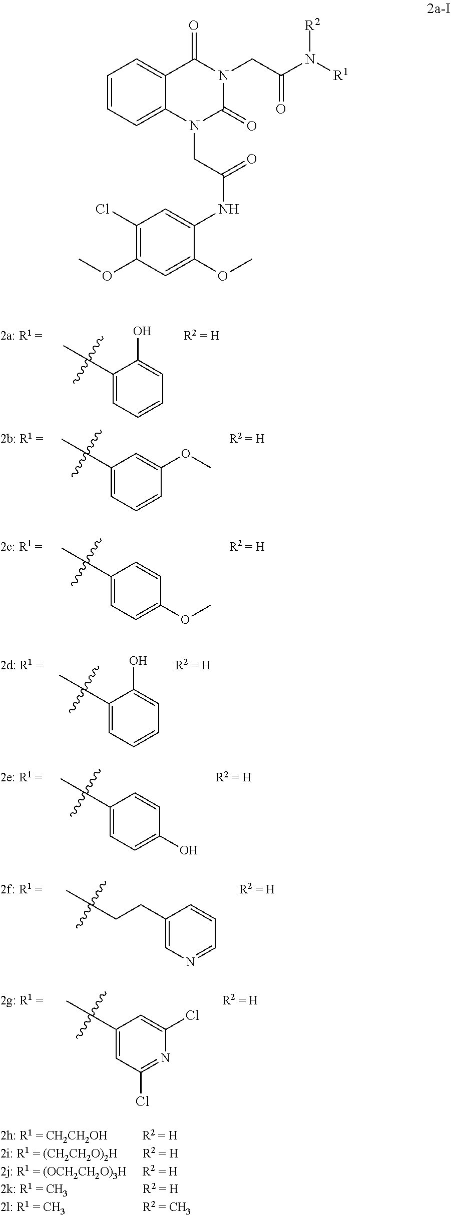

TABLE 1
Binding potencies (IC₅₀ ± SD) of
compounds 2a-I and 4a-h toward S1PR2.

| Compound | IC₅₀ (nm) |
|---|---|
| JTE-013 | 63.4 ± 4.0 |
| 2a | 6.3 ± 0.9 |
| 2b | 39.1 ± 7.0 |
| 2c | 5.7 ± 0.5 |
| 2d | 4.8 ± 0.5 |
| 2e | 2.6 ± 0.3 |
| 2f | 58.7 ± 9.0 |
| 2g | >1000 |
| 2h | 66.6 ± 10.3 |
| 2i | 91.4 ± 17.6 |
| 2j | 204 ± 96 |
| 2k | 376 ± 91 |
| 2l | 175 ± 13 |
| 4a | 54.9 ± 6.0 |
| 4b | >1000 |
| 4c | >1000 |
| 4d | >1000 |
| 4e | >1000 |
| 4f | >1000 |
| 4g | 287 ± 69 |
| 4h | 49.8 ± 4.9 |

### Example 96: S1PR2 Binding Assay of 11a-b, 17a-c, 21a-f, 25a-j, and 28a-g

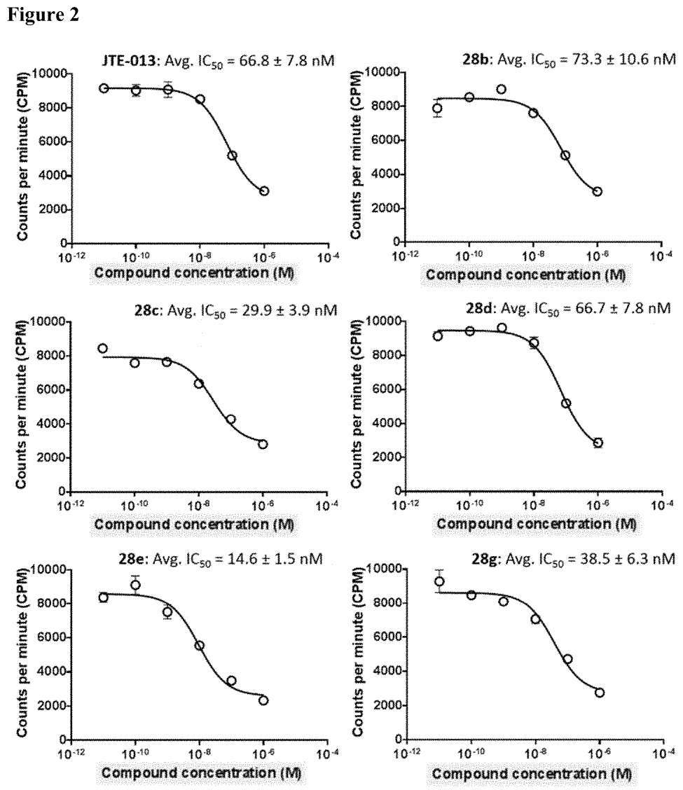

In vitro binding of newly synthesized compounds 11a-b, 17a-c, 21a-f, 25a-j, and 28a-g toward S1PR2 was determined by the above general protocol. The results are shown in Tables 1-2. The strategy of introducing alkoxy, heterocycle, or trifluoromethyl group in fragment A gave compounds 11a-b and 17a-c. The in vitro S1PR2 binding data showed that compound 17c, with trifluoromethyl group at 5-position exhibit moderate binding activity with an IC₅₀ value of 362.3 nM. The fluorine-containing alkoxy compounds, 11a and 11b showed low binding activities with IC₅₀>1000 nM. The substitution with another two N-containing heterocycles, 1-methyl-4-piperazine and 1H-pyrazole didn't improve the binding activity either; both 15a and 15b had IC₅₀>1000 nM. Subsequently, further exploration of new analogues focused on structural optimization of compound 17c. From one side, as shown in Scheme 5, we first retained trifluoromethyl group at 5-position in the fragment A and checked the impact of various substituted groups at 4′-position in fragment B (21a-f). Our binding data indicated that mono-methyl carbamide (CONHCH₃) and methylsulfonyl (SO₂CH₃) group improved the binding activity, compound 21c and 21f had IC₅₀ values of 278.5 and 270.3 nM, respectively. The other functional groups, like OCH₃, CONH₂, CON(CH₃)₂, and COOCH₃ in 21a, 21b, 21d, and 21e decreased the binding activity with IC₅₀>1000 nM. To investigate the impact of the side chain, compounds 25a-h were synthesized and tested. We observed that the replacement of 2-(ethyl)butyl chain with methyl, ethyl, or isopropyl chains caused the loss of biological activity, compounds 25a-f had IC₅₀>1000 nM. Interestingly, we observed compound 25g (IC₅₀=359.3 nM) and 25h (IC₅₀=296.5 nM) with a isobutyl side chain resulted in comparable binding activity compared to the 2-(ethyl)butyl compounds 21c and 21f. It suggested that the size and the steric hindrance of side chain lead to increase the S1PR2 binding activity. Another strategy to modify the fragment was to change the piperidine heterocycle moiety. The results revealed that the compounds 25i and 25j, with pyrrolidine and azetidine heterocycles, respectively, showed over 1000 nM of IC₅₀ values, which indicated the piperidine heterocycle was the favorable heterocycle moiety. From the other side, we replaced the trifluoromethyl group at 5-position with different alkoxy moieties and identified compounds 28a-g. The piperidine heterocycle moiety with isobutyl or 2-(ethyl)butyl side chain was retained because these two side chains were discovered as favorable pharmacophores in current analogues. As shown in Table 2, the aryloxy moieties containing compounds exhibited improved S1PR2 binding activity compared to compound 17c. Compound 28a showed an IC₅₀ value of 188.5 nM; compounds 28b and 28c, bearing a cyclization of carbamide group exhibited good S1PR2 binding potency that compound 28b had an IC₅₀ value of 73.3 nM and compound 28c had an IC₅₀ value of 29.9 nM; Compound 28d possessing a fluoroethyl carbamide group had an IC₅₀ value of 66.7 nM; compound 28e, containing a 4-(methylsulfonyl)benzene-thiol ether moiety was the most potent S1PR2 compound with an IC₅₀ value of 14.6 nM. Additionally, both of compounds 28f (IC₅₀=194.5 nM) and 28g (IC₅₀=38.5 nM) possessing an isobutyl side chain exhibited less potency for S1PR2 than the corresponding 28d and 28e, suggesting 2-(ethyl)butyl side chain is the most favorable moiety for S1PR2 binding activity. The representative competitive binding curves of compounds JTE-013, 28b, 28c, 28d, 28e, and 28g toward S1PR2 were shown in FIG. 2. Six different concentrations from 0.01 to 1000 nM were used to determine the binding potencies. As shown in the competitive binding curves, compound 28e has the best inhibition effect at 1.0 nM compared to the other compounds and it gave the best binding potency toward S1PR2 with an average IC₅₀ value of 14.6 nM.

From the in vitro binding data shown in Table 1 and Table 2, the following structure-activity relationship information was generated: a) when the 5-position substitution groups were introduced, the binding potency order of the substituents is aryloxy>CF₃>alkoxyl and N-containing heterocycles, thus the aryloxy group plays an important role in regulating the S1PR2 binding activity; b) when the 4′-position substitution groups were introduced, the stronger electronegativity of substituents offered higher S1PR2 binding activity with the order as SO₂CH₃>CONHCH₃>F>CONH₂, CON(CH₃)₂, COOCH₃, OCH₃; c) the piperidine ring was the most favorable heterocycle and the steric hindrance of side chains resulted in increased S1PR2 binding potency with the order as 2-(ethyl)butyl>isobutyl>isopropyl, ethyl, and methyl.

Because compounds 28b, 28c, 28d, 28e, and 28g showed high S1PR2 binding potency with IC₅₀<100 nM, their binding potencies toward the other S1P receptor subtypes, S1PR1, 3, 4, and 5 were also assessed to determine their in vitro binding selectivity for S1PR2. As shown in Table 2, all five compounds had no significant binding toward the other four subtypes S1PR1,3,4, and 5 (IC₅₀>1000 nM), indicating that they are highly selective for S1PR2.

TABLE 1
Structures and binding potencies (IC₅₀ ± SD) of compounds 11a-b, 17a-c,
21a-f, and 25a-j toward S1PR2.

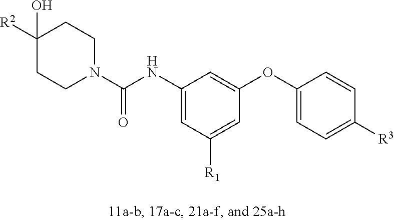

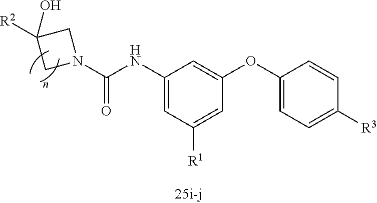

| | | | | | S2PR2 |
|---|---|---|---|---|---|
| Compd. | n | R¹ | R² | R³ | IC₅₀ (nM) |
| JTE- | — | — | — | — | 66.8 ± 7.8 |
| 013 | | | | | |
| 11a | — | OCH₂CH₂F | 2- | F | >1000 |
| | | | (ethyl)butyl | | |
| 11b | — | O(CH₂)₃F | 2- | F | >1000 |
| | | | (ethyl)butyl | | |
| 17a | — | 1- | 2- | F | >1000 |
| | | methylpiperazine | (ethyl)butyl | | |
| 17b | — | 1H-pyrazole | 2- | F | >1000 |
| | | | (ethyl)butyl | | |
| 17c | — | CF₃ | 2- | F | 362.3 ± |
| | | | (ethyl)butyl | | 85.0 |
| 21a | — | CF₃ | 2- | OCH₃ | >1000 |
| | | | (ethyl)butyl | | |
| 21b | — | CF₃ | 2- | CONH₂ | >1000 |
| | | | (ethyl)butyl | | |
| 21c | — | CF₃ | 2- | CONHCH₃ | 278.5 ± |
| | | | (ethyl)butyl | | 27.2 |
| 21d | — | CF₃ | 2- | CON(CH₃)₂ | >1000 |
| | | | (ethyl)butyl | | |
| 21e | — | CF₃ | 2- | COOCH₃ | >1000 |
| | | | (ethyl)butyl | | |
| 21f | — | CF₃ | 2- | SO₂CH₃ | 270.3 ± |
| | | | (ethyl)butyl | | 72.0 |
| 25a | — | CF₃ | methyl | CONHCH₃ | >1000 |
| 25b | — | CF₃ | methyl | SO₂CH₃ | >1000 |
| 25c | — | CF₃ | ethyl | CONHCH₃ | >1000 |
| 25d | — | CF₃ | ethyl | SO₂CH₃ | >1000 |
| 25e | — | CF₃ | isopropyl | CONHCH₃ | >1000 |
| 25f | — | CF₃ | isopropyl | SO₂CH₃ | >1000 |
| 25g | — | CF₃ | isobutyl | CONHCH₃ | 359.3 ± |
| | | | | | 50.1 |
| 25h | — | CF₃ | isobutyl | SO₂CH₃ | 296.5 ± |
| | | | | | 32.3 |
| 25i | 1 | CF₃ | isobutyl | CONHCH₃ | >1000 |
| 25j | 2 | CF₃ | isobutyl | CONHCH₃ | >1000 |

TABLE 2
Structures and binding affinities (IC₅₀ ± SD) of 28a-g toward S1PRs.

| | | | | | | | 28a-g |
|---|---|---|---|---|---|---|---|
| 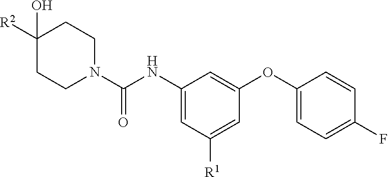 | | | | | | | |
| | | | IC₅₀ (nM) | | | | |
| Compd. | R¹ | R² | S1PR1 | S1PR2 | S1PR3 | S1PR4 | S1PR5 |
| JTE-013 | — | — | >1000 | 66.8 ± 7.8 | >1000 | >1000 | >1000 |
| S1P^{b} | — | — | 1.4 ± 0.3 | 3.6 ± 0.5 | 0.4 ± 0.2 | 151 ± 82 | 3.1 ± 1.1 |
| 28a | 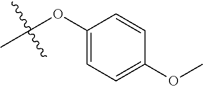 [SMILES: *Oc1ccc(OC)cc1] | 2-(ethyl)butyl | N.T^{c}. | 188.5 ± 32.9 | N.T. | N.T. | N.T. |
| 28b | 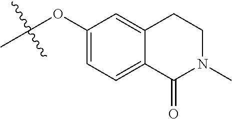 [SMILES: *Oc1ccc2c(c1)CCN(C)C2=O] | 2-(ethyl)butyl | >1000 | 73.3 ± 10.6 | >1000 | >1000 | >1000 |
| 28c | 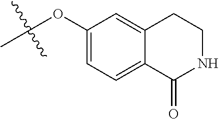 [SMILES: *Oc1ccc2c(c1)CCNC2=O] | 2-(ethyl)butyl | >1000 | 29.9 ± 3.9 | >1000 | >1000 | >1000 |
| 28d | 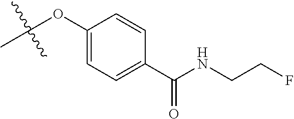 [SMILES: *Oc1ccc(C(=O)NCCF)cc1] | 2-(ethyl)butyl | >1000 | 66.7 ± 7.8 | >1000 | >1000 | >1000 |
| 28e | 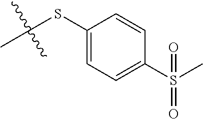 [SMILES: *Sc1ccc(S(C)(=O)=O)cc1] | 2-(ethyl)butyl | >1000 | 14.6 ± 1.5 | >1000 | >1000 | >1000 |
| 28f | 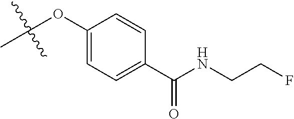 [SMILES: *Oc1ccc(C(=O)NCCF)cc1] | isobutyl | N.T. | 194.5 ± 35.9 | N.T. | N.T. | N.T. |
| 28g | 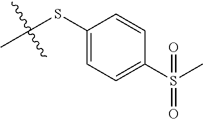 [SMILES: *Sc1ccc(S(C)(=O)=O)cc1] | isobutyl | >1000 | 38.5 ± 6.3 | >1000 | >1000 | >1000 |

### Example 97: Saturation Binding Studies of [¹²⁵I]34

We determined the dissociation constant, K_{d} (nM) of [¹²⁵I]34 through a saturation radioligand binding study using escalating concentrations of [¹²⁵I]34 to directly measure its binding toward sphingosine-1-phosphate receptor 2. Human recombinant sphingosine-1-phosphate receptor 2 lysophospholipid receptor membrane was diluted with assay buffer (50 mM HEPES Na, pH 7.5, 5 mM MgCl₂, 1 mM CaCl₂, and 0.5% fatty acid-free bovine serum albumin) and placed in a 96-well poly-L-lysine microplate (50 μL, 0.5 μg/well). Then, a 50 μL aliquot of increasing concentrations of [¹²⁵I]34 (0.2, 0.5, 1.0, 2.0, 4.0, 6.0, 8.0, 10.0, 16.0, and 20.0 nM) was added to each well; each concentration of [¹²⁵I]34 was calculated from the radioactivity and specific activity. For the determination of total versus nonspecific binding, an additional 50 μL of assay buffer and 30 μM of JTE-013, a well-known sphingosine-1-phosphate receptor 2 inhibitor was added to each well, respectively. The mixture was incubated at room temperature for 1 h on a shaker and then transferred onto 96-well glass fiber filtration plates. The unbound activity was filtered and rapidly washed away using 200 μL/well of ice-cold assay buffer for 5 times. After drying under the vacuum, the filter bound radionuclide was measured by a Beckman 8000 automated gamma counter. Data analysis was accomplished using GraphPad Prism (GraphPadSoftware, Inc., San Diego, Calif.). The specific binding was calculated by subtracting the non-specific binding from the total binding. The binding assay data were curve-fit to a one-site binding equation to generate the dissociation constant (K_{d}) of the [¹²⁵I]34.

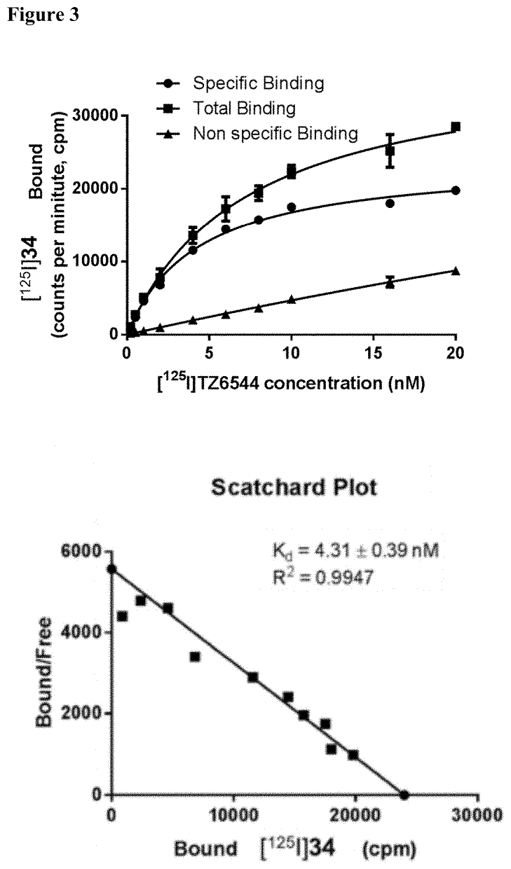

FIG. 3 shows binding of [¹²⁵I]34 to S1PR2 membrane was saturable (total binding) and blocked by 10 μM of 34 (non-specific binding). The specific binding data fit a straight line on a Scatchard plot, indicating that a one-site binding model fitted well for [¹²⁵I]34 binding to human S1PR2 cell membrane with the calculated K_{d} value of 4.31±0.39 nM.

### Example 98: Determining the IC₅₀ Values of Known sphingosine-1-phosphate Receptor 2 Compounds Using [¹²⁵I]34

[¹²⁵I]34 was used to carry out a competitive binding assay for reference compounds to determine if it is a suitable radioligand for measuring sphingosine-1-phosphate receptor 2 binding affinity. The IC₅₀ values of several reported sphingosine-1-phosphate receptor 2 ligands including JTE-013, S1P, and TZ34125 were determined using [¹²⁵]34 according to the following procedure. Human recombinant sphingosine-1-phosphate receptor 2 lysophospholipid receptor membrane was diluted with assay buffer (50 mM HEPES Na, pH 7.5, 5 mM MgCl₂, 1 mM CaCl₂, and 0.5% fatty acid-free bovine serum albumin) and placed in a 96-well poly-L-lysine microplate (50 μL, ˜1.0 μg/well). Then, 50 μL of different concentrations (0.01, 0.05, 0.1, 0.5, 1.0, 5.0, 10.0, 50.0, 100, 500, 1000 nM) of each ligand and 50 μL of [¹²⁵]34 (6.0 nM) was added to each well. The mixture was incubated for 1 h on a shaker at room temperature and then transferred onto a 96-well glass fiber filtration plates. The unbound activity was filtered and rapidly washed using ice-cold assay buffer (5×200 μL/well). After drying under reduced pressure, the filter bound radionuclide was measured by a Beckman 8000 automated gamma counter. The binding potency (IC₅₀) was calculated using GraphPad Prism.

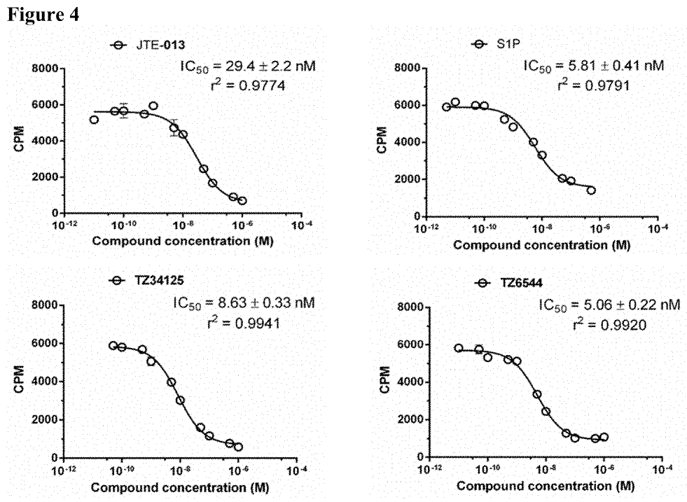

When using [¹²⁵]34 as the radioligand to determine the IC₅₀ of known sphingosine-1-phosphate receptor 2 ligands, we found that the IC₅₀ values of JTE-013, S1P, TZ34125, and 34 binding toward sphingosine-1-phosphate receptor 2 were 29.4, 5.81, 8.63, and 5.06 nM, respectively, as shown in FIG. 4. We also reported the IC₅₀ values of these compounds determined using [³²P]S1P, were 58.4, 3.6, 9.5 and 2.8 nM, respectively (Table 3). The IC₅₀ values determined using [¹²⁵]34 are comparable to that using the [³²P]S1P binding assay described above; although JTE-013 and 34 show ˜two-fold difference, we previously found different batches of [³²P]S1P can generate a two-fold IC₅₀ value change

TABLE 3
IC₅₀ (±SD nM) values of compounds binding toward
S1PR2 determined using either [³²P]S1P or [¹²⁵I]34.

| | [³²P]S1P competition | [¹²⁵I]34 competition |
|---|---|---|
| Compounds | assay IC₅₀ (nM) | assay IC₅₀ (nM) |
| JTE-013 | 58.4 ± 7.4 | 29.4 ± 2.2 |
| S1P | 3.6 ± 0.5 | 5.8 ± 0.4 |
| TZ34125 | 9.5 ± 0 | 8.6 ± 0.3 |
| 34^{d} | 2.8 ± 0.5 | 4.4 ± 0.2 |

### Example 99: General Procedures for Studies Using a Streptozotocin-Induced Rat Model of Diabetes

All rodent studies were performed in compliance with the United States Public Health Service (PHS) Policy on Humane Care and Use of Laboratory Animals as described in the Guide for the Care and Use of Laboratory Animals, under protocols approved by the Washington University in St Louis School of Medicine Institutional Animal Care and Use Committee. A pilot biodistribution study was carried out in a small number of rats at early time points post-injection in order to minimize the amount of mixed (radioactive and biological) waste generated by in vivo work. Safety procedures for working with streptozotocin in research animals were additionally reviewed and approved by the Institutional Biosafety Committee. Streptozotocin (STZ) is an N-nitro derivative of glucosamine that is toxic to the insulin-producing pancreatic β-cells, and is widely used to generate rodent models of type 1 diabetes. Adult male rats were purchased from Charles River Laboratories (Wilmington, Mass.) and allowed to acclimate in the institutional animal facility for at least a week before being used. For the 12-week, chronic model of streptozotocin-induced type 1 diabetes, Sprague Dawley rats (˜8-10 weeks old) were fasted overnight and IV injected with streptozotocin (55 mg/kg) in sodium citrate buffer (1 mL/kg); sham control rats were similarly injected with citrate buffer. Following treatment, rats were given drinking water supplemented with sucrose (15 g/L) for 48 h to limit early mortality as stores of insulin are released from damaged pancreatic islet cells. Control and treated rats were weighed and blood glucose levels checked weekly to monitor the progression of diabetes. Diabetic rats were euthanized after 12-weeks and tissues harvested; kidneys from each rat were harvested tissues were fixed in 10% formalin and paraffin embedded for subsequent staining and immunohistochemistry or snap frozen and stored at −80° C. until used in the in vitro studies described below. Control and streptozotocin-treated Wistar rats were used 10 days after treatment for the preliminary pilot acute ex vivo biodistribution study described below.

### Example 100: Ex Vivo Biodistribution Study of [¹²⁵I]34

As described above, adult male Wistar rats from Charles River were used for a pilot study to evaluate the distribution of [¹²⁵I]34 in normal rats at 5 and 30 min post injection and in acute streptozotocin-induced diabetic rats 30 min post-injection (n=4 for each group). The acute streptozotocin-induced diabetic rats were treated 10 days before the study as described above. Body weights and blood glucose confirmed that they were hyperglycemic. The concentrated solution of [¹²⁵I]34 was diluted to ˜0.037 MBq/100 μL which was formulated for injection in 10% ethanol in 5% polyoxyethylated 12-hydroxystearic acid (Kolliphor® HS 15, Millipore Sigma, Billerica, Mass.) and normal saline solution, in order to ensure that the radioactivity was solubilized. Rats were injected via the lateral tail vein under 2-3% isoflurane/oxygen anesthesia, then allowed to recover until euthanized under isoflurane/oxygen anesthesia at the appropriate time point post-injection. Tissues including blood, heart, lung, liver, kidney, muscle, fat, pancreas, spleen, kidney, liver, thyroid, thymus, brain, and small intestines were collected, weighed, and counted in a Beckman Gamma 8000 counter, and counts were corrected for background and decay corrected. Tracer uptake in tissues and organs was calculated as percent injected dose per gram of tissue (% ID/g).

The pilot biodistribution study of [¹²⁵I]34 in normal and streptozotocin-induced acute type 1 diabetic rats is displayed in Table 4. At 30 minutes post-injection, little difference was seen between the diabetic rats and the control animals. At 5 min, the initial uptake of [¹²⁵I]34 shows reasonable levels in blood, heart, lung, kidney, and liver with % ID/g values of 0.19, 0.39, 0.64, 0.58, and 2.20, respectively. Brain uptake is very low (0.01% ID/g), suggesting that [¹²⁵I]34 does not cross the blood brain barrier. The 30 minute rat biodistribution data shows clearance from most organs, including heart, kidney and pancreas in both control and diabetic rats, but much higher levels of radioactivity in the small intestine, indicating hepatobiliary clearance. Activity in the thyroid is low (% ID/organ data not shown), suggesting that the compound is metabolically stable to in vivo.

TABLE 4
Biodistribution study of [¹²⁵I]34 in Wistar rats
(% ID/g values, mean ± SD, n = 4).

| | Uptake (ID %/gram) | | |
|---|---|---|---|
| | 5 min | 30 min | 30 min |
| Organs | control | control | acute diabetic |
| Blood | 0.19 ± 0.05 | 0.08 ± 0.02 | 0.08 ± 0.05 |
| Heart | 0.39 ± .08 | 0.14 ± 0.02 | 0.12 ± 0.06 |
| Lung | 0.64 ± 0.01 | 0.22 ± 0.04 | 0.20 ± 0.09 |
| Thyroid | 0.32 ± 0.11 | 0.53 ± 0.09 | 0.25 ± 0.06 |
| Muscle | 0.12 ± 0.01 | 0.08 ± 0.01 | 0.06 ± 0.02 |
| Fat | 0.07 ± 0.02 | 0.09 ± 0.01 | 0.11 ± 0.04 |
| Pancreas | 0.40 ± 0.04 | 0.14 ± 0.01 | 0.12 ± 0.04 |
| Spleen | 0.29 ± 0.06 | 0.06 ± 0.01 | 0.05 ± 0.02 |
| Kidney | 0.58 ± 0.02 | 0.15 ± 0.02 | 0.15 ± 0.06 |
| Liver | 2.20 ± 0.32 | 0.36 ± 0.07 | 0.36 ± 0.14 |
| Thymus | 0.10 ± 0.01 | 0.10 ± 0.02 | 0.08 ± 0.03 |
| Brain | 0.01 ± 0.00 | 0.01 ± 0.00 | 0.01 ± 0.00 |
| Small intestines | 1.09 ± 0.23 | 5.32 ± 0.27 | 2.59 ± 0.58 |

### Example 101: In Vitro Autoradiography Studies of [¹²⁵I]34

To check that changes of S1PR2 expression can be detected using [¹²⁵I]34 in the STZ induced diabetes rats, in vitro autoradiography studies of [¹²⁵]34 were performed.

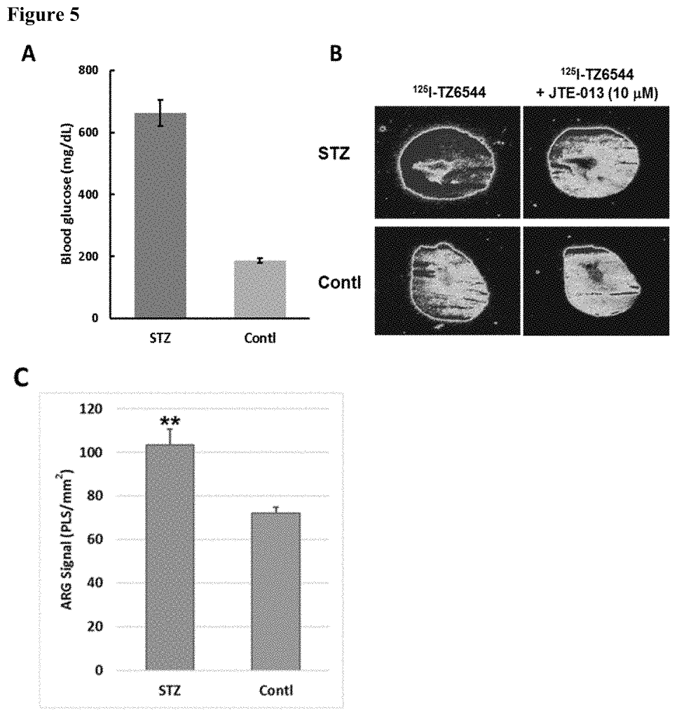

Male Sprague Dawley (SD) rats (8 weeks old,) received streptozotocin (STZ) via tail vein injections at a dose of 55 mg/kg in 50 mM sodium citrate buffer 12 weeks prior to euthanasia and tissue harvesting. Body weight and blood glucose levels were measured weekly in control and treated animals, where elevated blood glucose was indicative of chronic diabetes (see FIG. 5a). After 12 weeks, the rats were euthanized and one kidney was removed, snap-frozen, and sectioned (14 microns) with a Leica cryostat.

The autoradiography experiment was performed by incubating frozen cross sections of rat kidney tissue and 1.85 KBq/slide of [¹²⁵I]34 in 500 μL of binding buffer (50 mM HEPES-Na pH 7.5, 5 mM MgCl₂, and 1 mM CaCl₂, 0.5% fatty acid-free bovine serum albumin) to check the uptake of the radioactivity. Blocking studies of radiotracer were performed by incubating the above solution with 10 μM of JTE-013, a S1PR2 specific inhibitor. After incubation for 60 minutes at room temperature, the kidney sections were washed 2 minutes each using the following buffers sequentially: 1×TBST, 15% ethanol in 1×TBST, 30% ethanol in 1×TBST, 1×TBST (1×TBST buffer: 20 mM of Tris, 150 mM of NaCl, 0.1% of Tween 20). Then, the section slides were placed into a cassette and exposed to a phosphor sensor sheet for 24 hours. Autoradiography signal was visualized by phosphor-imaging of FLA7000. Autoradiography signal on the renal tissues (cortex and medulla, not including the renal papilla) was measured and quantified with Multi Gouge program. The data was background-corrected and expressed as photo-stimulated luminescence signals per square millimeter (PSL/mm²) as shown in FIG. 5B. For statistical comparison, the student t-test (unpaired) was applied using MS Excel and p<0.05 was considered statistically significant (see FIG. 5C.)

Representative in vitro autoradiography of frozen kidney sections is shown in FIGS. 5B and 5C. The kidney radioactivity of [¹²⁵I]34 was 103.5 PSL/mm² (n=5) for the chronic diabetic rats, and 72.0 PSL/mm², (n=4) for the control rats. The binding of [¹²⁵I]34 to the kidney of diabetic rats was increased 44% compared to that observed in the control rats. In addition, the binding of [¹²⁵I]34 to kidney tissue in both groups could be blocked using 10 μM of JTE-013, indicating that the binding is sphingosine-1-phosphate receptor 2 specific.

### Example 102: Immunostaining Study

To confirm if the kidney uptake of [¹²⁵I]34 is caused by increased S1PR2 expression, immunostaining studies were performed. The kidney from the diabetic rats was fixed with 10% formalin and embedded in paraffin. The tissue was then cut into 5 μm sections. Sections were deparaffinized, and endogenous peroxidase activity was quenched before the slides were incubated in a blocking buffer. Upon the completion of the blocking, the slides were incubated with a 1:100 dilution of a rabbit anti-rat S1PR2 antibody (ThermoFisher, Waltham, Mass.) overnight at 4° C. The primary antibody binding was detected using an anti-rabbit HRP-DAB staining kit (R&D Systems) according to the manufacturers instructions. A Nikon E600 microscope coupled with a charge-coupled device camera was used to obtain all photomicrographs.

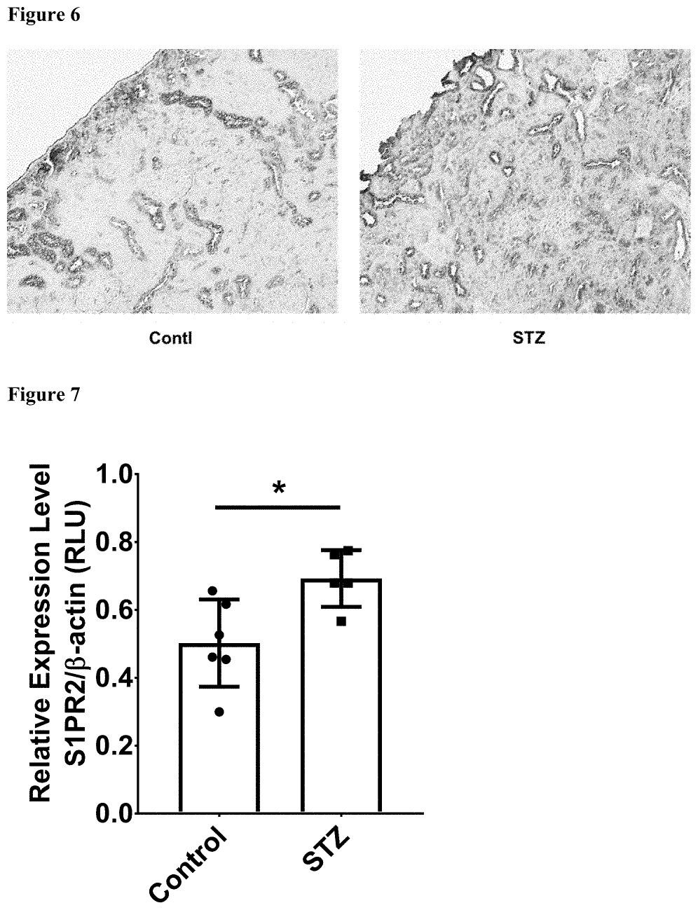

The immunostaining of S1PR2 was performed to confirm the increased expression of S1PR2 in the kidney of diabetic rats. As shown in FIG. 6, positive staining for S1PR2 was observed in the kidney of both control and diabetic rats. The STZ group showed higher staining intensity, indicating the expression of S1PR2 in the kidney of diabetic rats is upregulated compared to controls.

The amount of sphingosine-1-phosphate receptor 2 in the kidney was also evaluated by ELISA analysis. ELISA is a commonly used analytical biochemistry assay to detect the expression of protein in cells and tissues. As shown in FIG. 7, the level of sphingosine-1-phosphate receptor 2 was statistically higher in the chronic diabetic rats than the control rats (p=0.0195, unpaired t-test).

### Example 103: Biodistribution Study of [¹¹C]2c

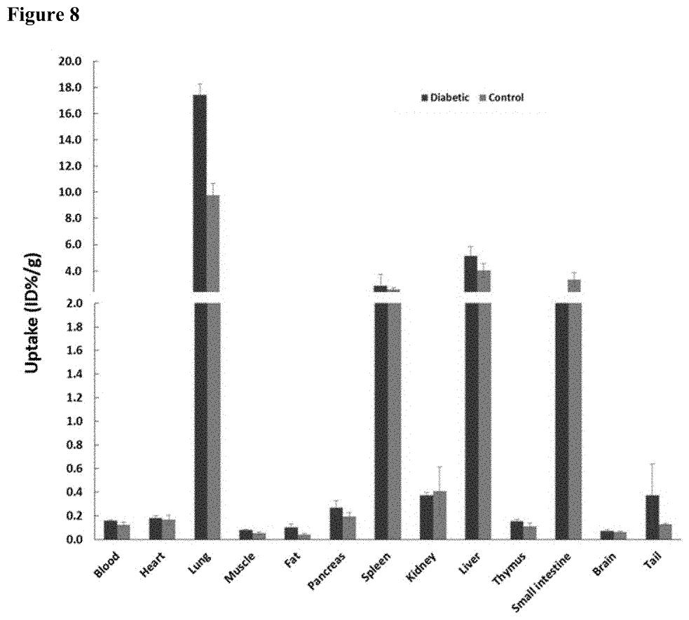

A biodistribution study was conducted in female SJL EAE mice and control mice (at 9 weeks, n=4 for each group) to determine the tissue accumulation of the radiotracer post-injection of the dose. The biodistribution data of [¹¹C]2c (shown in FIG. 8) showed increased uptake of [¹¹C]2c in brain, blood, heart, lung, and pancreas in EAE mice, and the brain uptake of [¹¹C]2c in EAE mice (% ID/g=0.699) is higher than the control (% ID/g=0.452).

### Example 104: Immunization Study of Inflammation Disease in Mice

Experimental autoimmune encephalomyelitis (EAE) was induced in 10 week old female SJL/J mice via active immunization with proteolipid protein (PLP₁₃₉₋₁₅₁, 200 μg) (GenScript USA Inc.) emulsified in complete Freund's adjuvant containing Mycobacterium tuberculosis (H37Ra; Difco Laboratories). In addition, mice were injected with 50 ng pertussis toxin (List Biologicals Laboratories Inc.) at the time of immunization and 2 days after. Mice were graded for clinical manifestations of EAE by the following criteria: 1, tail weakness; 2, difficulty righting; 3, complete inability to move one hind limb; 4, complete hind limb paralysis; 5, moribund or dead. Subcutaneous osmotic pumps (Alzet) were implanted on day 7 postimmunization with PLP₁₃₉₋₁₅₁. Compound 2c (TZ59-104) (1 and 5 mg/kg), JTE-013 (1.5 mg/kg), or vehicle (20% DMSO, 5% HS-15 in sterile water) were loaded into osmotic pumps according to the manufacturer's instructions. The infusion rate was 0.25 μl/h and consistent administration started at 2 days and continued for 28 days post implantation of the pumps.

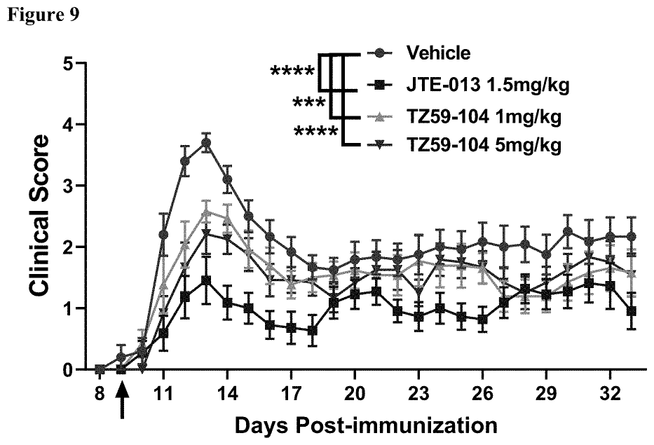

Clinical EAE data were analyzed by Mann-Whitney U test using the Prism software (GraphPad). A p value of less than 0.05 was considered statistically significant. The mice were monitored for symptoms, which were graded using EAE Classical Disease scoring: 1: Limp Tail; 2: Disrupted righting reflex; 2.5: Knuckling; 3: Complete inability to move one hind limb; 3.5: Complete paralysis in one hind limb+Partial paralysis in alternate hind limb; 4: Complete hind limb paralysis; 5: Moribund/Death. The clinical score for 2 weeks post treatment are shown in FIG. 9.

Treatment with 2c (TZ59-104), at 1 and 5 mg/kg, beginning at day 9 postimmunization led to significant improvement of EAE disease course (p<0.0002, p<0.0001). Treatment with 1.5 mg/kg also significantly reduced EAE severity (p<0.0001).

### Example 105: Synthesis and Binding Assay of Additional Compounds

Table 5 shows compounds prepared by the synthesis methods described above with IC₅₀ values measured by binding assays performed as described above.

TABLE 5
Structures and Binding Affinities of new analogues

| | | IC₅₀ (nM) | | | | |
|---|---|---|---|---|---|---|
| Name | Structure | S1PR1 | S1PR2 | S1PR3 | S1PR4 | S1PR5 |
| TZ51- 011 | 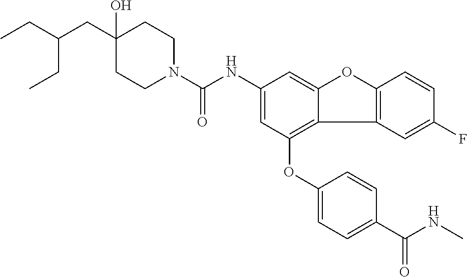 [SMILES: CCC(CC)CC1(O)CCN(C(=O)Nc2cc(Oc3ccc(C(=O)NC)cc3)c3c(c2)oc2ccc(F)cc23)CC1] | n/a | 472 ± 244 | n/a | n/a | n/a |
| TZ51- 018 | 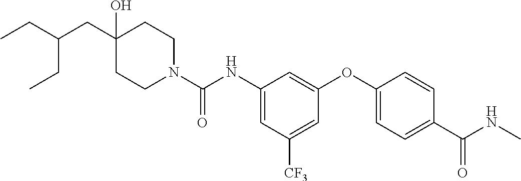 [SMILES: CCC(CC)CC1(O)CCN(C(=O)Nc2cc(Oc3ccc(C(=O)NC)cc3)cc(C(F)(F)F)c2)CC1] | n/a | >1000 | n/a | n/a | n/a |
| TZ51- 019 | 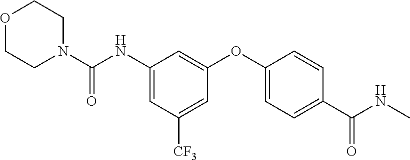 [SMILES: CNC(=O)c1ccc(Oc2cc(NC(=O)N3CCOCC3)cc(C(F)(F)F)c2)cc1] | n/a | >1000 | n/a | n/a | n/a |
| TZ51- 020 | 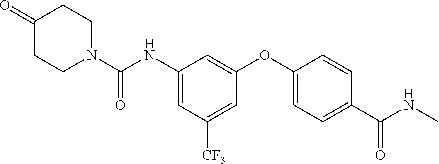 [SMILES: CNC(=O)c1ccc(Oc2cc(NC(=O)N3CCC(=O)CC3)cc(C(F)(F)F)c2)cc1] | n/a | >1000 | n/a | n/a | n/a |
| TZ51- 022 | 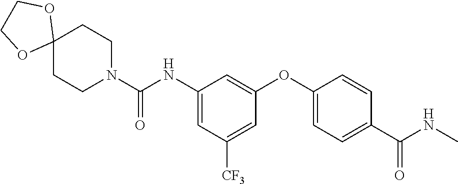 [SMILES: CNC(=O)c1ccc(Oc2cc(NC(=O)N3CCC4(CC3)OCCO4)cc(C(F)(F)F)c2)cc1] | n/a | >1000 | n/a | n/a | n/a |
| TZ51- 024 | 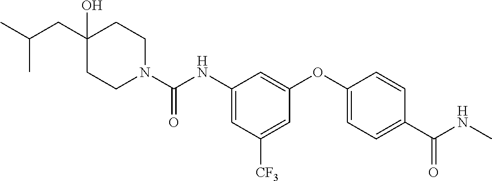 [SMILES: CNC(=O)c1ccc(Oc2cc(NC(=O)N3CCC(O)(CC(C)C)CC3)cc(C(F)(F)F)c2)cc1] | >1000 | 284 ± 27 | >1000 | n/a | n/a |
| TZ51- 032 | 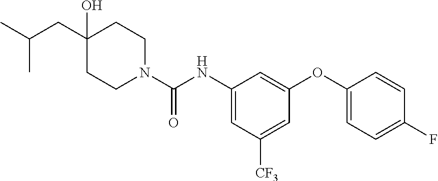 [SMILES: CC(C)CC1(O)CCN(C(=O)Nc2cc(Oc3ccc(F)cc3)cc(C(F)(F)F)c2)CC1] | >1000 | 165 ± 35 | >1000 | n/a | n/a |
| TZ51- 033 | 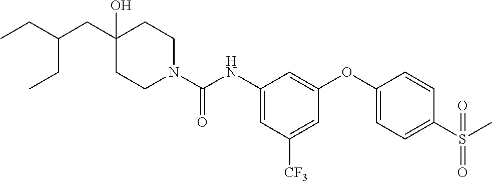 [SMILES: CCC(CC)CC1(O)CCN(C(=O)Nc2cc(Oc3ccc(S(C)(=O)=O)cc3)cc(C(F)(F)F)c2)CC1] | >1000 | 296 ± 32 | >1000 | n/a | n/a |
| TZ51- 035 | 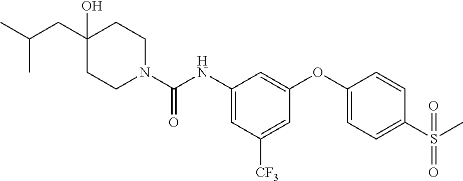 [SMILES: CC(C)CC1(O)CCN(C(=O)Nc2cc(Oc3ccc(S(C)(=O)=O)cc3)cc(C(F)(F)F)c2)CC1] | >1000 | 270 ± 72 | >1000 | n/a | n/a |
| TZ51- 041 | 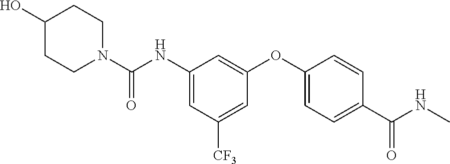 [SMILES: CNC(=O)c1ccc(Oc2cc(NC(=O)N3CCC(O)CC3)cc(C(F)(F)F)c2)cc1] | n/a | >1000 | n/a | n/a | n/a |
| TZ51- 044 | 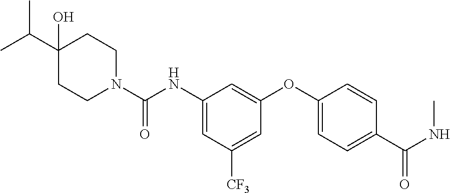 [SMILES: CNC(=O)c1ccc(Oc2cc(NC(=O)N3CCC(O)(C(C)C)CC3)cc(C(F)(F)F)c2)cc1] | n/a | >1000 | n/a | n/a | n/a |
| TZ51- 045 | 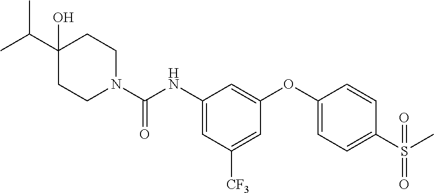 [SMILES: CC(C)C1(O)CCN(C(=O)Nc2cc(Oc3ccc(S(C)(=O)=O)cc3)cc(C(F)(F)F)c2)CC1] | n/a | >1000 | n/a | n/a | n/a |
| TZ51- 046 | 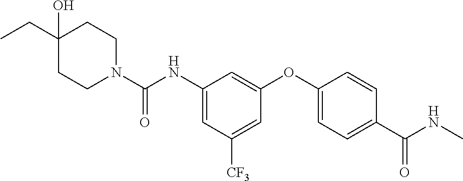 [SMILES: CCC1(O)CCN(C(=O)Nc2cc(Oc3ccc(C(=O)NC)cc3)cc(C(F)(F)F)c2)CC1] | n/a | >1000 | n/a | n/a | n/a |
| TZ51- 047 | 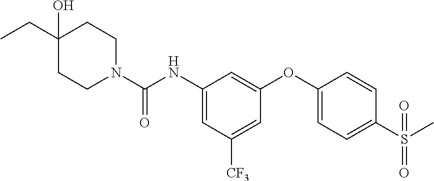 [SMILES: CCC1(O)CCN(C(=O)Nc2cc(Oc3ccc(S(C)(=O)=O)cc3)cc(C(F)(F)F)c2)CC1] | n/a | >1000 | n/a | n/a | n/a |
| TZ51- 057 | 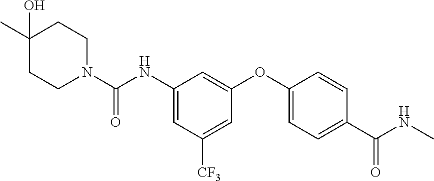 | n/a | >1000 | n/a | n/a | n/a |
| TZ51- 058 | 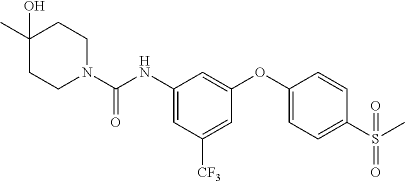 | n/a | >1000 | n/a | n/a | n/a |
| TZ51- 059 | 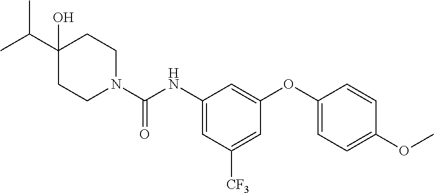 [SMILES: COc1ccc(Oc2cc(NC(=O)N3CCC(O)(C(C)C)CC3)cc(C(F)(F)F)c2)cc1] | n/a | >1000 | n/a | n/a | n/a |
| TZ51- 061 | 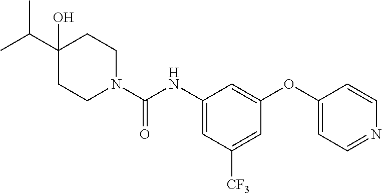 [SMILES: CC(C)C1(O)CCN(C(=O)Nc2cc(Oc3ccncc3)cc(C(F)(F)F)c2)CC1] | n/a | >1000 | n/a | n/a | n/a |
| TZ51- 073 | 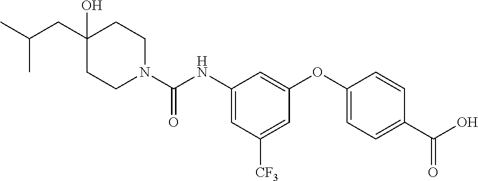 [SMILES: CC(C)CC1(O)CCN(C(=O)Nc2cc(Oc3ccc(C(=O)O)cc3)cc(C(F)(F)F)c2)CC1] | n/a | >1000 | n/a | n/a | n/a |
| TZ51- 095 | 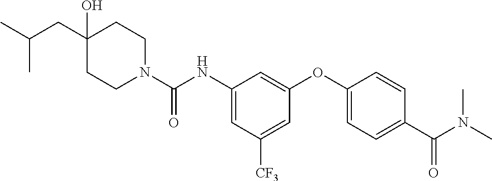 [SMILES: CC(C)CC1(O)CCN(C(=O)Nc2cc(Oc3ccc(C(=O)N(C)C)cc3)cc(C(F)(F)F)c2)CC1] | n/a | >1000 | n/a | n/a | n/a |
| TZ51- 096 | 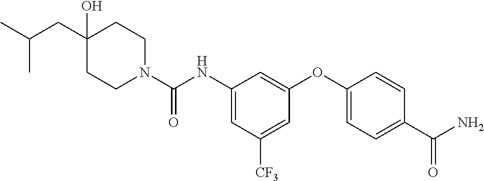 [SMILES: CC(C)CC1(O)CCN(C(=O)Nc2cc(Oc3ccc(C(N)=O)cc3)cc(C(F)(F)F)c2)CC1] | n/a | >1000 | n/a | n/a | n/a |
| TZ51- 097 | 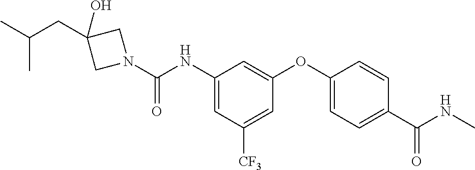 [SMILES: CNC(=O)c1ccc(Oc2cc(NC(=O)N3CC(O)(CC(C)C)C3)cc(C(F)(F)F)c2)cc1] | n/a | >1000 | n/a | n/a | n/a |
| TZ51- 098 | 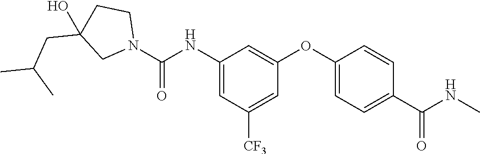 [SMILES: CNC(=O)c1ccc(Oc2cc(NC(=O)N3CCC(O)(CC(C)C)C3)cc(C(F)(F)F)c2)cc1] | n/a | >1000 | n/a | n/a | n/a |
| TZ51- 108 | 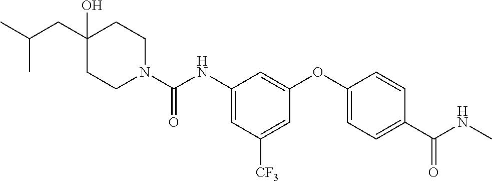 [SMILES: CNC(=O)c1ccc(Oc2cc(NC(=O)N3CCC(O)(CC(C)C)CC3)cc(C(F)(F)F)c2)cc1] | n/a | >1000 | n/a | n/a | n/a |
| TZ51- 112 | 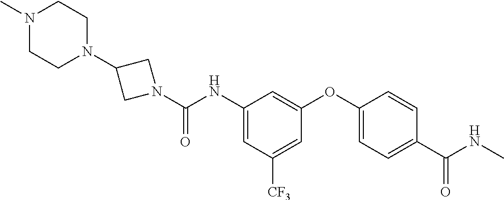 | n/a | >1000 | n/a | n/a | n/a |
| TZ51- 121 | 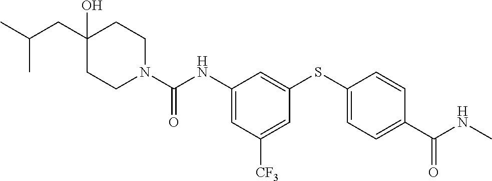 [SMILES: CNC(=O)c1ccc(Sc2cc(NC(=O)N3CCC(O)(CC(C)C)CC3)cc(C(F)(F)F)c2)cc1] | n/a | 421 ± 90 | n/a | n/a | n/a |
| TZ51- 133 | 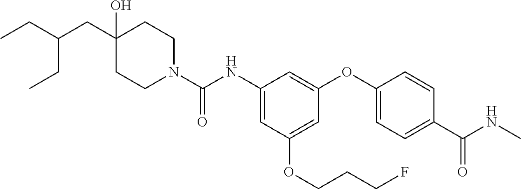 [SMILES: CCC(CC)CC1(O)CCN(C(=O)Nc2cc(OCCCF)cc(Oc3ccc(C(=O)NC)cc3)c2)CC1] | n/a | >1000 | n/a | n/a | n/a |
| TZ51- 139 | 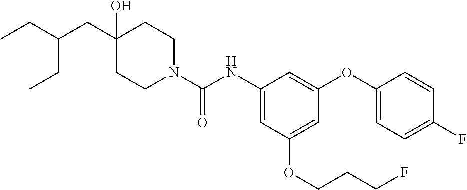 [SMILES: CCC(CC)CC1(O)CCN(C(=O)Nc2cc(OCCCF)cc(Oc3ccc(F)cc3)c2)CC1] | n/a | >1000 | n/a | n/a | n/a |
| TZ51- 149 | 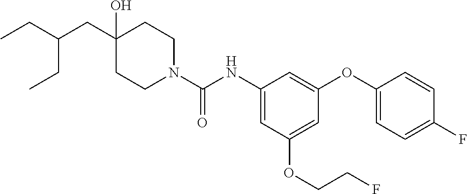 [SMILES: CCC(CC)CC1(O)CCN(C(=O)Nc2cc(OCCF)cc(Oc3ccc(F)cc3)c2)CC1] | n/a | >1000 | n/a | n/a | n/a |
| TZ51- 150 | 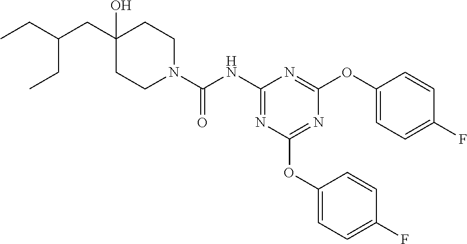 [SMILES: CCC(CC)CC1(O)CCN(C(=O)Nc2nc(Oc3ccc(F)cc3)nc(Oc3ccc(F)cc3)n2)CC1] | n/a | >1000 | n/a | n/a | n/a |
| TZ57- 007 | 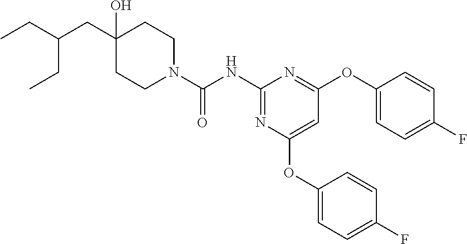 [SMILES: CCC(CC)CC1(O)CCN(C(=O)Nc2nc(Oc3ccc(F)cc3)cc(Oc3ccc(F)cc3)n2)CC1] | n/a | >1000 | n/a | n/a | n/a |
| TZ57- 013 | 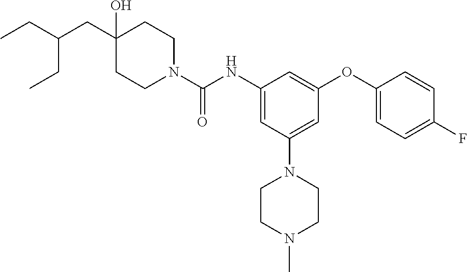 [SMILES: CCC(CC)CC1(O)CCN(C(=O)Nc2cc(Oc3ccc(F)cc3)cc(N3CCN(C)CC3)c2)CC1] | n/a | >1000 | n/a | n/a | n/a |
| TZ57- 016 | 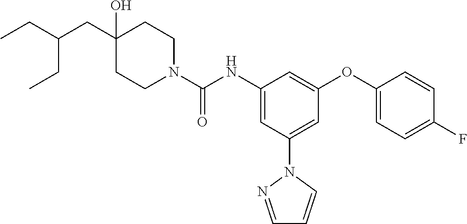 [SMILES: CCC(CC)CC1(O)CCN(C(=O)Nc2cc(Oc3ccc(F)cc3)cc(-n3cccn3)c2)CC1] | n/a | >1000 | n/a | n/a | n/a |
| TZ57- 028 | 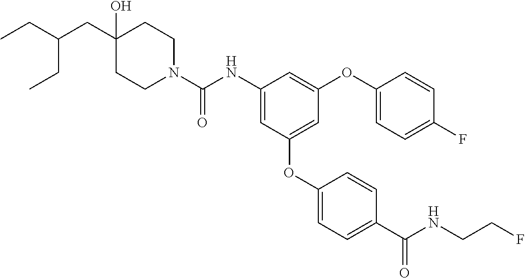 [SMILES: CCC(CC)CC1(O)CCN(C(=O)Nc2cc(Oc3ccc(F)cc3)cc(Oc3ccc(C(=O)NCCF)cc3)c2)CC1] | >1000 | 66.7 ± 7.8 | >1000 | n/a | n/a |
| TZ57- 029 | 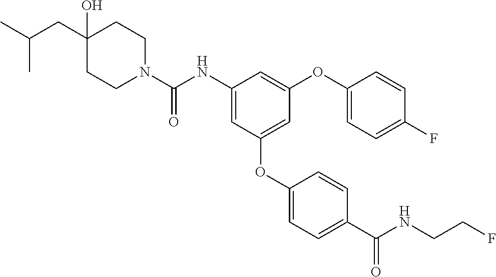 [SMILES: CC(C)CC1(O)CCN(C(=O)Nc2cc(Oc3ccc(F)cc3)cc(Oc3ccc(C(=O)NCCF)cc3)c2)CC1] | >1000 | 195 ± 36 | >1000 | n/a | n/a |
| TZ57- 031 | 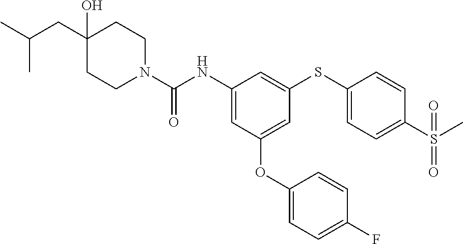 [SMILES: CC(C)CC1(O)CCN(C(=O)Nc2cc(Oc3ccc(F)cc3)cc(Sc3ccc(S(C)(=O)=O)cc3)c2)CC1] | >1000 | 38.5 ± 6.3 | >1000 | n/a | n/a |
| TZ57- 032 | 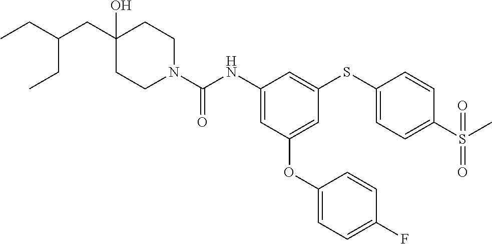 [SMILES: CCC(CC)CC1(O)CCN(C(=O)Nc2cc(Oc3ccc(F)cc3)cc(Sc3ccc(S(C)(=O)=O)cc3)c2)CC1] | >1000 | 14.6 ± 1.5 | >1000 | n/a | n/a |
| TZ57- 033 | 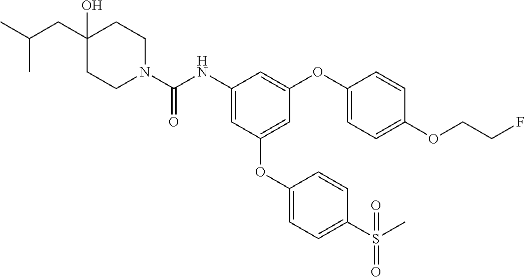 [SMILES: CC(C)CC1(O)CCN(C(=O)Nc2cc(Oc3ccc(OCCF)cc3)cc(Oc3ccc(S(C)(=O)=O)cc3)c2)CC1] | >1000 | 525 ± 119 | >1000 | n/a | n/a |
| TZ57- 034 | 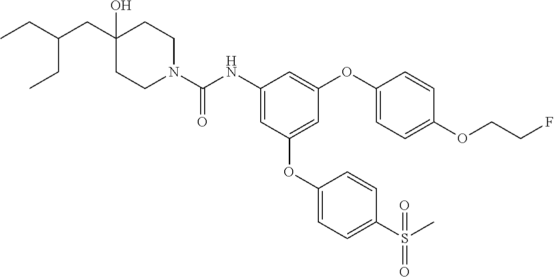 [SMILES: CCC(CC)CC1(O)CCN(C(=O)Nc2cc(Oc3ccc(OCCF)cc3)cc(Oc3ccc(S(C)(=O)=O)cc3)c2)CC1] | >1000 | 418 ± 118 | >1000 | n/a | n/a |
| TZ57- 040 | 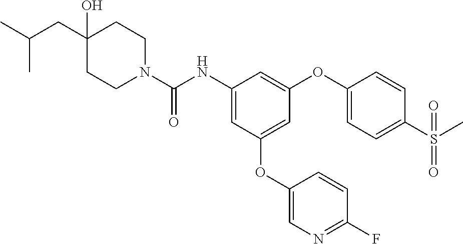 [SMILES: CC(C)CC1(O)CCN(C(=O)Nc2cc(Oc3ccc(S(C)(=O)=O)cc3)cc(Oc3ccc(F)nc3)c2)CC1] | n/a | 211 ± 32 | n/a | n/a | n/a |
| TZ57- 041 | 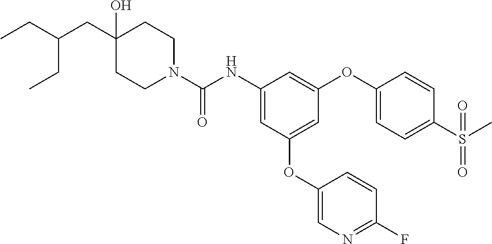 [SMILES: CCC(CC)CC1(O)CCN(C(=O)Nc2cc(Oc3ccc(S(C)(=O)=O)cc3)cc(Oc3ccc(F)nc3)c2)CC1] | n/a | 198 ± 45 | n/a | n/a | n/a |
| TZ57- 045 | 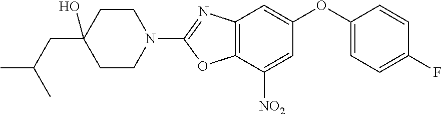 [SMILES: CC(C)CC1(O)CCN(c2nc3cc(Oc4ccc(F)cc4)cc([N+](=O)[O-])c3o2)CC1] | n/a | >1000 | n/a | n/a | n/a |
| TZ57- 047 | 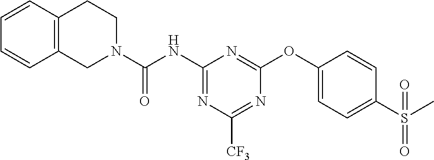 [SMILES: CS(=O)(=O)c1ccc(Oc2nc(NC(=O)N3CCc4ccccc4C3)nc(C(F)(F)F)n2)cc1] | n/a | >1000 | n/a | n/a | n/a |
| TZ57- 054 | 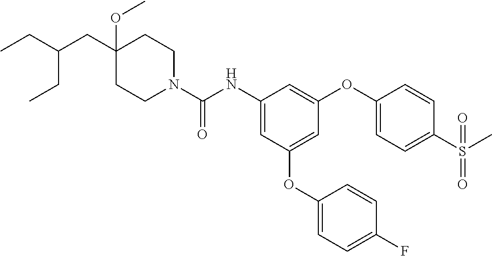 [SMILES: CCC(CC)CC1(OC)CCN(C(=O)Nc2cc(Oc3ccc(F)cc3)cc(Oc3ccc(S(C)(=O)=O)cc3)c2)CC1] | n/a | >1000 | n/a | n/a | n/a |
| TZ57- 062 | 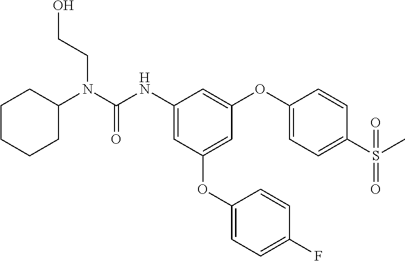 [SMILES: CS(=O)(=O)c1ccc(Oc2cc(NC(=O)N(CCO)C3CCCCC3)cc(Oc3ccc(F)cc3)c2)cc1] | n/a | >1000 | n/a | n/a | n/a |
| TZ57- 063 | 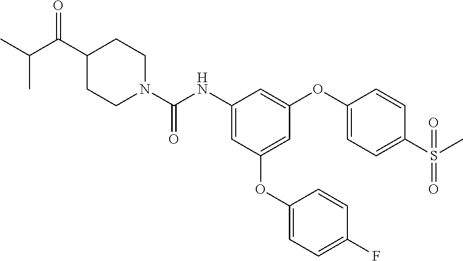 [SMILES: CC(C)C(=O)C1CCN(C(=O)Nc2cc(Oc3ccc(F)cc3)cc(Oc3ccc(S(C)(=O)=O)cc3)c2)CC1] | n/a | >1000 | n/a | n/a | n/a |
| TZ57- 065 | 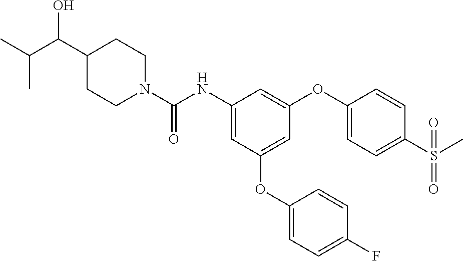 [SMILES: CC(C)C(O)C1CCN(C(=O)Nc2cc(Oc3ccc(F)cc3)cc(Oc3ccc(S(C)(=O)=O)cc3)c2)CC1] | n/a | >1000 | n/a | n/a | n/a |
| TZ57- 080 | 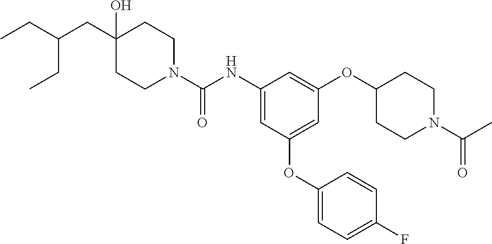 [SMILES: CCC(CC)CC1(O)CCN(C(=O)Nc2cc(Oc3ccc(F)cc3)cc(OC3CCN(C(C)=O)CC3)c2)CC1] | n/a | >1000 | n/a | n/a | n/a |
| TZ57- 081 | 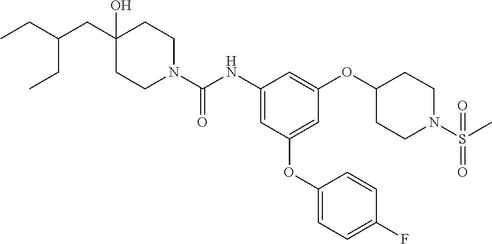 [SMILES: CCC(CC)CC1(O)CCN(C(=O)Nc2cc(Oc3ccc(F)cc3)cc(OC3CCN(S(C)(=O)=O)CC3)c2)CC1] | n/a | 292 ± 121 | n/a | n/a | n/a |
| TZ57- 089 | 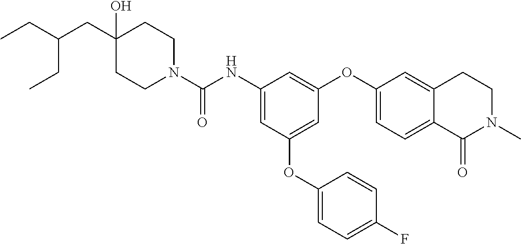 [SMILES: CCC(CC)CC1(O)CCN(C(=O)Nc2cc(Oc3ccc(F)cc3)cc(Oc3ccc4c(c3)CCN(C)C4=O)c2)CC1] | >1000 | 73 ± 11 | >1000 | n/a | n/a |
| TZ57- 090 | 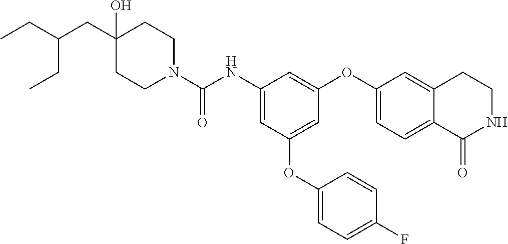 [SMILES: CCC(CC)CC1(O)CCN(C(=O)Nc2cc(Oc3ccc(F)cc3)cc(Oc3ccc4c(c3)CCNC4=O)c2)CC1] | >1000 | 30 ± 4 | >1000 | n/a | n/a |
| TZ57- 091 | 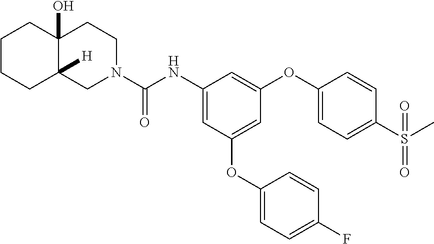 | n/a | >1000 | n/a | n/a | n/a |
| TZ57- 096 | 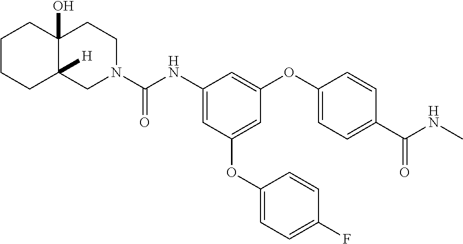 | n/a | >1000 | n/a | n/a | n/a |
| TZ57- 103 | 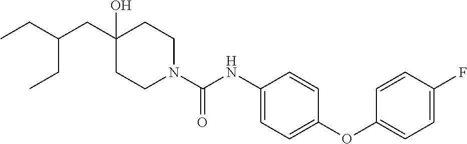 [SMILES: CCC(CC)CC1(O)CCN(C(=O)Nc2ccc(Oc3ccc(F)cc3)cc2)CC1] | n/a | >1000 | n/a | n/a | n/a |
| TZ57- 104 | 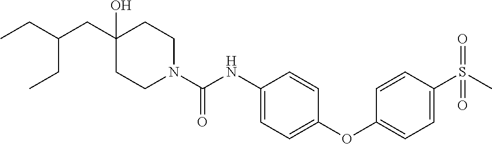 [SMILES: CCC(CC)CC1(O)CCN(C(=O)Nc2ccc(Oc3ccc(S(C)(=O)=O)cc3)cc2)CC1] | n/a | >1000 | n/a | n/a | n/a |
| TZ57- 112 | 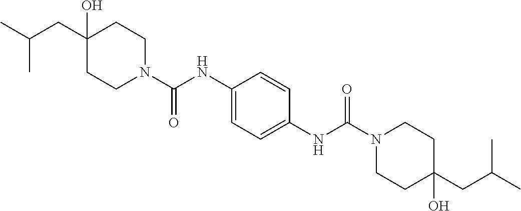 [SMILES: CC(C)CC1(O)CCN(C(=O)Nc2ccc(NC(=O)N3CCC(O)(CC(C)C)CC3)cc2)CC1] | n/a | >1000 | n/a | n/a | n/a |
| TZ57- 115 | 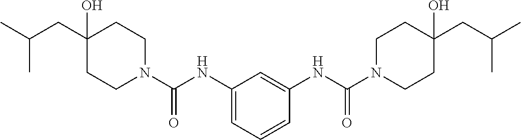 [SMILES: CC(C)CC1(O)CCN(C(=O)Nc2cccc(NC(=O)N3CCC(O)(CC(C)C)CC3)c2)CC1] | n/a | >1000 | n/a | n/a | n/a |
| TZ57- 119 | 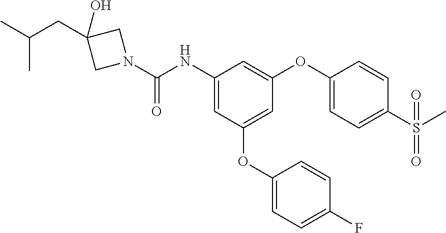 [SMILES: CC(C)CC1(O)CN(C(=O)Nc2cc(Oc3ccc(F)cc3)cc(Oc3ccc(S(C)(=O)=O)cc3)c2)C1] | n/a | >1000 | n/a | n/a | n/a |
| TZ57- 120 | 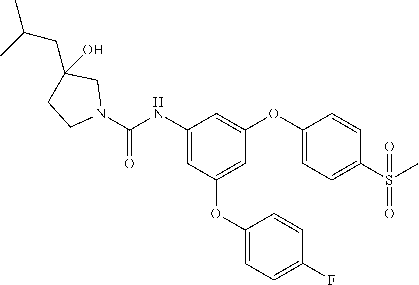 [SMILES: CC(C)CC1(O)CCN(C(=O)Nc2cc(Oc3ccc(F)cc3)cc(Oc3ccc(S(C)(=O)=O)cc3)c2)C1] | n/a | 122 ± 29 | n/a | n/a | n/a |
| TZ57- 121 | 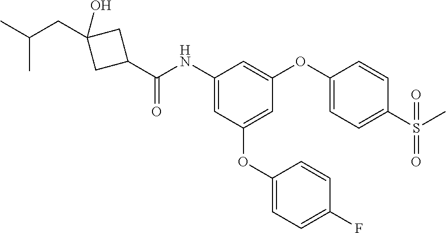 [SMILES: CC(C)CC1(O)CC(C(=O)Nc2cc(Oc3ccc(F)cc3)cc(Oc3ccc(S(C)(=O)=O)cc3)c2)C1] | n/a | 654 ± 349 | n/a | n/a | n/a |
| TZ57- 122 | 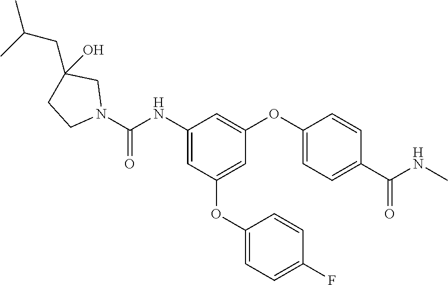 [SMILES: CNC(=O)c1ccc(Oc2cc(NC(=O)N3CCC(O)(CC(C)C)C3)cc(Oc3ccc(F)cc3)c2)cc1] | n/a | 77 ± 16 | n/a | n/a | n/a |
| TZ57- 150 | 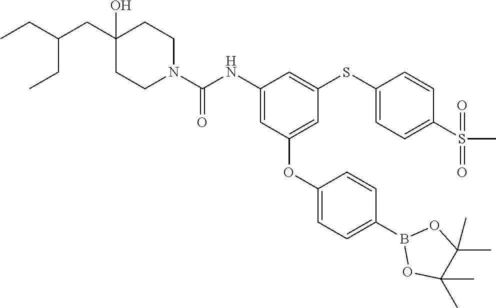 [SMILES: CCC(CC)CC1(O)CCN(C(=O)Nc2cc(Oc3ccc(B4OC(C)(C)C(C)(C)O4)cc3)cc(Sc3ccc(S(C)(=O)=O)cc3)c2)CC1] | n/a | >1000 | n/a | n/a | n/a |
| TZ57- 155 | 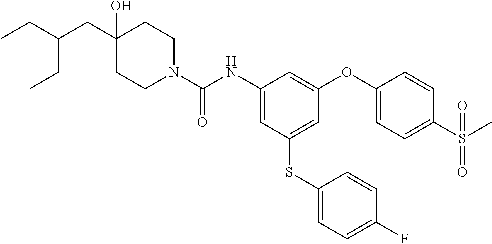 [SMILES: CCC(CC)CC1(O)CCN(C(=O)Nc2cc(Oc3ccc(S(C)(=O)=O)cc3)cc(Sc3ccc(F)cc3)c2)CC1] | n/a | 143 ± 30 | n/a | n/a | n/a |
| TZ57- 158 | 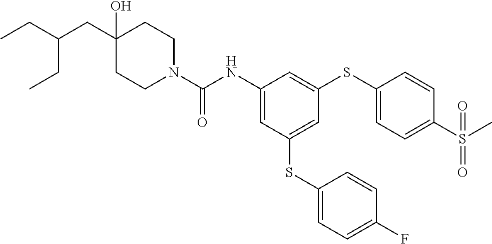 [SMILES: CCC(CC)CC1(O)CCN(C(=O)Nc2cc(Sc3ccc(F)cc3)cc(Sc3ccc(S(C)(=O)=O)cc3)c2)CC1] | n/a | 67 ± 10 | n/a | n/a | n/a |
| TZ59- 008 | 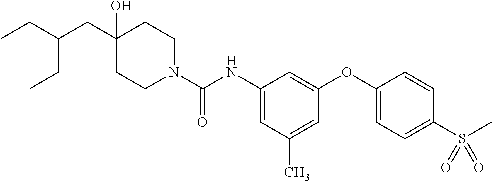 [SMILES: CCC(CC)CC1(O)CCN(C(=O)Nc2cc(C)cc(Oc3ccc(S(C)(=O)=O)cc3)c2)CC1] | n/a | >1000 | n/a | n/a | n/a |
| TZ59- 014 | 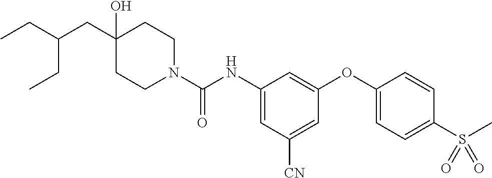 [SMILES: CCC(CC)CC1(O)CCN(C(=O)Nc2cc(C#N)cc(Oc3ccc(S(C)(=O)=O)cc3)c2)CC1] | n/a | >1000 | n/a | n/a | n/a |
| TZ59- 023 | 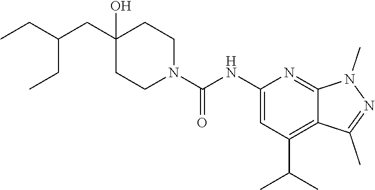 [SMILES: CCC(CC)CC1(O)CCN(C(=O)Nc2cc(C(C)C)c3c(C)nn(C)c3n2)CC1] | n/a | >1000 | n/a | n/a | n/a |
| TZ59- 039 | 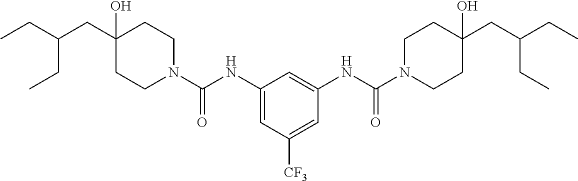 [SMILES: CCC(CC)CC1(O)CCN(C(=O)Nc2cc(NC(=O)N3CCC(O)(CC(CC)CC)CC3)cc(C(F)(F)F)c2)CC1] | n/a | >1000 | n/a | n/a | n/a |
| TZ59- 042 | 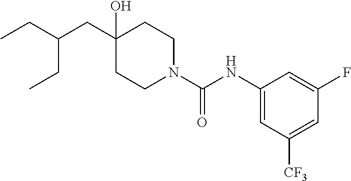 [SMILES: CCC(CC)CC1(O)CCN(C(=O)Nc2cc(F)cc(C(F)(F)F)c2)CC1] | n/a | >1000 | n/a | n/a | n/a |
| TZ59- 044 | 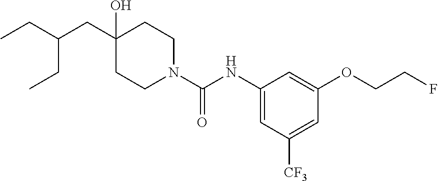 [SMILES: CCC(CC)CC1(O)CCN(C(=O)Nc2cc(OCCF)cc(C(F)(F)F)c2)CC1] | n/a | >1000 | n/a | n/a | n/a |
| TZ59- 046 | 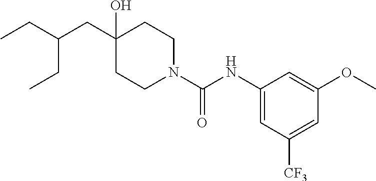 [SMILES: CCC(CC)CC1(O)CCN(C(=O)Nc2cc(OC)cc(C(F)(F)F)c2)CC1] | n/a | >1000 | n/a | n/a | n/a |
| TZ59- 061 | 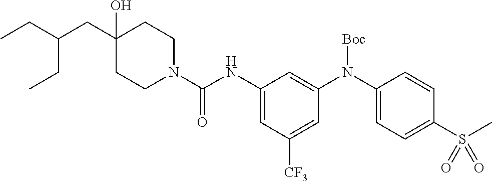 [SMILES: CCC(CC)CC1(O)CCN(C(=O)Nc2cc(N(C(=O)OC(C)(C)C)c3ccc(S(C)(=O)=O)cc3)cc(C(F)(F)F)c2)CC1] | n/a | >1000 | n/a | n/a | n/a |
| TZ59- 062 | 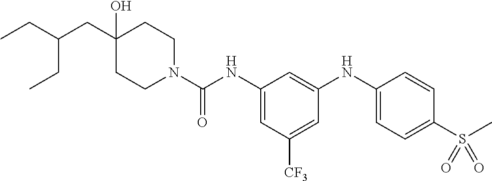 [SMILES: CCC(CC)CC1(O)CCN(C(=O)Nc2cc(Nc3ccc(S(C)(=O)=O)cc3)cc(C(F)(F)F)c2)CC1] | n/a | >1000 | n/a | n/a | n/a |
| TZ59- 080 | 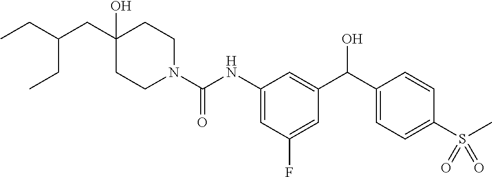 [SMILES: CCC(CC)CC1(O)CCN(C(=O)Nc2cc(F)cc(C(O)c3ccc(S(C)(=O)=O)cc3)c2)CC1] | n/a | >1000 | n/a | n/a | n/a |
| TZ59- 089 | 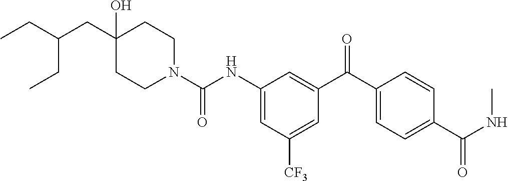 [SMILES: CCC(CC)CC1(O)CCN(C(=O)Nc2cc(C(=O)c3ccc(C(=O)NC)cc3)cc(C(F)(F)F)c2)CC1] | n/a | >1000 | n/a | n/a | n/a |
| TZ59- 092 | 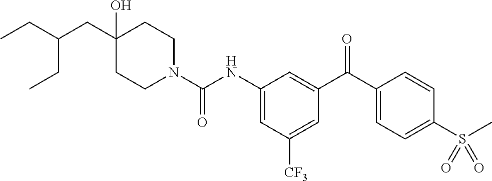 [SMILES: CCC(CC)CC1(O)CCN(C(=O)Nc2cc(C(=O)c3ccc(S(C)(=O)=O)cc3)cc(C(F)(F)F)c2)CC1] | n/a | 700 ± 111 | n/a | n/a | n/a |
| TZ59- 093 | 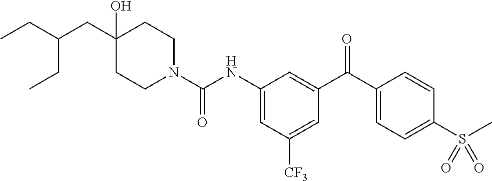 [SMILES: CCC(CC)CC1(O)CCN(C(=O)Nc2cc(C(=O)c3ccc(S(C)(=O)=O)cc3)cc(C(F)(F)F)c2)CC1] | n/a | >1000 | n/a | n/a | n/a |
| TZ59- 104 | 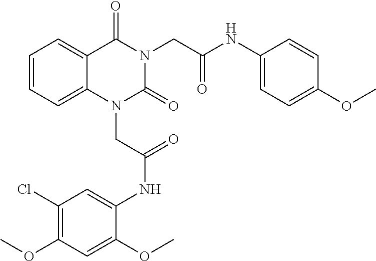 | >1000 | 5.7 ± 0.5 | >1000 | n/a | n/a |
| TZ59- 110 | 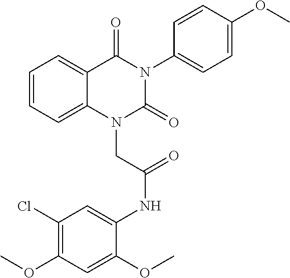 | n/a | >1000 | n/a | n/a | n/a |
| TZ59- 112 | 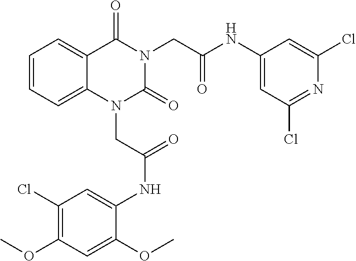 [SMILES: COc1cc(OC)c(NC(=O)Cn2c(=O)n(CC(=O)Nc3cc(Cl)nc(Cl)c3)c(=O)c3ccccc32)cc1Cl] | n/a | >1000 | n/a | n/a | n/a |
| TZ59- 122 | 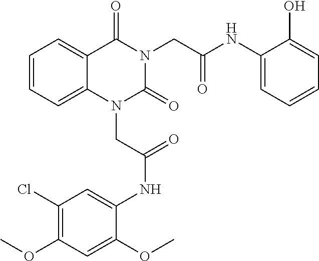 [SMILES: COc1cc(OC)c(NC(=O)Cn2c(=O)n(CC(=O)Nc3ccccc3O)c(=O)c3ccccc32)cc1Cl] | n/a | 4.8 ± 0.5 | n/a | n/a | n/a |
| TZ59- 124 | 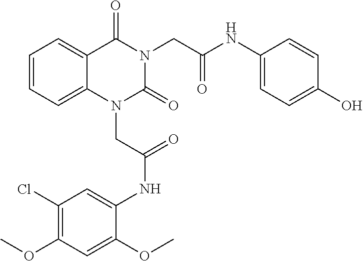 [SMILES: COc1cc(OC)c(NC(=O)Cn2c(=O)n(CC(=O)Nc3ccc(O)cc3)c(=O)c3ccccc32)cc1Cl] | n/a | 2.6 ± 0.3 | n/a | n/a | n/a |
| TZ59- 130 | 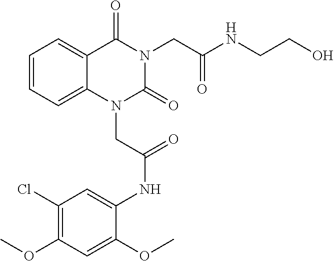 [SMILES: COc1cc(OC)c(NC(=O)Cn2c(=O)n(CC(=O)NCCO)c(=O)c3ccccc32)cc1Cl] | n/a | 66.6 ± 10.3 | n/a | n/a | n/a |
| TZ59- 131 | 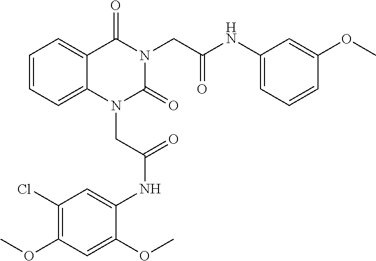 | n/a | 39.1 ± 7.0 | n/a | n/a | n/a |
| TZ59- 132 | 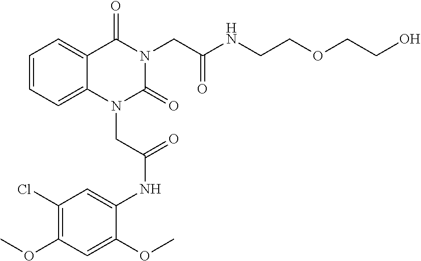 [SMILES: COc1cc(OC)c(NC(=O)Cn2c(=O)n(CC(=O)NCCOCCO)c(=O)c3ccccc32)cc1Cl] | n/a | 91.4 ± 17.6 | n/a | n/a | n/a |
| TZ59- 133 | 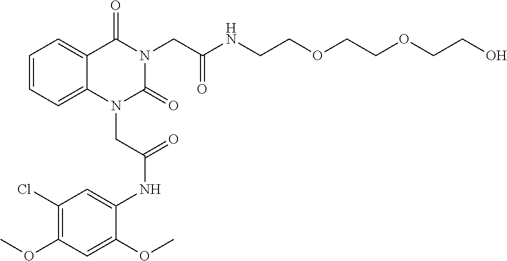 [SMILES: COc1cc(OC)c(NC(=O)Cn2c(=O)n(CC(=O)NCCOCCOCCO)c(=O)c3ccccc32)cc1Cl] | n/a | 204 ± 96 | n/a | n/a | n/a |
| TZ59- 134 | 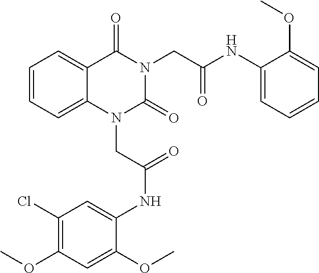 [SMILES: COc1cc(OC)c(NC(=O)Cn2c(=O)n(CC(=O)Nc3ccccc3OC)c(=O)c3ccccc32)cc1Cl] | >1000 | 6.3 ± 0.9 | >1000 | >1000 | >1000 |
| TZ59- 135 | 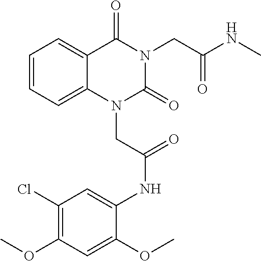 | n/a | 376 ± 91 | n/a | n/a | n/a |
| TZ59- 136 | 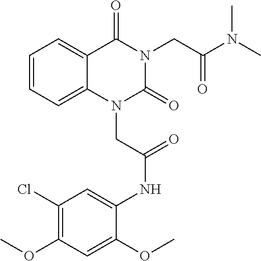 | n/a | 175 ± 13 | n/a | n/a | n/a |
| TZ62- 004 | 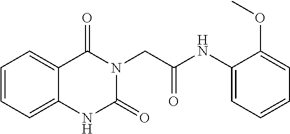 [SMILES: COc1ccccc1NC(=O)Cn1c(=O)[nH]c2ccccc2c1=O] | n/a | >1000 | n/a | n/a | n/a |
| TZ62- 007 | 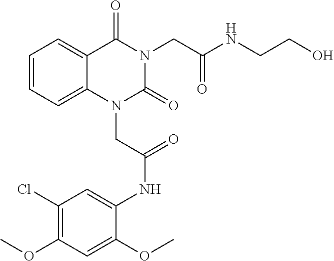 [SMILES: COc1cc(OC)c(NC(=O)Cn2c(=O)n(CC(=O)NCCO)c(=O)c3ccccc32)cc1Cl] | n/a | >1000 | n/a | n/a | n/a |
| TZ62- 008 | 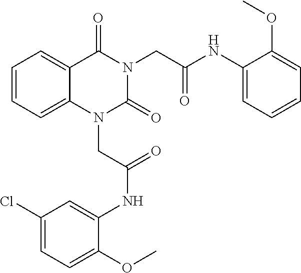 [SMILES: COc1ccccc1NC(=O)Cn1c(=O)c2ccccc2n(CC(=O)Nc2cc(Cl)ccc2OC)c1=O] | n/a | 54.9 ± 6.0 | n/a | n/a | n/a |
| TZ62- 014 | 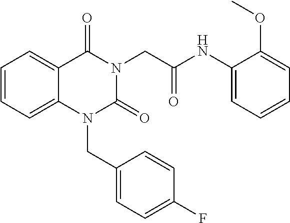 [SMILES: COc1ccccc1NC(=O)Cn1c(=O)c2ccccc2n(Cc2ccc(F)cc2)c1=O] | n/a | >1000 | n/a | n/a | n/a |
| TZ62- 016 | 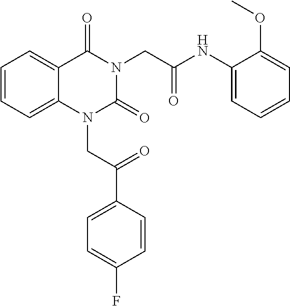 [SMILES: COc1ccccc1NC(=O)Cn1c(=O)c2ccccc2n(CC(=O)c2ccc(F)cc2)c1=O] | n/a | >1000 | n/a | n/a | n/a |
| TZ62- 017 | 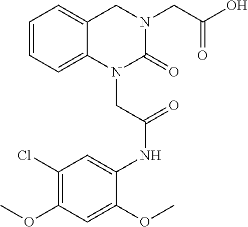 [SMILES: COc1cc(OC)c(NC(=O)CN2C(=O)N(CC(=O)O)Cc3ccccc32)cc1Cl] | n/a | >1000 | n/a | n/a | n/a |
| TZ62- 018 | 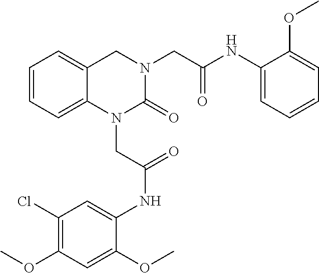 | n/a | >1000 | n/a | n/a | n/a |
| TZ62- 019 | 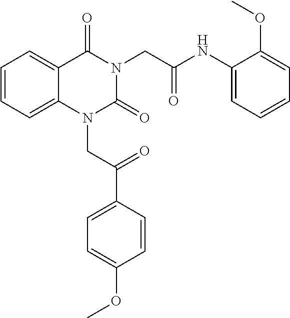 [SMILES: COc1ccc(C(=O)Cn2c(=O)n(CC(=O)Nc3ccccc3OC)c(=O)c3ccccc32)cc1] | n/a | >1000 | n/a | n/a | n/a |
| TZ62- 020 | 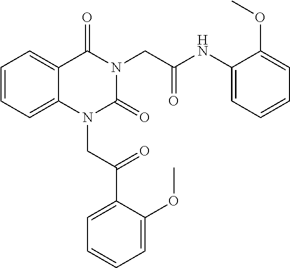 [SMILES: COc1ccccc1NC(=O)Cn1c(=O)c2ccccc2n(CC(=O)c2ccccc2OC)c1=O] | n/a | >1000 | n/a | n/a | n/a |
| TZ62- 026 | 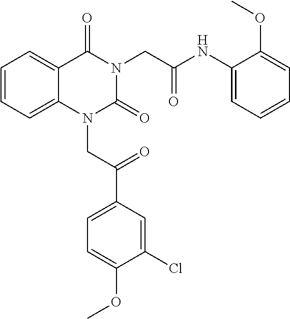 [SMILES: COc1ccc(C(=O)Cn2c(=O)n(CC(=O)Nc3ccccc3OC)c(=O)c3ccccc32)cc1Cl] | n/a | 287 ± 69 | n/a | n/a | n/a |
| TZ62- 038 | 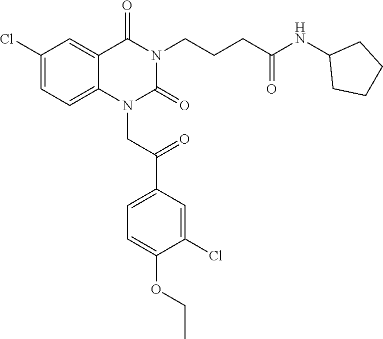 [SMILES: CCOc1ccc(C(=O)Cn2c(=O)n(CCCC(=O)NC3CCCC3)c(=O)c3cc(Cl)ccc32)cc1Cl] | n/a | 293 ± 69 | n/a | n/a | n/a |
| TZ62- 039 | 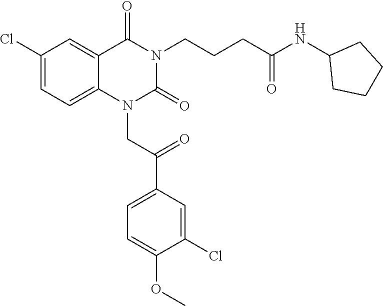 [SMILES: COc1ccc(C(=O)Cn2c(=O)n(CCCC(=O)NC3CCCC3)c(=O)c3cc(Cl)ccc32)cc1Cl] | n/a | >1000 | n/a | n/a | n/a |
| TZ62- 048 | 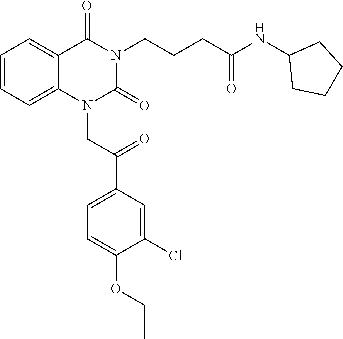 [SMILES: CCOc1ccc(C(=O)Cn2c(=O)n(CCCC(=O)NC3CCCC3)c(=O)c3ccccc32)cc1Cl] | n/a | 85 ± 10 | n/a | n/a | n/a |
| TZ62- 049 | 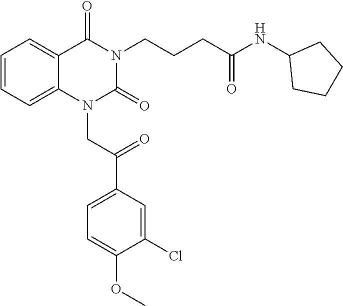 [SMILES: COc1ccc(C(=O)Cn2c(=O)n(CCCC(=O)NC3CCCC3)c(=O)c3ccccc32)cc1Cl] | n/a | 396 ± 78 | n/a | n/a | n/a |
| TZ62- 050 | 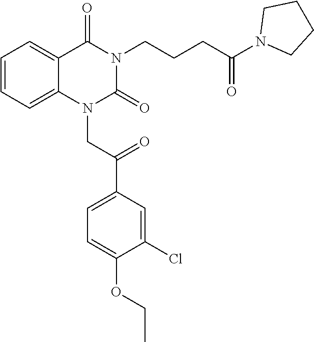 [SMILES: CCOc1ccc(C(=O)Cn2c(=O)n(CCCC(=O)N3CCCC3)c(=O)c3ccccc32)cc1Cl] | n/a | >1000 | n/a | n/a | n/a |
| TZ62- 051 | 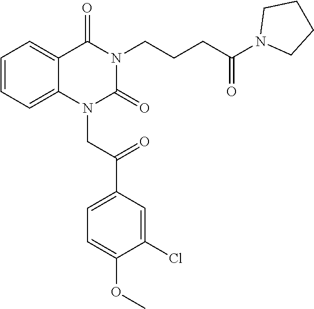 [SMILES: COc1ccc(C(=O)Cn2c(=O)n(CCCC(=O)N3CCCC3)c(=O)c3ccccc32)cc1Cl] | n/a | >1000 | n/a | n/a | n/a |
| TZ62- 052 | 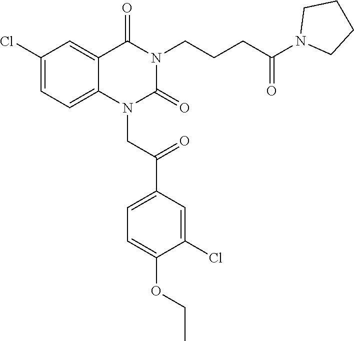 [SMILES: CCOc1ccc(C(=O)Cn2c(=O)n(CCCC(=O)N3CCCC3)c(=O)c3cc(Cl)ccc32)cc1Cl] | n/a | >1000 | n/a | n/a | n/a |
| TZ62- 053 | 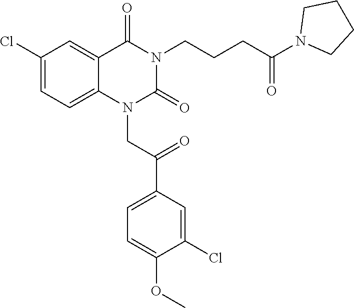 [SMILES: COc1ccc(C(=O)Cn2c(=O)n(CCCC(=O)N3CCCC3)c(=O)c3cc(Cl)ccc32)cc1Cl] | n/a | >1000 | n/a | n/a | n/a |
| TZ62- 077 | 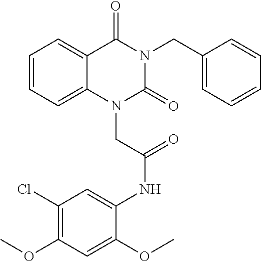 [SMILES: COc1cc(OC)c(NC(=O)Cn2c(=O)n(Cc3ccccc3)c(=O)c3ccccc32)cc1Cl] | n/a | >1000 | n/a | n/a | n/a |
| TZ62- 078 | 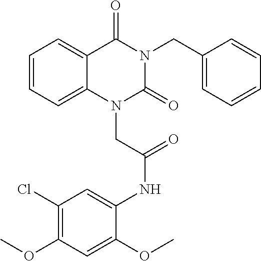 [SMILES: COc1cc(OC)c(NC(=O)Cn2c(=O)n(Cc3ccccc3)c(=O)c3ccccc32)cc1Cl] | n/a | >1000 | n/a | n/a | n/a |
| TZ62- 082 | 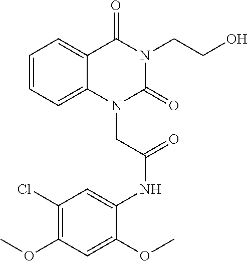 [SMILES: COc1cc(OC)c(NC(=O)Cn2c(=O)n(CCO)c(=O)c3ccccc32)cc1Cl] | n/a | >1000 | n/a | n/a | n/a |
| TZ62- 083 | 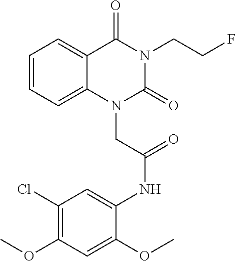 [SMILES: COc1cc(OC)c(NC(=O)Cn2c(=O)n(CCF)c(=O)c3ccccc32)cc1Cl] | n/a | >1000 | n/a | n/a | n/a |
| TZ62- 084 | 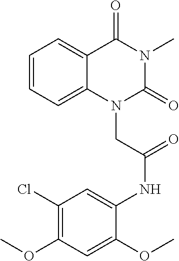 | n/a | >1000 | n/a | n/a | n/a |
| TZ62- 087 | 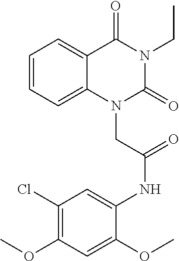 [SMILES: CCn1c(=O)c2ccccc2n(CC(=O)Nc2cc(Cl)c(OC)cc2OC)c1=O] | n/a | >1000 | n/a | n/a | n/a |
| TZ62- 090 | 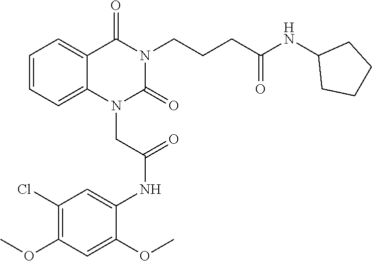 [SMILES: COc1cc(OC)c(NC(=O)Cn2c(=O)n(CCCC(=O)NC3CCCC3)c(=O)c3ccccc32)cc1Cl] | n/a | 76 ± 19 | n/a | n/a | n/a |
| TZ62- 091 | 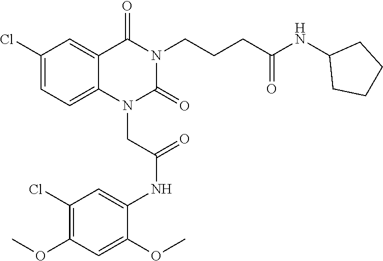 [SMILES: COc1cc(OC)c(NC(=O)Cn2c(=O)n(CCCC(=O)NC3CCCC3)c(=O)c3cc(Cl)ccc32)cc1Cl] | n/a | >1000 | n/a | n/a | n/a |
| TZ62- 092 | 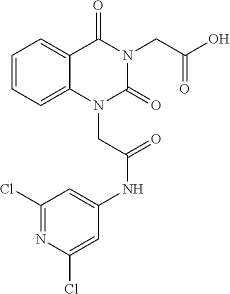 [SMILES: O=C(O)Cn1c(=O)c2ccccc2n(CC(=O)Nc2cc(Cl)nc(Cl)c2)c1=O] | n/a | >1000 | n/a | n/a | n/a |
| TZ62- 093 | 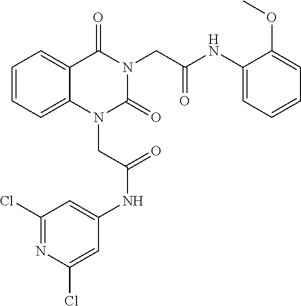 [SMILES: COc1ccccc1NC(=O)Cn1c(=O)c2ccccc2n(CC(=O)Nc2cc(Cl)nc(Cl)c2)c1=O] | n/a | >1000 | n/a | n/a | n/a |
| TZ62- 097 | 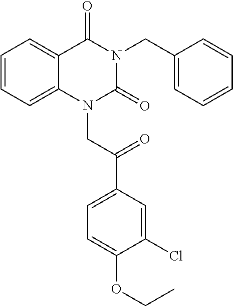 [SMILES: CCOc1ccc(C(=O)Cn2c(=O)n(Cc3ccccc3)c(=O)c3ccccc32)cc1Cl] | n/a | >1000 | n/a | n/a | n/a |
| TZ62- 098 | 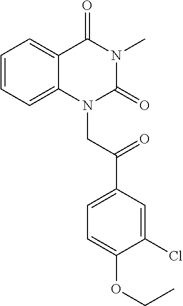 [SMILES: CCOc1ccc(C(=O)Cn2c(=O)n(C)c(=O)c3ccccc32)cc1Cl] | n/a | >1000 | n/a | n/a | n/a |
| TZ62- 099 | 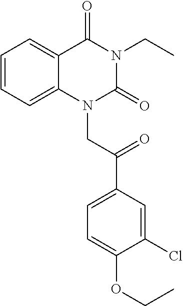 [SMILES: CCOc1ccc(C(=O)Cn2c(=O)n(CC)c(=O)c3ccccc32)cc1Cl] | n/a | >1000 | n/a | n/a | n/a |
| TZ62- 100 | 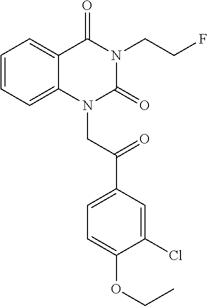 [SMILES: CCOc1ccc(C(=O)Cn2c(=O)n(CCF)c(=O)c3ccccc32)cc1Cl] | n/a | >1000 | n/a | n/a | n/a |
| TZ62- 102 | 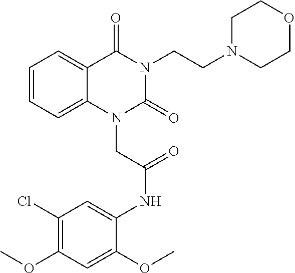 [SMILES: COc1cc(OC)c(NC(=O)Cn2c(=O)n(CCN3CCOCC3)c(=O)c3ccccc32)cc1Cl] | n/a | >1000 | n/a | n/a | n/a |
| TZ62- 114 | 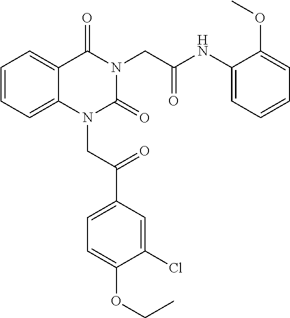 [SMILES: CCOc1ccc(C(=O)Cn2c(=O)n(CC(=O)Nc3ccccc3OC)c(=O)c3ccccc32)cc1Cl] | n/a | 49.8 ± 4.9 | n/a | n/a | n/a |
| TZ62- 120 | 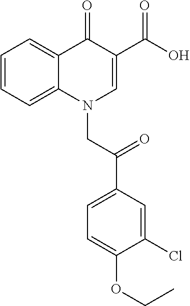 [SMILES: CCOc1ccc(C(=O)Cn2cc(C(=O)O)c(=O)c3ccccc32)cc1Cl] | n/a | 270 ± 210 | n/a | n/a | n/a |
| TZ62- 122 | 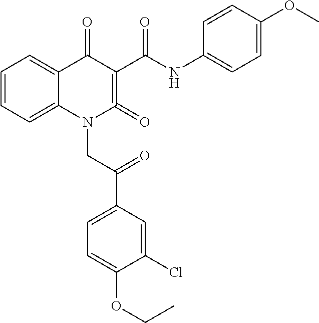 | n/a | >1000 | n/a | n/a | n/a |
| TZ62- 125 | 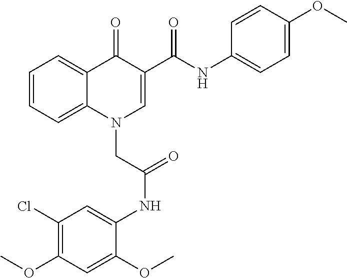 | n/a | >1000 | n/a | n/a | n/a |
| TZ65- 032 | 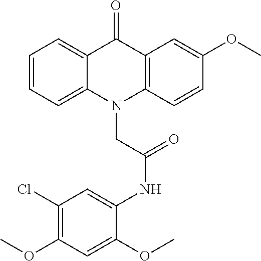 | n/a | >1000 | n/a | n/a | n/a |
| TZ65- 033 | 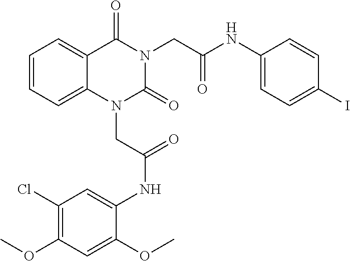 [SMILES: COc1cc(OC)c(NC(=O)Cn2c(=O)n(CC(=O)Nc3ccc(I)cc3)c(=O)c3ccccc32)cc1Cl] | n/a | 16.9 ± 5.6 | n/a | n/a | n/a |
| TZ65- 044 | 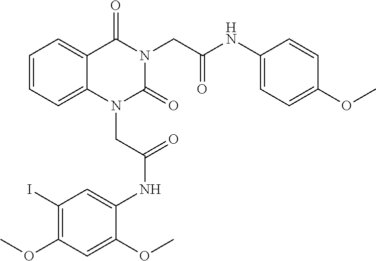 | n/a | 2.8 ± 0.5 | n/a | n/a | n/a |
| TZ65- 049 | 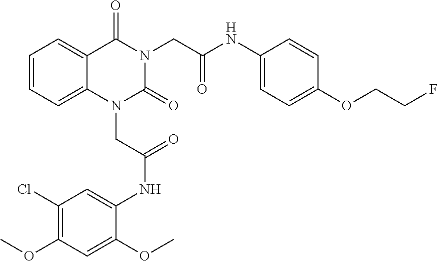 [SMILES: COc1cc(OC)c(NC(=O)Cn2c(=O)n(CC(=O)Nc3ccc(OCCF)cc3)c(=O)c3ccccc32)cc1Cl] | n/a | 10 ± 2.4 | n/a | n/a | n/a |

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compounds, compositions and methods without departing from the scope of the invention, it is intended that all matter contained in the above description and shown in the accompanying drawings shall be interpreted as illustrative and not in a limiting sense.

1.-66. (canceled)

67. A compound of Formula I, or a salt thereof: 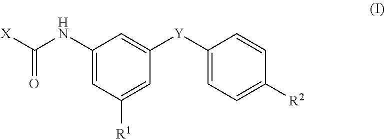
wherein
R¹ is hydrogen, substituted or unsubstituted alkyl, halo, substituted or unsubstituted alkoxy, cyano, substituted or unsubstituted nitrogen containing aryl, substituted or unsubstituted nitrogen containing cycloalkyl, or Y—R³;
R² is —CON(R⁴)₂, halo, —SO₂R⁴, substituted or unsubstituted alkoxy, or —COOH;
R³ is substituted or unsubstituted aryl, substituted or unsubstituted nitrogen containing aryl, or substituted or unsubstituted nitrogen containing cycloalkyl;
Y is —O—, —S—, —N(R⁴)—, —C(O)—, —CH(OH)—;
R⁴ is hydrogen, tert-butyloxycarbonyl (BOC), or a C1 to C6 alkyl;
X is: 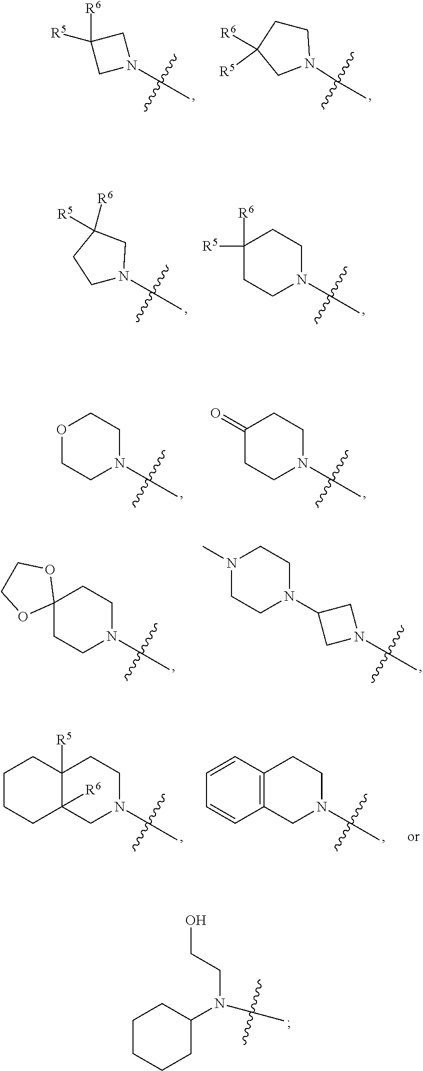
and
R⁵ and R⁶ are each independently hydrogen, hydroxyl, substituted or unsubstituted alkyl, substituted or unsubstituted alkoxyl, or substituted or unsubstituted nitrogen-containing heterocycloalkyl.

68. The compound of claim 66, wherein R¹ is Y—R³, halo, C₁ to C₃ alkyl, haloalkyl, haloalkoxy, cyano,

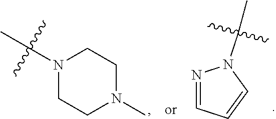

69. The compound of claim 66, wherein R¹ is, —CF₃, F, methyl, —OCH2CH2CH2F, —OCH2CH2F, —CN, 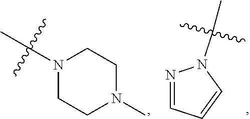
or Y—R³.

70. The compound of claim 66, wherein R³ is selected from the group consisting of: 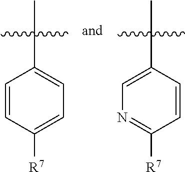
wherein R⁷ is halo, —SO₂R⁴, 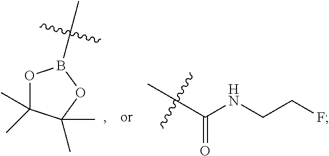
and R⁴ is hydrogen or a C1-C6 alkyl.

71. The compound of claim 66, wherein R³ is selected from the group consisting of

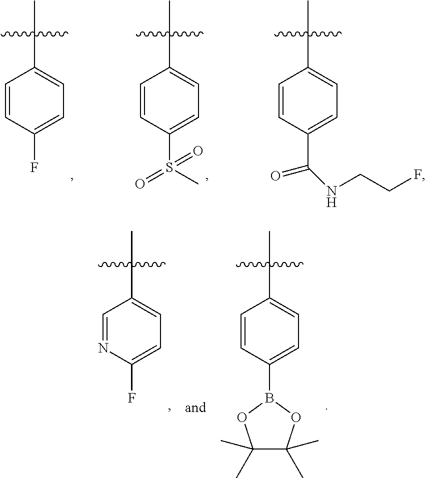

72. The compound of claim 66, wherein Y is —O—, —S—, —C(O)—, —C(OH)—, —N(H)— or —N(BOC)—.

73. The compound of claim 66, wherein R² is —CON(R⁴)₂, F, —SO₂CH₃, alkoxy, haloalkoxy, or —COOH.

74. The compound of claim 66, wherein R² is —C(O)NH₂, —C(O)NHCH₃, —C(O)N(CH₃)₂, F, —SO₂CH₃, —OCH₃, or —OCH₂CH₂F.

75. The compound of claim 66, wherein R⁵ and R⁶ are each independently hydrogen, substituted or unsubstituted C1 to C6 alkyl, hydroxyl, or alkoxy.

76. The compound of claim 66, wherein R⁵ and R⁶ are each independently hydrogen, —OH, 2-(ethyl)butyl, isobutyl, isopropyl, methyl, ethyl, propyl, butyl, 4-methylpentan-2-onyl, or C1 to C6 hydroxyalkyl.

77. The compound of claim 66, wherein X is selected from the group consisting of:

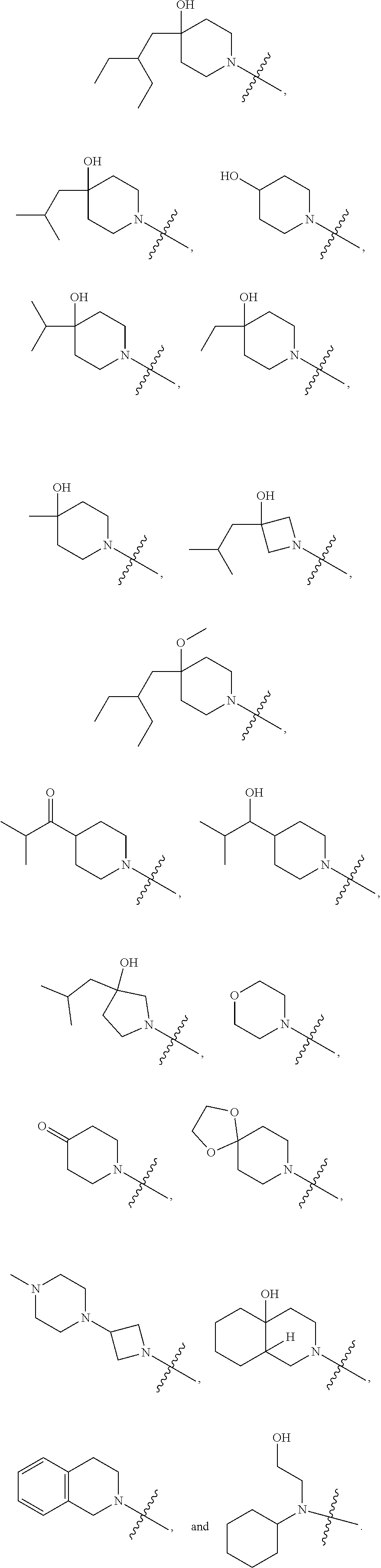

78. The compound of claim 66, wherein the compound is of Formula (II) or a salt thereof: 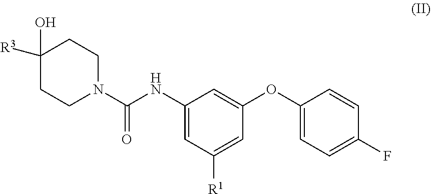
wherein
R¹ is —CF₃, 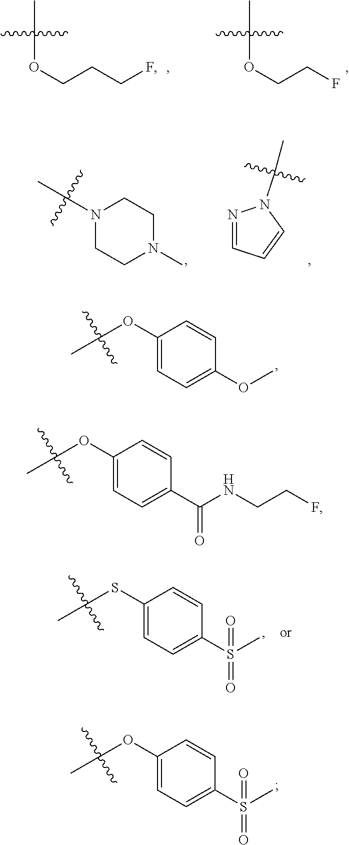
and
R³ is 2-(ethyl)butyl, isobutyl, isopropyl, methyl, or ethyl.

79. The compound of claim 66, wherein the compound is selected from the group consisting of:

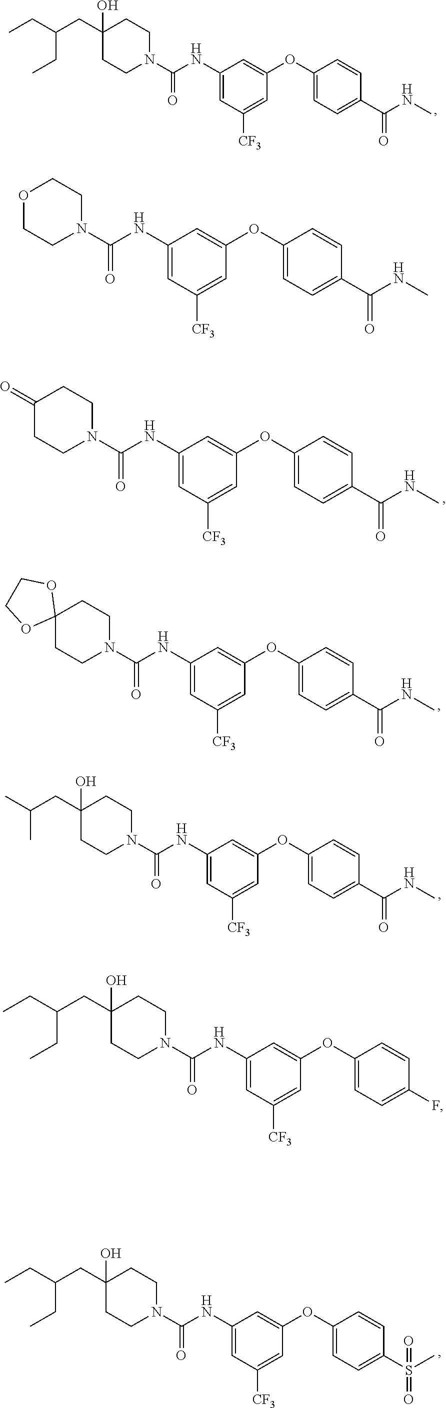

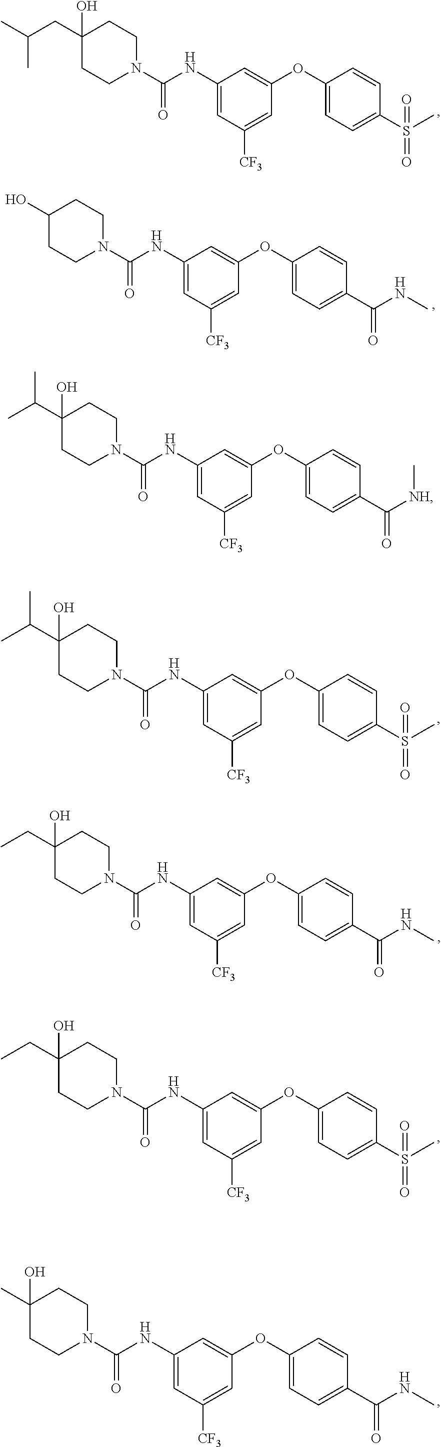

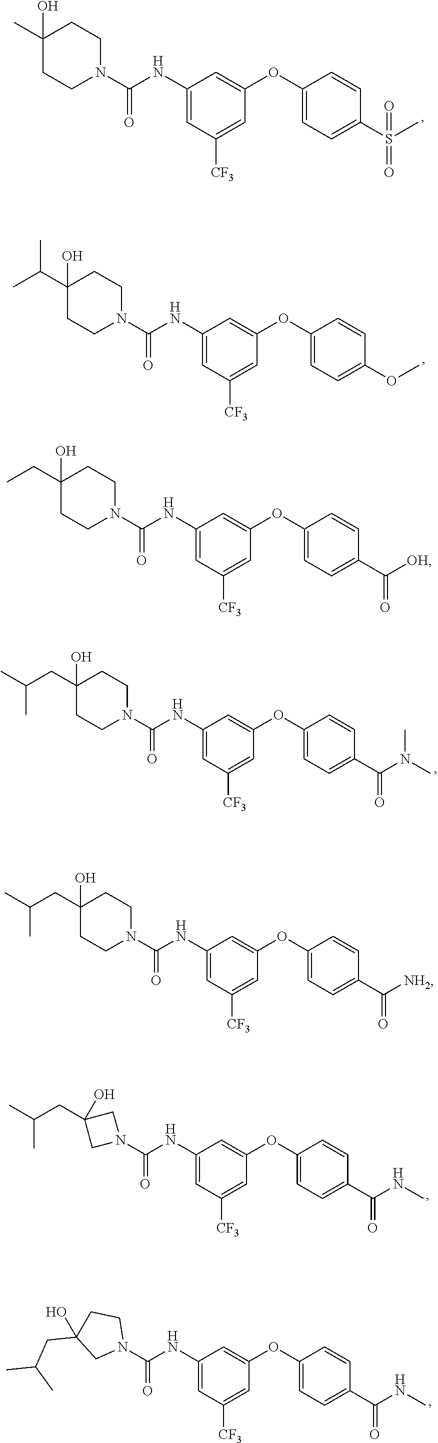

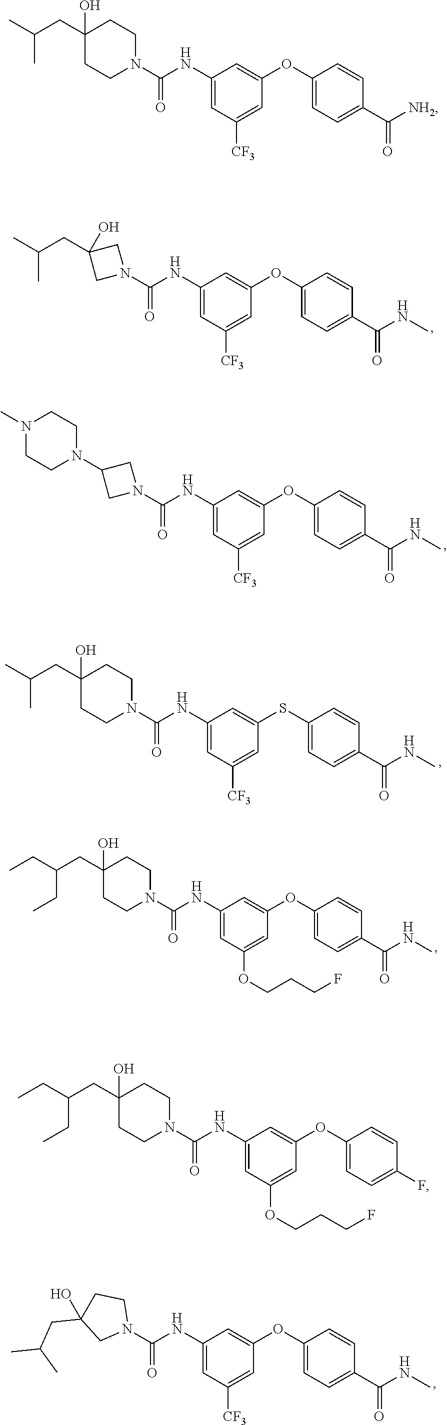

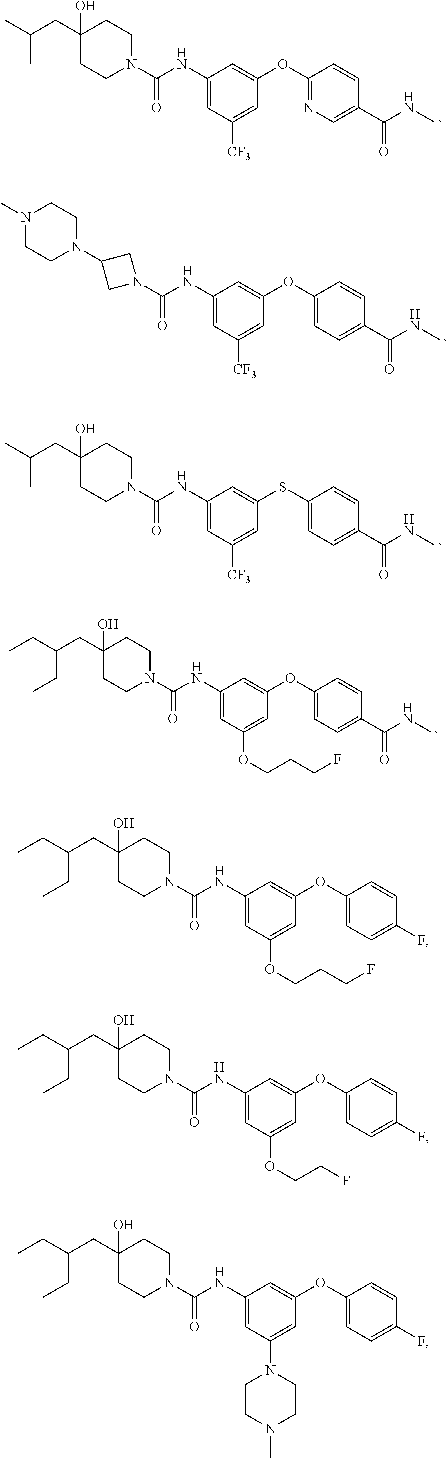

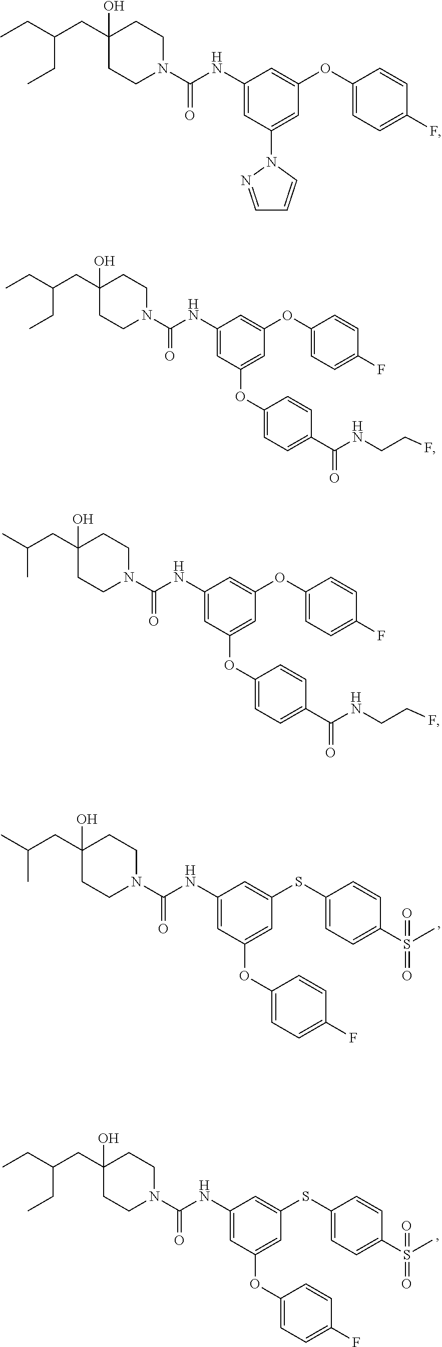

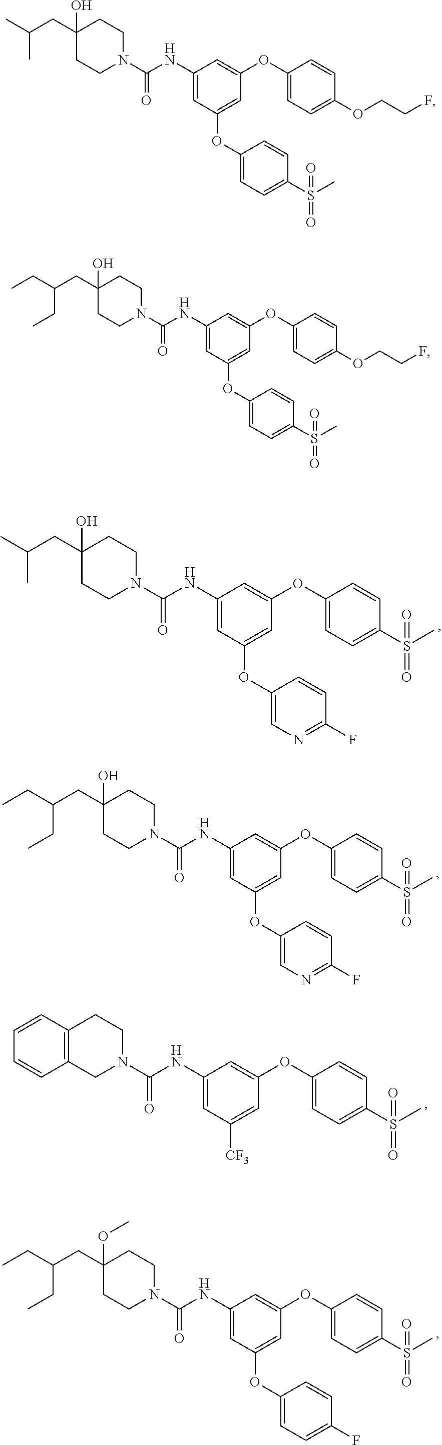

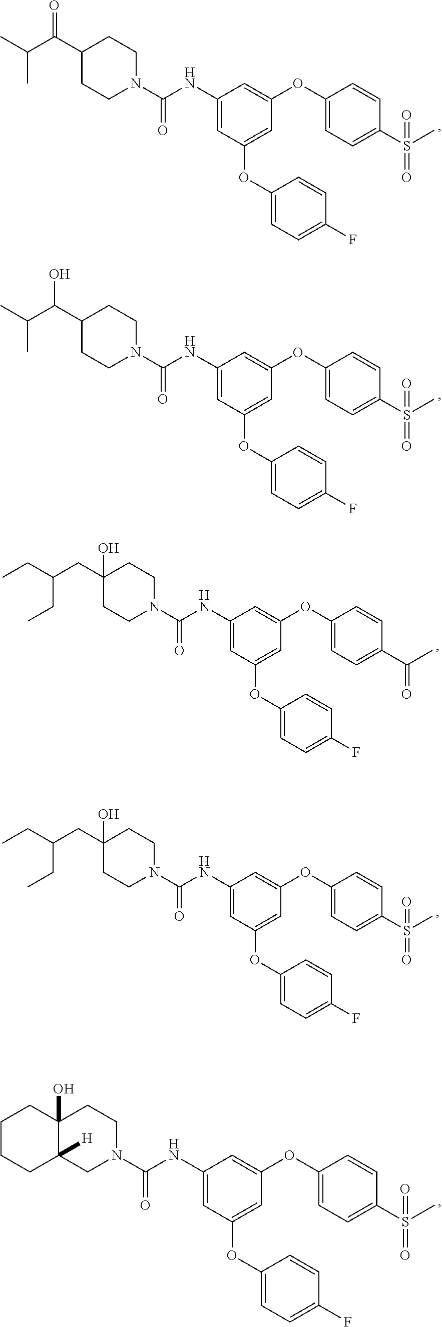

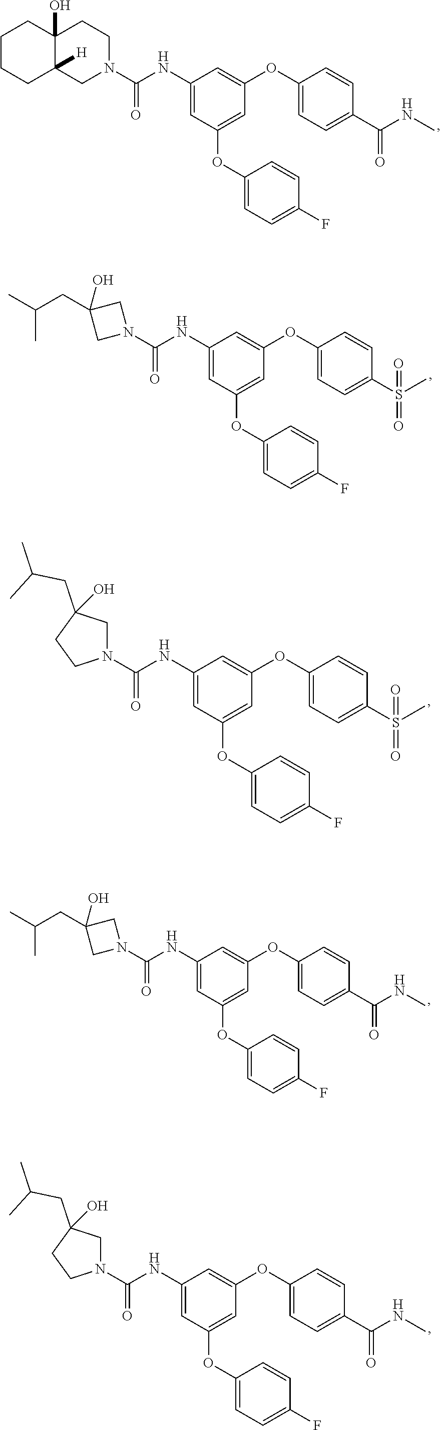

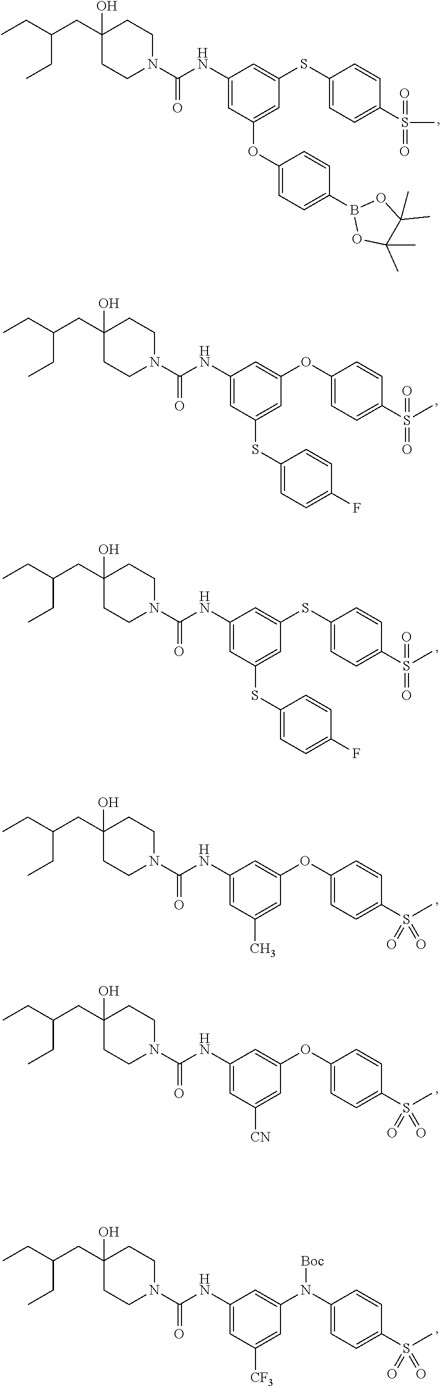

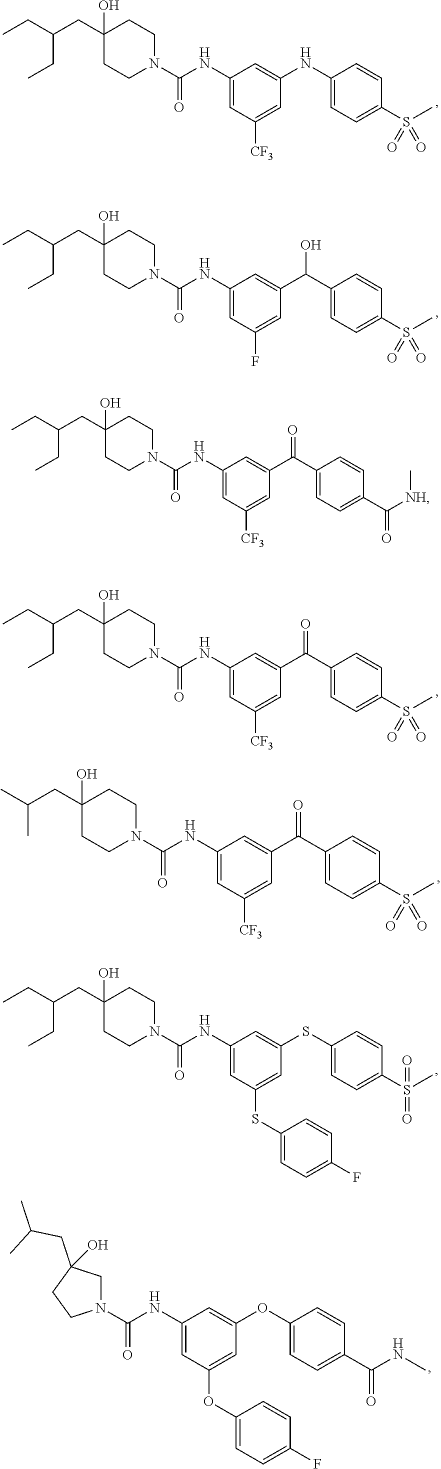

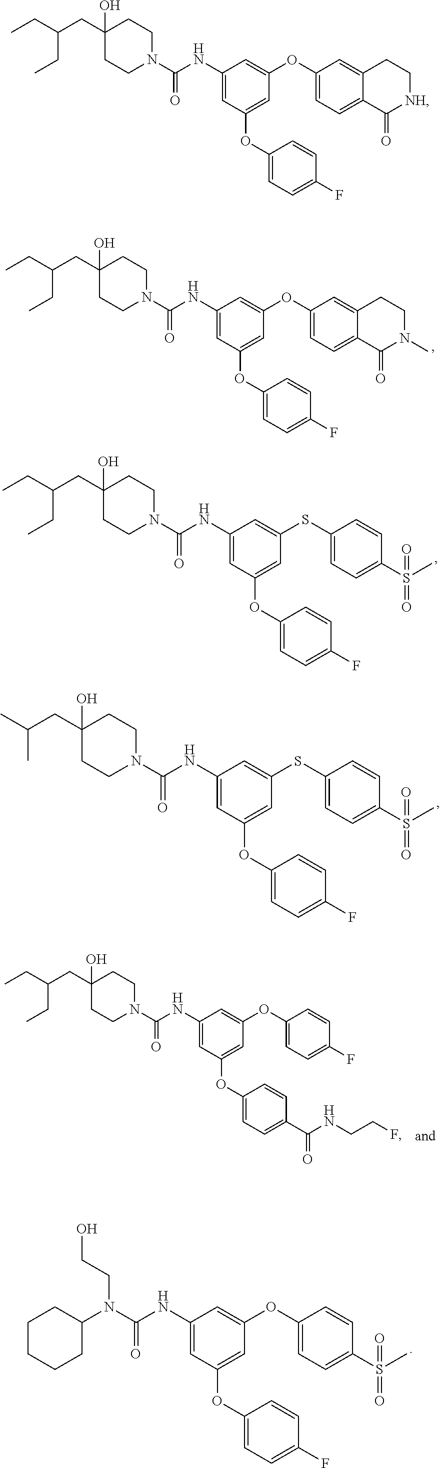

80. An imaging agent comprising a compound of claim 66 and a reporting group.

81. A pharmaceutical composition comprising a compound of claim 66 and at least one pharmaceutically acceptable excipient.

82. A method of treating anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and/or lung fibrosis in a subject in need thereof, the method comprising administering to the subject a therapeutically effective amount of a compound of claim 66.

83. A method of imaging a target in a subject, the method comprising:
administering the imaging agent of claim 80 and
detecting the imaging agent in the subject, wherein detecting the imaging agent comprises imaging with positron emission tomography (PET) imaging, single photon emission computed tomography (SPECT) imaging, mass spectrometry, gamma imaging, magnetic resonance imaging (MRI), magnetic resonance spectroscopy, fluorescence spectroscopy, CT, ultrasound, or X-ray.

84. A method of determining an efficacy of a therapeutic agent in a subject comprising:
administering a compound of claim 66 to the subject;
evaluating S1PR2 expression in the subject;
treating the subject for a disease associated with increased S1PR2 expression; and
reevaluating the S1PR2 expression in the subject.

85. A method of diagnosing or monitoring an S1PR2 associated disease, disorder or condition in a mammal comprising administering the imagining agent claim 80 to the mammal and detecting the imaging agent, wherein the S1PR2 associated disease disorder or condition is selected from the group consisting of anaphylaxis, cancer, central nervous system diseases, fibrosis diseases, diabetes, diabetic pulmonary fibrosis, diabetic nephropathy, inflammation, inflammation diseases, inflammatory response in multiple sclerosis, multiple sclerosis, liver fibrosis, and lung fibrosis.

86. A method of quantifying S1PR2 expression in a mammalian brain or central nervous system comprising administering the imaging agent of claim 80 to the mammal and detecting the imaging agent in the mammal.

87. A compound of Formula (III), or a salt thereof: 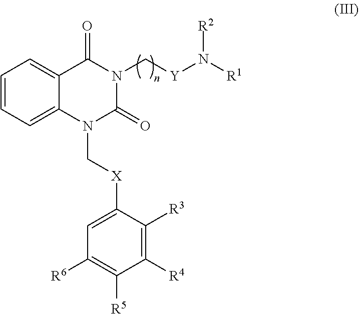
wherein
Y is —CH₂—, or —C(O)—;
R¹ and R² are each independently hydrogen, substituted or unsubstituted alkyl, substituted or unsubstituted alkoxy, substituted or unsubstituted cycloalkyl, substituted or unsubstituted heterocycloalkyl, substituted or unsubstituted aryl, substituted or unsubstituted heteroaryl, or are each alkyl and together form a 4 to 6 membered ring;
n is an integer between 1 and 10;
X is —C(O)— or —C(O)N(H)—; and
R³, R⁴, R⁵, and R⁶ are each independently hydrogen, halo, substituted or unsubstituted alkyl, or substituted or unsubstituted alkoxy.

88. A compound selected from the group consisting of: 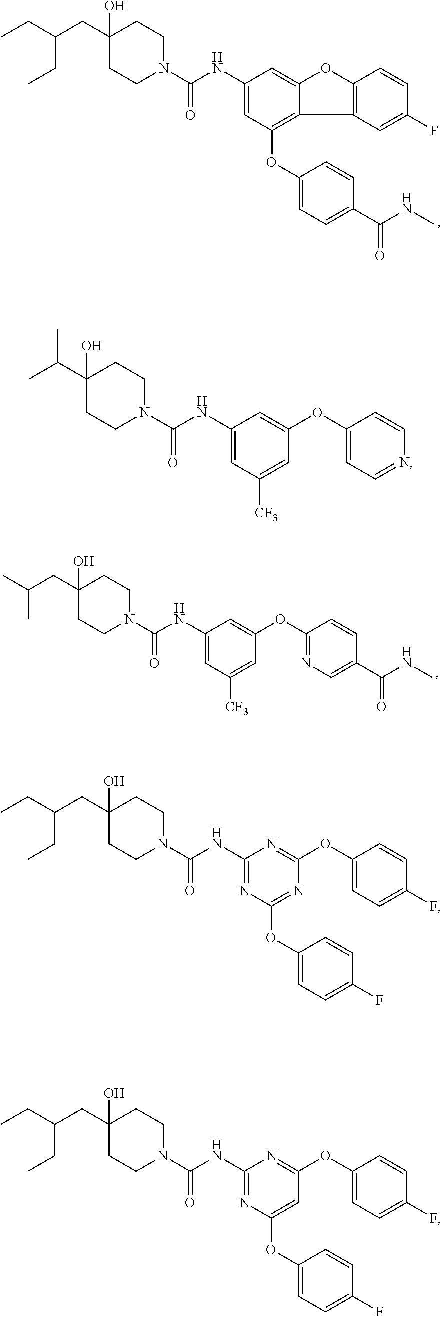 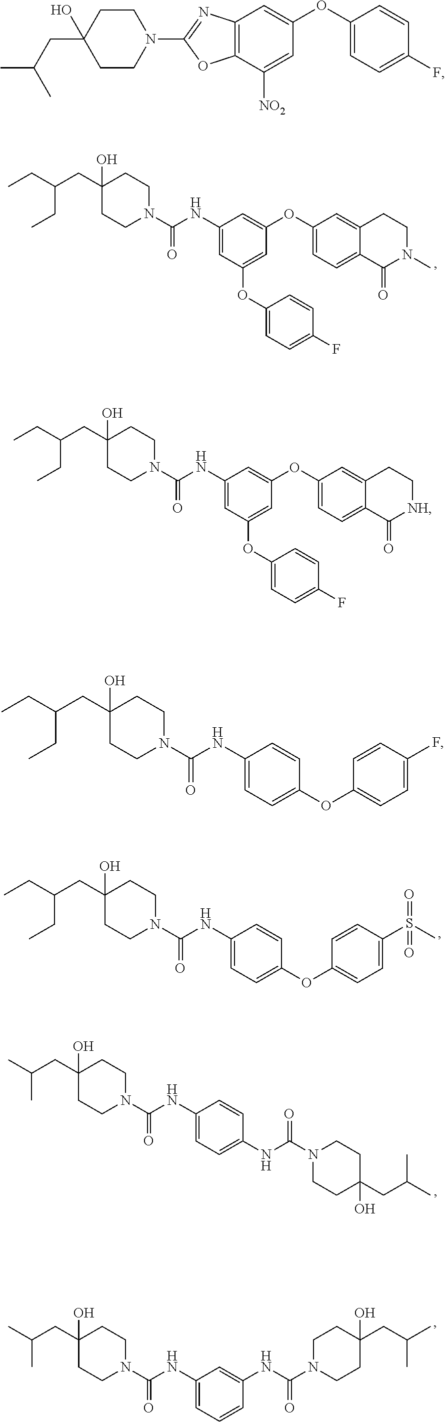 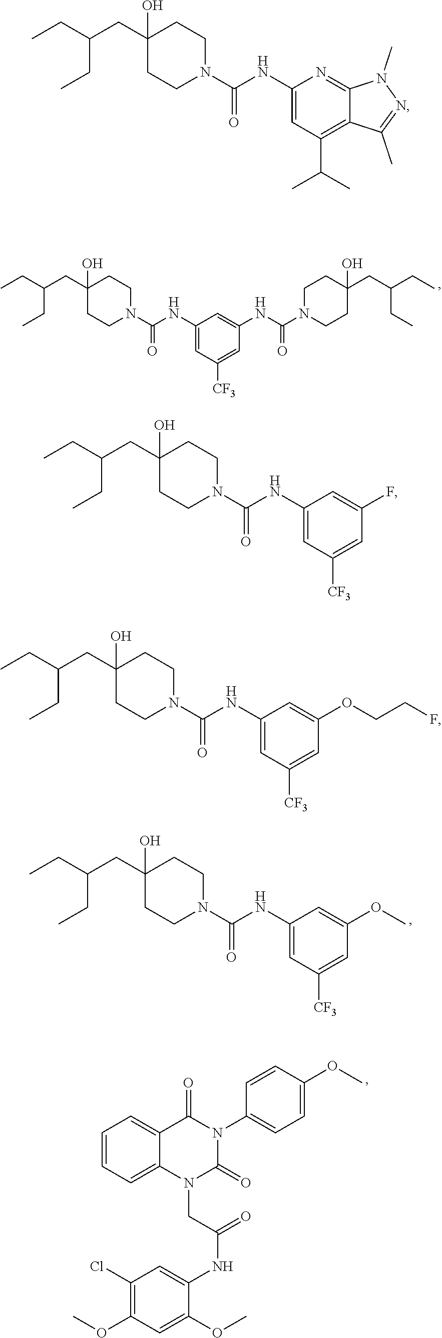 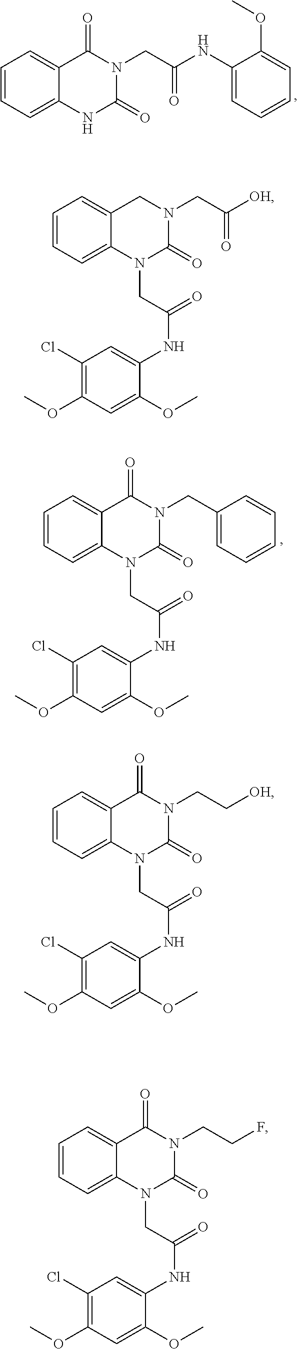 [SMILES: COc1ccccc1NC(=O)Cn1c(=O)[nH]c2ccccc2c1=O ; COc1cc(OC)c(NC(=O)CN2C(=O)N(CC(=O)O)Cc3ccccc32)cc1Cl ; COc1cc(OC)c(NC(=O)Cn2c(=O)n(Cc3ccccc3)c(=O)c3ccccc32)cc1Cl ; COc1cc(OC)c(NC(=O)Cn2c(=O)n(CCO)c(=O)c3ccccc32)cc1Cl ; COc1cc(OC)c(NC(=O)Cn2c(=O)n(CCF)c(=O)c3ccccc32)cc1Cl] 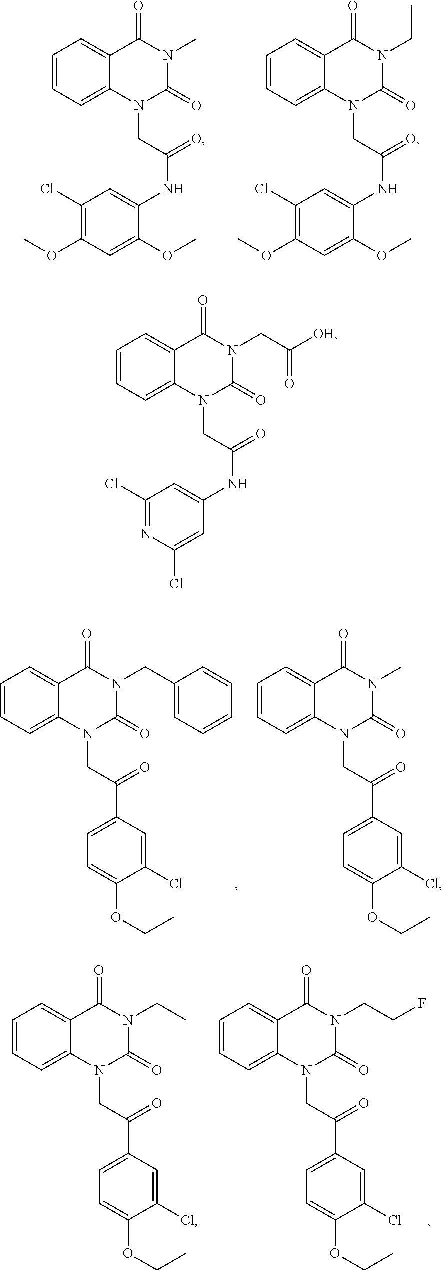 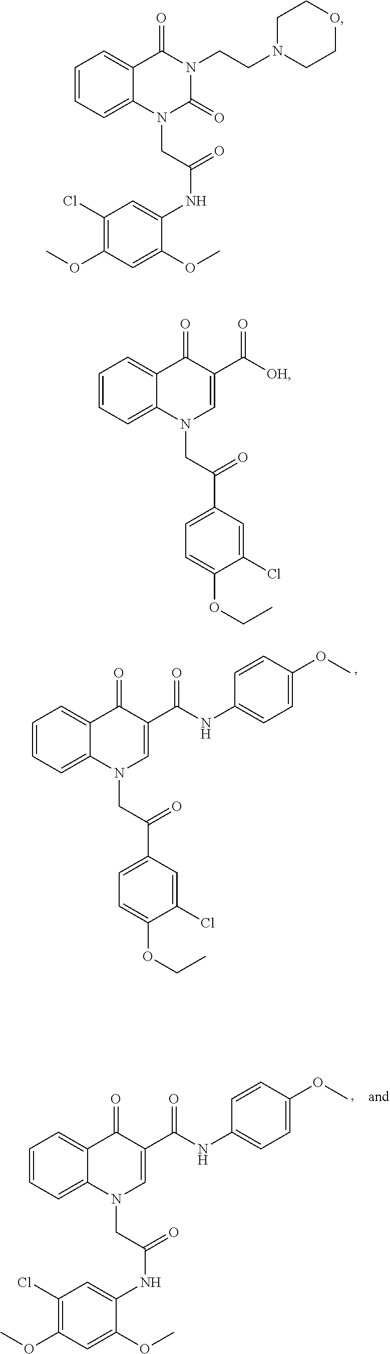 [chemical structure]
